(19) United States
(12) Patent Application Publication
(10) Pub. No.: US 2024/0350632 A1
(43) Pub. Date: Oct. 24, 2024

(54) ENGINEERED IMMUNE CELLS FOR TREATING DISORDERS, COMPOSITIONS AND METHODS THEREOF

(71) Applicant: iCell Gene Therapeutics Inc., Stony Brook, NY (US)

(72) Inventors: Yupo MA, Stony Brook, NY (US); Kevin PINZ, Stony Brook, NY (US); Masayuki WADA, Stony Brook, NY (US)

(21) Appl. No.: 18/520,972

(22) Filed: Nov. 28, 2023

Related U.S. Application Data
(60) Provisional application No. 63/428,259, filed on Nov. 28, 2022.

(51) Int. Cl. A61K 39/00; A61P 37/06

(52) U.S. Cl. CPC A61K 39/464412; A61K 39/4611; A61K 39/4613; A61K 39/464411; A61P 37/06; A61K 2239/21; A61K 2239/22

(57) ABSTRACT

The present invention relates to the treatment or management of autoimmune disorders, organ rejection and cancers utilizing CAR constructs that bind to T-cell surface antigens or B-cell or plasma surface antigens or both.

### CROSS-REFERENCE TO RELATED APPLICATION(S)

The present non-provisional application claims the benefit of U.S. Provisional Application No. 63/428,259, filed on Nov. 28, 2022, the specification of which is incorporated by reference herein, in its entirety for all purposes.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Nov. 27, 2023, is entitled “2541-15_US_ST26.xml”, and is 244,895 bytes in size.

### FIELD OF THE INVENTION

The present invention relates to the treatment or management of autoimmune disorders, organ rejection and cancers.

### BACKGROUND

T cells, a type of lymphocyte, play a central role in cell-mediated immunity. They are distinguished from other lymphocytes, such as B cells and natural killer cells (NK cells), by the presence of a T-cell receptor (TCR) on the cell surface.

Autoimmune disorders occur when the immune system of a subject attacks healthy cells, tissues, or organs of the subject's own body. In some cases, these disorders can result from abnormal recognition of antigens on the subject's own tissues (“self-antigens”) by T lineage cells (“autoreactive T-cells”) via their T-cell receptors (TCRs). A similar process occurs in a host after receiving an organ from a donor. This is also known as “organ rejection.”

For example, diabetes mellitus type I (T1D) is an autoimmune disease that results from autoreactive T-cell mediated destruction of the insulin producing 3-cells in the pancreatic islets of Langerhans, with subsequent insulin deficiency and hyperglycemia. This is primarily mediated by T-cells specific for islet autoantigens. There is currently no cure for T1D, and there is an annual increase in incidence of roughly 2-3%. U.S. data indicates roughly 22.9 cases per 100,000 people younger than 65. T1D is diagnosed by a fasting blood glucose level over 126 mg/dL and a random blood glucose sample of over 200 mg/dL. T1D patients suffer associated hypoglycemia and ketoacidosis because of their condition and may be life-threatening. Microvascular complications of T1D manifest as retinopathy, neuropathy, nephropathy, cognitive impairments, as well as increased risk of atherosclerosis and thrombosis. Standard of care clinical management aims to reduce HbA_{1c} and reduce the risk of hyperglycemia. Globally, T1D is an emerging public health burden, and its onset is most common among individuals under the age of 15. Unfortunately, there are no disease-modifying prevention or intervention strategies for halting T1D progression in routine clinical use. Patients are dependent on exogenous insulin administration to maintain glucose homeostasis, and this has been the case since it was first used in humans approximately 100 years ago. Yet despite the evidence establishing the importance of glycemic control in reducing complications, lifelong adherence to daily management regimes is both difficult for the individual and has suboptimal outcomes. Therefore, developing methods of conserving or reinstating endogenous insulin output constitutes the ultimate objective of T1D research. Given the absence of breakthrough improvement in the past century, there is a clear and present unmet clinical disease in this space.

An ideal innovative cure for diabetes would involve the arrest and potential reversal of the destruction of the β-cell mass without prominent side effects, resulting in long-term independence from exogenous insulin.

Similarly, treatment protocols for IBD (inflammatory bowel disease) have been focused on clinical management of the disease and have remained largely unchanged for decades. As such, there exists a present clinical unmet need for delivering innovative treatment for IBD patients, whereby treatment is more efficacious, safe, and long-lasting.

Numerous other autoimmune diseases, including cancers, plague the population and there are very few treatment options—all of which have serious side effect profiles. There is strong need for safer, more efficacious treatment options for autoimmune diseases and cancers.

Therefore, existing treatments for autoimmune disorders are not always effective in the induction or maintenance of remission and/or may have undesirable side effects. Thus, reasserting the clear and present need for further therapies for the treatment and management of autoimmune disorders. The herein novel invention resets the immune system by depleting disease causing auto- T-cells, B-cells, plasma cells and re-population lymphocyte counts from bone marrow stem cells.

### SUMMARY

The present invention relates to methods of treating or managing a subject having an autoimmune disorder or organ rejection utilizing CAR that binds to T-cell surface antigen, but not limited to CD2, CD3, CD4, CD5, CD7 and an antigen that is present on the surface of B-cells or plasma cells, but not limited to CD19, CD20, CD22, CD38, CS1 (CD319, SLAMF7), GPRC5D (G protein-coupled receptor, class C, group 5, member D) and BCMA. In preferred embodiment, the dual CAR where the one CAR unit targets for T-cells by selecting at least one of the target antigens from the following group: CD2, CD3, CD4, CD5, and CD7; and the other CAR unit targets for B-cells by selecting at least one of the target antigens from the following group: CD19, CD20, CD22; or plasma cells by selecting at least one of the target antigens from the following group or target antigen BCMA, CD38, CS1 (CD319, SLAMF7), GPRC5D and CD138.

In another embodiment, a dual CAR which can be a bispecific tandem CAR, compound CAR, cistronic chimeric antigen receptor CAR and bispecific CAR is utilized in the methods.

The structure of compound CAR (cCAR) and methods of generating cCAR are described in PCT/US2016/039306, PCT/US2016/068349, PCT/US2018/038529, all of which are hereby incorporated by reference in their entirety.

In one embodiment, the disclosure provides a method of treating a cell proliferative disease. The method includes administering to a patient in need thereof a therapeutically effective amount of an engineered cell expressing a CAR polypeptide having a CD2, CD3, CD4, CD5, CD7, CD8, or CD52 antigen recognition domain.

In one embodiment, the disclosure provides a method of treating an autoimmune disease. The method includes administering to a patient in need thereof a therapeutically effective amount of an engineered cell expressing a CAR polypeptide having a CD2, CD3, CD4, CD5, CD7, CD8, or CD52 antigen recognition domain.

In one embodiment, the present invention provides a method of treating or managing an autoimmune disorder, the method whereby a CAR comprised by selecting one of the target antigens from the following group: CD2, CD3, CD4, CD5, or CD7. In further embodiments, the CAR T-cells deplete autoreactive T-cells against host tissues.

In preferred embodiments, the autoimmune disorder is caused by T lineage cells (e.g. autoreactive T lineage cells). In the preferred embodiments, the T lineage cells, e.g. autoreactive T lineage cells, are T-cells possessing TCRs directed against healthy tissue including pancreatic β-cells. In further embodiments, the CAR T-cells deplete autoreactive T-cells against healthy host tissues.

In another embodiment, the autoimmune disorder is caused by T lineage cells (e.g. autoreactive T lineage cells) result in inflammation and damage to healthy tissue, such as infiltration of the lamina propria by inflammatory CD4+ T-cell populations in IBD (Crohn's disease, ulcerative colitis). In further embodiments, the CAR T-cells deplete autoreactive T-cells against healthy host tissues.

In another embodiment, the autoimmune disorder is caused by T lineage cells (e.g. autoreactive T lineage cells) result in inflammation and damage to healthy tissue, such as infiltration by autoreactive T cells in psoriasis. In further embodiments, the CAR T-cells deplete autoreactive T-cells against healthy host tissues.

In another embodiment, graft-versus-host diseases caused by donor T lineage cells result in inflammation and damage to recipient healthy tissue. In further embodiments, the CAR T-cells deplete donor autoreactive T-cells against healthy host tissues.

In preferred embodiments, the autoimmune disorder is caused by both T lineage cells (e.g. autoreactive T lineage cells) and autoreactive antibody produced by B-cells or plasma cells.

In another embodiment, the auto-rejection of organ transplantation caused by autoreactive T-cells or autoreactive antibody produced by B-cells or plasma cells.

In another embodiment, the auto-rejection of organ transplantation caused by both autoreactive T-cells and autoreactive antibody produced by B-cells or plasma cells.

In another embodiment, the present disclosure provides an engineered cell having at least one chimeric antigen receptor polypeptide and an enhancer.

In one embodiment, the present disclosure provides an engineered cell having at least two distinct chimeric antigen receptor polypeptides and an enhancer.

As used herein, an enhancer includes a biological molecule that promotes or enhances the activity of the engineered cell having the chimeric antigen receptor polypeptide. Enhancers include cytokines. In another embodiment, enhancers include IL-2, IL-7, IL-12, IL-15, IL-18, IL-10, IL-21, PD-1, PD-L1, CSF1R, CTAL-4, TIM-3, and TGFR beta, receptors for the same, TNF-alpha., IL-15 or IL-15/IL-15sushi or IL-15/IL-15sushi anchor, and functional fragments thereof.

In one embodiment, the present disclosure provides an engineered T-cell, NK-cell having an enhancer selected from the following, IL-15 or IL-15/IL-15sushi or IL-15/IL-15sushi anchor. In this further embodiment, the additional enhancer promotes the CAR T-cell proliferation and persistency.

In another embodiment, the present disclosure provides a method of reducing the number of autoreactive T lineage cells or autoreactive B lineage cells thereof comprising administering a composition comprising (i) an engineered or modified immune cells and (ii) an IL-7, IL-15, IL-/IL-15sushi, IL-15/IL-15 sushi anchor, IL-10, CCL-119 or CCL-21 to said host in need thereof.

The most common cells of the immune system includes, but not limited to, lymphocytes (T cells, B cells, and NK cells), neutrophils, and monocytes/macrophages.

In another embodiment, the present disclosure provides a method of reducing the number of autoreactive T lineage cells or autoreactive B lineage cells thereof comprising administering a composition comprising (i) an engineered or modified NK or T cell and (ii) an IL-7, IL-15, IL-/IL-15sushi, IL-15/IL-15 sushi anchor, CCL-119 or CCL-21 to said host in need thereof.

In yet another embodiment, a method for ex vivo expansion of NK cells and T cells comprising: 1) isolation of NK or T cells; 2) introduction of at least one CAR; 3) introduction of at least one of enhancers selected from a group of IL-7, IL-15, IL-15sushi, IL-15/IL-15anchor, CCL-19 (CCL19) and CCL-21 (CCL21) and 3) expansion of NK or T cells. In some embodiments, the autoimmune disorder is selected from T1D, MS, IBD, celiac disease, Asthma, systemic lupus erythematosus, IgA nephropathy, IgG4 related disease, membranous nephropathy, Myasthenia gravis, Neuromyelitis optica, Pemphigus vulgaris, anti-PAD4-activating rheumatoid arthritis, Sensitized/preformed antibodies in solid organ transplant, Guillain-Barre Syndrome (Acute inflammatory demyelinating polyneuropathy—AIDP), Chronic inflammatory demyelinating polyneuropathy (CIDP), Immune thrombocytopenic purpura, rheumatoid arthritis, and ANCA-associated vasculitis (AAV). In preferred embodiments, the autoimmune disorder is T-cell mediated autoimmune disease.

In some embodiments, the autoimmune disorder is newly diagnosed (e.g. newly diagnosed T1D, MS, or IBD). In some embodiments, the autoimmune disorder is relapsed or refractory (e.g. relapsed or refractory T1D, MS, IBD).

In another embodiment, the CAR targeting CD7 surface antigen can deplete the autoreactive immune cell expressing the CD7 surface antigen. The CD7+ population will be depleted (approximately >90 T lymphocytes) and an unexpected finding was elucidated in that the CD7-population of T lymphocytes expand to maintain the total T-cell population and prevent infection. Such an occurrence acts as an immune system reset for the T-cell immune system thus treating T-cell mediated autoreactive disease.

In another embodiment, a CD7 monoclonal antibody targeting CD7 surface antigen can deplete the autoreactive immune cell expressing the CD7 surface antigen. The CD7+ population will be depleted (approximately >90 T lymphocytes) and an unexpected finding was elucidated in that the CD7− population of T lymphocytes expand to maintain the total T-cell population and prevent infection. Such an occurrence acts as an immune system reset for the T-cell immune system thus treating T-cell mediated autoimmune disorders.

In another embodiment, anti-CD7 CAR targets T lymphocyte lineage cells, particularly T-regulatory cells (Treg cells), which enhance the T-cell expansion. In further embodiments, the CD7CAR can be utilized as a pre-treatment for CAR T-cell cellular therapy. In such embodiments, there exists an unexpected finding, the CD7CAR displays less toxicity and no significant reduction of T-cell number in the treated subject.

In another embodiment, anti-CD7 CAR targets T lymphocyte lineage cells, particularly T-regulatory cells (Treg cells), which enhance the T-cell expansion. In further embodiments, the CD7CAR can be utilized as a pre-treatment for CAR T-cell cellular therapy utilized in combination with pre-treatment pharmaceuticals (cyclophosphamide, fludarabine). In such embodiments, there exists an unexpected finding, the CD7CAR displays less toxicity and no significant reduction of T-cell number in the treated subject.

In another embodiment, the subject can be pre-administered anti-CD7 CAR to deplete T-cell and Treg expressing CD7 surface antigen, followed by administration of a targeted CAR.

In some embodiment, the subject can be pre-administered anti-CD7 CAR to improve CAR T or NK kinetics and initial response and decrease rejection.

In another embodiment, the subject can be pre-administering an anti-CD7 monoclonal antibody can use a combination of cyclophosphamide and fludarabine to improve CAR T and NK cell expansion and decrease rejection.

In another embodiment, the subject can be administering an anti-CD7 monoclonal antibody can use a combination of cyclophosphamide and fludarabine to improve CAR T and NK cell expansion and decrease rejection.

In preferred embodiments, the autoimmune disorder is caused by T lineage cells (e.g. autoreactive T lineage cells). In the preferred embodiments, the T lineage cells, e.g. autoreactive T lineage cells, are T-cells possessing TCRs directed against healthy tissue including pancreatic β-cells. In further embodiments, autoreactive T lineage cells against pancreatic β-cells can be depleted by an anti-CD7 monoclonal antibody.

In another embodiment, the subject can be administered a dual CAR containing CD7CAR and a targeted CAR in a construct.

In another embodiment, the dual CAR may be a tandem CAR, compound CAR, cistronic chimeric antigen receptor CAR and bispecific CAR.

In further embodiments, the anti-CD7 CAR elucidated an unexpected finding whereby greater than 90% of CD7 positive T lineage lymphocytes were depleted and the minority population (2-10%) of T lineage lymphocytes negative for CD7 surface expression expanded along with the CAR T-cell population. The unexpected finding of normal T lineage lymphocyte population numbers following anti-CD7 CAR treatment provided for T-cell immune function for the subject, thus preventing infection.

In certain embodiment, the CD7CAR can be combined with a CAR that requires CAR T-cell expansion. The CD7CAR can be combined with a CAR to select from the following target antigens: at least one of this group, but not limited to, GD2, GD3, ROR1, PSMA, PSCA (prostate stem cell antigen), MAGE A3, Glycolipid, glypican 3, F77, GD-2, WT1, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, MUC1, MUC2, MUC3, MUC4, MUC5, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, CDH3, TTK, TOMM34, URLC10, KOC1, UBE2T, TOPK, ECT2, MESOTHELIN, NKG2D, P1A, GM2, CD30, MMG49 epitope, EGFRvIII, CD33, CD123, CLL-1, immunoglobin kappa and lambda, CD38, CD52, CD47, CD200, CD70, CD19, CD20, CD22, CD38, BCMA, CS1, NKG2D receptor, April receptor, BAFF receptor, TACI, CD3, CD4, CD8, CD5, CD2, GPRC5D (G protein-coupled receptor, class C, group 5, member D) and CD138. The target antigens can also include viral or fungal antigens, such as E6 and E7 from the human papillomavirus (HPV) or EBV (Epstein Barr virus) antigens.

In another embodiment, the present disclosure provides an engineered cell having at least one of recombinant IL-15, IL-15RA, IL-15sushi, IL-15/IL-15RA, IL15-RA/IL-15, IL-15/IL-15sushi, IL15sushi/IL-15, functional fragment thereof, or combination thereof; and at least one distinct CAR polypeptide wherein the antigen recognition domain includes NY-ESO-1, alpha fetoprotein (AFP), glypican-3 (GPC3), BCMA, BAFF-R, BCMA, TACI, LeY, CD5, CD7, CD2, CD3, CD4, CD45, CD13, CD14, CD15, CD19, CD20, CD22, CD33, CD41, CD61, CD64, CD68, CD117, CD123, CD138, CD267, CD269, CD38, Flt3 receptor, ROR1, PSMA, MAGE A3, Glycolipid, F77, GD-2, WT1, CEA, HER-2/neu, MAGE-3, MAGE-4, MAGE-5, MAGE-6, CA 19-9, CA 72-4, NY-ESO, FAP, ErbB, c-Met, MART-1, CD30, EGFRvIII, immunoglobin kappa and lambda, CD38 and CS1. The target antigens can also include viral or fungal antigens, such as E6 and E7 from the human papillomavirus (HPV) or EBV (Epstein Barr virus) antigens. In further embodiment, the antigen recognition polypeptides (scFv) and corresponding polynucleotides for CD2, CD3, CD5, CD7, and CD52 as well as IL-15/IL-15sushi and IL-15sushi are described in more detail publications in PCT Application NO. PCT/US2016/39306 and PCT/US2016/019953, the contents of which are incorporated herein by reference.

In another embodiment, the present disclosure provides an engineered cell to further express other regulatory factors of immune function, such as CCL21, IL-2, IL-4, IL-12, IL-13, IL-17, IL-18, IP-10, CCL4, Flt3L, interferon-gamma, MIP-1 alpha, GM-CSF, M-CSF, TGF-beta, and TNF-alpha.

In another embodiment, the present disclosure provides an engineered cell having at least one of recombinant IL-15, IL-15RA, IL-15sushi, IL-15/IL-15RA, IL15-RA/IL-15, IL-15/IL-15sushi, IL15sushi/IL-15, functional fragment thereof, or combination thereof; and at least one distinct CAR polypeptide wherein the antigen recognition domain includes NY-ESO-1, alpha fetoprotein (AFP), glypican-3 (GPC3), BCMA, BAFF-R, BCMA, TACI, LeY, CD5, CD7, CD2, CD3, CD4, CD45, CD13, CD14, CD15, CD19, CD20, CD22, CD33, CD41, CD61, CD64, CD68, CD117, CD123, CD138, CD267, CD269, CD38, Flt3 receptor, ROR1, PSMA, MAGE A3, Glycolipid, F77, GD-2, WT1, CEA, HER-2/neu, MAGE-3, MAGE-4, MAGE-5, MAGE-6, CA 19-9, CA 72-4, NY-ESO, FAP, ErbB, c-Met, MART-1, CD30, EGFRvIII, immunoglobin kappa and lambda, CD38 and CS1. The target antigens can also include viral or fungal antigens, such as E6 and E7 from the human papillomavirus (HPV) or EBV (Epstein Barr virus) antigens. In further embodiment, the antigen recognition polypeptides (scFv) and corresponding polynucleotides for CD2, CD3, CD5, CD7, and CD52 as well as IL-15/IL-15sushi and IL-15sushi are described in more detail publications in PCT Application NO. PCT/US2016/39306 and PCT/US2016/019953, the contents of which are incorporated herein by reference.

In another embodiment, the present disclosure provides an engineered cell to further express other regulatory factors of immune function, such as CCL21, IL-2, IL-4, IL-12, IL-13, IL-17, IL-18, IP-10, CCL4, Flt3L, interferon-gamma, MIP-1 alpha, GM-CSF, M-CSF, TGF-beta, and TNF-alpha.

In another embodiment, the subject can be administered anti-CD7 CAR to deplete T-cell and Treg expressing CD7 surface antigen, followed by administration of a targeted CAR with a targeted antigen at least one of this group, but not limited to, GD2, GD3, ROR1, PSMA, PSCA (prostate stem cell antigen), MAGE A3, Glycolipid, glypican 3, F77, GD-2, WT1, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, MUC1, MUC2, MUC3, MUC4, MUC5, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, CDH3, TTK, TOMM34, URLC10, KOC1, UBE2T, TOPK, ECT2, MESOTHELIN, NKG2D, P1A, GM2, CD30, MMG49 epitope, EGFRvIII, CD33, CD123, CLL-1, immunoglobin kappa and lambda, CD38, CD52, CD47, CD200, CD70, CD19, CD20, CD22, CD38, BCMA, CS1, NKG2D receptor, April receptor, BAFF receptor, TACI, CD3, CD4, CD8, CD5, CD2, and CD138. The target antigens can also include viral or fungal antigens, such as E6 and E7 from the human papillomavirus (HPV) or EBV (Epstein Barr virus) antigens.

One aspect provided is the methods of the generation or isolation of CD7-negative T or NK cells (CD7− T or NK cells) used for CAR T or NK cell therapy. The methods comprise (1) natural selection of CD7 CAR construct transduced T or NK cells; (2) isolation of peripheral CD7-negative T or NK cells; (3) anti-CD7 scFv fused to an ER (endoplasmic reticulum) retention sequence, KDEL, entraps intracellular CD7 protein within the secreting pathway and (4) CD7 gene Knockout of T or NK cells.

In a certain embodiment, the CD7CAR can be combined in a single construct with another CAR to enhance CAR T or NK -cell expansion (see FIG. 8). The CD7CAR creates CD7-negative T or NK population, which enhances the CAR T or NK cell killing and its persistence as well as reducing adverse toxicity events associated with CAR T or NK cell expansion. The CD7CAR can be combined with a CAR wherein a CAR target is selected from the following target antigens: at least one of this group, but not limited to, GD2, GD3, ROR1, PSMA, PSCA (prostate stem cell antigen), MAGE A3, Glycolipid, glypican 3, F77, GD-2, WT1, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, MUC1, MUC2, MUC3, MUC4, MUC5, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, CDH3, TTK, TOMM34, URLC10, KOC1, UBE2T, TOPK, ECT2, MESOTHELIN, NKG2D, P1A, GM2, CD30, MMG49 epitope, EGFRvIII, CD33, CD123, CLL-1, Delta-like protein 3 (DLL3), immunoglobin kappa and lambda, CD38, CD52, CD47, CD200, CD70, CD19, CD20, CD22, CD38, BCMA, CS1, NKG2D receptor, April receptor, BAFF receptor, TACI, CD3, CD4, CD8, CD5, CD2, GPRC5D (G protein-coupled receptor, class C, group 5, member D) and CD138. The target antigens can also include viral or fungal antigens, such as E6 and E7 from the human papillomavirus (HPV) or EBV (Epstein Barr virus) antigens.

In one embodiment, creation of CD7-negative population T cells or NK cells by natural self-selection approach by directly transducing a CD7 CAR and then the CD7-negative population is used to generate a CAR targeting cancers or autoimmune diseases.

In one embodiment, CD7-negative T cell or NK cell population is used to generate a CAR targeting or hematologic malignancies or solid tumors in order to reduce CAR T associated with cytokine release syndrome and neurotoxicity.

In one embodiment, CD7-negative T cell or NK cell population is used to generate a CAR targeting hematologic malignancies or solid tumors, and CD7-negative T cell or NK cell increase CAR killing efficacy.

In one embodiment, CD7-negative T cell or NK cell population is used to generate a CAR targeting hematologic malignancies or solid tumors, and CD7-negative T cell or NK cells enhance the CAR persistency or penetration to tumors or tissues.

In certain embodiments, the CD7-negative T or NK cells are used to generate a CAR to select from the following target antigens: at least one of this group, but not limited to, GD2, GD3, ROR1, PSMA, PSCA (prostate stem cell antigen), MAGE A3, Glycolipid, glypican 3, F77, GD-2, WT1, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, MUC1, MUC2, MUC3, MUC4, MUC5, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, Delta-like protein 3 (DLL3), CDH3, TTK, TOMM34, URLC10, KOC1, UBE2T, TOPK, ECT2, MESOTHELIN, NKG2D, P1A, GM2, CD30, MMG49 epitope, EGFRvIII, CD33, CD123, CLL-1, immunoglobin kappa and lambda, CD38, CD52, CD47, CD200, CD70, CD19, Claudin 18.2, CD20, CD22, CD38, BCMA, CS1, NKG2D receptor, April receptor, BAFF receptor, TACI, CD3, CD4, CD8, CD5, CD2, GPRC5D (G protein-coupled receptor, class C, group 5, member D) and CD138. The target antigens can also include viral or fungal antigens, such as E6 and E7 from the human papillomavirus (HPV) or EBV (Epstein Barr virus) antigens.

In one embodiment, methods of treating or preventing a disease or condition in a subject in need thereof are provided. The methods comprise administering a therapeutically effective amount of the pharmaceutical composition, CAR T cells of the invention.

In one embodiment, a CD7-negative T or NK cell enhance a CAR targeting cancers wherein the cancer is selected from the group consisting of a gastric cancer, a colon cancer, a lung cancer, a hepatocellular cancer, a melanoma, a breast cancer, a bladder cancer, an ovarian cancer, a cervical cancer, a renal cell cancer, a glioma, a glioblastoma and other solid tumors, and a B cell lymphoma/leukemia, a Hodgkin's lymphoma, a multiple myeloma, a T-cell lymphoblastic leukemia/lymphoma, a T cell lymphoma/leukemia, an acute myeloid leukemia, a chronic myeloid leukemia, myelodysplastic syndromes, a chronic myeloid proliferative neoplasms, NK cell neoplasms and other liquid tumors.

In another embodiment, a compound chimeric antigen receptor dual CAR is utilized where one chimeric antigen receptor (CAR) unit targets for T-cells by selecting one of the target antigens from the following group: CD2, CD3, CD4, CD5, and CD7; and the other CAR unit targets for B-cells by selecting one of the target antigens from the following group: target antigen CD19, CD20, CD22; or plasma cells by selecting one of the target antigens from the following group or target antigen BCMA, CD38, CS1, GPRC5D and CD138.

### BRIEF DESCRIPTION OF DRAWINGS

FIGS. 1A-ID. Production of CD4CAR T cells. (FIG. 1A), experimental design. (FIG. 1B), CB buffy coat cells were activated 2 days with anti-CD3 antibody and IL-2. Cells were transduced with either GFP (middle) or CD4CAR (right) lentiviral supernatant. After 7 days of incubation, cells were analyzed by flow cytometry with goat anti-mouse Fab2 or goat IgG antibodies conjugated with biotin and followed by streptavidin-PE. Non-transduced, labeled CB cells are shown on the left. (FIG. 1C), CD4CAR T cells deplete the CD4⁺ population during T cell expansion. CB buffy coat cells were activated for 2 days with anti-CD3 antibody and IL-2. CB buffy coat contains two subsets of T cells, CD8+ cytotoxic T cells and CD4⁺ helper T cells (left). Cells were transduced with either GFP (middle) or CD4CAR (right) lentiviral supernatant. After β-day culture, cells were analyzed by flow cytometry with mouse-anti-human CD4 (FITC) and CD8 (APC) antibodies. Non-transduced PMBCs were also labeled (left). (FIG. 1D), Most CD4CAR T cells have a central memory-like phenotype. CB buffy coat cells were activated 2 days with anti-CD3 antibody. Cells were transduced with CD4CAR lentiviral supernatant. After 6-day expansion, CD8+ cells were analyzed for CD62L, CD45RO and CD45RA phenotypes by flow cytometry (N=3).

FIGS. 2A-2D. CD4CAR T cells eliminate T-cell leukemic cells in co-culture assays. (FIG. 2A), CD4CAR T cells eliminate KARPAS 299 T-cell leukemic cells in co-culture. Activated human CB buffy coat cells transduced with either GFP (middle) or CD4CAR (right) lentiviral supernatant were incubated with KARPAS 299 cells at a ratio of 2:1. After 24 hours co-culture, cells were stained with mouse anti-human CD4 (APC) and CD8 (PerCp) antibodies and analyzed by flow cytometry for T cell subsets (N=3). (FIG. 2B) and (2C), CD4CAR T cells eliminate primary T- cell leukemic cells in co-culture. Activated human CB buffy coat cells transduced with either GFP (middle) or CD4CAR (right) lentiviral supernatant were incubated with primary T-cell leukemia cells from Sezary syndrome and PTCLs (FIG. 2C) at a ratio of 2:1. After 24 hours of co-culture, cells were analyzed by flow cytometry with mouse-anti-human CD4 (FITC) and CD8 (APC) antibodies (N=3). Human primary cells alone are also labeled (left). (FIG. 2D) CD4CAR T cells were unable to lyse CD4⁻ negative lymphoma cells (SP53, a B-cell lymphoma cell line). Activated human CB buffy coat cells transduced with either GFP (middle) or CD4CAR (right) lentiviral supernatant were incubated with SP53 mantle cell lymphoma cells which were pre-stained with the membrane dye CMTMR, at a ratio of 2:1. After 24 hours co-culture, cells were stained with mouse anti-human CD3 (PerCp) and then analyzed by flow cytometry (N=2). SP53 cells alone, pre-stained with CMTMR were also labeled (left).

FIGS. 3A-3B. CD4CAR T cells derived from PBMCs are highly enriched for CD8+T and specifically kill CD4⁻ expressing leukemic cell lines. (FIG. 3A) CD4CAR T cells derived from PBMCs are highly enriched for CD8+ T cells. PMBC buffy coat cells constituting T cells, CD8+ and CD4⁺ (left) were activated for 2 days with anti-CD3 antibody and IL-2, then transduced with either GFP (middle) or CD4CAR (right) lentiviral supernatant. After 3 days of culture, cells were labeled and analyzed by flow cytometry for T cell subsets. Non-transduced PMBCs were also labeled (left). (FIG. 3B) CD4CAR T cells specifically kill KARPAS 299 cells. PMBC T cells transduced with either GFP control or CD4CAR lentiviral supernatant were incubated with CFSE-stained KARPAS 299 at the ratios of 2:1, 5:1 and 10:1, respectively. After overnight incubation at 37° C., dye 7AAD was added, and the cells were analyzed by flow cytometry. Percent killing of target cells is measured by comparing survival of target cells relative to the survival of negative control cells (SP53 cells, a B-cell lymphoma cell line stained with CMTMR).

FIGS. 4A-4D. CD4CAR T cells efficiently mediate anti-leukemic effects in vivo with different modes. NSG mice received 2.5 Gy for sub-lethal irradiation. Twenty-four hours after irradiation, mice were injected subcutaneously with either 1×10⁶ (in FIG. 4A) or 0.5×10⁶ (in FIG. 4B and FIG. 4C) KARPAS 299 cells. Injected mice were treated with different courses and schedules of CD4CAR T cells or control T cells. N=5 for each group of injected mice. (FIG. 4A), a low dose of 2×10⁶ of CD4CAR T cells was injected on day 3 followed by a large dose, 8×10⁶, of CD4CAR T cells on day 22 after upon observed acceleration of tumor growth. (FIG. 4B), two large doses of CD4CAR T cells, 8×10⁶ and 5.5×10⁶ were injected on day 3 and 10 respectively. (FIG. 4C), a repeat low dose (2.5×10⁶) of CD4CAR T cells was injected every 5 days for a total of four administrations. (FIG. 4D), overall survival of mice treated with the indicated CD4CAR T cells or control GFP T cells. N=10.

FIG. 5. Co-culture specificity and dose response killing curve. CD4CAR NK cells lyse CD4⁻ expressing leukemic cell lines in a dose dependent and specific manner. CD4CAR NK and vector control cells were incubated with an equal ratio of CFSE-stained “on-target” (Karpas 299 or CCRF-CEM) cells and CMTMR-stained “off target” MOLT4 cells at 1:4, 1:2, and 1:1 effector to target ratios. After 24 hours, 7-AAD dye was added and remaining live cells were analyzed by flow cytometry. Percent killing of target cells was measured by comparing CD4⁺ Karpas 299 or CCRF-CEM cell survival in CD4CAR NK cell co-cultures relative to that in vector control NK cell co-cultures.

FIGS. 6A-6B. CD4CAR NK cells eliminate CD4⁺ T-cells isolated from human cord blood at an effector to target ratio of 2:1, but do not affect hematopoietic stem cell/progenitor compartment output. (FIG. 6A) Co-culture assays were performed at an effector to target ratio of 2:1 for 24 hours, after which, cells were stained with mouse anti-human CD56 and CD4 antibodies. Target cells were incubated alone as a control (left). NK cells were transduced with either vector control (center) or CD4CAR (right) lentiviral supernatant and incubated with CD4+ T-cells obtained from human cord blood. (N=2) (FIG. 6B) CD4CAR NK cells were incubated at co-culture effector:target ratios of 2:1 and 5:1 respectively with 500 CD34+ cord blood cells for 24 hours in NK cell media supplemented with IL-2. Experimental controls used were CD34+ cells alone, and non-transduced NK cells were co-cultured at respective 2:1 and 5:1 effector:target ratios with CD34+CB cells. Hematopoietic compartment output was assessed via formation of erythroid burst-forming units (BFU-E) and number of granulocyte/monocyte colony-forming units (CFU-GM) at Day 16. CFU statistical analysis was performed via 2-way ANOVA with alpha set at 0.05.

FIGS. 7A-7D. CD4CAR NK cells demonstrate anti-leukemic effects in vivo. NSG mice were sublethally irradiated and intradermally injected with luciferase-expressing Karpas 299 cells (Day 0) to induce measurable tumor formation. On day 1 and every 5 days for a total of 6 courses, mice were intravenously injected with 5×10⁶ CD4CAR NK cells or vector control NK control cells. (FIG. 7A) On days 7, 14, and 21, mice were injected subcutaneously with RediJect D-Luciferin and subjected to IVIS imaging. (FIG. 7B) Average light intensity measured for the CD4CAR NK injected mice was compared to that of vector control NK injected mice. (FIG. 7C) On day 1, and every other day after, tumor size area was measured and the average tumor size between the two groups was compared. (FIG. 7D) Percent survival of mice was measured and compared between the two groups.

FIGS. 8A-8C. Generation of CD5CAR. The DNA gene construct and the translated protein construct for CD5CAR, and anchored CD5 scFv antibody and a cartoon demonstrating the creation and function of CD5CAR. The DNA construct of the third generation CD5CAR construct from 5′ to 3′ reads: Leader sequence, the anti-CD5 extracellular single chain variable fragment (Anti-CD5 ScFv), the hinge region, the trans-membrane region, and the three intracellular signaling domains that define this construct as a 3rd generation car; CD28, 4-1BB and CD3ξ. The DNA construct of the anchored CD5 scFv antibody is the same as the CD5CAR construct without the intracellular signaling domains, as is the translated protein product for anchored CD5 scFv antibody. The translated protein constructs contain the anti-CD5 ScFv that will bind to the CD5 target, the hinge region that allows for appropriate positioning of the anti-CD5 ScFv to allow for optimal binding position, and the trans-membrane region. The complete CD5CAR protein also contains the two co-stimulatory domains and an intracellular domain of CD3 zeta chain. This construct is considered as a 3rd generation CAR: CD28, 4-1BB, and CD3ξ (FIG. 8A). Western blot analysis demonstrates the CD5CAR expression in HEK293 cells. HEK293 cells which had been transduced with GFP (as negative control) or CD5CAR lentiviruses for 48 h were used for Western blot analysis using CD3ξ antibody to determine the expression of CD5CAR. Left lane, the GFP control HEK293 cells, with no band as expected. The right lane showing a band at about 50 kDa, the molecular weight that we expected based on the CD5CAR construct (FIG. 8B). Flow cytometry analysis for CD5CAR expression on T cells surface for lentiviral transduced CD5CAR T cells. This analysis was performed on the double transduced CD5CAR T cells at day 8 after the second lentiviral transduction. Isotype control T cell population (negative control) vs. transduced T cells expressing CD5 CAR showing 20.53% on T cells by flow cytometry using goat anti-mouse F(AB′)2-PE (FIG. 8C).

FIG. 9. Comparisons of single and double transductions with CD5 CAR lentiviruses in the downregulation of surface CD5 expression on the T cells. The downregulation of extracellular CD5 protein versus GFP T-cell control over 8 days following lentiviral transduction is analyzed. The single transduced CD5CAR T-cells do not show complete downregulation of CD5 from cell surface by day 8, with a maximum decrease in CD5 protein expression on day 6. In the double transduced population, we note the decrease in the absolute number of CD5+, CD3+ double positive CD5CAR T-cells over time, from 24.44% on day 0 to a near complete reduction of CD5 expression on day 4. In contrast, the GFP T-cell control maintains a CD5+, CD3+ double positive population above 95% from day 2 through day 8.

FIGS. 10A-10B. CD5CAR cells effectively lyse T -ALL cell lines that express CD5, and do not lyse a T leukemic cell line that does not express CD5. (FIG. 10A) Flow cytometry analysis of T-ALL cell lines alone (left column), in co-culture with GFP vector transduced T-cells (middle row) and in co-culture with CD5CAR transduced T-cells (right row). Each cell line is seen in each row, The CD5+T-ALL cell lines in the top and middle rows (CCRF-CEM and Molt-4) with the CD5 negative cell line seen as the bottom row (KARPAS 299). KARPAS 299 is a CD5 negative T cell lymphoma. The incubation time for all co-cultures was 24 hrs, with an effector:target cell ratio of 5:1. The cell lysis compared to GFP control was over 78% for both CD5 T ALL leukemic cell lines, compared to that for the GFP control. (FIG. 10B) This bar graph denotes the T cell lysis achieved by the CD5CAR T-cells when compared to the GFP T-cells co-culture described in FIG. 10A. There was no lysis observed in CD5 CAR T cells co-cultures with KARPAS 299, which is CD5 negative (n=3 independent experiments done in duplicate).

FIGS. 11A-11D. CD5CAR cells effectively lyse T- cell acute lymphoblastic leukemic cells from patient samples that express CD5. (FIG. 11A) Flow cytometry analysis of T-ALL cells alone (left column), in co-culture with GFP T-cells (middle row) and in co-culture with CD5CAR T-cells (right row). Each patient cells are given a row and are numbered to maintain patient confidentiality. The incubation time for all co-cultures was 24 hrs, with an effector:target cell ratio of 5:1. The cell lysis compared to GFP control was over 71.3% for the T-ALL -1 compared to control. The rest of the cell lines demonstrated positive cell lysis as well, but to a lesser degree, between 33-47%. This may be related to the CD5 expression for each leukemic sample, which is discussed below. (FIG. 11B) This bar graph denotes the T cell lysis achieved by the CD5CAR T-cells when compared to the GFP T-cell co-culture. We observe a different CD5 positivity for T-ALL 1 and T-ALL 3. (FIG. 11C) The difference of mean fluorescent intensity (MFI) was determined by flow cytometry analysis. (FIG. 11D). Flow cytometry analysis of the levels of CD5 expression on a panel of four patient sample T-ALL cell populations.

FIG. 12. Analysis of CD5CAR T-cell killing ability for patient T-ALL cells (T-ALL-8) in details. Flow cytometry analysis demonstrating CD5CAR T-cell killing ability for patient's T-ALL cells. The control GFP-T cell and T-ALL-8 cell co-culture are seen on the left, and the CD5CAR co-culture with T-ALL 8 is seen on the right. We note avid lysis of all CD5 positive cells, both CD34 positive (circled in right columns) and CD34 negative (circled in left columns, T cells), with no lysis noted for CD5 negative cells. When compared to GFP control, CD5CAR T cells lyse at minimum 93.1% of CD5 positive T-ALL-8 cells when compared to GFP control. Experiment was done in duplicate. In addition, CD5CAR T cells essentially eliminate the T cell population (CD5+CD34−, circled).

FIGS. 13A-13B. CD5CAR T cells effectively eliminate normal GFP labeled T cells. (FIG. 13A) CD5CAR T cells kill normal T cells in a dose dependent manner. CD5CAR T cells or CD123CAR T cells (control) were co-cultured with GFP labeled T cells at 0.25:1, 0.5:1 and 1:1 effector to target ratios. After 24 hours, remaining live GFP T cells were analyzed by flow cytometry. Percent killing of target cells was measured by comparing GFP T cell survival in CD5 co-cultures relative to that in control CD123CAR T cells as T cells do not express CD123. (FIG. 13B) Co-culture killing curve based on the data from 13A.

FIGS. 14A-14C. Co-culture assays were performed to determine if normal T cells maintained CD5 expression when they were co-cultured with CD5CAR or anchored CD5 scFv T cells or CD123CAR (control) for 2 days (FIG. 14A and FIG. 14B) or 4 days (FIG. 14C) at a ratio of 1:1. CCRF-CEM or Molt-4 T ALL cells were transduced with lentiviruses expressing CD5CAR or anchored CD5 scFv. After the second transduction, the transduced leukemic cells were analyzed for CD5 expression by flow cytometry.

FIGS. 15A-15D. CD5CAR T cells demonstrate profound anti-leukemic effects in vivo. NSG mice were sublethally irradiated and, after 24 hours, intravenously injected with 1×10⁶ luciferase-expressing CCRF-CEM cells (Day 0) to induce measurable tumor formation. On day 3 and 4, mice were intravenously injected with 5×10⁶ CD5CAR T cells or vector control T cells. These injections were repeated on Days 6 and 7, for a total of 2.0×10⁷ cells per mouse. On days 5, 8, 10 and 13, mice were injected subcutaneously with RediJect D-Luciferin and subjected to IVIS imaging. (FIG. 15A) On days 5, 8, 10 and 13, mice were injected subcutaneously with RediJect D-Luciferin and subjected to IVIS imaging. (FIG. 15B) Average light intensity measured for the CDSCAR T injected mice was compared to that of vector control T injected mice. (FIG. 15C) Percentage of tumor cells killed in mice treated with CDSCAR T cells relative to control. (FIG. 15D), Peripheral blood was drawn from mice on Day 15 and percentages of leukemic cells were determined and compared to that of vector control or normal injected mice.

FIGS. 16A-16C. The CD5CAR NK cells (NK-92) effectively eliminate CCRF-CEM T-ALL cell line in vitro. (FIG. 16A) and (FIG. 16B), T-lymphoblast cell line CCRF-CEM expressing CD5 was co-cultured with CD5 CAR NK cells in the indicated E:T (effector:target) cell ratios for 24 hours. Target populations were quantified with flow cytometry using CD56 and CD5 to separate the NK-CAR and target cell population respectively. Cell survival is expressed relative to transduced vector control NK cells and each bar graph represents the average statistics for duplicate samples with N=2. (FIG. 16C) CD5CAR NK cells eliminate CCRF-CEM cells in a dose-dependent manner. T-lymphoblast cell line, CCRF-CEM expressing CD5 was co-cultured with CD5CAR NK cells in the indicated E:T (effector:target) cell ratios with the lower bound of the E:T ratio reduced. Saturation is achieved with an E:T ratio of 2:1 and co-culturing under reduced ratios results in a dosage-dependent manner of CD5 elimination. Complete elimination of CCRF-CEM was achieved at 5:1.

FIGS. 17A-17B. CD5CAR NK cells demonstrate potent anti-leukemic effects in vivo. NSG mice were sublethally irradiated and, after 24 hours, intravenously injected with 1×10⁶ luciferase-expressing CCRF-CEM cells (Day 0) to induce measurable tumor formation. On day 3 and 4, mice were intravenously injected with 5×10⁶ CD5CAR NK cells or vector control NK cells. These injections were repeated on Days 6 and 7, for a total of 2.0×10⁷ cells per mouse. (FIG. 17A), on day 5, mice were injected subcutaneously with RediJect D-Luciferin and subjected to IVIS imaging. (FIG. 17B), Percentage of tumor cells killed in mice treated with CD5CAR NK cells relative to control.

FIGS. 18A-18B. Organization of CD3CAR and its expression. (18A) Schematic representation of the organization of CD3CAR in lentiviral vectors. CAR expression is driven by a SFFV (spleen focus-forming virus) promoter and as a 3^{rd} generation construct, contains a leader sequence, the anti-CD3scFv, a hinge domain (H), a transmembrane domain (TM), two co-stimulatory domains of CD28 and 4-BB and the intracellular signaling domain of CD3 zeta. (FIG. 18B) HEK-293FT cells were transduced with lentiviral plasmids for GFP (lane 1) and CD3CAR (lane 2) for Western blot analysis at 48h post transduction and probed with mouse anti-human CD3zeta antibody (FIG. 18B).

FIGS. 19A-19D. CD3CAR NK cells eliminate CD3-expressing T-ALL cell lines in vitro. (FIG. 19A), T-lymphoblast cell line Jurkat expressing approximately 80% CD3 was co-cultured with CD3CAR NK cells in the indicated E:T (effector:target) cell ratios for 6 hours. (FIG. 19B) Sorted (CCRF-CD3) or unsorted CCRF-CEM (CCRF-CEM) cells were co-cultured with CD3CAR NK cells for 24 hours. Target populations were quantified with flow cytometry using CD56 and CD3 to separate the NK-CAR and target cell population respectively. Cell survival is expressed relative to transduced vector control NK cells and each bar graph represents the average statistics for duplicate samples with N=2 experiments. (FIG. 19C) The CD3CAR NK cells display robust killing ability for primary CD3+ leukemic cells from patient samples. SPT-1 (Sezary syndrome) patient cells were CD3 positive and were co-cultured with CD3CAR NK cells in the indicated E:T (effector:target) cell ratios for 24 hours. Target populations were quantified with flow cytometry using CD56 and CD3 to separate the NK-CAR and target cell population respectively. While SPT-1 is a heterogenous cell population, the broad CD3+ expressing population is eliminated by the CD3NK-CAR. (FIG. 19D) PT4 (unclassified PTCLs) patient cells were CD3+CD7−, and were co-cultured with CD3CAR NK cells in the indicated E:T (effector:target) cell ratios for 24 hours. Target populations were gated and quantified as seen in FIG. 19D. PT4 leukemia cells are typed CD3+CD7− and are effectively eliminated by the CD3CAR NK cells. The broad CD3+ population is also affected by the CD3CAR NK cells.

FIGS. 20A-20C. CD3CAR NK cells demonstrate profound anti-leukemic effects in vivo. NSG mice were sublethally irradiated and, after 24 hours, intravenously injected with 1×10⁶ luciferase-expressing Jurkat cells (Day 0) to induce measurable tumor formation. On day 3 and 4 mice were intravenously injected with 5×10⁶ CD3CAR NK cells or vector control NK cells each day. These injections were repeated on Days 6 and 7, and again on Day 10, for a total of 2.5×10⁷ cells per mouse. On days 4, 7, 9, and 13, mice were injected subcutaneously with RediJect D-Luciferin and subjected to IVIS imaging (FIG. 20A). (FIG. 20B) Average light intensity measured for the CD3CAR NK injected mice was compared to that of vector control NK cell injected mice. (FIG. 20C) Percentage of tumor cells killed in mice treated with CD3CAR NK cells relative to control.

FIG. 21. Three pairs of sgRNA per gene are designed with CHOPCHOP to target CD2, CD3, CD5 and CD7. Three pairs of sgRNA were designed with CHOPCHOP to target the gene of interest. Gene-specific sgRNAs were then cloned into the lentiviral vector (Lenti U6-sgRNA-SFFV-Cas9-puro-wpre) expressing a human Cas9 and puromycin resistance genes linked with an E2A self-cleaving linker. The U6-sgRNA cassette is in front of the Cas9 element. The expression of sgRNA and Cas9puro is driven by the U6 promoter and SFFV promoter, respectively.

FIGS. 22A-22D. Generation of stable CD5-deficient CCRF-CEM and MOLT-4 T cells using CRISPR/Cas9 lentivirus system. (FIG. 22A) Flow cytometry analysis demonstrating the loss of CD5 expression in CCRF-CEM T-cells with CRISPR/Cas9 KD using two different sgRNAs, Lenti-U6-sgCD5a-SFFV-Cas9puro (sgCD5A) and Lenti-U6-sgCD5b-SFFV-Cas9puro (sgCD5B) after puromycin selection. Wild type control is seen in the left most scatter plot. Because the CRISPR/Cas9 KD technique with sgRNA CD5A was more successful at CD5 protein downregulation, this population (denoted by the circle and arrow) was selected for sorting, purification and analysis in FIG. 22B. (FIG. 22B) Flow cytometry analysis data indicating the percentage of purely sorted stable CD5 negative CCRF-CEM cells transduced using the scCD5A CRISPR/Cas9 technique. We note the >99% purity of CD45 positive, CD5 negative CCRF sgCD5A T-cells. (FIG. 22C) Flow cytometry analysis demonstrating the loss of CD5 expression in MOLT-4 T-cells with CRISPR/Cas9 KD using two different sgRNA sequences (sequence CD5A and CD5B, middle and right columns) after puromycin treatment. Wild type control is seen the leftmost scatter plot. Because the CRISPR/Cas9 KD technique with primer CD5A was more successful at CD5 protein downregulation, this population (denoted by the circle and arrow) was selected for sorting, purification and analysis in FIG. 22D. (FIG. 22D) Flow cytometry analysis data indicating the percentage of purely sorted stable CD5 negative MOLT-4 cells transduced using the scCD5A CRISPR/Cas9 technique. We note the >99% purity of CD45 positive, CD5 negative MOLT-4 sgCD5A T-cells.

FIGS. 23A-23D. Generation and cell sorting of stable CD7 loss in CCRF-CEM cells or NK-92 cells using CRISPR/Cas9 lentivirus system. The percentage of CD7 loss in CCRF-CEM (FIG. 23A and FIG. 23B) or NK-92(FIG. 23C and FIG. 23D) using sgCD7A (Lenti-U6-sgCD7a-SFFV-Cas9-puro) and sgCD7B (Lenti-U6-sgCD7b-SFFV-Cas9-puro) was determined by flow cytometric analysis with CD45 and CD7 antibodies after puromycin treatment. The values of insert in figures showed percentage of positive and negative expressing CD45 or CD7 among analysis. Right panel indicated the percentage purity of sorted stable CD7 negative cells in CCRF-CEM (FIG. 23C) or in NK-92 cells (FIG. 23D) prepared from CD7 negative cells transduced using sgCD7A or sgCD7D CRISPR lentiviruses.

FIG. 24A-24C. CD2CAR NK cells eliminate T-cell leukemic cells in co-culture assays. (FIG. 24A) CD2CAR NK cells eliminate leukemic cells from T-ALL patient's cells in co-culture. NK-92 cells transduced with either GFP (top) or CD2CAR (bottom) lentiviral supernatant were incubated with primary human T-ALL cells, SAMPL1 (PT1) at a ratio of 5:1 (1 for 100,000 cells). After 24 hours co-culture, cells were stained with mouse anti-human CD2 (APC) antibodies and analyzed by flow cytometry (N=2). (FIG. 24B) CD2CAR NK cells eliminate a T-ALL cell line, CCRF leukemic cells in co-culture. NK-92 cells transduced with either GFP (top) or CD2CAR (bottom) lentiviral supernatant were incubated with CCRF cells at a ratio of 5:1 (1 for 100,000 cells). CCRF cells were pre-stained with cell tracker dye (CMTMR). After 24 hours co-culture, cells were stained with mouse anti-human CD2 (APC) antibodies and analyzed by flow cytometry (N=2). (FIG. 24C) Percentage of target cells (CCRF or PT1) lysed compared to GFP NK experimental control. At a 5:1 ratio and 24 hours co-culture, CD2CAR NK cells were able to eliminate about 60% of CD2 positive leukemic cells in co-culture assays.

FIGS. 25A-25B. CD7CAR NK ⁷⁻-92 cells effectively lyse T cell ALL cell line T cells that express CD7. To avoid self-killing, CD7 deficient NK-92 (NK ⁷⁻-92) cells were generated and transduced with CD7CAR. Two transduced preparations of CD7CAR NK ⁷⁻-92 cells, #A and #B were used to test their killing ability. (FIG. 25A) Flow cytometry analysis of CCRF-CEM cells alone (left column), in co-culture with GFP NK ⁷⁻-92 cells (middle column), and in co-culture with CD7CAR-NK-92-cells, #A and B #(right columns). FIG. (25B) bar graphs based on data obtained from A.

FIG. 26. Schematic representation of recombinant lentiviral vectors encoding CD7CAR (also called CD7 RTX CAR). CD7CAR consists of a humanized anti-CD7 scFv, CD8 hinge and transmembrane regions, and a CD28 co-activator fused to the CD3zeta signaling domain. The hinge region of CD7CAR also contains two RTX-binding epitopes.

FIG. 27A-27D: (27A) Characterization of CD7CAR. Staining with goat-anti-mouse F(Ab′)2-Pe revealed a CAR expression of approximately ˜70% at day 8 post-viral infection. (FIG. 27B) Staining with anti-human CD34 (used to detect the RTX-binding epitope) also demonstrated a transduction efficiency of ˜80%. (27C) Staining with anti-human CD3 and anti-human CD7 demonstrate that the CD7CAR T-cells retain CD3 expression but lose expression of CD7. (FIG. 27D) CD7CAR T-cells are able to expand at rates similar to control T-cells despite losing CD7 expression.

FIG. 28. CD7CAR demonstrates potent cytotoxicity against CD7+ cell lines in vitro. CEM-CCRF cells are ˜90% CD7+(bottom right panel). Control T-cells (left panels) or CD7-RTX T-cells (right panels) were placed with CEM-CCRF cells in an 18-h co-culture at E:T ratios of 1:1 (first row), or 2:1 (second row). Target CD7+ cells are circled in each panel. The results of the co-culture experiments demonstrate that CD7CAR T-cells lyse 99.88% and 99.82% of the CEM-CCRF cells relative to control at 1:1 and 2:1 respectively.

FIG. 29A-29D. CD7CAR improves outcome in in vivo model of T-ALL. (FIG. 29A) NSG mice were sub-lethally irradiated and then intravenously injected on Day 1 with 1.0×10⁶ luciferase-expressing CEM-CCRF cells. Five days later, mice were injected with 10×10⁶ control or CD7CAR T-cells. Mice were injected with RediJect D-Luciferin on Days 5, 10, 13, 16, and 19 and subjected to IVIS imaging. Dorsal view. (FIG. 29B) Total flux (photons/second) was measured and indicated a statistically significant difference in tumor burden between the two groups as early as day 8. (FIG. 29C) The flux of the CD7CAR-treated mice had 41.3% (dorsal) less tumor by day 8, which increased to 99.6% (dorsal) by day 19. (FIG. 29D) While all the control mice required euthanasia due to hindlimb paralysis and hunchback by day 24-26, the CD7CAR-treated mice survived significantly longer, remaining alive on Day 43. Kaplan-Meier survival analysis curve (p=0.0026).

FIG. 30. depicted is prior to CAR T cell therapy, the patient leukemic blast distribution; peripheral blood by flow cytometry yields 0.39%, bone marrow morphology is 24% and bone marrow by flow cytometry is 6.13%. The patient received the standard preconditioning therapy, fludarabine and cytarabine. Four days post preconditioning treatment the first dose of CD7 CAR T cell treatment was administered at 1×10\hat{ }6/kg and in the subsequent two days 2.0×10\hat{ }6/kg dose was administered. The patient achieved complete remission. MRD (minimal residual disease) negative in the peripheral blood and bone marrow for leukemic blasts.

FIG. 31. shows 6 days (left panel) prior to CAR T cell therapy, T cell populations were positive for CD7 surface protein. Following CAR T cell treatment, the T cell populations comprised of almost all CD3+CD7-negative cells (right three panels).

FIG. 32. the summary indicates that the absolute CBC (complete blood count), lymphocyte subtype, chimerism, percentage CAR T expression in BM and PB. See diagram/boxes. Bottom panel the hexagonal shape indicates absence of leukemic cells (right and middle panels). Bottom right, bone marrow flow cytometry analysis indicating near complete absence of CD7 surface protein in all T cells. WBC: white blood cell; NEU: neutrophils; LYM: lymphocyte; HGB: Hemoglobin; PLT: platelet; CIK: cytokine-induced killer cells; PB: peripheral blood; BM: bone marrow; GVHD: Graft-versus-host disease.

FIG. 33. Construction of anti-TAA-anit-CD3-ENG in order to expression of anti-TAA-anti-CD3 engager. Viral construct contains the cDNA encoding an immunoglobulin heavy-chain leader peptide preceding an anti-TAA scFv and anti-CD3 scFv with a short serine-glycine linker and myc-tag in tandem. TAA is called tumor associated antigen. G4S linker: GGGGS

FIG. 34. Construction of CD7 CAR and a CAR, X that requires CAR T expansion and reduction of toxicity. The CD7CAR can be combined with a CAR, X to select from the following target antigens: at least one of this group, but not limited to, GD2, GD3, ROR1, PSMA, PSCA (prostate stem cell antigen), MAGE A3, Glycolipid, glypican 3, F77, GD-2, WT1, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, MUC1, MUC2, MUC3, MUC4, MUC5, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, CDH3, TTK, TOMM34, URLC10, KOC1, UBE2T, TOPK, ECT2, MESOTHELIN, NKG2D, P1A, GM2, CD30, MMG49 epitope, EGFRvIII, CD33, CD123, CLL-1 (CD371), immunoglobin kappa and lambda, CD38, CD52, CD47, CD200, CD70, CD19, CD20, CD22, CD38, BCMA, CS1, NKG2D receptor, April receptor, BAFF receptor, TACI, CD3, CD4, CD8, CD5, CD2, GPRC5D (G protein-coupled receptor, class C, group 5, member D) and CD138. The target antigens can also include viral or fungal antigens, such as E6 and E7 from the human papillomavirus (HPV) or EBV (Epstein Barr virus) antigens. A schematic representation of CD7 compound CAR (cCAR). The construct comprises a promoter driving the expression of two units of CARs linked by a cleaving site such as P1A, E2A, T2A and F2A. Upon cleavage of the linker peptide, the cCARs split and engage upon targets expressing CD7 and any another antigen. As a novel CD7 cCAR construct, the co-stimulatory domains of the construct may include, but is not limited to, 4-1BB or CD28. Co-stimulatory domain in each CAR can be the same or different. CD7CAR consists of an anti-CD7 scFv, CD8 hinge (H) and transmembrane (TM) regions, and a co-activator fused to the CD3zeta signaling domain. The hinge region of CD7CAR also contains two CD20 RTX-binding epitopes. Expression is driven by a single promoter.

FIG. 35. A schematic representation of cCAR construct (CD7-CD19 cCAR). The construct comprises a single promoter driving the expression of two modular units of CARs linked by a P2A peptide. Upon cleavage of the linker, the cCARs split and engage upon targets expressing CD7 and/or CD19. As a novel cCAR construct, the co-stimulatory domains of the construct may include, but is not limited to, 4-1BB on the CD7 CAR segment and a CD28 region on the CD19 CAR. Co-stimulatory domain in each CAR can be the same or different. Each CAR consists of a scFv, CD8 hinge (H) and transmembrane (TM) regions, and a co-activator fused to the CD3zeta signaling domain. The hinge region of CD7CAR also contains two CD20 RTX-binding epitopes. The CD7-CD19 cCAR is designed to target autoreactive T cells expressing CD7 and autoreactive B cells expressing CD19.

FIG. 36A-36D. (FIG. 36A) Expression of BCMA-CD19-VAC (BVMA-CD19-IL-15/IL15sushi) cCAR T-cells. Staining with goat-anti-mouse F(Ab′)2-Pe revealed a CAR expression of approximately ˜28.18% by flow cytometry analysis. The viruses were obtained from a BCMA-CD19 CAR stable producer RD114 cell line. (FIG. 36B): Five days following tumor cell injection, 3 mice each group were intravenously injected with a course of 10×10⁶ BCMA-CD19-IL-15/IL-15sushi T cells or vector control T cells. On days 5, 8, 11, 14 and 17, mice were injected subcutaneously with RediJect D-Luciferin (Perkin Elmer) and subjected to IVIS imaging to measure tumor burden. (FIG. 36C): Five days following REH tumor cell injection, 3 mice each were intravenously injected with a course of 10×10⁶ BCMA-CD19-IL-15/IL-15sushi T cells or vector control T cells. On days 5, 11, 14, 17 and 20, mice were injected subcutaneously with RediJect D-Luciferin (Perkin Elmer) and subjected to IVIS imaging to measure tumor burden. (FIG. 36D). ELISA quantification of autoantibody levels. Levels of disease-causing autoantibodies (D-7 to D360) and complement (D-7 to D360) (A) AHA (B) ANA (C) anti-U1-snRNP (D) anti-nucleosome (E) anti-dsDNA (F) anti-ribosome (G) anti-SSA/Ro52 (H) anti-Sm (I) anti-SSA/Ro60 (J) complement C3 (K) complement C4. Note: D:day.

FIGS. 37A-37D. Expression of CLL1-CD33 VAC (also called CLL1-CD33-IL-15/IL15sush) cCAR T-cells (FIG. 37A). Expression of BCMA-CD19 VAC (also called BCMA-CD19-IL-15/IL15sushi) cCAR (FIG. 37B). Expression of CD7 RTX CAR (FIG. 37C). Flow cytometry analysis by staining with goat-anti-mouse F(Ab′)2-Pe showed that ˜31.32% of T-cells expressed the CLL-1-CD33 VAC cCAR, ˜61% of T-cells expressed the BCMA-CD19 VAC cCAR and 70% of T-cells expressed CD7 RTX CAR. The CD7+ T cell population was completely depleted (FIG. 37D, lower right panel). The expression was driven by the MMLV promoter. CAR expression in control cells (top left panel) and transduced T cells (circled, right) using the F(Ab′)2 antibody

FIG. 38. A schematic representation of a BCMA-CD7-IL-15/IL-15sushi cCAR construct. Links by P2A and T2A schematic to generates a cCAR co-expressing IL-15/IL-15sushi in a single construct. The construct consists of a promoter driving the expression of two complete units of CAR unites (cCAR) and secreted IL-15/IL-15sushi. Upon cleavage of the linker (P2A and T2A), the CD7 CAR and BCMA CAR split and engage upon a target (s). Each CAR has at least six domains including a leader sequence (signal domain), scFv, hinge region, transmembrane domain, costimulatory domain (including, but not limited to, CD28 or 4-1BB) and intracellular signaling, CD3 zeta chain. CD7-negative T cells or NK cells can be generated by CD7 CAR construct via a natural selection. Secreted IL-15/IL-15sushi enhances CAR T expansion and persistency. A safety switch (rituximab) has been incorporated on the hinge region of CD7 CAR.

FIG. 39A-39B. (FIG. 39A) An example of generation of CD7− negative T cells expressing cCAR. Flow cytometry results of activated human T cells transduced with BCMA-CD7Q-IL15/IL15sushi retroviral vector. Three days after transduction (and 5 days after transduction), non-transduced (left panels) and transduced (right panels) T cells were harvested, washed, and labeled for goat anti-mouse F(Ab′)2, and mouse anti-human CD3 and CD7 antibodies. Cells were fixed in 2% formalin and analyzed by flow cytometry (NovoCyte, Agilent). After transduction, almost all positive T cells become CD7-negative T cells with CAR expression. top panels: nearly 42% of CAR T cells expressed F(Ab′2) phenotype, and low panels: all T cells are CD7-negative (right) after CAR transduction as compared to control showing most T cells are CD7 positive (left). (FIG. 39B). Co-culture experiments comparing lysis of U937 expressing -BCMA(U-BCMA) cells, a human tumor cell line (U937) that is synthetically expressing CD269 (BCMA) antigen on the cell surface, by BCMA-CD7Q-IL-15/IL15sushi CAR T cells were performed at E:T ratio of 2:1 (upper panels) and 5:1 (lower panels) for 24 hours. CCRF-CEM cells were pre-labeled with CMTMR, a membrane dye, to distinguish them phenotypically from T cells. Target cells alone are shown on the left. Cells were analyzed by flow cytometry using anti-CD7 and anti-CD3 labeling. Populations encircled highlight target cell lysis. During 24-hour co-culture experiments, BCMA-CD7Q-IL-15/IL15sushi CAR T cells showed profound killing (nearly 90%) of target U-BCMA cells at a 2:1 ratio of effector:target.

FIG. 40. Schematic representation of a CD7-CD19-IL-15/IL-15sushi cCAR construct. Links by P2A and T2A schematic to generates a cCAR co-expressing IL-15/IL-15sushi in a single construct. The construct consists of a promoter driving the expression of two complete units of CAR unites and secreted IL-15/IL-15sushi. Upon cleavage of the linker (P2A and T2A), the CD7 CAR and CD19 CAR split and engage upon a target (s). Each CAR has at least six domains including a leader sequence (signal domain), scFv, hinge region, transmembrane domain, costimulatory domain (including, but not limited to, CD28 or 4-1BB) and intracellular signaling, CD3 zeta chain. CD7-negative T cells or NK cells can be generated by the CD7 CAR construct via a natural selection. Secreted IL-15/IL-15sushi enhances CART expansion and persistency in vivo. A safety switch (rituximab) has been incorporated on the hinge region of CD7 CAR.

FIG. 41. Another example of generation of CD7-negative T cells expressing a cCAR. Flow cytometry results of activated human T cells transduced with CD19-CD7-IL15/IL15sushi (also called CD7-CD19-Vac) retroviral vector. Three days after transduction (and 5 days after transduction), non-transduced (left panels) and transduced (right panels) T cells were harvested, washed, and labeled for goat anti-mouse F(Ab′)2, and mouse anti-human CD3 and CD7 antibodies. Cells were fixed in 2% formalin and analyzed by flow cytometry (NovoCyte, Agilent). After transduction, almost all T cells are CD7-negative T cells with CAR expression. top panels: nearly 71% of CAR T cells expressed F(Ab′2) phenotype, and low panels: all T cells are CD7-negative (right) after CAR transduction as compared to control showing most T cells are CD7 positive (left).

FIG. 42. CD7-negative T cells with a CD19 CAR showing remarkable killing activities. Activated CD7 positive T cells were transduced with CD7-CD19-IL-15/IL15sushi CAR T cells and the resulting T cells become CD7-negative and contain CD19 CAR and CD7 CAR. These CD7-negative T cells containing CD19 CAR demonstrate a near complete elimination of tumor cell line REH expressing CD19 antigen even at a low ratio of effector T and targeted tumor cells at 1:1. Flow cytometry analysis of control T cells (center panels), and CD7-CD19-IL-15/IL-15sushi CAR T cells (right panels), against the REH cell line after 18-hour co-culture at 1:1 (top panel) and 2:1 (low panel) effector:target ratio. T cells are displayed red. Target cells are circled. REH cells alone are on the far left.

FIG. 43. CD7-negative T cells with CD7 CAR showing remarkable killing activities. Activated CD7 positive T cells are transduced with CD7-CD19-IL-15/IL15sushi CAR T cells and the resulting CD7 positive T cells became CD7-negative and contain CD7 CAR as well as CD19 CAR. These CD7-negative T cells containing CD7 CAR demonstrate a near complete elimination of tumor cell line CCRF-CEM expressing CD7 antigen even at a low ratio of effector T and targeted tumor cells at 1:1. Flow cytometry analysis of control T cells (center panels), and CD7-CD19-IL-15/IL-15sushi CAR T cells (right panels), against the CCRF-CEM cell line after 18-hour co-culture at 1:1 (top panel) and 2:1 (low panel) effector:target ratio. T cells are displayed red. Target cells are circled. CCRF-CEM cells alone are on the far left.

FIG. 44. Construction of CD7 CAR and a CAR, X that requires CAR T expansion and reduction of toxicity. The CD7CAR can be combined in a single construct with another CAR, X to select from the following target antigens: at least one of this group, but not limited to, GD2, GD3, ROR1, PSMA, PSCA (prostate stem cell antigen), MAGE A3, Glycolipid, glypican 3, F77, GD-2, WT1, CEA, HER-2/neu, MAGE-3, MAGE-4, MAGE-5, MAGE-6, alpha-fetoprotein, CA 19-9, CA 72-4, NY-ESO, FAP, ErbB, c-Met, MART-1, MUC1, MUC2, MUC3, MUC4, MUC5, KIF20A, Survivin, AFP-1, gp100, MUC1, PAP-10, PAP-5, TRP2-1, SART-1, VEGFR1, VEGFR2, NEIL3, MPHOSPH1, DEPDC1, FOXM1, CDH3, TTK, TOMM34, URLC10, KOC1, UBE2T, TOPK, ECT2, MESOTHELIN, NKG2D, P1A, GM2, CD30, MMG49 epitope, EGFRvIII, CD33, CD123, CLL-1 (CD371), immunoglobin kappa and lambda, CD38, CD52, CD47, CD200, CD70, CD19, CD20, CD22, CD38, BCMA, CS1, NKG2D receptor, April receptor, BAFF receptor, TACI, CD3, CD4, CD8, CD5, CD2, GPRC5D (G protein-coupled receptor, class C, group 5, member D) and CD138. The target antigens can also include viral or fungal antigens, such as E6 and E7 from the human papillomavirus (HPV) or EBV (Epstein Barr virus) antigens. A schematic representation of CD7 compound CAR (cCAR). The construct comprises a promoter driving the expression of two units of CARs linked by a cleaving site such as P1A, E2A, T2A and F2A. Upon cleavage of the linker peptide, the cCARs split and engage upon targets expressing CD7 and any another antigen. As a novel CD7 cCAR construct, the co-stimulatory domains of the construct may include, but is not limited to, 4-1BB or CD28. Co-stimulatory domain in each CAR can be the same or different. CD7CAR consists of an anti-CD7 scFv, CD8 hinge (H) and transmembrane (TM) regions, and a co-activator fused to the CD3zeta signaling domain. The hinge region of CD7CAR also contains two CD20 RTX-binding epitopes. Expression is driven by a single promoter. CD7-negative T cells or NK cells can be generated by a natural selection approach with transduction of a cCAR containing a CD7 CAR construct.

FIG. 45. Anti-BCMA VHH discovery strategy. Overview of the anti-BCMA VHH discovery workflow.

FIG. 46A-46C. Protein binding assay using ELISA. Interaction of BCMA VHHs antibodies, BCMA4 (BC4) and BCMA5 (BC5) with NIH-CoVnb-112 with BCMA (human) or Cyno BCMA (Cynomolgus) protein (FIG. 46A and FIG. 46B). TACI and BFFR was used for negative control (FIG. 46C).

FIG. 47. Cell based assay for determination of BC4 and BC5 binding to the surface BCMA protein using CHO cells. Cell surface BCMA VHHs binding affinity were analyzed with BCMA-expressing CHO cells using flow cytometry analysis.

FIG. 48. Schematic diagram to elucidate the construct (BC4-RTX-IL-15/IL-15sushi). A complete CAR unit against BCMA (using only the variable heavy chain domain (VHH) as the antigen recognition sequence), with a rituximab binding domain in the hinge region and IL-15/IL15sushi domain of the IL-15 alpha receptor, is assembled on an expression vector and their expression is driven by the retroviral promoter. Anti-BC4 CAR is linked with IL-15/IL15sushi with the P2A self-cleaving sequence. The IL-15/IL15sushi portion is composed of IL-2 signal peptide fused to IL-15 and linked to sushi domain via a 26-amino acid poly-proline linker.

FIGS. 49A-49B. Co-culture experiments comparing lysis of U937-BCMA cells, a human tumor cell line that is synthetically expressing BCMA (CD269) on the cell surface, by BC4-RTX-IL-15/IL-15sushi CAR T cells were performed at E:T ratio of 2:1 (top) and 5:1(bottom) for 24 hours. U937-BCMA cells were pre-labeled with CMTMR, a membrane dye, to better distinguish them from T cells (FIG. 49A). Cells were analyzed by flow cytometry using anti-CD269 and anti-CD3 labeling. Populations encircled highlight target cell lysis. (FIG. 49B) During 24-hour co-culture experiments, BC4-RTX-IL-15/IL-15sushi CAR T cells showed profound killing (84%) of target U937-BCMA cells at a 2:1 ratio of effector:target.

FIGS. 50A-50B. Co-culture experiments comparing lysis of U937-BCMA cells, a human tumor cell line that is synthetically expressing BCMA (CD269) on the cell surface, by BC5-RTX-IL-15/IL-15sushi CAR T cells were performed at E:T ratio of 2:1 (top) and 5:1(bottom) for 24 hours (FIG. 50A). U937-BCMA cells were pre-labeled with CMTMR, a membrane dye, to better distinguish them from T cells. Cells were analyzed by flow cytometry using anti-CD269 and anti-CD3 labeling. Populations encircled highlight target cell lysis. During 24-hour co-culture experiments, BC5-RTX-IL-15/IL-15sushi CAR T cells showed profound killing (82%) of target U937-BCMA cells at a 5:1 ratio of effector:target. The construct of BC5-RTX-IL-15/IL-15sushi uses a similar way to that of BC4-RTX-IL-15/IL-15sushi (FIG. 50B).

FIGS. 51A-51C. Co-culture assay for BC4-CD19c-tan-CAR-T-cells against BCMA-expressing U937 cells at effector to target ratios of 2:1 for 22 h. (FIG. 51A) Target cells were pre-stained with cell tracker dye (CMTMR) to distinguish Effector and Target cells clearly for analysis. Dots circled indicate the leftover target cells by co-culture assay with effector T-cells. (FIG. 51B) The right bar graph indicates the % target cell lysis of BCMA-expressing U937 (duplicate) compared to control T cells in a co-culture assay. (FIG. 51C). BC4-CD19c-tan-CAR transduced cells group eliminate endogenous CD19 positive cells population after 2 days recovery from transduction CAR-viruses in cells. Flow cytometry analysis showed the control cells sample contains CD19 positive population (dots circled on left panel). On the other hand, CAR virus transduced sample group could not detect CD19 positive population (right panel). To evaluate BC4-CD19c-tan-CAR transduced T-cells can eliminate endogenous CD19 positive cells, especially B-cells, in donor cells after CAR-viruses transduction. To elucidate this question, 2 days after recovery control-T cells and CAR transduced T-cells (Day 6 after activation of T-cells) were labeled using CD45-PerCP, CD3-PE and CD19-APC antibodies for flow cytometry analysis. Once gated by CD45 positive population and FSC, and then plot by CD19 and CD45 to confirm the presence of CD19 positive cell population. Flow cytometry analysis showed the control cells sample contains CD19 positive population (dots circled on left panel). On the other hand, CAR virus transduced sample group could not detect CD19 positive population (right panel).

FIGS. 52A-52C. (FIG. 52A) CD19 based CARs deplete Reh cells in vivo and IL-15/IL-15sushi conjugates augment anti-tumor response. Mice were injected with Reh tumor cells (0.5×10⁶cells/mouse) expressing luciferase on Day 1. On Day 3 IVIS was conducted to assay the appearance of circulating Reh cells. On Day 4, control T-cells, CD19b CAR, and CD19b-IL15/IL-15sushi CAR T-cells were injected (˜7.5×10⁶ total cells/mouse) and on day 6 through 22, IVIS imaging was conducted to assay semi-quantitative assessment of tumor burden and subsequent tumor depletion and control of cell growth by T-cells. Here, both CAR T treatments demonstrated similar efficacy, with the IL-15 secreting CAR demonstrating comparable or better control of the Reh tumor growth when compared to standard CART19 cells. (FIG. 52B) Comparison CD19b-CAR-T vs CD19b-IL-15/IL15sushi CAR-T against REH cells over long term. (TOP) Similar experimental scheme with identical IVIS methodology to above, however mice were followed until signs of tumor relapse were seen. Here, after day 30, we observed that aggressive Reh tumor relapse began to occur in standard CART19 treated mice. Clusters of tumor (indicated by circled regions on the IVIS imaged mice) are seen in most CART19 mice, with a single CD19b-IL-15/IL-15sushi CART treated mice also showing tumor growth by day 22. However, after day 30, all CART19 mice show signs of severe tumor relapse, while CD19b-IL-15/IL-15sushi CART treated mice show no sign of tumor. Even the relapsed mouse on day 22 was absolved of its tumor by day 32, signifying that CD19b-IL-15/IL-15sushi CART cells were still in effective circulation. (FIG. 52C). Line graph summarizing IVIS trend values estimating tumor growth over time for each treatment cohort. Past day 30, the tumor burden for the standard CD19b CAR (CART19) treated mice rises precipitously resulting in highly significant increases in tumor burden compared to the CD19b-IL-15/IL-15sushi CART treatment group which remained largely tumor free. Values are displayed for both views of the mice (dorsal image acquisition views).

FIGS. 53A-53B. pX-CD19c-CD7Q tandem CAR (Q, also called RTX). (FIG. 53A) Activated human T cells transduced with CD19c-CD7Q retroviral vector, 3 days after transfection. PBMC from healthy human donors was activated 48 hours in the presence of mouse anti-human CD3 antibody. Cells (1.0×10e6) were transduced with either 1 mL control supernatant (left panels) or supernatant from transfected cells (right panels) at a 1:1 ratio, and incubated for 24 hours. A second identical transduction was performed for another 24 hours. Following the second transduction, T cells were washed and suspended in T cell media supplemented with 300 IU/mL IL-2. After 24 hours incubation following the second transduction, T cells were labeled for flow cytometry analysis, with mouse anti-human CD3 and CD7 antibodies to detect T cells and CD7 phenotype, respectively. CD7 expression in control T cells (circled, lower left) and transduced T cells (circled, lower right). (FIG. 53B) Co-culture experiment comparing lysis of CD19-expressing REH cells by the same CD19c-CD7Q CAR T cells from Panel A were performed at effector:target cells ratio of 2:1 for 16 hours. Cells were analyzed by flow cytometry using anti-CD19 and anti-CD3 labeling. Populations encircled highlight target cell lysis.

FIGS. 54A-54B. CD19b-CD7Q-IL-15/IL15sushi tandem CAR. (FIG. 54A) Activated human T cells transduced with pX-CD19b-CD7Q-IL-15/IL15sushi retroviral vector from transfected H29 cells, 3 days after transfection. PBMC from healthy human donors was activated 48 hours in the presence of mouse anti-human CD3 antibody. Cells (1.0×10e6) were transduced with either 1 mL control supernatant (left panels) or supernatant from transfected cells (right panels) at a 1:1 ratio, and incubated for 24 hours. A second identical transduction was performed for another 24 hours. Following the second transduction, T cells were washed and suspended in T cell media supplemented with 300 IU/mL IL-2. After 24 hours incubation following the second transduction, T cells were labeled for flow cytometry analysis, with mouse anti-human CD3 and CD7 antibodies to detect T cells and CD7 phenotype, respectively. CD7 expression in control T cells (circled, lower left) and transduced T cells (circled, lower right). (FIG. 54B). Co-culture experiment comparing lysis of CD19-expressing REH cells by the same CD19b-CD7Q-IL-15/IL15sushi CAR T cells from Panel A were performed at effector:target cells ratio of 2:1 for 16 hours. Cells were analyzed by flow cytometry using anti-CD19 and anti-CD3 labeling. Populations encircled highlight target cell lysis.

FIG. 55. CD19b-BC4 tandem CAR. Activated human T cells transduced with CD19c-BC4 CAR retroviral vector, 3 days after transfection. Co-culture experiment comparing lysis of CD19-expressing REH cells by the same CD19b-BC4 CAR T cells were performed at effector:target cells ratio of 2:1 for 16 hours. Cells were analyzed by flow cytometry using anti-CD19 and anti-CD3 labeling. Populations encircled highlight target cells. CD19b-BC4 tandem CAR deleted REH cells expressing CD19.

### DETAILED DESCRIPTION OF THE INVENTION

The term “autoimmune disease” as used herein is defined as a disorder that results from an autoimmune response. An autoimmune disease is the result of an inappropriate and excessive response to a self-antigen. Examples of autoimmune diseases include but are not limited to, achalasia, Addison's disease, acute inflammatory demyelinating polyneuropathy—AIDP, adult Still's disease, agammaglobulinemia, alopecia areata, amyloidosis, ankylosing spondylitis, anti-GBM/anti-TBM nephritis, anti-PAD4-activating rheumatoid arthritis, antiphospholipid syndrome, asthma, atopic dermatitis, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune thrombocytopenia, autoimmune urticarial, axonal & neuronal neuropathy (AMAN), Balo disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss Syndrome (CSS) or eosinophilic granulomatosis (EGPA), cicatricial pemphigoid, Cogan's syndrome, cold agglutinin disease, congenital heart block, coxsackie myocarditis, CREST syndrome, Crohn's disease, dermatitis, dermatitis herpetiformis, dermatomyositis, Devic's disease (neuromyelitis optica), diabetes mellitus, discoid lupus, Dressier's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, Goodpasture's syndrome, granulomatosis with polyangiitis, Graves' disease, Guillain-Barre syndrome, Hashimoto's disease, Hashimoto's thyroiditis, autoimmune hemolytic anemia, Henoch-Schonlein purpura (HSP), herpes gestationis or pemphigoid gestationis (PG), Hidradenitis Suppurativa (HS) (Acne Inversa), hypogammalglobulinemia, idiopathic membranous nephropathy, idiopathic thrombocytopenic purpura, IgA nephropathy, IgG4-related disease, IgG4-related sclerosing disease, IgG neuropathy, IgM polyneuropathy, immune thrombocytopenic purpura (ITP), inclusion body myositis (IBM), inflammatory bowel disease (IBD), interstitial cystitis (IC), juvenile arthritis, juvenile diabetes (type 1 diabetes), juvenile myositis (JM), Kawasaki disease, Lambert-Eaton syndrome, leukocytoclastic vasculitis, Lichen planus, Lichen sclerosus, ligneous conjunctivitis, linear IgA disease (LAD), lupus, lyme disease chronic, membranous nephropathy, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, pemphigus vulgaris, pemphigus foliaceus, peripheral neuropathy, perivenous encephalomyelitis, pernicious anemia (PA), POEMS syndrome, polyarteritis nodosa, polyglandular syndromes types I, II, and III, polymyalgia rheumatic, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's syndrome, reactive Arthritis, reflex sympathetic dystrophy, relapsing polychondritis, restless legs syndrome (RLS), retroperitoneal fibrosis, rheumatic fever, rheumatoid arthritis, juvenile rheumatoid arthritis, sarcoidosis, Schmidt syndrome, scleritis, scleroderma, sensitized/preformed antibodies in solid organ transplant, Sjogren's syndrome, sperm & testicular autoimmunity, stiff person syndrome (SPS), systemic lupus erythematosus (SLE), subacute bacterial endocarditis (SBE), Susac's syndrome, sympathetic ophthalmia (SO), Takayasu's arteritis, temporal arteritis/Giant cell arteritis, thrombocytopenic purpura, thrombotic thrombocytopenic purpura (TTP), Tolosa-Hunt syndrome (THS), transverse myelitis, type 1 diabetes, ulcerative colitis (UC), undifferentiated connective tissue disease (UCTD), uveitis, vasculitis, vitiligo, Vogt-Koyanagi-Harada disease; and Wegener's disease. Guillain-Barr syndrome, Hashimoto's disease, hemolytic anemia, systemic lupus erythematosus, multiple sclerosis, N-methyl-D-aspartate receptor (NMDAR) encephalitis, myelin-oligodendrocyte glycoprotein (MOG) spectrum disorders (MOGSD), neuromyelitis optica spectrum (NMOSD), myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, and ulcerative colitis. In preferred embodiments, the autoimmune disorder is not IgG4-related disease. In preferred embodiments, the autoimmune disorder is AAV (e.g. relapsed or refractory AAV), SLE (e.g. relapsed or refractory SLE), or rheumatoid arthritis (e.g. relapsed or refractory rheumatoid arthritis). In particularly preferred embodiments, the autoimmune disorder is AAV (e.g. relapsed or refractory AAV) or rheumatoid arthritis (e.g. relapsed or refractory rheumatoid arthritis). Immune cells or immunomodulatory cells including, but not limited to, T cells, macrophage, NK cells and NK T cells have been used for treatment of autoimmune disorders, autoreactive cells targeted to cells, tissues, and organs, infectious diseases and cancers. In the preferred embodiment, T-cells engineered to express CAR unit or units targeted toward disease have indicated profound specificity and cytotoxicity. In another embodiment, immune cells or immunomodulatory cells including but not limited to T-cells, macrophage, NK cells and dendritic cells are engineered to express CAR unit or units targeted toward disease have indicated profound specificity and cytotoxicity.

In certain embodiments, NK cells are derived from human peripheral blood mononuclear cells (PBMC), leukapheresis products (PBSC), human embryonic stem cells (hESCs), induced pluripotent stem cells (iPSCs), bone marrow, or umbilical cord blood.

The potential disadvantages of using NK cells in a cellular therapy include a lack of persistency that may reduce long-term efficacy.

In one embodiment, the present disclosure comprises a method of modified NK cells with long-lived or long persistency in vivo for treating a disease. Surprisingly, it is found that NK cells co-expressing IL-15/IL-15sushi or IL-15/IL-15 sushi anchor can extend survival for a long period of time.

IL-15 is a pleiotropic cytokine that is associated with a huge range of immunology and plays an important role in both adaptive and innate immunity.

In the preferred embodiment, autoimmune diseases are targeted, for example T1D, a disorder whereby β-cells are targeted leading to insulin deficiency and subsequent systemic disease. A clinical need is present whereby Anti-CD3 mAb treatments have been developed however, high treatment dose has resulted in severe toxicity and AEs, whereas low-dose anti-CD3 mAbs are not efficacious. Furthermore, comprehensive killing of CD3+ surface antigen populations will deplete the subject's T-cells in a way that is incompatible with life as the subject will be at risk of severe infection. Additionally autoimmune diseases could be due to autoreactive T cells, B cells and plasma cells. Given this unmet clinical needs, the preferred embodiment, the dual CAR where the one CAR unit, CD7 CAR targets for T-cells and the other CAR unit targets for B-cells by selecting one of the target antigens from the following group: CD19, CD20, CD22; or plasma cells by selecting one of the target antigens from the following group or target antigen BCMA, CD38, CD138, CS1. In further embodiment, the dual CAR can be a compound (cCAR) described in more detail publications in PCT Application Nos. PCT/US2016/039306, PCT/US2016/068349 and PCT/US2018/038529, the contents of which are incorporated herein by reference.

In further embodiment, the dual CAR can be a bispecific tandem CAR (tandem CAR) described in more detail publication (ONCOLOGY REPORTS 00: OR-227153 Mohanty, 0000), the contents of which are incorporated herein by reference. Dual CAR is a considered dual-targeting chimeric antigen receptor, which includes compound CAR (cCAR) and bispecfic tandem CAR. cCAR may be generated by cloning two complete units of CARs into the same vector wherein two CAR constructs are expressed from the same vector. Each unit of CAR has its extracellar ligand-binding domain (such as antigen binding domain), hinge region, transmembrane domain, at least one co-stimulatory and CD3 zeta signaling domain.

Bispecific tandem CAR has a dual domain to bind two different antigens within one Bi-CAR. The structure of a dual domain is formed with scFvl-scFv2 or VHH1-VHH2. Bispecific tandem CAR shares the co-stimulatory domain(s) and CD3 zeta chain.

In some embodiments, the dual CAR where the one CAR unit targets for T-cells by selecting one of the target antigens from the following group: CD2, CD3, CD4, CD5, and CD7; and the other CAR unit targets for B-cells by selecting one of the target antigens from the following group: CD19, CD20, CD22; or plasma cells by selecting one of the target antigens from the following group or target antigen BCMA, CD38, CD138, CS1, GPRC5D.

In another embodiment, the present invention is to provide engineered T-cells, macrophage, NK cells, and dendritic cells, possessing one or two CAR units, co-expressing with an immune function-enhancing factor have a high immunity-inducing activity against autoimmune disease, infectious diseases, and cancers. The present disclosure also provides methods to generate engineered T-cells, macrophage, NK cells, and dendritic cells that can secrete an immune function-enhancing factor that reprograms the immune system to combat infectious diseases and cancers.

In the preferred embodiment, the construct consists of a promoter driving the expression the cCAR where the one CAR unit targets for T-cells by selecting one of the target antigens from the following group: CD2, CD3, CD4, CD5, and CD7; and the other CAR unit targets for B-cells by selecting one of the target antigens from the following group: CD19, CD20, CD22; or plasma cells by selecting one of the target antigens from the following group or target antigen BCMA, CS1, CD138, GPRC5D, CD38; with units joined by a P2A or T2A or T2P or F2A self-cleaving peptide. Upon cleavage of this peptide, the one CAR unit and the other CAR unit split. In another embodiment, the construct consists of a promoter driving the expression the cCAR where the one CAR unit targets for T-cells by selecting one of the target antigens from the following group: CD2, CD3, CD4, CD5, and CD7; and the other CAR unit targets for B-cells by selecting one of the target antigens from the following group: CD19, CD20, CD22; or plasma cells by selecting one of the target antigens from the following group including BCMA, CD38, CS1, CD138 and GPRC5D; with units joined by a self-cleaving peptide. Upon cleavage of this peptide, the one CAR unit and the other CAR unit split. An enhancer, secreted IL-15/IL-15sushi is also linked by a self-cleavage peptide at the site that flanks either end of the two distinct CAR units. Self-cleaving peptide can be selected from one of the groups of P2A or T2A or T2P or F2A.

In another embodiment, the construct consists of a promoter driving the expression the cCAR where the one CAR unit, CD7 CAR targets for T-cells; and the other CAR unit targets for B-cells by selecting one of the target antigens from the following group: CD19, CD20, CD22; or plasma cells by selecting one of the target antigens from the following group or target antigen BCMA, CD38, CD138, CS1, GPRC5D; with units joined by a self-cleaving peptide. Upon cleavage of this peptide, the one CAR unit and the other CAR unit split.

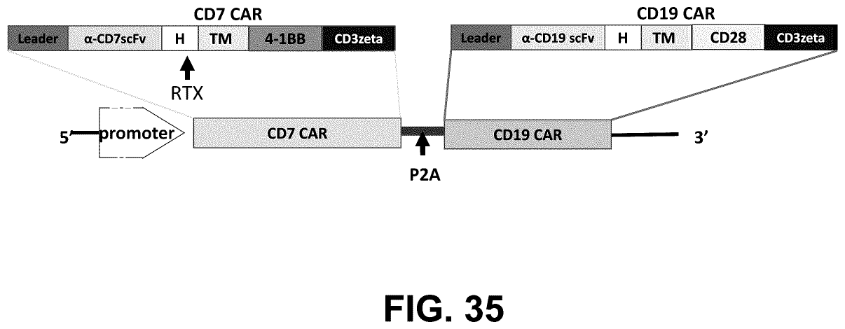

In another embodiment, the construct consists of a promoter driving the expression the cCAR (CD7-CD19 cCAR) where the one CAR unit, CD7 CAR targets for T-cells; and the other CAR unit, CD19 CAR targets for B-cells with units joined by a P2A self-cleaving peptide. Upon cleavage of this P2A peptide, the one CAR unit and the other CAR unit split (See figure 35).

In another embodiment, the construct consists of a promoter driving the expression the cCAR (CD7-CD20 cCAR) where the one CAR unit, CD7 CAR targets for T-cells; and the other CAR unit, CD20 CAR targets for B-cells with units joined by a P2A self-cleaving peptide. Upon cleavage of this P2A peptide, the one CAR unit and the other CAR unit split.

In the preferred embodiment, autoimmune diseases are targeted whereby autoreactive T cells are targeted by CD7 CAR.

In the preferred embodiment, autoimmune diseases are targeted whereby autoreactive T cells are targeted by CD5 CAR.

In the preferred embodiment, autoimmune diseases are targeted whereby autoreactive T cells are targeted by CD4 CAR.

In the preferred embodiment, autoimmune diseases are targeted whereby memory T cells including central memory T cells (TCM), effector memory T cells (TEM), and tissue-resident memory T cells (TRM) are targeted by CD4 CAR.

In the preferred embodiment, autoimmune diseases are targeted whereby autoreactive T cells are targeted by CD3 CAR.

In the preferred embodiment, the promoter driving a CAR expressing can be SFFV, human elongation factor 11a (EF) promoter and immunodeficiency virus (HIV) long terminal repeat (LTR) promoter, MoMuLV promoter.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor—1 a (EF- 1 a). However, other constitutive promoter sequences may also be used, including, but not limited to the simian virus (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosure should not be limited to the use of constitutive promoters, inducible promoters are also contemplated as part of the disclosure. The use of an inducible promoter provides a molecular switch capable of turning on expression of the polynucleotide sequence, which is operatively linked when such expression is desired, or turning off the expression when expression is not desired. Examples of inducible promoters include, but are not limited to a metalothionine promoter, a glucocorticoid promoter, a progesterone promoter, and a tetracycline promoter. Expression of chimeric antigen receptor polynucleotide may be achieved using, for example, expression vectors including, but not limited to, at least one of a SFFV (spleen-focus forming virus) or human elongation factor 11a (EF) promoter, CAG (chicken beta-actin promoter with CMV enhancer) promoter human elongation factor 1a (EF) promoter. Examples of less-strong/lower-expressing promoters utilized may include, but is not limited to, the simian virus 40 (SV40) early promoter, cytomegalovirus (CMV) immediate-early promoter, Ubiquitin C (UBC) promoter, and the phosphoglycerate kinase 1 (PGK) promoter, or a part thereof. Inducible expression of chimeric antigen receptor may be achieved using, for example, a tetracycline responsive promoter, including, but not limited to, TRE3GV (Tet-response element, including all generations and preferably, the 3rd generation), inducible promoter (Clontech Laboratories, Mountain View, CA) or a part or a combination thereof.

In the preferred embodiment, the CAR unit comprises a leader sequence, a hinge (H) region, and a transmembrane domain (TM). The self-cleavage peptides of the construct may include, but is not limited to P2A, T2A, F2A and E2A.

In another embodiment, the secreting protein(s) of the construct may also include, but is not limited to, IL-15/IL-15sushi, IL-15, IL-21, IL-18, IL-7, IL-10, and IL-12. The secreting enhancer, such as IL-15/IL-15sushi enhances T or NK cell expansion and persistency. The soluble IL-15/IL-15sushi fusion is stable and functions as an unexpected and powerful immunomodulatory for T/NK cells and their neighbor disease reducing immune response cells. The soluble IL-15/IL-15sushi fusion is also able to enhance T/NK cell persistency and stimulate T/NK cell functions of anti-pathogen or anti-tumor activities. The soluble IL-15/IL-15sushi fusion also provides vaccine-like effects by reprogramming the body's immune system to fight infections, autoimmune disease, and cancers.

The soluble IL-15/IL-15sushi fusion is stable and functions as an unexpected and powerful immunomodulatory for T/NK cells and their neighbor tumor immune response cells. The soluble IL-15/IL-15sushi fusion enhances T/NK cell persistency and stimulates T/NK cell functions in anti-pathogen, anti-autoimmune or anti-tumor activities. The soluble IL-15/IL-15sushi fusion provides vaccine-like effects by reprogramming the body's immune system to fight infections and cancers through stimulating immune cell expansion and their functions.

The construct consists of a promoter driving the expression of an IL-15/IL-15sushi anchor (also called anchor) or an IL-15/IL-15sushi anchor is linked to a CAR via a peptide self-cleavage site. The IL-15/IL-15sushi anchor is composed of a signal peptide fused to IL-15 and linked to sushi domain of IL-15 alpha receptor via a 26-amino acid poly-proline linker, two copies of rituximab epitopes (stop), hinge (H) region and a transmembrane domain (TM). IL-15/IL-15sushi is anchored on the surface of T or NK cells, which results in enhancing NK and T cell expansion and persistency.

IL-15/IL-15sushi is anchored on the surface of T or NK cells, which results in enhancing NK and T cell expansion and persistency.

Links by P2A and T2A schematic showing both cCAR-T and IL-15/IL-15sushi in a single construct. The construct consists of a SFFV promoter driving the expression of two modular units of CARs and an enhancer, IL-15/IL-15sushi. Upon cleavage of the linkers, the cCARs and IL-15/IL-15sushi split and engage upon targets expressing CD33 and/or CD123. The CD3-zeta signaling domain completes the assembly of this CAR-T. The enhancers include, but not limited to, IL-15/IL-15sushi on cCAR.

In one embodiment, the construct consists of a promoter driving the expression of two modular units of CARs and an enhancer, IL-15/IL-15sushi via a peptide cleavage site. Upon cleavage of the linkers, the cCARs and IL-15/IL-15sushi split. Two copies of rituximab is fused to the hinge region. The CD3-zeta signaling domain completes the assembly of this CAR-T. The enhancers include, but not limited to, IL-15/IL-15sushi on cCAR.

The peptide self-cleavage peptides of the construct may include, but is not limited to, P2A, T2A, F2A and E2A. The secreting protein(s) of the construct may also include, but is not limited to, IL-2, IL-15/IL-15sushi, IL-15, IL-21, IL-18, IL-7 and IL-12. The secreting enhancer, such as IL-15/IL-15sushi enhances T or NK cell expansion and persistency. The soluble IL-15/IL-15sushi fusion is stable and functions as an unexpected and powerful immunomodulatory for T/NK cells and their neighbor tumor immune response cells. The soluble IL-15/IL-15sushi fusion are stable and enhances T/NK cell persistency and stimulates T/NK cell functions in anti-pathogen or anti-tumor activities. The soluble IL-15/IL-15sushi fusion provides vaccine-like effects by reprogramming body's immune system to fight infections, autoimmune disease, and cancers. The construct consists of a promoter driving the expression of a rituximab safety switch and secreting IL-7 and an IL-15/IL-15sushi anchor linked by P2A and T2A self-cleavage peptides, respectively. Upon cleavage of the P2A and T2A peptides, enhancers, rituximab safety switch protein and IL-7 and IL-15/IL-15suhi anchor are separated. Rituximab safety protein comprises a leader sequence, an immunoglobulin FAB light chain tag, two copies of rituximab epitopes, a hinge (H) region, a transmembrane domain (TM). Secreting IL-7 (enhancer) comprises a leader sequence and IL-7 protein. The IL-15/IL-15sushi anchor is composed of a signal peptide fused to IL-15 and linked to sushi domain of IL-15 alpha receptor via a 26-amino acid poly-proline linker, hinge (H) region and a transmembrane domain (TM). IL-15/IL-15sushi is anchored on the surface of T or NK cells, which results in enhancing NK and T cell expansion and persistency. Secreting IL-7 enhances IL-15/IL-15sushi anchor's functions in immune cell expansion and persistency.

Organ rejection including cellular rejection, tissue rejection, organ rejection result from autoreactive immune cells. Transplantation rejection of cells, tissues, organs are treated with dual CAR to delete autoreactive T, B and plasma cells, where the one CAR unit, CD7 CAR targets for T-cells; and the other CAR unit targets for B-cells by selecting one of the target antigens from the following group: CD19, CD20, CD22; or plasma cells by selecting one of the target antigens from the following group or target antigen BCMA, CD38, CD138, CS1, GPRC5D.

One skilled in the art would expect the anti-CD7 CAR T-cell construct would be incompatible with the subject's life as it would deplete CD7-positive T-cell populations. An unexpected finding was elucidated in that anti-CD7 CAR T-cells deplete CD7-positive populations approximately (greater than 90%) of T-cells, however, (2-10%) of T-cells exist as CD7 negative and this cell population expands to maintain a normal T-cell count upon measurement. CD7-positive T-cells mature to the CD7-negative phenotype and the depletion of CD7-positive T-cells will serve as a reset so to alleviate the T-cell mediated autoimmune diseases, or organ rejections or cellular rejections.

Anti-CD3 monoclonal antibody constructs exhibit limited efficacy, result in T-cell aplasia, have limited systemic distribution, and cause severe toxicity incompatible with life. Therefore, anti-CD3 monoclonal antibody therapies are insufficient to treat T-cell mediated disease. The anti-CD7 CAR construct does not result significantly in T-cell aplasia, as administration of the CD7CAR treatment is accompanied by expansion of CD7 negative T-cell populations (2-10%) to maintain total T-cell numbers sufficient to fight infection and compatible with host life. The anti-CD7 CAR treatment depletes T-cells expressing CD7 surface antigen and therefore acts as an immune reset and treatment for T-cell mediated autoimmune disorders/disease.

Allogeneic or autologous NK cells induce a rapid immune response but disappear relatively rapidly from the circulation due to their limited lifespan and poor persistency.

The T- or NK-cells are an ideal platform with a CAR or dual CAR against autoreactive cells, tumors or infections if these cells can persist a relatively long period of time. However, the life expectancy of NK cells in vivo is very short, with a lifespan of one or two weeks. Ideally, the NK cell persistency should be one or two months to be considered adequate for therapy. IL-15 functions through a trimeric IL-15R complex, which contains a high affinity binding α-chain (IL-15 Rα) and the common IL-2R β- and γ-chains. IL-15 secreting from a cell bind to IL-Ra associated with IL-15 receptor β- and γ-chains on the surface of cells.

Allogeneic or autologous NK cells induce a rapid immune response but disappear relatively rapidly from the circulation due to their limited lifespan.

Constitutive expression of a high level of IL-15 in mice could cause leukemia (Fehniger et al, J Exp Med. 2001 Jan. 15; 193(2):219-31). IL-15Rα (full-length of IL-15 receptor alpha subunit) accelerates leukemia development in T cells when constitutive co-expression with IL-15 (Sato et al, Blood. 2011 Apr. 14; 117(15): 4032-4040)

Due to the important role of CD2, CD3, CD5 and CD7 antigens in T cell and NK cell based killing mechanisms, the skilled person generating new CAR cells would not use a cell engineered to delete endogenous expression of CD2, CD3, CD5 and CD7 antigens. Moreover, the skilled person would not be motivated to even consider generating CD2, CD3, CD5 or CD7-specific CAR cells targeting these antigens involved in immune responses. Thus, it was completely unexpected that a T cell which is deficient in CD2, CD3, CD5, or CD7 antigen expression is effective in CAR T cell therapies targeting those antigens.

The inventors disclose the method to improve immune cell functions while preventing tumor formation.

In the preferred embodiment, the CD7CAR depletes autoreactive T-cells expressing CD7 surface antigen and thus depleting the source of T-cell mediated disease. An unexpected finding was elucidated there exists a minute T-cell population negative for surface expression of CD7 which expands in a rapid period of time to replace the subjects T-cell count to relative normal levels, and therefore maintain the capacity to combat disease. This unexpected finding serves as an immune cell reset to deplete disease causing T-cells and expand non autoreactive lymphocyte populations.

In the preferred embodiment, the CD7CAR achieves depletion of CD7 expressing T lineage lymphocytes with an excellent safety profile, failing to induce major adverse events.

In the preferred embodiment, the compound CAR (cCAR) where the one CAR unit targets for T-cells by selecting one of the target antigens from the following group: CD2, CD3, CD4, CD5, and CD7; and the other CAR unit targets for B-cells by selecting one of the target antigens from the following group: CD19, CD20, CD22; or plasma cells by selecting one of the target antigens from the following group or target antigen BCMA, CD38, CD138, CS1, GPRC5D.

In another embodiment, a single CAR unit targets T-cells by selecting one of the target antigens from the following group: CD2, CD3, CD4, CD5, and CD7.

A 65 amino acid sequence of the extracellular portion of IL-15Rα, called sushi domain involves the binding of IL-15. It has been known that the cytoplasmic domain of IL-15 receptor a chain is critical for normal IL-15Rα functions.

The invention discloses a method of fusing IL-15 to the sushi domain instead of the full of IL-15Rα to form an IL-15/IL-15sushi fusion. In further disclosures, the signaling cytoplasmic domain of IL-15Rα is not included in the IL-15/IL-15sushi fusion. In a further disclosure, IL-15/IL-15sushi fusion is expressed and anchored on the surface of a cell, which is called IL-15/IL-15sushi anchor.

In accordance with the present disclosure, it was surprisingly found that cells expressing secreted IL-15/IL-15sushi do not observe leukemia formation in human clinical trials after more than two-year observation.

In some embodiments, IL-15/IL-15sushi fusion is expressed as a protein precursor secreted from a cell.

A protein precursor, is an inactive protein that can be turned into an active form by post-translational modification.

IL-15 is responsible for vaccine-like effects by promotion and proliferation of T cells and innate cells including NK cells. IL-15 has a very short biological half-life of about 2 hours. Our addition of the sushi domain to form an IL-15/IL-15sushi complex increases this half-life of IL-15, up to ten-fold leading to longer persistency.

In some embodiments, it is preferred that a low level and longer biologic half-life of IL-15 is preferred in vivo.

It was surprisingly found that only picogram quantities of IL-15/IL-15sushi were produced by immune cells transduced with IL-15/IL-15sushi without evidence of autonomous growth in vitro or leukemic transformation in human clinical trials after at least a 2-year observation.

In accordance with the present disclosure, the inventors have also found that immune cells transduced with secreted IL-15/IL-15sushi are superior in persistency and immunity-inducing effect to the conventional immune cells in vivo.

In order to increase this efficiency, a different leader sequence, IL-2 is used to replace the wild-type IL-15 leader sequence to achieve higher levels of secretion. Furthermore, it is known that IL-15 has a short biological half-life. Furthermore, it is known that IL-15 has a short biological half-life. The sushi domain is incorporated to increase IL-15 half-life up to ten-fold by forming an IL-15/IL-15sushi complex, leading to longer persistency.

Prior to the art, it has been found that IL-15Rα (full-length of IL-15 receptor alpha subunit) accelerates T cell leukemia development when constitutively co-expressed with IL-15 (Sato et al, Blood. 2011 Apr. 14; 117 (15), 4032-4040) in transgenic mouse models (Sato et al, Blood. 2011 Apr. 14; 117(15): 4032-4040).

The present disclosure describes an IL-15/IL-15sushi anchor having IL-15/IL-15 sushi expression on the surface of an immunomodulatory cell to enhance its functions. This IL-15/IL-sushi anchor comprises of a 65 amino acid segment of the extracellular portion of IL-15 sushi domain involving the binding of IL-15. The invention lacks the cytoplasmic functional domain and most of the extracellular domain of IL-15Rα, in order to avoid leukemic formation.

However, this omission is compensated for by the incorporation into the design of either secreted IL-7 or IL-15 or IL-15/IL-15sushi. This secretion can be easily controlled using a safety switch, thereby turning off expression in adverse conditions.

In some embodiments, the invention discloses a method of establishment of a NK cell platform or with a CAR for a universal therapy with improved persistency of NK cells and their killing activities using secreting IL-15/IL-15sushi fusion. NK cells co-expressing secretory IL-15/IL-15sushi can be used as a universal platform for treatment of a variety of diseases.

In one embodiment, the present disclosure provides an engineered cell expressing IL-15/IL-15sushi anchor.

In further embodiment, the extension of NK cell persistency can be achieved by co-expressing the IL-15/IL-15sushi anchor.

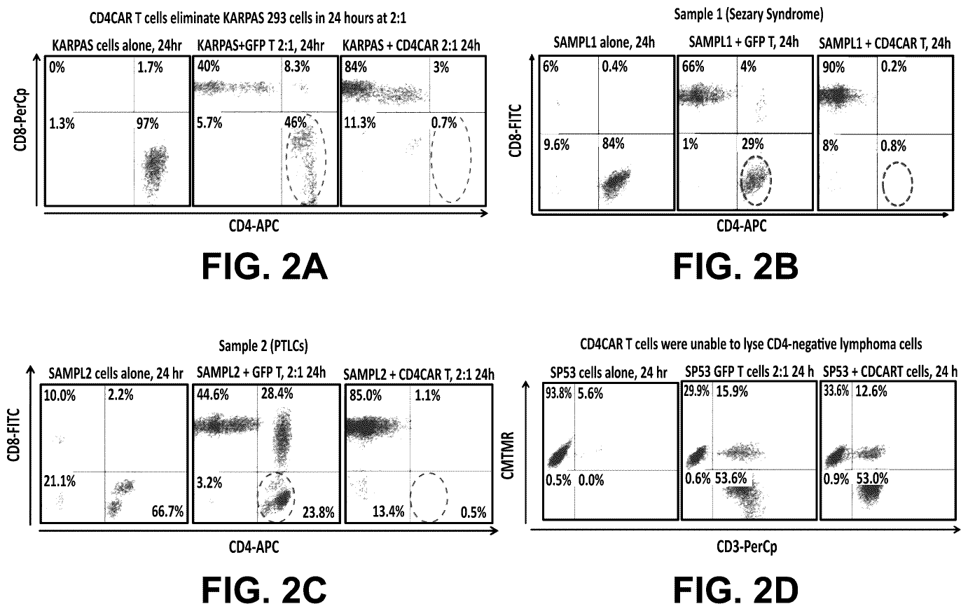

In one embodiment, the present also disclosure provides an IL-15/IL-15sushi anchor having an IL-15/IL-15 sushi, a signal peptide, a hinge region and a transmembrane domain (see FIG. 2A).

### CAR Organization

A “signal peptide” includes a peptide sequence that directs the transport and localization of the peptide and any attached polypeptide within a cell, e.g. to a certain cell organelle (such as the endoplasmic reticulum) and/or the cell surface.

The signal peptide is a peptide of any secreted or transmembrane protein that directs the transport of the polypeptide of the disclosure to the cell membrane and cell surface, and provides correct localization of the polypeptide of the present disclosure. In particular, the signal peptide of the present disclosure directs the polypeptide of the present disclosure to the cellular membrane, wherein the extracellular portion of the polypeptide is displayed on the cell surface, the transmembrane portion spans the plasma membrane, and the active domain is in the cytoplasmic portion, or interior of the cell.

In one embodiment, the signal peptide is cleaved after passage through the endoplasmic reticulum (ER), i.e. is a cleavable signal peptide. In an embodiment, the signal peptide is human protein of type I, II, III, or IV. In an embodiment, the signal peptide includes an immunoglobulin heavy chain signal peptide.

The hinge sequence may be obtained including, for example, from any suitable sequence from any genus, including human or a part thereof. Such hinge regions are known in the art. In one embodiment, the hinge region includes the hinge region of a human protein including CD-8 alpha, CD28, 4-1BB, OX40, CD3-zeta, T cell receptor a or 3 chain, a CD3 zeta chain, CD28, CD3c, CD45, CD4, CD5, CD8, CD9, CD16, CD22, CD33, CD37, CD64, CD80, CD86, CD134, CD137, ICOS, hemagglutinin (HA) of influenza virus, glycosylphosphatidylinositol (GPI)-anchored protein, CD154 and functional derivatives thereof, and combinations thereof.

In one embodiment, the hinge region includes the CD8a hinge region.

In one embodiment, the hinge region includes the HA hinge region.

In some embodiments, the hinge region includes one selected from, but is not limited to, immunoglobulin (e.g. IgGI, IgG2, IgG3, IgG4, and IgD).

The transmembrane domain includes a hydrophobic polypeptide that spans the cellular membrane. In particular, the transmembrane domain spans from one side of a cell membrane (extracellular) through to the other side of the cell membrane (intracellular or cytoplasmic). The transmembrane domain may be in the form of an alpha helix or a beta barrel, or combinations thereof. The transmembrane domain may include a polytopic protein, which has many transmembrane segments, each alpha-helical, beta sheets, or combinations thereof. The transmembrane sequence may be obtained including, for example, from any suitable sequence from any genus, including human or a part thereof. Such transmembrane regions are known in the art. In one embodiment, the transmembrane region includes the transmembrane region of a human protein including a T-cell receptor a or 3 chain, a CD3 zeta chain, CD28, CD3c, CD45, CD4, CD5, CD8, CD9, CD16, CD22, CD33, CD37, CD64, CD80, CD86, CD134, CD137, ICOS, CD154, hemagglutinin (HA) of influenza virus, and functional derivatives thereof, and combinations thereof.

In one embodiment, the transmembrane region includes the CD8a transmembrane region.

In one embodiment, the transmembrane region includes the HA transmembrane region.

In an embodiment, the CAR polypeptide further includes one or more co-stimulatory domains. In an embodiment, the co-stimulatory domain is a functional domain from a protein including CD27, CD2, CD7, CD28, CD30, CD40, PD-1, CD258, OX40, Natural killer Group 2 member C (NKG2C), Natural killer Group 2 member D (NKG2D), B7-H3, a ligand that binds to CD83, ICAM-1, LFA-1 (CD11a/CD 18), ICOS and 4-1BB (CD137), active fragments thereof, functional derivatives thereof, and combinations thereof. Such co-stimulatory domains are known in the art.

In an embodiment, the signaling domain includes the polypeptide of a functional domain of CD3 zeta, common FcR gamma (FCER1G), Fc gamma RIIIA, FcR beta (Fc Epsilon Rib), CD3 gamma, CD3 delta, CD3 epsilon, CD79a, CD79b, DNAX-activating protein 10 (DAP 10), DNAX-activating protein 12 (DAP 12), active fragments thereof, functional derivatives thereof, and combinations thereof. Such signaling domains are known in the art.

In one embodiment, the antigen recognition domain includes fragment antigen-binding fragment (Fab). In another embodiment, the antigen recognition domain includes a single-chain variable fragment (scFv). scFV is a fusion protein of the variable regions of the heavy (VH) and light chains (VL) of immunoglobulins, connected with a short linker peptide. Such antigen recognition domain including a single-chain variable fragment is known in the art.

In another embodiment, the antigen recognition domain includes Camelid single domain antibody, or portions thereof. In one embodiment, Camelid single-domain antibodies include heavy-chain antibodies found in camelids, or VHH antibody. A VHH antibody of camelid (for example camel, dromedary, llama, and alpaca) refers to a variable fragment of a camelid single-chain antibody (See Nguyen et al, 2001; Muyldermans, 2001), and also includes an isolated VHH antibody of camelid, a recombinant VHH antibody of camelid, or a synthetic VHH antibody of camelid. Such single-domain antibodies are known in the art.

In some embodiments, NK cells co-expressing IL-15/IL-15sushi or IL-15/IL-15sushi anchor can be scaled up and used as an off-the-shelf product.

In some embodiments, NK cells co-expressing both IL-15/IL-15sushi and IL-15/IL-15sushi anchor can be scaled up and used as an off-the-shelf product. In such embodiments, NK cells comprising the enhancer are expressed in a single polypeptide molecule having a high efficiency peptide cleavage sites including, but not limited to, P2A, T2A, F2A and E2A. In a further embodiment, NK cells comprising the enhancer are expressed in a single open reading frame (ORF) under the control of a strong promoter.

In some embodiments, NK cells co-expressing both IL-7 and IL-15/IL-15sushi anchor can be scaled up and used as an off-the-shelf product. In such embodiments, NK cells comprising the enhancer are expressed in a single polypeptide molecule having a high efficiency peptide cleavage sites including, but not limited to, P2A, T2A, F2A and E2A. In a further embodiment, NK cells comprising the enhancer are expressed in a single open reading frame (ORF) under the control of a strong promoter.

Examples of high efficiency cleavage sites include porcine teschovirus-1 2A (P2A), FMDV 2A (abbreviated herein as F2A); equine rhinitis A virus (ERAV) 2A (E2A); and Thoseaasigna virus 2A (T2A), cytoplasmic polyhedrosis virus 2A (BmCPV2A) and flacherie Virus 2A (BmIFV2A), or a combination thereof. In a preferred embodiment, the high efficiency cleavage site is P2A. High efficiency cleavage sites are described in Kim J H, Lee S-R, Li L-H, Park H-J, Park J-H, Lee K Y, et al. (2011) High Cleavage Efficiency of a 2A Peptide Derived from Porcine Teschovirus-1 in Human Cell Lines, Zebrafish and Mice. PLoS ONE 6(4): e18556, the contents of which are incorporated herein by reference.

The expression vector may be a bicistronic or multicistronic expression vector. Bicistronic or multicistronic expression vectors may include (1) multiple promoters fused to each of the open reading frames; (2) insertion of splicing signals between genes; fusion of genes whose expressions are driven by a single promoter; (3) insertion of proteolytic cleavage sites between genes (self-cleavage peptide); and (iv) insertion of internal ribosomal entry sites (IRESs) between genes.

In one embodiment, NK cells co-expressing IL-15/IL-15 sushi or IL-15/IL-15sushi anchor are capable of continuing supportive cytokine signaling, which is critical to their survival post-infusion in a patient.

In one embodiment, NK cells co-expressing IL-7 or IL-15/IL-15sushi anchor are capable of continuing supportive cytokine signaling, which is critical to their survival post-infusion in a patient.

In further embodiment, the extension of NK cell survival can be achieved by co-expressing a cytokine selected from a group of IL-7, IL-15, IL-15/IL-15 anchor, IL-15/IL-15RA, IL-12, IL-18 and IL-21.

Surprisingly, it was found that an immune cell co-expressing IL-15/IL-15sushi in human clinical trials revealed a significant elevation of CD8+ T cells and NK cells associated with increased anti-tumor activity and reduced disease relapses.

In some embodiments, IL-15 can be an IL-15N72D mutant and fused to the soluble domain of IL-15Rα (sushi) to form stable complexes in solution, and this complex exhibits increased biological activity compared to the non-complexed IL-15. The Mutant IL-15N72D can increase IL-15 biological activity (US20120177595 A1).

In some embodiments, a NK cell is packed with different immune defense mechanisms that: 1) alter NK cell responses to infections or tumors by mounting attacks on the targeted cells; 2) enhance NK persistency; 3) reprogram body's immune system to combat infectious diseases or cancers,

In some embodiments, a NK cell expresses at least either a cytokine(s) and/or chemokine(s). Co-expressing cytokines in a NK cell can be selected from a group of cytokines including, but not limited to: IL-15/IL-15sushi, IL-15/IL-15sush anchor, IL-2, IL-4, IL-7, IL-10, IL-12, IL-18, IL-21, GM-CSF, and TGF-β. Co-expressing chemokines in a NK cell can also be selected from a group of chemokines including, but limited to: CCL2, CCL3, CCL4, CCL5, CCL7, CCL8, CCL19, CXCL1, CXCL2, CXCL9, CXCL10, or CXCL12 or CCL-21.

In some embodiments, NK cells co-express IL-15/IL-15 anchor with at least one cytokine selected from a group of cytokines including, but not limited to, IL-15, IL-15/IL-15sushi, IL-2, IL-4, IL-7, IL-10, IL-12, IL-18, IL-21, GM-CSF, and TGF-β.

In some embodiments, an engineered cell co-expresses IL-15/IL-15 anchor with IL-15sushi.

In some embodiments, an engineered cell co-expresses IL-15/IL-15 anchor with IL-15.

In some embodiments, an engineered cell co-expresses IL-15/IL-15 anchor with IL-7.

T-Antigen Deficient T and NK Cells T cell lymphomas or T cell leukemias express specific antigens, which may represent useful targets for these diseases. For instance, T cell lymphomas or leukemias express CD7, CD2, CD3 and CD5. However, CD7, CD2, CD3, and CD5 are also expressed in CAR T or NK cells (except for CD3 and CD5), which offset their ability of targeting these antigens. The self-killing might occur in T cells or NK cells armed with CARs targeting any one of these antigens. This makes generation of CARs targeting these antigens difficult. Therefore, it may be necessary to inactivate an endogenous antigen in a T or NK cell when it is used as a target to arm CARs. In another embodiment, the engineered cell is further modified to inactivate cell surface polypeptide to prevent engineered cells from acting on other engineered cells. For example, one or more of the endogenous CD2, CD3, CD4, CD5, and CD7 genes of the engineered cells may be knocked out or inactivated. In a preferred embodiment, the engineered cell is a natural killer cell having at least one of the endogenous CD2 and CD7 genes knocked out or inactivated.

In another preferred embodiment, the engineered cell is a T-cell having at least one of the endogenous CD2, CD3, CD4, CD5, CD7, and CD8 genes knocked out or inactivated. In another preferred embodiment, the engineered cell is a NK cell having at least one of the endogenous CD2 and CD7 genes knocked out or inactivated.

In one embodiment, the engineered cell expressing a CAR having a particular antigen recognition domain will have the gene expressing that antigen inactivated or knocked out. For example, a T-cell having a CD2 CAR will have an inactivated or knocked out CD2 antigen gene. In another embodiment, an engineered cell (e.g. NK cell or T-cell) having a CAR with a CD4 antigen recognition domain will be modified so that the CD4 antigen is not expressed on its cell surface. In another embodiment, an engineered cell (e.g. NK cell or T-cell) having one CAR with a CD2 antigen recognition domain and another CAR with a CD7 antigen recognition domain may have both the CD2 antigen gene and the CD7 antigen gene knocked out or inactivated.

TABLE 1
cell surface antigens of Natural Killer cells and T-cells.

| | Natural Killer cells | T-cells |
|---|---|---|
| CD2 | + | + |
| CD4 | − | + |
| CD3 | − | + |
| CD5 | − | + |
| CD7 | + | + |
| CD8 | − | + |

Methods to knock out or inactivate genes are commonly known in the art. For example, CRISPR/Cas9 system, zinc finger nuclease (ZFNs) and TALE nucleases (TALENs), epitope base editing and meganucleases may be used to knock out or inactivate the CD2, CD3, CD4, CD5, CD7, CD8, and CD52 genes of the engineered cells.

The introduction of CARs can be fulfilled before or after the inactivation of CD2, CD5, CD3, CD7 by expanding in vitro engineered T cells prior to administration to a patient.

In particular embodiments, the inactivation of CD2, CD3, CD5, CD7 can be achieved by one of the following means:
(1) Expressing anti-CD5 scFv on T cell surface linked to a transmembrane domain via a hinge region. This may result in the conversion of CD2 or CD3, or CD5 or CD7-positive T cells to negative T cells.
(2) In some embodiments, a scFv (single-chain antibody) against CD2, or CD3 or CD5 or CD7 is derived from a monoclonal or polyclonal antibody binding to its intracellular protein and blocks the transport of protein to the cell surface. In a preferred embodiment, anti-CD2 or CD3, CD5, or CD7 scFv linked to an ER (endoplasmic reticulum) retention sequence, KDEL. When it is expressed intracellularly and retained to the ER or Golgi, the anti scFv entraps its binding protein within the secretion pathway, which results in the prevention of the protein proper cell surface location in a T cell.
(3) A natural self-selection approach via directly transducing a CAR construct of CD2 or CD3 or CD5 or CD7.

### Sources of Cells

The engineered cells may be obtained from peripheral blood, cord blood, bone marrow, tumor infiltrating lymphocytes, lymph node tissue, or thymus tissue. The host cells may include placental cells, embryonic stem cells, induced pluripotent stem cells, or hematopoietic stem cells. The cells may be obtained from humans, monkeys, chimpanzees, dogs, cats, mice, rats, and transgenic species thereof. The cells may be obtained from established cell lines.

The above cells may be obtained by any known means. The cells may be autologous, syngeneic, allogeneic, or xenogeneic to the recipient of the engineered cells.

The term “autologous” refer to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenic ally. The term “xenogeneic” refers to a graft derived from an animal of a different species.

The term “syngeneic” refers to an extremely close genetic similarity or identity especially with respect to antigens or immunological reactions. Syngeneic systems include for example, models in which organs and cells (e.g. cancer cells and their non-cancerous counterparts) come from the same individual, and/or models in which the organs and cells come from different individual animals that are of the same inbred strain.

In certain embodiments, T and NK cells are derived from human peripheral blood mononuclear cells (PBMC), leukapheresis products (PBSC), human embryonic stem cells (hESCs), induced pluripotent stem cells (iPSCs), bone marrow, or umbilical cord blood.

The potential disadvantages of using NK cells as therapy include a lack of persistency that may reduce long-term efficacy.

In some embodiments, engineered cells can immune cells or non-immune cells. Non-immune cells, for instance, can be red blood cells as a carrier to carry cytokines or chemokines to the infected and cancer tissues.

In accordance with the present disclosure, red blood cells as a carrier provide a readily available cell to be engineered to contain at least one cytokine or chemokine selecting from a group of cytokines or chemokines including, but not limited to, IL-15, IL-15/IL-15sush, IL-15/IL-15RA (full length of IL-15 receptor a), IL-15/IL-15 anchor, IL-2, IL-7, IL-12, IL-18, IL-21, CCL2, CCL3, CCL4, CCL5, CCL7, CCL8, CCL19, CXCL1, CXCL2, CXCL9, CXCL10, CXCL12 and CCL-21 polypeptide disclosed

In an embodiment, the engineered cell includes Natural Killer cells. Natural killer cells are well known in the art. In one embodiment, natural killer cells include cell lines, such as NK-92 cells. Further examples of NK cell lines include NKG, YT, NK-YS, HANK-1, YTS cells, and NKL cells.

In an embodiment, the engineered cell includes macrophages.

NK cells mediate anti-tumor effects without the risk of GvHD and are short-lived relative to T-cells. Accordingly, NK cells would be exhausted shortly after destroying targeted cells, decreasing the need for an inducible suicide gene on a construct that would ablate the modified cells.

In accordance with the present disclosure, it was surprisingly found that NK cells provide a readily available cell to be engineered to contain at least one cytokine selecting from a group of cytokines including IL-15, IL-15/IL-15sush, IL-15/IL-15RA (full length of IL-15 receptor α), IL-15/IL-15 anchor, IL-2, IL-7, IL-12, IL-18 and IL-21 polypeptide disclosed herein. Allogeneic or autologous NK cells induce a rapid immune response but disappear relatively rapidly from the circulation due to their limited lifespan. Thus, applicants surprisingly discovered that there is reduced concern of persisting side effects using NK cell-based therapy.

According to one aspect of the present invention, NK cells can be transfected with cytokine polynucleotides and expanded in accordance to the present invention. NK cells can be derived from cord blood, peripheral blood, iPS cells and embryonic stem cells. According to one aspect of the present invention, NK-92 cells may be expanded and transfected with cytokine polynucleotides. NK-92 is a continuously growing cell line that has features and characteristics of natural killer (NK) cells (Arai, Meagher et al. 2008). NK-92 cell line is IL-2 dependent and has been proven to be safe(Arai, Meagher et al. 2008) and feasible. A pure population of NK-92 carrying the cytokine polynucleotide of interest may be obtained by sorting.

In some embodiments, the engineered cell includes an inducible suicide gene (“safety switch”) or a combination of safety switches, which may be assembled on a vector, such as, without limiting, a retroviral vector, lentiviral vector, adenoviral vector or plasmid. Introduction of a “safety switch” greatly increases safety profile. The “safety switch” may be an inducible suicide gene, such as, without limiting, caspase 9 gene, thymidine kinase, cytosine deaminase (CD) or cytochrome P450. Other safety switches for elimination of unwanted modified NK or T-cells involve expression of CD20 or CD20 epitopes or CD52 or CD19 or truncated epidermal growth factor receptor in T cells. All possible safety switches have been contemplated and are embodied in the present invention.

In one embodiment, the engineered cell includes a rituximab safety switch for elimination of unwanted modified immune cells. In a further embodiment, two rituximab binding sequences are incorporated to the hinge region of CAR.

In one embodiment, the engineered cell co-expresses a rituximab epitope expression construct with IL-15/IL-15sushi through a peptide cleavage sequence selected from one of group of P2A, T2A, E2A and F2A. In a further embodiment, the rituximab epitope expression construct comprises of a signal peptide, two epitope domains of rituximab, CD8a hinge region and CD8a transmembrane domain.

Rituximab, originating as a CD20 targeted chimeric antibody, was developed by IDEC pharmaceuticals for treatment of malignancy.

### Methods of Generating Engineered Cells

Any of the polynucleotides disclosed herein may be introduced into an engineered cell by any method known in the art.

In some embodiments of the present invention, any of the engineered cells disclosed herein may be constructed in a transposon system (also called a “Sleeping Beauty”), which integrates the gene or DNA into the host genome without a viral vector.

In one embodiment, to achieve enhanced safety profile or therapeutic index, the any of the engineered cells disclosed herein be constructed as a transient DNA or RNA-modified “biodegradable” version or derivatives, or a combination thereof. The RNA- or DNA modified versions of the present invention may be electroporated into T cells or NK cells.

### Vectors

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the patient either in vivo or ex vivo. A number of retroviral systems are known in the art. In some embodiments, adenovirus vectors are used. A number of adenovirus vectors are known in the art. In one embodiment, lentivirus vectors are used.

Viral vector technology is well known in the art and is described, for example, in Sambrook et al, (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient and unique restriction endonuclease sites, and one or more selectable markers, (e.g., WO 01/96584; WO 01/29058; and U.S. Pat. No. 6,326,193).

Lentiviral vectors have been well known for their capability of transferring genes into human NK cells with high efficiency, but expression of the vector-encoded genes is dependent on the internal promoter that drives their expression. There are a wide range of promoters with different strength and cell-type specificity. Gene therapies rely on the ability of cells to express an adequate level of a protein and maintain expression over a long period of time. The EF-la promoter has been commonly selected for the gene expression.

The present invention provides an expression vector containing a strong promoter for high level gene expression in NK cells or T cells. In further embodiment, the inventor discloses a strong promoter useful for high level expression of a gene in NK cells or T-cells. In particular embodiments, a strong promoter relates to the SFFV or EF-la promoter, which is selectively introduced in an expression vector to obtain high levels of expression and maintain expression over a long period of time in NK cells or T cells. Expressed genes prefer a cytokine or chemokine and NK or T-cell co-stimulatory factors used for immunotherapy.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor—1 a (EF- 1 a). However, other constitutive promoter sequences may also be used, including, but not limited to the simian virus (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the disclosure should not be limited to the use of constitutive promoters, inducible promoters are also contemplated as part of the disclosure. The use of an inducible promoter provides a molecular switch capable of turning on expression of the polynucleotide sequence, which is operatively linked when such expression is desired, or turning off the expression when expression is not desired. Examples of inducible promoters include, but are not limited to a metalothionine promoter, a glucocorticoid promoter, a progesterone promoter, and a tetracycline promoter. Expression of chimeric antigen receptor polynucleotide may be achieved using, for example, expression vectors including, but not limited to, at least one of a SFFV (spleen-focus forming virus) or human elongation factor 11a (EF) promoter, CAG (chicken beta-actin promoter with CMV enhancer) promoter human elongation factor 1a (EF) promoter. Examples of less-strong/lower-expressing promoters utilized may include, but is not limited to, the simian virus 40 (SV40) early promoter, cytomegalovirus (CMV) immediate-early promoter, Ubiquitin C (UBC) promoter, and the phosphoglycerate kinase 1 (PGK) promoter, or a part thereof. Inducible expression of chimeric antigen receptor may be achieved using, for example, a tetracycline responsive promoter, including, but not limited to, TRE3GV (Tet-response element, including all generations and preferably, the 3rd generation), inducible promoter (Clontech Laboratories, Mountain View, CA) or a part or a combination thereof.

In a preferred embodiment, the promoter is an SFFV promoter or a derivative thereof. It has been unexpectedly discovered that SFFV promoter provides stronger expression and greater persistence in the transduced cells in accordance with the present disclosure.

“Expression vector” refers to a vector including a recombinant polynucleotide comprising expression control sequences operatively linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. The expression vector may be a bicistronic or multicistronic expression vector. Bicistronic or multicistronic expression vectors may include (1) multiple promoters fused to each of the open reading frames; (2) insertion of splicing signals between genes; fusion of genes whose expressions are driven by a single promoter; (3) insertion of proteolytic cleavage sites between genes (self-cleavage peptide); and (iv) insertion of internal ribosomal entry sites (IRESs) between genes.

In one embodiment, the disclosure provides an engineered cell having at least one chimeric antigen receptor polypeptide or polynucleotide.

An “engineered cell” means any cell of any organism that is modified, transformed, or manipulated by addition or modification of a gene, a DNA or RNA sequence, or protein or polypeptide. Isolated cells, host cells, and genetically engineered cells of the present disclosure include isolated immune cells, such as NK cells and T cells that contain the DNA or RNA sequences encoding a cytokine or a chimeric antigen receptor or chimeric antigen receptor complex and express the chimeric receptor on the cell surface. Isolated host cells and engineered cells may be used, for example, for enhancing an NK cell activity or a T lymphocyte activity for treatment of infectious diseases or cancers.

In one embodiment, the disclosure provides lipids or lipid materials used to deliver mRNA encoding CAR or a cytokine. Lipids or lipid materials used to deliver mRNA are described herein or as known to one ordinary skill in the art (Liu et al., Front Bioeng Biotechnol. 2021 Jul. 27; 9:718753).

### The Invention Provides a Method for Management and Treatment of Autoimmune Disorders.

In some embodiments, the present disclosure encompasses the administration of an anti-CD7 CAR construct such that individuals with onset or relapsed/refractory T1D, but those who do not meet the diagnostic criteria set forth by the American Diabetes Association or the Immunology of Diabetes Society to prevent or delay the onset of T1D and/or to prevent or delay the need for administration of insulin to such patients.

In further embodiments, high-risk factors for identification of predisposed patients include having first- or second-degree relatives with diagnosed T1D, an impaired fasting glucose level (e.g., at minimum one measure of a glucose level of 100-125 mg/dl after fasting (8 hours with no food), an impaired glucose tolerance in response to a 75 g OGTT (e.g., at minimum one measure of a 2-hr glucose level of 140-199 mg/dl in response to a 75 g OGTT), an HLA type of DR3, DR4 or DR7 in a Caucasian patient, an HLA type of DR3 or DR4 in a person of African descent, an HLA type of DR3, DR4 or DR9 in a person of Japanese descent, exposure to viruses (e.g., coxsackie B virus, enteroviruses, adenoviruses, rubella, cytomegalovirus, Epstein-Barr virus), a positive diagnosis according to art accepted criteria of at least one other autoimmune disorder (e.g., thyroid disease, celiac disease), and/or the detection of autoantibodies, particularly ICAs and T1D-associated autoreactive antibodies or T-cells, in the serum or other tissues. In some embodiments, the patient identified as predisposed to developing T1D has at least one of the risk factors described herein and/or as known in the art. The present disclosure also encompasses identification of subjects predisposed to development of T1D, wherein said subject presents a combination of two or more, three or more, four or more, or more than five of the risk factors disclosed herein or known in the art.

Serum autoantibodies or autoreactive T-cells associated with T1D or with a predisposition for the development of T1D are islet-cell autoantibodies (e.g., anti-ICA512 autoantibodies), glutamic acid decarbamylase autoantibodies (e.g., anti-GAD65 autoantibodies), IA2 antibodies, ZnT8 antibodies and/or anti-insulin autoantibodies or autoreactive T-cells.

The function of insulin producing β-cells prior to, during, and after therapy may be assessed by methods described herein or by any method known to one of ordinary skill in the art. For example, the Diabetes Control and Complications Trial (DCCT) research group has established the monitoring of percentage glycosylated hemoglobin (HA1 and HAlc) as the standard for evaluation of blood glucose control (DCCT, 1993, N. Engl. J. Med.329:977-986). Alternatively, characterization of daily insulin needs, C-peptide levels/response, hypoglycemic episodes, and/or FPIR may be used as markers of β-cell function or to establish a therapeutic index (See Keymeulen et al., 2005, N. Engl. J. Med.352:2598-2608; Herold et al., 2005, Diabetes 54:1763-1769; U.S. Pat. Appl. Pub. No. 2004/0038867 A1; and Greenbaum et al., 2001, Diabetes 50:470-476, respectively).

In some embodiments, the patients predisposed to develop T1D can be a non-diabetic subject who is a relative of a patient with T1D. In some embodiments, the non-diabetic subject has 2 or more diabetes-related autoantibodies or autoreactive T-cells selected from islet cell antibodies (ICA), insulin autoantibodies (IAA), and antibodies to glutamic acid decarboxylase (GAD), tyrosine phosphatase (IA-2/ICA512) or ZnT8.

In some embodiments, the non-diabetic subject has abnormal glucose tolerance on oral glucose tolerance test (OGTT). Abnormal glucose tolerance on OGTT is defined as a fasting glucose level of 110-125 mg/dL, or 2 hour plasma of ≥140 and <200 mg/dL, or an intervening glucose value at 30, 60, or 90 minutes on OGTT>200 mg/dL.

### Combination Therapy

The compositions and methods of this disclosure can be used to generate a population of T lymphocyte or NK cells that deliver both primary and co-stimulatory signals for use in immunotherapy in the treatment of autoimmune disorders. In further embodiments, the present invention for clinical aspects is combined with other agents effective in the treatment of autoimmune disorders, such as immunosuppressing agents and steroids.

Administration of any of the engineered cells described herein may be supplemented with the co-administration of an enhancing agent. Examples of enhancing agents include immunomodulatory drugs that enhance immune cell activities, such as, but not limited to agents that target immune-checkpoint pathways, inhibitors of colony stimulating factor-1 receptor (CSF1R) for better therapeutic outcomes. Agents that target immune-checkpoint pathways include small molecules, proteins, or antibodies that bind inhibitory immune receptors CTLA-4, PD-1, and PD-L1, and result in CTLA-4 and PD-1/PD-L1 blockades. As used herein, enhancing agent includes enhancer as described above.

Administration of any of the engineered cells described herein may be supplemented with the co-administration of an enhancing agent. Examples of engineered cell enhancing agents can be selected from the group of an anti-CD40 antibody or CD40 ligand, an anti-OX 40 antibody, an anti-4-1BB antibody, a TNFR2-blocking antibody, an anti-CTLA4 antibody, a PD-LI inhibitor, and a CpG oligonucleotide (CpG ODNs, TLR9 agonists).

In accordance with the present disclosure, an engineered cell can be used to express a CAR (chimeric antigen receptor) on its surface involving the treatment of a disease.

In accordance with the present disclosure, an engineered cell can be used to express T-cell receptors (TCRs) on its surface involving the treatment of a disease. TCR-engineered T (TCR-T) cells have promises against tumors and infection agent.

On this basis, the present disclosure also provides a method of providing long-term durable remission in patients by administering an engineered cell having a TCR polypeptide disclosed herein and co-expression of IL-15/IL-15sushi or IL-15/IL-15sushi anchor to increase the sensitivity of TCR recognition of target cancer cells or recruit innate immune cells to cancer cells or enhance TCR T cell persistency.

On this basis, the present disclosure also provides a method of providing long-term durable remission in patients by administering an engineered cell having a CAR polypeptide disclosed herein and co-expression of IL-15/IL-15sushi or IL-15/IL-15sushi anchor to increase the sensitivity of CAR recognition of target cancer cells or recruit innate immune cells to cancer cells or enhance CAR persistency.

Antigen-directed CAR immunotherapy, such as, but not limited to, CD19, CD20, CD22, CD2, CD3, CD4, CD5, CD7, CD52, CD38, CD33, CD30, CD123, GD2, CD45, CLL-1, BCMA, CS1, BAFF, TACI, and APRIL CAR.

In one embodiment, the target of the antigen recognition domain for CARs is selected from the group of, but not limited to, GD2, GD3, interleukin 6 receptor, ROR1, PSMA, PSCA (prostate stem cell antigen), MAGE A3, Glycolipid, glypican 3, F77, GD-2, WT1, CEA, HER-2/neu, DLL3, EGFR, folate receptor-alpha, EpCAM, CD171, CD117, mesothelin, GM2, DR5, EGFR, EpCAM, EpHA2, ER-alfa, gp100, LMP1, IL-13R, VEGFR-2, PSMA, PSCA, PD-L, MAGE-3, MAGE-4, MAGE-5, MAGE-6, alpha-fetoprotein, CA 19-9, CA 72-4, NY-ESO, FAP, ErbB, c-Met, MART-1, MUC1, MUC2, MUC3, MUC4, MUC5, MMG49 epitope, CD30, EGFRvIII, CLDN, CLDN18, CLDN18, CLDN18.2, CD33, CD123, CLL-1(CD371), NKG2D, NKG2D receptors, immunoglobin kappa and lambda, CD38, CD52, CD47, CD200, CD70, CD19, CD20, CD22, CD38, BCMA (CD269), CS1(SLAMF7, CD319), GPRC5D, BAFF receptor, TACI, CD3, CD4, CD8, CD5, CD7, CD2, and CD138.

As used herein, “patient” includes mammals. The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human. A patient includes subject. A patient in need thereof includes patients suffering from a disease that would benefit from the claimed methods of treatment or a patient at risk for developing a disease that would benefit from the claimed methods of treatment includes subject.

For example, high risk for developing type 1 diabetes is defined as:

(i) Having at least one diabetes related autoantibodies confirmed to be present on at least one occasion. This includes anti-GAD65, anti-ICA512, anti-insulin (MIAA), and ICA (islet cell antibody); (ii) Having an abnormal glucose tolerance by OGTT (oral glucose tolerance test); (iii) Having fasting plasma glucose ≥110 mg/dL and <126 mg/dl; (iv) and/or 2 hour plasma glucose ≥140 mg/dL, and <200 mg/dl; (v) and/or 30, 60, or 90 minute value on OGTT≥200 mg/dl.

In certain embodiments, the patient is a human 0 to 6 months old, 6 to 12 months old, 1 to 5 years old, 5 to 10 years old, 5 to 12 years old, 10 to 15 years old, 15 to 20 years old, 13 to 19 years old, 20 to 25 years old, 25 to 30 years old, 20 to 65 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old.

The terms “effective amount” and “therapeutically effective amount” of an engineered cell as used herein mean a sufficient amount of the engineered cell to provide the desired therapeutic or physiological or effect or outcome. Such, an effect or outcome includes reduction or amelioration of the symptoms of cellular disease. Undesirable effects, e.g. side effects, are sometimes manifested along with the desired therapeutic effect; hence, a practitioner balances the potential benefits against the potential risks in determining what an appropriate “effective amount” is. The exact amount required will vary from patient to patient, depending on the species, age and general condition of the patient, mode of administration and the like. Thus, it may not be possible to specify an exact “effective amount”. However, an appropriate “effective amount” in any individual case may be determined by one of ordinary skill in the art using only routine experimentation. Generally, the engineered cell or engineered cells is/are given in an amount and under conditions sufficient to reduce proliferation of target cells.

Following administration of the delivery system for treating, inhibiting, or preventing an autoimmune disease, the efficacy of the therapeutic engineered cell can be assessed in various ways well known to the skilled practitioner. For instance, one of ordinary skill in the art will understand that a therapeutic engineered cell delivered in conjunction with the chemo-adjuvant is efficacious in treating or inhibiting autoreactive cells in a patient by observing that the therapeutic engineered cell reduces the autoimmune cell load or prevents a further increase in autoimmune cell load.

Throughout this specification, quantities are defined by ranges, and by lower and upper boundaries of ranges. Each lower boundary can be combined with each upper boundary to define a range. The lower and upper boundaries should each be taken as a separate element. Reference throughout this specification to “one embodiment,” “an embodiment,” “one example,” or “an example” means that a particular feature, structure or characteristic described in connection with the embodiment or example is included in at least one embodiment of the present embodiments. Thus, appearances of the phrases “in one embodiment,” “in an embodiment,” “one example,” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus.

Further, unless expressly stated to the contrary, “or” refers to an inclusive “of” and not to an exclusive “or”. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

As used herein, a XXXX antigen recognition domain is a polypeptide that is selective for XXXX. “XXXX” denotes the target as discussed herein and above. For example, a CD7 antigen recognition domain is a polypeptide that is specific for CD7

As used herein, CDXCAR refers to a chimeric antigen receptor having a CDX antigen recognition domain.

Allogeneic or autologous NK cells induce a rapid immune response but disappear relatively rapidly from the circulation due to their limited lifespan and poor persistency.

The NK cell can be an effective platform against autoreactive cells, tumors or infections if these cells can persist a relatively long period of time. However, the life expectancy of NK cells in vivo is very short, with a lifespan of one or two weeks. Ideally, the NK cell persistency should be one or two months to be considered adequate for therapy.

In certain embodiments, the “subject” or “patient” is a human. In some embodiments, the “subject” or “patient” is less than 18 years old. In some embodiments, the “subject” or “patient” is 18 years old or older. In some embodiments, the subject is in need of induction of remission. In some embodiments, the subject is in need of maintenance of remission.

As used herein, the terms “treat,” “treating” or “treatment,” and the like refer to obtaining a desired pharmacologic and/or physiologic effect. Preferably, the effect is therapeutic, i.e., the effect partially or completely cures a disease and/or adverse symptom attributable to the disease. Alternatively, the pharmacologic and/or physiologic effect may be prophylactic, i.e., the effect completely or partially prevents a disease or symptom thereof.

As used herein, the terms “manage”, “managing” or “management”, and the like refer to suppressing and/or delaying the progression and/or worsening of a disease and/or adverse symptoms attributable to the disease.

The present invention relates to the treatment or management of an autoimmune disorder with bispecific) antibodies that bind to an antigen that binds to CD7 or an antigen that bind T cells (CD2, CD3, CD4, CD5) and an antigen that is present on the surface of B-cells or Plasma cells (e.g. CD19, CD20, BCMA, CS1(SLAMF7, CD319), GPRC5D, CD138). In some embodiments, the multispecific (e.g. bispecific) antibodies of the invention are capable of selectively binding to the cells causing the autoimmune disorder, for example to BCMA-expressing cells or CD19 expressing cells or CD7 expressing cells causing the autoimmune disorder.

As used herein, the term “autoreactive T lineage cells” refers to T lineage cells capable of recognizing antigens on the subject's own tissues (“self-antigens”). The autoreactive T lineage cells may have TCRs targeting healthy tissue. In some embodiments, the autoreactive B lineage cells are plasmablasts, plasma cells, memory B cells, or any combination thereof.

The term “autoimmune disease” as used herein is defined as a disorder that results from an autoimmune response. An autoimmune disease is the result of an inappropriate and excessive response to a self-antigen. Examples of autoimmune diseases include but are not limited to, achalasia, Addison's disease, acute inflammatory demyelinating polyneuropathy—AIDP, adult Still's disease, agammaglobulinemia, alopecia areata, amyloidosis, ankylosing spondylitis, anti-GBM/anti-TBM nephritis, anti-PAD4-activating rheumatoid arthritis, antiphospholipid syndrome, asthma, atopic dermatitis, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune thrombocytopenia, autoimmune urticarial, axonal & neuronal neuropathy (AMAN), Balo disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss Syndrome (CSS) or eosinophilic granulomatosis (EGPA), cicatricial pemphigoid, Cogan's syndrome, cold agglutinin disease, congenital heart block, coxsackie myocarditis, CREST syndrome, Crohn's disease, dermatitis, dermatitis herpetiformis, dermatomyositis, Devic's disease (neuromyelitis optica), diabetes mellitus, discoid lupus, Dressier's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, Goodpasture's syndrome, granulomatosis with polyangiitis, Graves' disease, Guillain-Barre syndrome, Hashimoto's disease, Hashimoto's thyroiditis, autoimmune hemolytic anemia, Henoch-Schonlein purpura (HSP), herpes gestationis or pemphigoid gestationis (PG), Hidradenitis Suppurativa (HS) (Acne Inversa), hypogammalglobulinemia, idiopathic membranous nephropathy, idiopathic thrombocytopenic purpura, IgA nephropathy, IgG4-related disease, IgG4-related sclerosing disease, IgG neuropathy, IgM polyneuropathy, immune thrombocytopenic purpura (ITP), inclusion body myositis (IBM), inflammatory bowel disease (IBD), interstitial cystitis (IC), juvenile arthritis, juvenile diabetes (type 1 diabetes), juvenile myositis (JM), Kawasaki disease, Lambert-Eaton syndrome, leukocytoclastic vasculitis, Lichen planus, Lichen sclerosus, ligneous conjunctivitis, linear IgA disease (LAD), lupus, lyme disease chronic, membranous nephropathy, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, pemphigus vulgaris, pemphigus foliaceus, peripheral neuropathy, perivenous encephalomyelitis, pernicious anemia (PA), POEMS syndrome, polyarteritis nodosa, polyglandular syndromes types I, II, and III, polymyalgia rheumatic, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's syndrome, reactive Arthritis, reflex sympathetic dystrophy, relapsing polychondritis, restless legs syndrome (RLS), retroperitoneal fibrosis, rheumatic fever, rheumatoid arthritis, juvenile rheumatoid arthritis, sarcoidosis, Schmidt syndrome, scleritis, scleroderma, sensitized/preformed antibodies in solid organ transplant, Sjogren's syndrome, sperm & testicular autoimmunity, stiff person syndrome (SPS), systemic lupus erythematosus (SLE), subacute bacterial endocarditis (SBE), Susac's syndrome, sympathetic ophthalmia (SO), Takayasu's arteritis, temporal arteritis/Giant cell arteritis, thrombocytopenic purpura, thrombotic thrombocytopenic purpura (TTP), Tolosa-Hunt syndrome (THS), transverse myelitis, type 1 diabetes, ulcerative colitis (UC), undifferentiated connective tissue disease (UCTD), uveitis, vasculitis, vitiligo, Vogt-Koyanagi-Harada disease; and Wegener's disease. In preferred embodiments, the autoimmune disorder is not IgG4-related disease. In preferred embodiments, the autoimmune disorder is AAV (e.g. relapsed or refractory AAV), SLE (e.g. relapsed or refractory SLE), or rheumatoid arthritis (e.g. relapsed or refractory rheumatoid arthritis). In particularly preferred embodiments, the autoimmune disorder is AAV (e.g. relapsed or refractory AAV) or rheumatoid arthritis (e.g. relapsed or refractory rheumatoid arthritis).

Autoimmune disorders amenable to treatment with the CD7 CAR of the invention include, without limitation, achalasia, Addison's disease, acute inflammatory demyelinating polyneuropathy—AIDP, adult Still's disease, agammaglobulinemia, alopecia areata, amyloidosis, ankylosing spondylitis, anti-GBM/anti-TBM nephritis, anti-PAD4-activating rheumatoid arthritis, antiphospholipid syndrome, asthma, atopic dermatitis, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune thrombocytopenia, autoimmune urticarial, axonal & neuronal neuropathy (AMAN), Balo disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss Syndrome (CSS) or eosinophilic granulomatosis (EGPA), cicatricial pemphigoid, Cogan's syndrome, cold agglutinin disease, congenital heart block, coxsackie myocarditis, CREST syndrome, Crohn's disease, dermatitis, dermatitis herpetiformis, dermatomyositis, Devic's disease (neuromyelitis optica), diabetes mellitus, discoid lupus, Dressier's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, Goodpasture's syndrome, granulomatosis with polyangiitis, Graves' disease, Guillain-Barre syndrome, Hashimoto's disease, Hashimoto's thyroiditis, autoimmune hemolytic anemia, Henoch-Schonlein purpura (HSP), herpes gestationis or pemphigoid gestationis (PG), Hidradenitis Suppurativa (HS) (Acne Inversa), hypogammalglobulinemia, idiopathic membranous nephropathy, idiopathic thrombocytopenic purpura, IgA nephropathy, IgG4-related disease, IgG4-related sclerosing disease, IgG neuropathy, IgM polyneuropathy, immune thrombocytopenic purpura (ITP), inclusion body myositis (IBM), inflammatory bowel disease (IBD), interstitial cystitis (IC), juvenile arthritis, juvenile diabetes (type 1 diabetes), juvenile myositis (JM), Kawasaki disease, Lambert-Eaton syndrome, leukocytoclastic vasculitis, Lichen planus, Lichen sclerosus, ligneous conjunctivitis, linear IgA disease (LAD), lupus, lyme disease chronic, membranous nephropathy, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, pemphigus vulgaris, pemphigus foliaceus, peripheral neuropathy, perivenous encephalomyelitis, pernicious anemia (PA), POEMS syndrome, polyarteritis nodosa, polyglandular syndromes types I, II, and III, polymyalgia rheumatic, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's syndrome, reactive Arthritis, reflex sympathetic dystrophy, relapsing polychondritis, restless legs syndrome (RLS), retroperitoneal fibrosis, rheumatic fever, rheumatoid arthritis, juvenile rheumatoid arthritis, sarcoidosis, Schmidt syndrome, scleritis, scleroderma, sensitized/preformed antibodies in solid organ transplant, Sjogren's syndrome, sperm & testicular autoimmunity, stiff person syndrome (SPS), systemic lupus erythematosus (SLE), subacute bacterial endocarditis (SBE), Susac's syndrome, sympathetic ophthalmia (SO), Takayasu's arteritis, temporal arteritis/Giant cell arteritis, thrombocytopenic purpura, thrombotic thrombocytopenic purpura (TTP), Tolosa-Hunt syndrome (THS), transverse myelitis, type 1 diabetes, ulcerative colitis (UC), undifferentiated connective tissue disease (UCTD), uveitis, vasculitis, vitiligo, Vogt-Koyanagi-Harada disease; and Wegener's disease. In preferred embodiments, the autoimmune disorder is not IgG4-related disease. In preferred embodiments, the autoimmune disorder is AAV (e.g. relapsed or refractory AAV), SLE (e.g. relapsed or refractory SLE), or rheumatoid arthritis (e.g. relapsed or refractory rheumatoid arthritis). In particularly preferred embodiments, the autoimmune disorder is AAV (e.g. relapsed or refractory AAV) or rheumatoid arthritis (e.g. relapsed or refractory rheumatoid arthritis). Autoimmune disorders amenable to treatment with CD7-CD19 cCAR of the invention include, without limitation, achalasia, Addison's disease, acute inflammatory demyelinating polyneuropathy—AIDP, adult Still's disease, agammaglobulinemia, alopecia areata, amyloidosis, ankylosing spondylitis, anti-GBM/anti-TBM nephritis, anti-PAD4-activating rheumatoid arthritis, antiphospholipid syndrome, asthma, atopic dermatitis, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune thrombocytopenia, autoimmune urticarial, axonal & neuronal neuropathy (AMAN), Balo disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss Syndrome (CSS) or eosinophilic granulomatosis (EGPA), cicatricial pemphigoid, Cogan's syndrome, cold agglutinin disease, congenital heart block, coxsackie myocarditis, CREST syndrome, Crohn's disease, dermatitis, dermatitis herpetiformis, dermatomyositis, Devic's disease (neuromyelitis optica), diabetes mellitus, discoid lupus, Dressier's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, Goodpasture's syndrome, granulomatosis with polyangiitis, Graves' disease, Guillain-Barre syndrome, Hashimoto's disease, Hashimoto's thyroiditis, autoimmune hemolytic anemia, Henoch-Schonlein purpura (HSP), herpes gestationis or pemphigoid gestationis (PG), Hidradenitis Suppurativa (HS) (Acne Inversa), hypogammalglobulinemia, idiopathic membranous nephropathy, idiopathic thrombocytopenic purpura, IgA nephropathy, IgG4-related disease, IgG4-related sclerosing disease, IgG neuropathy, IgM polyneuropathy, immune thrombocytopenic purpura (ITP), inclusion body myositis (IBM), inflammatory bowel disease (IBD), interstitial cystitis (IC), juvenile arthritis, juvenile diabetes (type 1 diabetes), juvenile myositis (JM), Kawasaki disease, Lambert-Eaton syndrome, leukocytoclastic vasculitis, Lichen planus, Lichen sclerosus, ligneous conjunctivitis, linear IgA disease (LAD), lupus, lyme disease chronic, membranous nephropathy, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, pemphigus vulgaris, pemphigus foliaceus, peripheral neuropathy, perivenous encephalomyelitis, pernicious anemia (PA), POEMS syndrome, polyarteritis nodosa, polyglandular syndromes types I, II, and III, polymyalgia rheumatic, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's syndrome, reactive Arthritis, reflex sympathetic dystrophy, relapsing polychondritis, restless legs syndrome (RLS), retroperitoneal fibrosis, rheumatic fever, rheumatoid arthritis, juvenile rheumatoid arthritis, sarcoidosis, Schmidt syndrome, scleritis, scleroderma, sensitized/preformed antibodies in solid organ transplant, Sjogren's syndrome, sperm & testicular autoimmunity, stiff person syndrome (SPS), systemic lupus erythematosus (SLE), subacute bacterial endocarditis (SBE), Susac's syndrome, sympathetic ophthalmia (SO), Takayasu's arteritis, temporal arteritis/Giant cell arteritis, thrombocytopenic purpura, thrombotic thrombocytopenic purpura (TTP), Tolosa-Hunt syndrome (THS), transverse myelitis, type 1 diabetes, ulcerative colitis (UC), undifferentiated connective tissue disease (UCTD), uveitis, vasculitis, vitiligo, Vogt-Koyanagi-Harada disease; and Wegener's disease. In preferred embodiments, the autoimmune disorder is not IgG4-related disease. In preferred embodiments, the autoimmune disorder is AAV (e.g. relapsed or refractory AAV), SLE (e.g. relapsed or refractory SLE), or rheumatoid arthritis (e.g. relapsed or refractory rheumatoid arthritis). In particularly preferred embodiments, the autoimmune disorder is AAV (e.g. relapsed or refractory AAV) or rheumatoid arthritis (e.g. relapsed or refractory rheumatoid arthritis). In the further embodiment, CD7-CD19 cCAR targeting CD7 positive T cells and CD19 positive T cells.

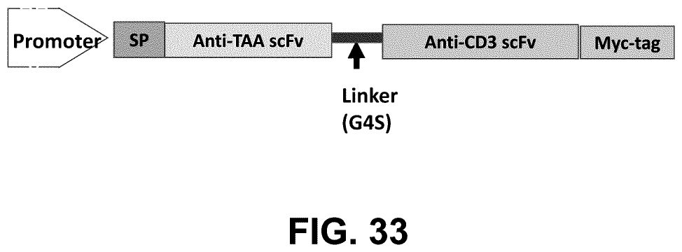

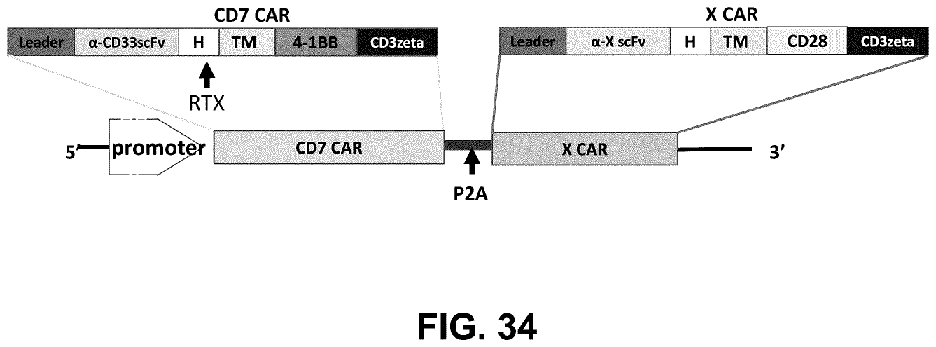

Bispecific antibody T cell engager (also called BiTE) is an engineered fusion protein consisting of two single chain variable fragments (scFvs) that are fused to contain bi-scFv functional domains wherein one target TAA (tumor associated antigen) expressed on tumor cells and the other targets CD3 chains on T cells. BiTE is capable of redirecting T cells to tumor cells expressing specific TAA, leading to tumor cell lysis (FIG. 33).

Both bispecific T cell engager and CAR T cells are widely used in targeting tumor cells and considered as revolutionized cancer treatments. However, some patients are still relapse and this may be due to the loss of targeted antigens on tumor cells (also called antigen escapes) or the short lifespan of bispecific antibodies after infusion. Additionally bispecific antibody T engager manufacturing is costly and time-consuming.

In one embodiment, the disclosure provides bispecific T cell engager used in targeting tumor cells or auto-reactive cells. Methods for bispecific T cell engager are as known to one ordinary skill in the art (Blanco et al., J Immunol 2003; 171:1070-1077; Compte et al., Cancer Gene Therapy (2007) 14, 380-388)

In one embodiment, an engineered cell having a CAR-BiTE construct expressing at least one distinct chimeric antigen receptor polypeptide (CAR) and a secreted bispecific T cell engager. In one embodiment, an engineered cell having at least two distinct chimeric antigen receptor polypeptides (CARs) and a secreted bispecific T cell engager.

In preferred embodiments, CAR T or NK cells are generated to secrete a soluble bispecific antibody T cell engager. CAR T cells are able to kill targeted cells and can also continuously produce bispecific antibody T cell engager. CAR T cells and bispecific work independently and each can launch strongly immune responses against targeted cells. In a further embodiment, the target cells can be auto-reactive cells or cancer cells.

CAR-BiTE construct is a complete CAR fused to a BiTE by a self-cleaving peptide, enabling independent expression. Expression of activated T cells transduced with CAR-BiTE lentiviral or retroviral vector assayed by flow cytometry (FACS) can reveal CAR expression, and the secreted BiTE can be detected by ELISA or Western blot assays.

CAR-BiTE construct can be a 2-unit CAR (also called compound CAR or cCAR) composed of a complete CAR fused to another complete CAR by a self-cleaving peptide, enabling independent expression of both CAR receptors separately on the T-cell surface. A 2-unit CAR is also fused to a secreted BiTE by another self-cleaving peptide.

Creation of compound CARs bearing different CAR units can be quiet challenging: (1) CAR-CAR interactions might have a deleterious effect and an appropriate CAR design is a key to offset this effect; (2) a compound CAR in a single construct could increase the length of the expression cassette, which may cause the reduction of the viral titer and level of protein expression; (3) an appropriate design to include various CAR body elements particularly to select a strategy to express multiple CARs in a single vector is required; (4) The hinge region in the CAR needs to is designed so that interaction of the hinge region between each CAR unit is avoided preferably; (5) two or more units of CARs expressing in a cell need to avoid CAR-CAR interaction. Applicants herein provide a novel and surprising CAR compositions and methods to overcome these hurdles.

In some embodiments, a chimeric antigen receptor (CAR) comprises signal domain (also called leader sequence), antigen recognition domain, a hinge domain, a transmembrane domain, and one or more co-stimulatory signaling domains and CD3 zeta signaling domain.

In one embodiment, CD7-negative T cells or NK cells can comprise a CAR capable of binding to different antigens present on a target cells.

In one embodiment, CD7-negative T cells or NK cells can comprise multiple CARs capable of binding to different antigens present on a target cells.

In one embodiment, CD7-negative T cells or NK cells can comprise a CAR and at least one of recombinant IL-15, IL-15RA, IL-15sushi, IL-15/IL-15RA, IL15-RA/IL-15, IL-15/IL-15sushi, IL15sushi/IL-15, functional fragment thereof, or combination thereof.

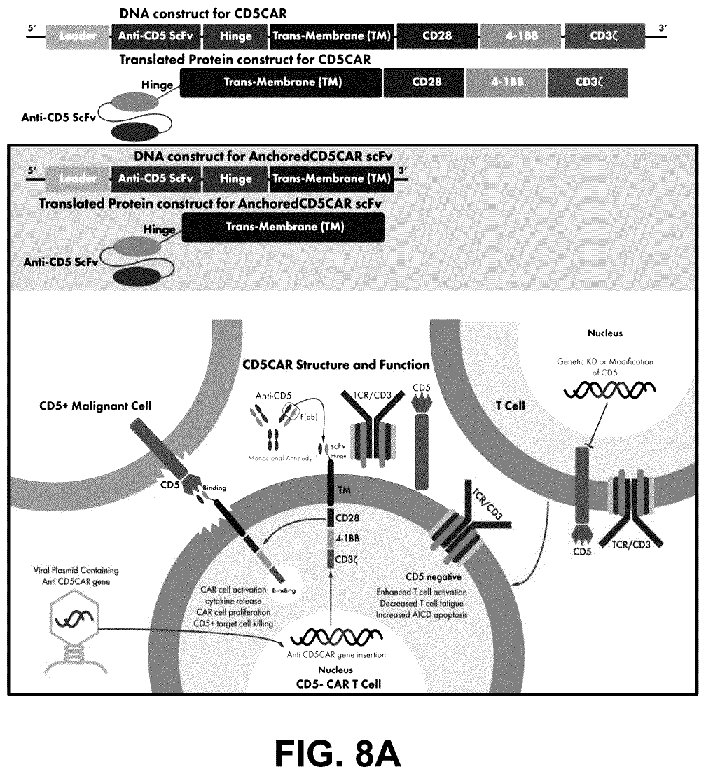

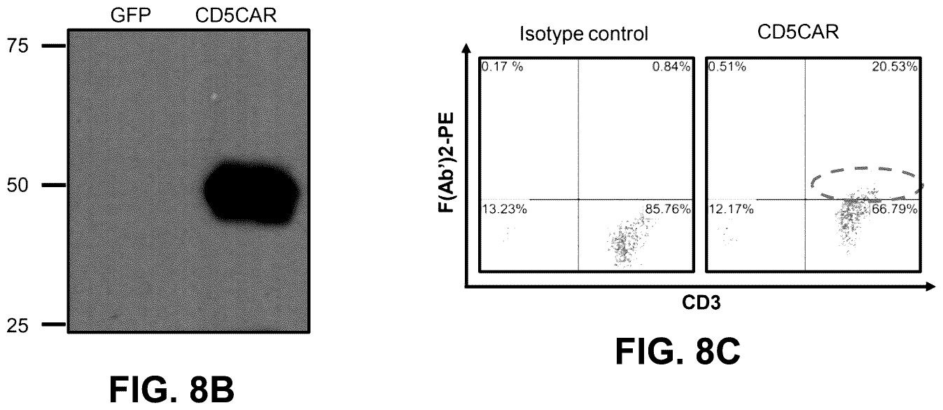

In another embodiment, the present disclosure provides a compound CAR (cCAR) (FIG. 8) containing CD7 CAR unit and the other CAR unit having the antigen recognition domain selecting from a group of NY-ESO-1, alpha fetoprotein (AFP), glypican-3 (GPC3), BCMA, BAFF-R, BCMA, TACI, LeY, CD5, CD7, CD2, CD3, CD4, CD45, CD13, CD14, CD15, CD19, CLL-1, CD20, CD22, CD33, CD41, CD61, CD64, CD68, CD117, CD123, CD138, CD267, CD269, CD38, Flt3 receptor, ROR1, PSMA, MAGE A3, Glycolipid, Claudin 18.2, F77, GD-2, MART-1, MUC1, MUC2, MUC3, MUC4, MUC5, MMG49 epitope, WTI, CEA, HER-2/neu, MAGE-3, MAGE-4, MAGE-5, MAGE-6, CA 19-9, CA 72-4, NY-ESO, FAP, ErbB, c-Met, MART-1, CD30, EGFRvIII, immunoglobin kappa and lambda, CD38 and CS1. The CAR target antigens can also include viral or fungal antigens, such as E6 and E7 from the human papillomavirus (HPV) or EBV (Epstein Barr virus) antigens. In further embodiment, CD7-negative T cells or NK cells are generated by a natural selection approach with transduction of a cCAR containing a CD7 CAR construct.

In another embodiment, the present disclosure provides a CD7-negative T cells or NK cells having at least one of recombinant IL-15, IL-15RA, IL-15sushi, IL-15/IL-15RA, IL15-RA/IL-15, IL-15/IL-15sushi, IL15sushi/IL-15, functional fragment thereof, or combination thereof; and at least one distinct CAR polypeptide wherein the antigen recognition domain includes NY-ESO-1, alpha fetoprotein (AFP), glypican-3 (GPC3), BCMA, BAFF-R, BCMA, TACI, LeY, CD5, CD7, CD2, CD3, CD4, CD45, CD13, CD14, CD15, CD19, CD20, CD22, CD33, CD41, CD61, CD64, CD68, CD117, CD123, CD138, CD267, CD269, CD38, Flt3 receptor, ROR1, PSMA, MAGE A3, Glycolipid, Claudin 18.2, F77, GD-2, WT1, CEA, HER-2/neu, MAGE-3, MAGE-4, MAGE-5, MAGE-6, CA 19-9, CA 72-4, NY-ESO, FAP, ErbB, c-Met, MART-1, CD30, CLL-1, MART-1, MUC1, MUC2, MUC3, MUC4, MUC5, MMG49 epitope, EGFRvIII, immunoglobin kappa and lambda, CD38 and CS1. The target antigens can also include viral or fungal antigens, such as E6 and E7 from the human papillomavirus (HPV) or EBV (Epstein Barr virus) antigens.

In further embodiment, the antigen recognition polypeptides (scFv) and corresponding polynucleotides for CD2, CD3, CD5, CD7, and CD52 as well as IL-15/IL-15sushi and IL-15sushi are described in more detail publications in PCT Application NO. PCT/US2016/39306 and PCT/US2016/019953, the contents of which are incorporated herein by reference.

In some embodiments, CD7-negative T or NK cells can be obtained from peripheral blood and cord blood or differentiated cells from induced pluripotent stem cells (also known as iPS cells or iPSCs) or stem cells.

### Examples

### Targeting of CD4 Expressing T Cells and T-Cell Malignancies Using CD4⁻ Specific Chimeric Antigen Receptor (CAR)-Engineered T-Cells

### Materials and Methods

### Blood Donors, Primary Tumor Cells and Cell Lines

Human lymphoma cells and peripheral blood mononuclear cells were obtained from residual samples. Umbilical cord blood cells were obtained from donors at Stony Brook University Hospital. SP53 and KARPAS 299 lymphoma cell lines were obtained from ATCC (Manassas, VA).

### Lentivirus Production and Transduction of T-Cells

To produce viral supernatant, 293FT cells were co-transfected with pMD2G and pSPAX viral packaging plasmids, and with either pRSC.CD4.3G or GFP Lentiviral vector, using Lipofectamine 2000 (Life Technologies, Carlsbad, CA) per manufacturer's protocol. Prior to lentiviral transduction, umbilical cord or peripheral blood mononuclear buffy coat cells were activated for two days in the presence of 300 IU/mL IL-2 and 1 μg/mL anti-human CD3 (Miltenyi Biotec, Germany).

### T-Cell Expansion

CAR-transduced T-cells were expanded for 7 days in T-cell media (50% AIM-V, 40% RPMI 1640, 10% FBS and 1× penicillin/streptomycin; all Gibco) supplemented with IL-2. Cells were counted every day and media was added every 2-3 days in order to maintain T-cell counts below 2×10⁶ cells/mL.

### CAR Immunophenotype

For the analysis of CAR cell immunophenotype, following 7 days of expansion, CD4CAR T-cells and GFP control cells were stained with CD45RO, CD45RA, CD62L and CD8 (all from BD Biosciences) for flow cytometry analysis.

### Co-Culture Target Cell Ablation Assays

CD4CAR T-cells or GFP T-cells (control) were incubated with target cells at ratios of 2:1, 5:1 and 10:1 (200,000, 500,000 or 1 million effector cells to 100,000 target cells, respectively) in 1 mL T-cell culture media, without IL-2 for 24h. Target cells were Cd4 expressing KARPAS 299 cells (anaplastic large T-cell lymphoma expressing CD4), leukemia cells from a patient with CD4⁺ T-cell leukemia—Sezary syndrome—and from a patient with CD4⁺ PTCL lymphoma. As a negative control, CD4CAR T-cells and GFP T-cells were also incubated with SP53 (mantle cell lymphoma) cells, which do not express CD4, in the same ratios in 1 mL separate reactions. After 24-hours of co-culture, cells were stained with mouse anti-human CD8 and CD4 antibodies. In the experiments with SP53 cells, SP53 cells were labeled with CMTMR (Life Technologies) prior to co-culture with T-cells, and T-cells were labeled with mouse anti-human CD3 (PerCp) after co-culture incubation.

### In Vivo Mouse Xenogenic Model

NSG mice (NOD.Cg-Prkdc^{scid} Il2rg^{tm1Wjl}/SzJ) from the Jackson Laboratory were used under a Stony Brook University IACUC-approved protocol. Mice were all male and between 8 and 12 weeks old. Three sets of in vivo experiments were performed with no blinding. For each set, 10 mice were irradiated with a sublethal (2.5 Gy) dose of gamma irradiation and assigned randomly to the treatment or control group. 24h later, mice were given one intradermal injection of 0.5×10⁶ or 1.0×10⁶ KARPAS 299 cells in order to form a measurable subcutaneous tumor within 7 days. Tumor size area was measured every other day. In the first set, three days after the injection of 1 million KARPAS 299 cells, 2 million CD4CAR T (5 mice) or 2 million GFP T control cells (5 mice) were administered to the mice intravenously (by tail vein injection). A second dose of 8 million cells was injected intravenously on Day 22. In the second set, 10 NSG mice was irradiated and injected with 0.5×10⁶ KARPAS 299 cells. On day 2, mice were injected intravenously with one course of 8 million CD4CAR T-cells (5 mice) and 8 million GFP T control cells (5 mice). A second dose of 5.5 million cells was injected intravenously on Day 10. In the third set, 10 NSG mice were irradiated and injected with 0.5×10⁶ KARPAS 299 cells. On day 1, mice were intravenously injected with 2.5×10⁶ CD4CAR T-cells or with GFP T control cells (5 mice per group). Intravenous injections were repeated every 5 days for a total of four courses.

### Results

### Characterization of CD4CAR

Cord Blood-derived CD4CAR T cells are highly enriched for CD8+ T cells and most of them bear a central memory T cell like immunophenotype.

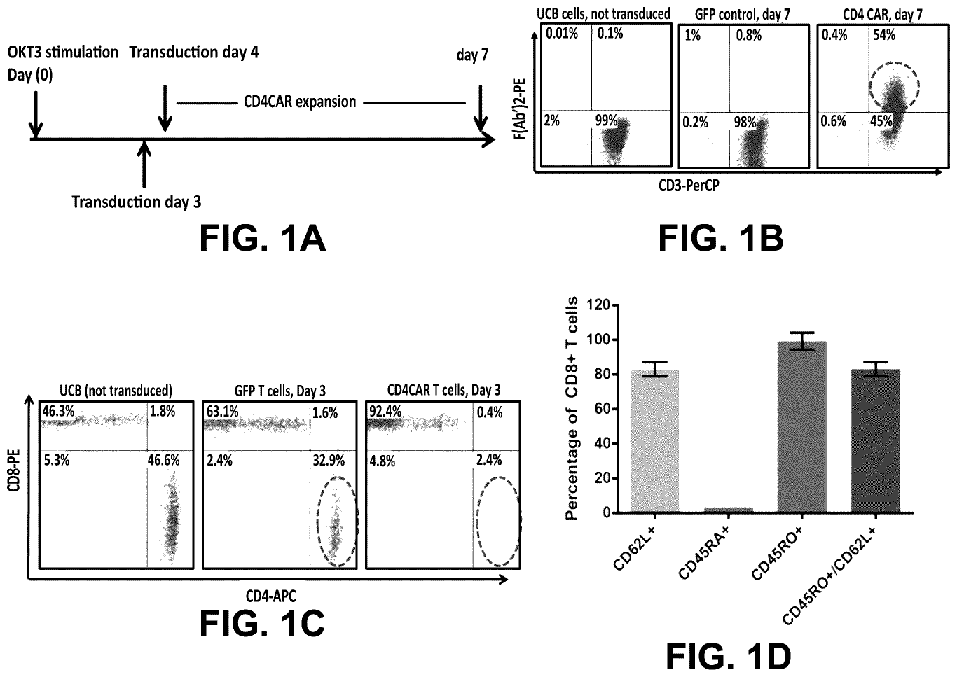

Human umbilical cord blood (CB) is an alternate source for allogeneic T cell therapy. Human CB buffy coat cells were activated and transduced with either CD4CAR or control (GFP) lentiviruses. After transduction, CD4CAR T cells and GFP T cells were expanded for 7 days, with a 20-fold increase in cell count observed for both CD4CAR and GFP T cells. At day 7, cells were analyzed by flow cytometry for T-cell subsets (FIG. 1A). Flow cytometry analysis showed that ˜54% of T-cells expressed the CD4CAR (FIG. 1B). Furthermore, we analyzed the CD4 and CD8 subsets during the course of T expansion following CD4CAR transduction. Consistent with previous findings, a small subset of CD8 cells was induced to express CD4 during T-cell activation with anti-CD3 and costimulatory molecules (FIG. 1C). As expected, the CD4⁺ T subset was almost completely depleted within 3 or 4 days following CD4CAR transduction as compared to GFP control, in which ˜33% of cells remained CD4⁺ (FIG. 1C). These data indicate that CD4CAR T cells exhibit potent anti-CD4 activity in vitro during T cell expansion.

We also evaluated the immunophenotype of CD4CAR T-cells at the end of each culture. Following stimulation, naïve T-cells lose CD45RA and gain CD45RO in order to become central memory T-cells. Flow cytometry analysis from 3 representative experiments showed that 96% of the expanded T-cells were CD45RO+, ˜83% were CD62L+ and −80% were CD8+CD45RO+CD62L+ whereas fewer than 4% were CD45RA+(FIG. 1D). The CD8+CD45RO+CD62L+ immunophenotype is consistent with the acquisition of a central memory-like phenotype, and low CD45RA+ expression confirms loss of naïve T-cell status.

CD4CAR T-cells derived from Cord Blood specifically kill CD4⁻ expressing leukemia/lymphoma including anaplastic large cell lymphoma, Sezary syndrome and unclassified PTCL lymphoma.

CD4CAR T-cells highly enriched for CD8+ T-cells were generated (FIG. 1C). The cells were then tested in vitro for anti-leukemic functions using the KARPAS 299 cell line. The KARPAS 299 cell line was initially established from the peripheral blood of a patient with anaplastic large T-cell lymphoma expressing CD4. Cytogenetic analysis has previously shown that KARPAS 299 cells have many cytogenetic abnormalities. During co-culture experiments, CD4CAR cells exhibited profound leukemic cell killing abilities (FIG. 2A). First, CB-derived CD4CAR T-cells were tested for their ability to ablate CD4 positive KARPAS 299 cells. Indeed, at 24h incubation and at a low E:T (effector: target) ratio of 2:1, CD4CAR cells successfully eliminated KARPAS 299 cells. As a control, the CD4CAR T-cells were also tested for their ability to ablate CD4 negative lymphoma cells. SP53 mantle cell lymphoma cell line is a human B-cell lymphoma cell line that does not express CD4. Flow cytometry analysis showed that CD4CAR T-cells were unable to lyse or eliminate SP53 mantle cell lymphoma (FIG. 2D).

Studies were also conducted using patient samples. Patient 1 presented with an aggressive form of CD4⁺ T cell leukemia, Sezary syndrome, which did not respond to standard chemotherapy. Patient 2 presented with an unspecified CD4⁺ PTCL lymphoma. Flow cytometry analysis of both patient samples revealed strong and uniform CD4 expression, with almost all leukemic cells expressing CD4 (FIG. 2B and FIG. 2C). As visualized by flow cytometry analysis, co-culture of patient samples with CD4CAR for 24 hours resulted in rapid and definitive ablation of CD4+ malignancies, with, once again, approximately 98% ablation observed for both Sezary syndrome and PTCL co-cultures, consistent with the ablation of KARPAS previously shown (FIG. 2B and FIG. 2C). Therefore, we show that, in a co-culture assay, CD4CAR T-cells efficiently eliminate two different types of aggressive CD4⁺ lymphoma/leukemia cells directly from patient samples even at the low E:T ratio of 2:1 (FIG. 2B and FIG. 2C). These data support that CD4 is a promising therapeutic target for CD4 positive T-cell leukemias and lymphomas, analogous to the role of CD19 in the targeting of B-cell malignancies via anti-CD19 CAR. Therefore, our patient sample and CD4CAR co-culture assay extends the notion of using CAR to target CD4 positive malignancies.

CD4CAR T-Cells Derived from PBMCs Specifically Kill CD4⁻ Expressing the Tumor Cell Line.

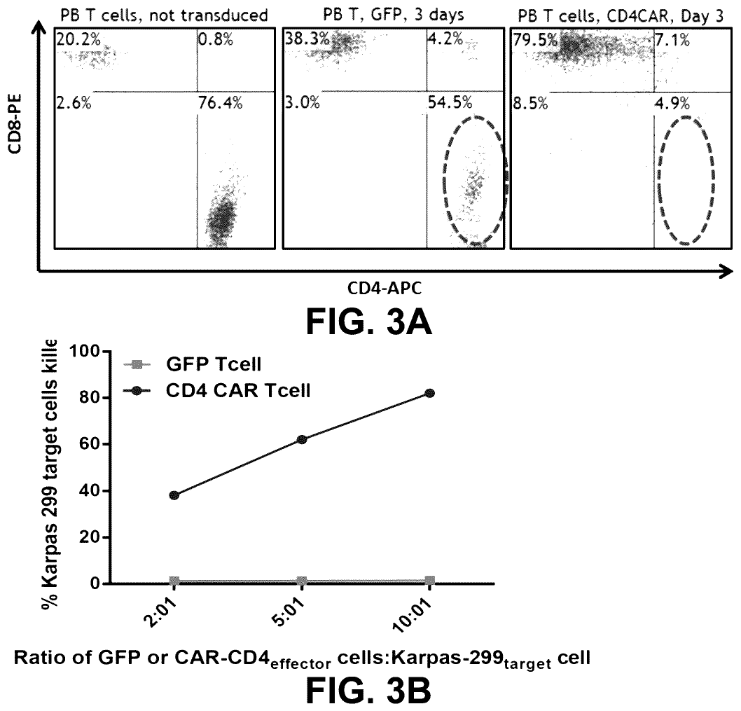

Since autologous adoptive CAR-T therapy is commonly used in the clinic, we then tested CD4CAR T-cells derived from PBMCs (peripheral blood mononuclear cells). PBMCs were activated and transduced with CD4CAR lentiviruses. The CD4 and CD8 sets were monitored by flow cytometry during cell expansion and compared to that of cells transduced with control GFP. The PBMCs derived CD4CAR T-cells were highly enriched for CD8+ T-cells as observed with CD4CAR T-cells derived from CB (FIG. 3A), indicative of the role of CD4CAR in the depletion of CD4³⁰. PBMC derived CD4CAR cells were subsequently tested in their ability to ablate CD4 positive leukemia/lymphoma cells, using the KARPAS 299 cell line. The ablation assay involved the co-culture of CD4CAR T-cells or GFP T-cells, with KARPAS 299 cells, and with the SP53 mantle cell lymphoma cell line negative control. Reactions were stopped after 24-hours: dead cells were stained with γ-AAD (γ-aminoactinomycin D) and live cells were analyzed by flow cytometry. KARPAS 299 cells incubated with CD4CAR T-cells overnight were eliminated at a rate of 38%, 62%, and 85%, at E:T ratios of 2:1, 5:1, and 10:1, respectively (FIG. 3B). Combined, these data demonstrate a strong dose-response relationship. When target cells were incubated with GFP control T-cells, no killing of KARPAS 299 cells was observed. These results demonstrate that CD4CAR T-cell ablation is specific to CD4⁺ targeting.

### CD4CAR T Cells Exhibit Significant Anti-Tumor Activity In Vivo.

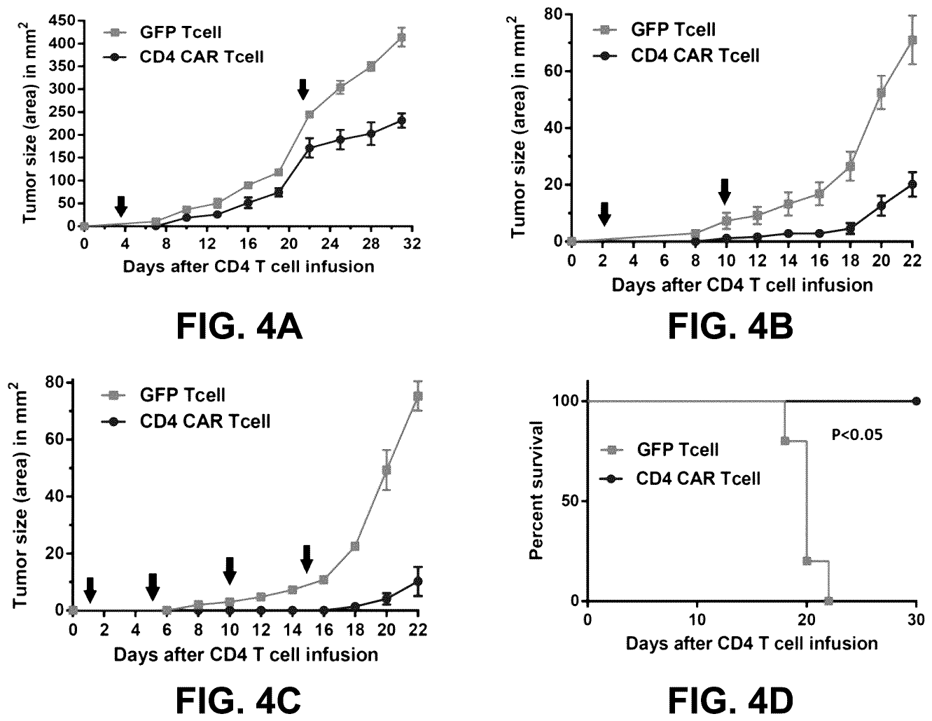

In order to evaluate in vivo anti-tumor activities, we developed a xenogeneic mouse model using the KARPAS 299 cell line. Multiple different settings were used to test CD4CAR T-cell efficacy in vivo. We first tested ability of the CD4CAR T-cells to delay the appearance of leukemia in the NSG mice with a single low dose. Prior to the injection, modified T-cells displayed ˜40 to 50% of cells expressing CD4CAR as demonstrated by flow cytometry analysis. Mice received intradermal injections of KARPAS 299 cells and then a low dose (2 million) of single systemic injection (intravenous administration) of CD4CAR T-cells was given. A single low dose of systemic CD4CAR T-cells administration to leukemia-bearing mice caused only transient regression or delayed the appearance of leukemic mass (FIG. 4A). When leukemia growth started to accelerate, an additional course of administration of 8×10⁶ CD4CAR T-cells remarkably arrested the leukemic growth (FIG. 4A).

To further test the efficacy of CD4CAR anti-leukemia activity, we administered two courses of relatively large doses of CD4CAR T-cells. Similarly, two injections totaling 13.5×10⁶ CD4CAR T-cells caused more pronounced leukemia growth arrest as compared to a lower CD4CAR dose but eventually the leukemic cell population recovered (FIG. 4B). Finally, we investigated the efficacy of multiple course injections of a low dose of CD4CAR T cells (each 2.5×10⁶ cells). We treated the mice bearing subcutaneous leukemia with repeat intravenous injections of CD4CAR T-cells, once every 4 or 5 days for total of 4 injections. After four courses of CD4CAR T-cell administration, one of four treated mice was tumor free and exhibited no toxic appearance. Multiple dose CD4CAR T-cell-treated mice displayed more significant anti-leukemic effect compared to single dose (FIG. 4C and FIG. 4A). Moreover, treatment with CD4CAR T-cells significantly prolonged the survival of mice bearing KARPAS 299 lymphoma as compared to treatment with the GFP-transduced control T-cells (FIG. 4D).

### Anti-CD4 Chimeric Antigen Receptor (CD4CAR) NK Cells Efficiently Target T-Cell Malignancies in Preclinical Models

### Methods Materials

### Primary Tumor Cells and Cell Lines

Human leukemia cells were obtained from residual samples on a protocol approved by the Institutional Review Board of Stony Brook University. Cord blood cells were also obtained under protocol from donors at Stony Brook University Hospital. Written, informed consent was obtained from all donors. Karpas 299, HL-60, CCRF-CEM, MOLT4 and NK-92 cell lines were obtained from ATCC (Manassas, VA). NK-92 cells were cultured in filtered NK cell media, defined as alpha-MEM without ribonucleosides and deoxyribonucleosides with 2 mM L-glutamine, 1.5 g/L sodium bicarbonate, 12.5% heat-inactivated horse serum, 12.5% heat-inactivated FBS, 1× Pen/Strep, 0.2% inositol, 0.02% folic acid, and 50 μM beta-mercaptoethanol, supplemented with IL-2 (300 IU/mL), unless otherwise specified. Karpas 299, CCRF-CEM, and MOLT4 cell lines were cultured in RPMI, 10% FBS, 1× Pen/Strep (Gibco, Waltham, MA, USA). HL-60 cells were cultured in IMDM, 10% FBS, 1× Pen/Strep (Gibco, Waltham, MA, USA).

### CAR Construct Generation

The CD4⁻ specific CAR (pRSC.SFFV.CD4.3G) was designed to contain an intracellular CD28 domain upstream of 4-1BB and CD3zeta domains, thereby making the construct a third-generation CAR.

### Lentivirus Production and Transduction

To produce viral supernatant, 293F T-cells were co-transfected with pMD2G and pSPAX viral packaging plasmids containing either pRSC.SFFV.CD4.3G or GFP lentiviral vector control, using Lipofectamine 2000 (Life Technologies, Carlsbad, CA) per manufacturer's protocol.

NK cells were cultured for a minimum of 2 days in the presence of 300 IU/mL IL-2 prior to transduction with viral supernatant. Transfection and transduction procedures are further described in Supplemental Data.

### CAR Detection on Transduced NK Cells

In order to determine CAR expression, NK cells were washed and suspended in FACs buffer (0.2% BSA in DPBS) 3 days after transduction. Normal goat IgG (Jackson Immunoresearch, West Grove, PA) was used to block nonspecific binding. Each NK cell sample was probed with Biotin-labeled polyclonal goat anti-mouse F(Ab′)² (1:250, Jackson Immunoresearch, West Grove, PA) for 30 minutes at 4° C. Cells were washed once, and resuspended in FACs buffer. Cells were then stained with PE-labeled streptavidin (1:250, Jackson Immuno Research, West Grove, PA) for 30 minutes at 4° C. Cells were washed with FACs buffer, and resuspended in 2% formalin. Flow cytometry was performed using a FACS Calibur instrument (Becton Dickinson, Franklin Lakes, NJ), and results were analyzed using Kaluza software (Beckman Coulter, Brea, CA).

### Co-Culture Assays

CD4CAR or vector control NK cells were incubated with CD4 expressing Karpas 299 cells (anaplastic large T-cell lymphoma), HL-60 cells (acute promyelocytic leukemia), CCRF-CEM cells (T-cell acute lymphoblastic leukemia: T-ALL), CD4⁺ T-cells isolated from human cord blood, or CD4 expressing primary human leukemic cells (adult Sézary syndrome and pediatric T-ALL) at ratios of 2:1 and 5:1 (200,000 and 500,000 effector cells to 100,000 target cells, respectively) in 1 mL of NK-cell culture media, without IL-2. After 24 hours of co-culture, remaining live cells were harvested and stained with mouse anti-human CD56 and CD4 antibodies, and were incubated at 4° C. for 30 minutes. CD56⁺ single positive denoted NK cells, and CD4⁺ single positive denoted target cells. All cells were washed with FACs buffer, suspended in 2% formalin, and analyzed by flow cytometry.

### Cytotoxicity Assay

CD4CAR or vector control NK cells were incubated with a 50:50 mix of on-target cells (CFSE-stained Karpas 299 cells and CMTMR-stained CCRF-CEM cells) and off-target CMTR-labelled MOLT4 cells at effector:target ratios of 1:1, 1:2, and 1:4 ratios in 1 mL of NK-cell culture media, without IL-2. After 24-hours, cells were stained with γ-AAD (BioLegend, San Diego, CA), washed with FACS buffer, and live γ-AAD negative cells were analyzed by flow cytometry.

### Xenogeneic Mouse Model

Male 12-week-old NSG mice (NOD.Cg-Prkdcsid Il2rgtm1Wjl/SzJ) were purchased from the Jackson Laboratory (Bar Harbor, ME) and used under a Stony Brook University IACUC-approved protocol. NSG mice were irradiated with a sublethal (2.5 Gy) dose of gamma irradiation. Twenty-four hours later, mice were intradermally injected with 0.5×10⁶ Karpas 299 cells that had been stably transduced to express luciferase, in order to cause a measurable subcutaneous tumor to form. On day 1, twenty-four hours following Karpas 299 cell injection, mice were intravenously injected via tail vein with 5×10⁶ CD4CAR NK cells or vector control NK cells (N=4 per group). Intravenous injections were repeated every 5 days for 6 courses total. Tumor size area was measured every other day. On days 7, 14, and 21 following Karpas 299 cell injection, mice were injected subcutaneously with 100 μL RediJect D-Luciferin (Perkin Elmer, Waltham, MA) and subjected to IVIS imaging (PerkinElmer, Waltham, MA). Images were analyzed using Caliper Life Sciences software (PerkinElmer, Waltham, MA).

### Statistics

Xenogeneic model sample sizes were estimated using 2-sample, 2-sided equality power analysis (90% power and <5% significance). Unpaired Student T tests were used to determine significance of tumor size area and light intensity. Survival curves were constructed using the Kaplan-Meier method and statistical analyses of survival was performed using a log-rank (Mantel-Cox) test with P<0.05 considered significant. Statistical analyses were performed using GraphPad Prism 6 software. Variance was determined to be similar between the treatment and control group prior to unpaired student-test.

### Results

### CD4CAR NK Cells Specifically Lyse CD4⁻ Expressing Tumor Cell Lines in Dose Dependent Manner.

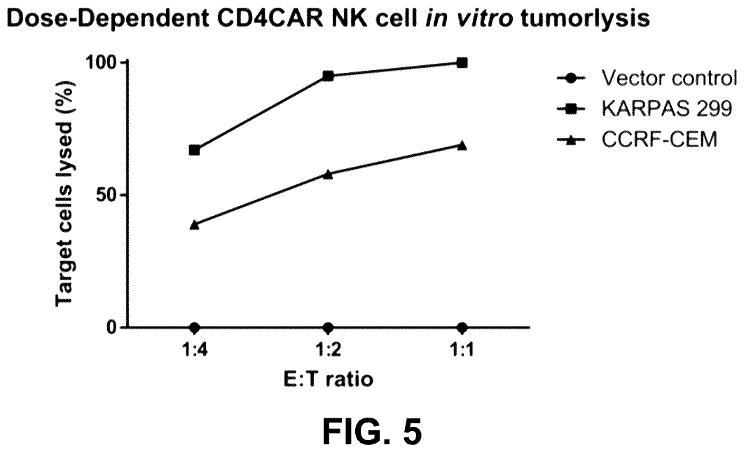

CD4CAR NK cells specifically lyse CD4⁺ Karpas 299 and CCRF-CEM leukemic cell lines in vitro in a dose-dependent manner at effector: target ratios of 1:4, 1:2, and 1:1 (FIG. 5). For each co-culture E:T ratio, CD4CAR NK effector cells or vector control NK effector cells were incubated with tumor cells that were comprised of equal numbers of on-target CD4⁺ cells, CFSE-stained Karpas 299 or CFSE-stained CCRF-CEM, and “off-target” CMTMR-stained CD4⁻, CD5⁺ MOLT4 acute lymphoblastic leukemia cells. The MOLT4 cells were included to account for variation in the starting cell numbers and for spontaneous target cell death. After 24 hours, live cells were analyzed by flow cytometry. Percent lysis of target cells was measured by comparing CD4⁺ target cell survival in CD4CAR NK co-culture to vector control NK co-culture. Karpas 299 cells were eliminated at rates of 67%, 95%, and 100%, at effector to target ratios of 1:4, 1:2, and 1:1, respectively (FIG. 5). And CCRF-CEM cells were eliminated at rates of 39%, 58%, and 69% respectively at the same E:T ratios (FIG. 5).

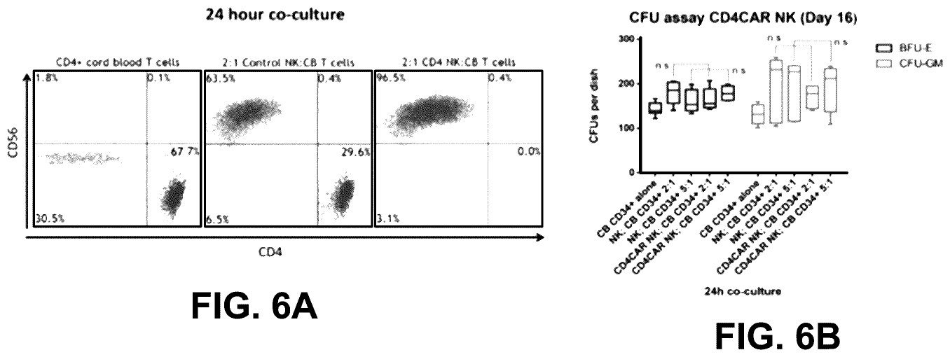

Additional co-culture studies were conducted using CD4⁺ T-cells isolated from cord blood. In these experiments, CD4CAR NK cells completely depleted CD4⁺ T-cells at an effector:target ratio of 2:1 after 24 hours of co-culture, with remaining cells 0.0% CD4⁺. As expected, after CD4⁺ cord blood cell co-culture with corresponding vector control NK cells (CD56⁺, CD4⁻), the CD4⁺ population remained largely intact (FIG. 6A), further confirming specific and robust CD4CAR NK-mediated depletion of CD4⁺ populations on healthy tissue.

### CD4CAR NK Cells do not Affect Stem Cell Output in Hematopoietic Compartment.

CFU (Colony-Forming-Unit) assay analysis revealed that CD4CAR NK cells did not significantly affect the CD34+ cord blood stem cell output of the hematopoietic compartment. Hematopoietic compartment output was assessed by the presence of erythroid progenitors and granulocyte/macrophage progenitors at Day 0, determined by number of erythroid burst-forming units (BFU-E) and number of granulocyte/monocyte colony-forming units (CFU-GM) at Day 16 (FIG. 6B). This finding is consistent with specific targeting of CD4, a mature T-cell marker, with limited impact on hematopoietic stem cells and early progenitors, and no evidence of lineage skewing, a measure of therapeutic safety.

### CD4CAR NK Cells Exhibit Significant Anti-Tumor Activity In Vivo

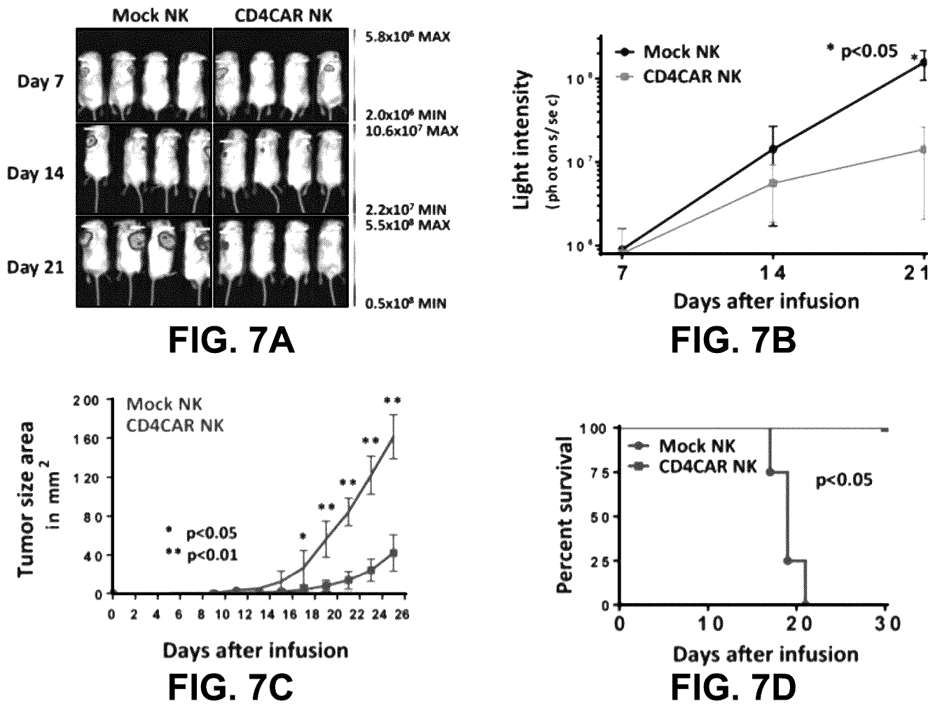

In order to evaluate the in vivo anti-tumor activity of CD4CAR NK cells, we developed a xenogeneic mouse model using NSG mice sublethally irradiated and intradermally injected with luciferase-expressing Karpas 299 cells to induce measurable tumor formation. On day 1, 24 hours following Karpas 299 cell injection, and every 5 days afterwards for a total of 6 courses, mice were intravenously injected with 5×10⁶ CD4CAR NK cells or vector control NK control cells per administration. On days 7, 14, and 21, mice were injected subcutaneously with RediJect D-Luciferin and underwent IVIS imaging to measure tumor burden (FIG. 7A). Average light intensity measured for the CD4CAR NK injected mice was compared to that of vector control NK injected mice (FIG. 7B). By Day 21, the CD4CAR NK injected mice had significantly less light intensity and therefore thus less tumor burden compared to vector control (p<0.01). On day 1, and every other day afterwards, tumor size area was measured and the average tumor size between the two groups was compared (FIG. 7C). Unpaired student T test analysis revealed that the average tumor size of CD4CAR NK injected mice was significantly smaller than that of vector control NK injected mice starting on day 17 (p<0.05) and continuing on days 19-25 (p<0.01). Next, we compared mouse survival across the two groups (FIG. 7D). All of the CD4CAR NK injected mice survived past day 30. However, percent survival of vector control NK injected mice started to decrease on day 17 with no survival by day 23. In summary, these in vivo data indicate that CD4CAR NK cells significantly reduce tumor burden and prolong survival in Karpas 299-injected NSG mice.

### Anti-CD5 Chimeric Antigen Receptor (CD5CAR) T Cells Efficiently Target CD5 Positive Hematologic Malignancies

An example of two mechanisms including naturally internalization and knockout that results in downregulation of the antibody target surface protein in immune cells was elucidated below. CD5 CAR is used as an example. CD5 anchor is generated to show the internalization by an anti-CD5 antibody on the surface of T cells. The DNA construct of the anchored CD5 scFv antibody is the same as the CD5CAR construct without the intracellular signaling domains, as is the translated protein product for CD5 scFv antibody anchoring on the T cell surface (FIG. 8)

### Generation of the Third Generation of CD5CAR

The construct for CD5CAR, as well as CD5 anchor scFv antibody were designed to test the function and mechanism of CD5CAR T-cells in terms of both the antibody mediated internalization and lysis of CD5 expressing cells and the ability of CD5CAR T-cells to down-regulate CD5 expression within their own CD5CAR T-cell population (FIG. 8A). To confirm the CD5CAR construct, the generated CD5CAR lentiviruses were transduced into HEK293 cells. After 48h treatment with CD5CAR or GFP-lentiviruses, the expression of CD5CAR in HEK293 cells was verified by Western blot analysis using CD3zeta antibody, which recognize C-terminal region of CD5CAR protein (FIG. 8B). The resulting band was the predicted size of CD5CAR protein in CD5CAR transduced HEK293 cells, but GFP transduced HEK293 cells did not exhibit any specific band by Western blot analysis. In order to evaluate the function of CD5CAR protein for future experiments, CD5CAR lentiviruses were transduced into activated human T-cells. The expression of CD5CAR on surface of T-cells was evaluated by flow cytometry analysis using goat anti-mouse F(ab′) antibody, which recognizes scFv region of CD5CAR protein. Flow cytometric analysis showed that about 20% of CD5CAR expression was observed on CD5CAR transduced T-cells compared to isotype control (FIG. 8C). These results indicated that we successfully generated CD5CAR expression T cell for following experiments. The DNA construct of the anchored CD5 scFv antibody is the same as the CD5CAR construct without the intracellular signaling domains, as is the translated protein product for anchored CD5 scFv antibody

### Down-Regulation of CD5 Expression for CAR Therapy

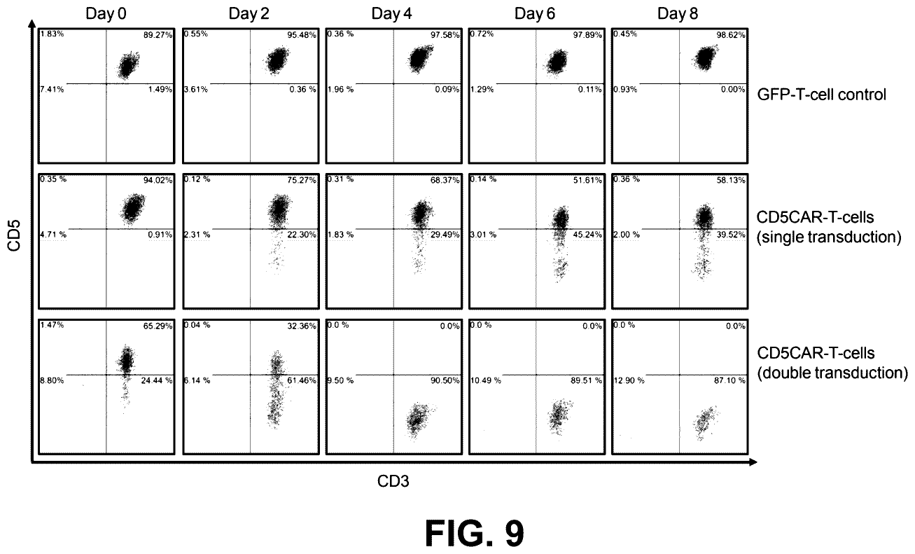

Prior to CD5CAR T-cell co-culture and animal assays, the expression of CD5 on the surface of CD5CAR T-cells is naturally down regulated to avoid self-killing within the CD5CAR T population. The downregulation of CD5 will prevent the self-killing of CAR T-cells within the CAR T-cell population, and the down-regulation of CD5 is associated with an increased killing ability of T-cells. A CAR that is produced within T-cells that has no CD5 expression could be a super-functional CAR, no matter the construct of the CAR itself. The steps for generation of CD5 CAR T-cells and the comparison of CD5 down-regulation using single or double transduction of CD5 CAR lentiviuses are shown in (FIG. 9). The single transduced CD5CAR T-cells with un-concentrated lent-CD5 CAR viruses did not show complete downregulation of CD5 protein from cell surface by day 8, with a maximum CD5 negative population up to 46% on day 6 (FIG. 9). In the double transduced population, about 90% of transduced T-cells became CD5 negative on day 4-day incubation. In contrast, the GFP T-cell control maintains a CD5+, CD3+ double positive population above 95% from day 2 through day 8 (FIG. 9).

### CD5CAR T-Cells Effectively Lyse T-Cell ALL Cell Lines.

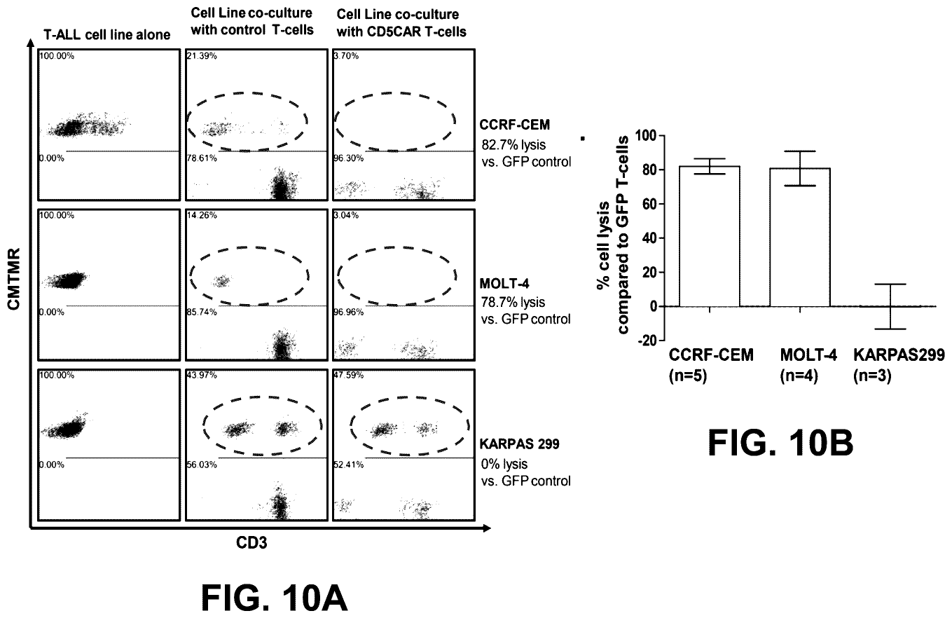

The killing ability of CD5CAR T-cells was first tested against T-cell ALL established cell lines CCRF-CEM and MOLT-4, and an anaplastic large cell leukemic cell line KARPAS 299 as shown in FIGS. 10A and 10B. An avid killing ability was seen for the two CD5+ cell lines when compared to GFP control, with target cell lysis above 75% for both lines. 0% lysis was observed in an anaplastic large cell line KARPAS 299, which is negative for CD5.

CD5CAR T-Cells Effectively Lyse T-Cell ALL Cells from Human Samples.

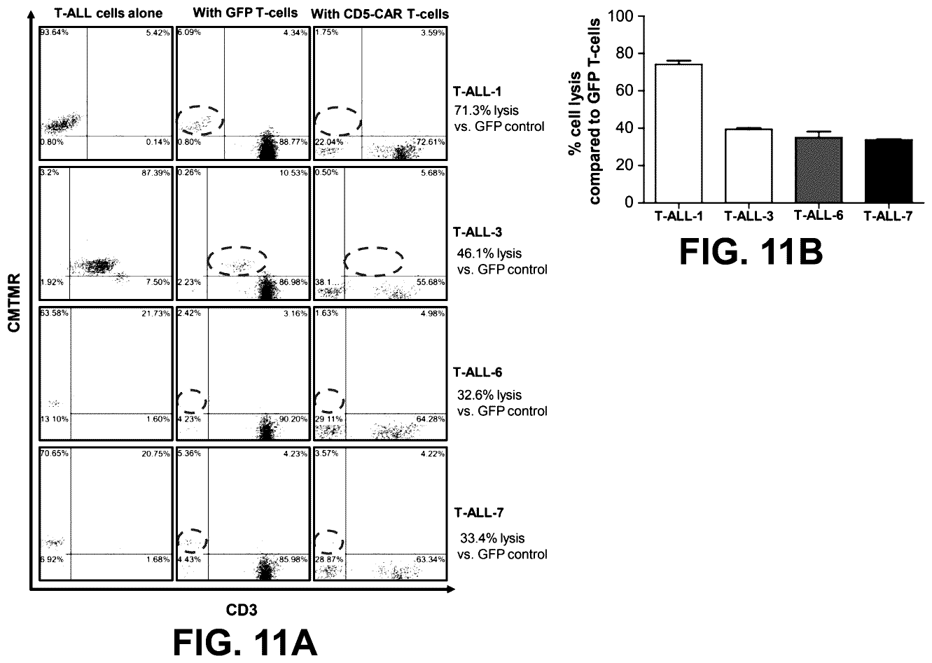

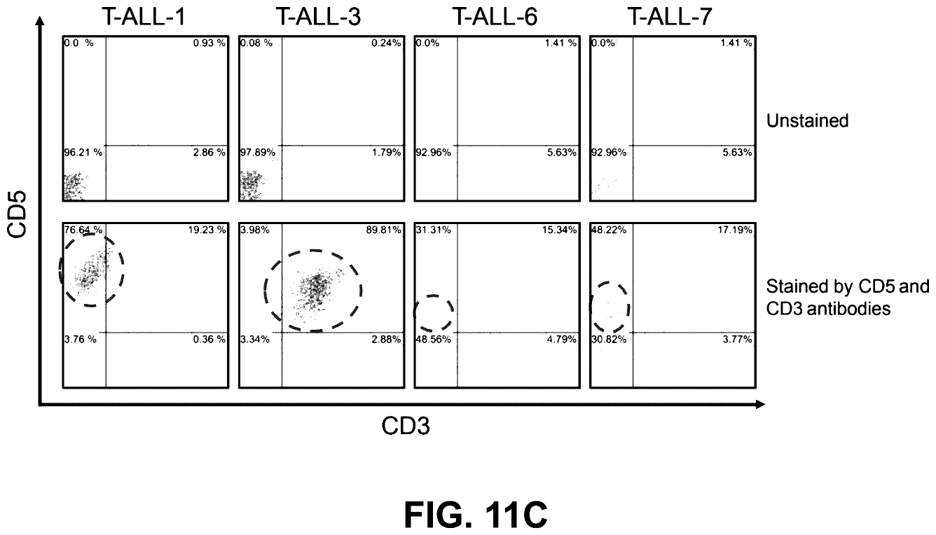

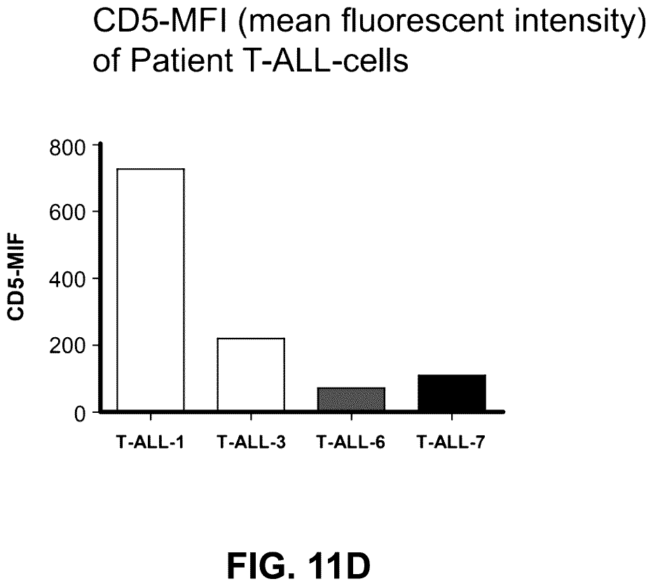

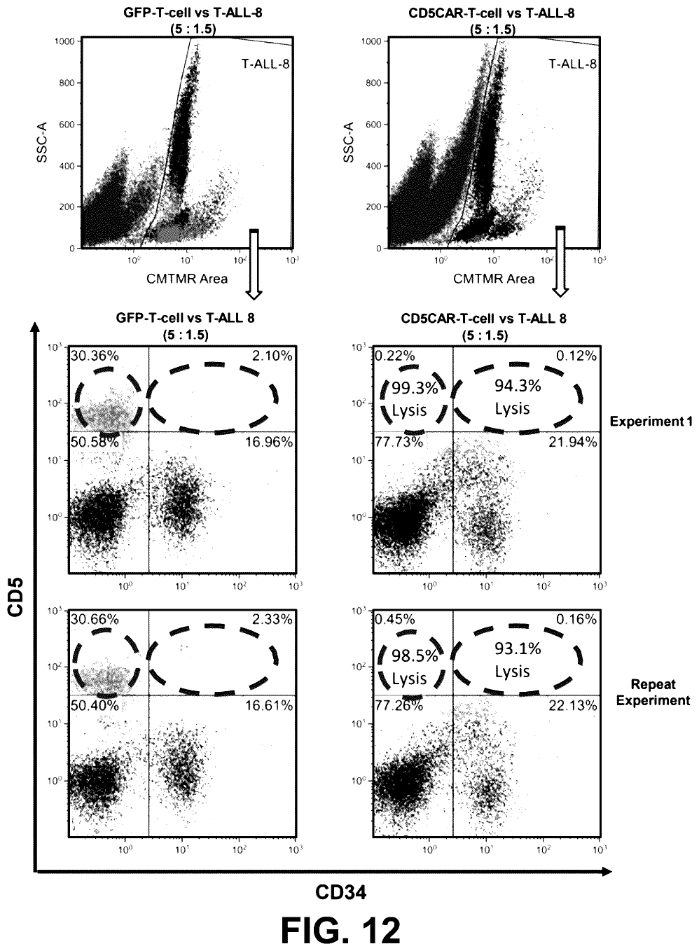

The CD5CAR ability to lyse patient sample T-ALL cells was also assessed using multiple patient samples and CD5CAR cell co-cultures were shown in FIG. 11 and FIG. 12. While there was an avid cell killing noted for the T-ALL 1 patient leukemic cells that was similar to the CD5 target cell lysis seen when CD5CAR cells targeted T cell ALL cell lines, three other patient leukemic cells showed comparatively weaker lysis of target cells (FIG. 11A. and FIG. 11B.).

### CD5CAR T-Cells Exhibit the Specificity and Potent Target Cell Killing.

As a control, the CD5CAR T-cells were also tested for their ability to ablate CD5 negative leukemic T-cells. Anaplastic large T-cell lymphoma line is the cell line that does not express CD5. Flow cytometry analysis showed that CD5CAR T-cells were unable to lyse or eliminate KARPAS 299 cells, as shown in FIG. 11A, lower panel.

A patient sample (T-ALL-8) with a high level of CD5 expression was obtained from a patient with a minimal disease of T-ALL. Co-culture was performed with CD5CAR and analyzed in detail as shown in FIG. 12. Three population cells including CD5+ normal T-cells, CD5+CD34+T-ALL cells and CD5−CD34+T-ALL cells were assessed by flow cytometry after co-culture. CD5CAR exhibited the specificity and potent target cell lysis ability with ≥93% of CD5 positive cell lysis for all CD5+ cell populations when compared to GFP control. The CD5CAR killed leukemic cells as efficiently as CD5 normal T-cells. Killing was not observed in the CD5 negative population. CD5CAR T-cells essentially eliminated the T-cell population (CD5+CD34−).

### CD5CAR T-Cells Effectively Eliminate Normal T-Cells.

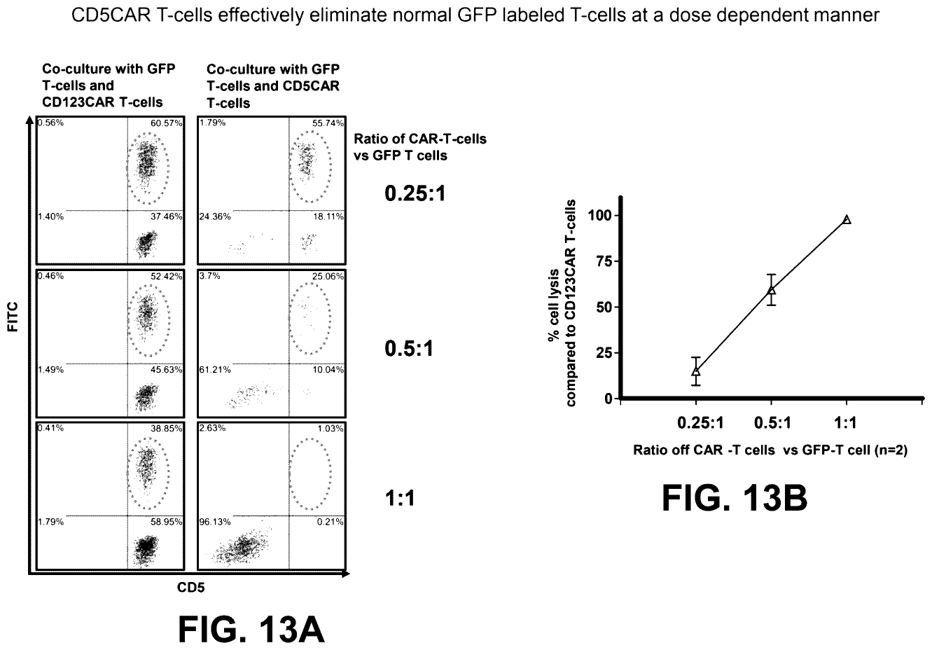

CD5CAR T-cells demonstrated effective elimination of normal T-cells in a dose dependent manner in a co-culture assay at low ratios (effector:target) of 0.25:1, 0.5:1 and 1:1 (FIG. 13). CD5CAR T-cells or CD123CAR-T (control) effector cells were incubated with GFP labeled T-cells. Percent killing of target cells was measured by comparing GFP T-cell survival in CD5CAR-T co-culture relative to that in CD123CAR-T control co-culture. Normal GFP T-cells were eliminated in a dose-response fashion for CD5CAR T-cells. CD5CAR T-cells effectively eliminated all GFP T-cells at effector to target ratio of 1:1 (FIG. 13). Since the CD5CAR T-cells effectively eliminated all normal T-cells, the feasibility of CD5CAR-T therapy should depend on the ability to provide transient rather than permanent. CD5CAR T-cells could be used as a novel conditioning regimen or a “bridge” for hematopoietic cell transplantation.

T-Cells Maintained CD5 Expression when they were Co-Cultured with CD5CAR or Anchored CD5 scFv T-Cells.

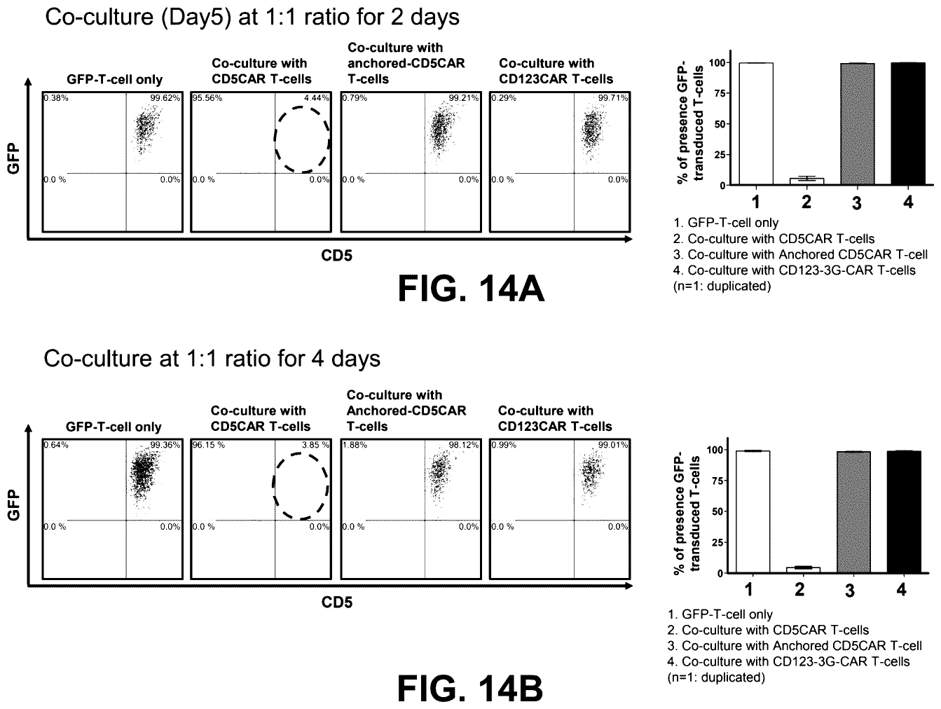

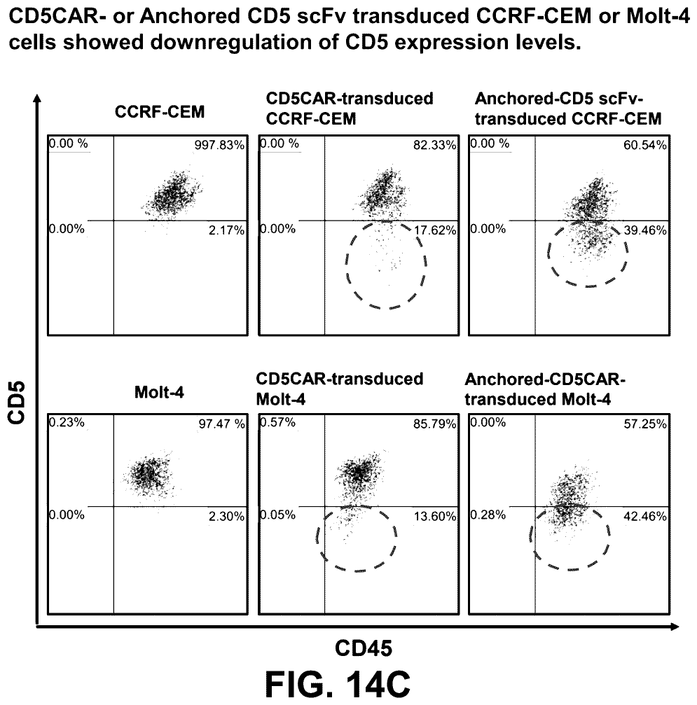

We then co-cultured transduced CD5CAR or CD5 anchored scFv and CD123CAR T-cells with GFP-labeled T-cells at the ratio of 1:1 (E:T) for 2 or 4 days. As shown in FIGS. 14A and 14B, CD5CAR T-cells effectively eliminated all GFP-T-cells. As expected, transduced CD5 anchored scFv or CD123CAR T-cells were unable to lyse GFP T-cells. In addition, GFP T-cells still expressed CD5 when co-cultured with transduced CD5 anchored scFv or CD123CAR T-cells. These studies indicate that CD5 antigen escape is unlikely to occur when employing CD5CAR for immunotherapy.

Down-Regulation of CD5 Expression in the T-ALL Cells when they were Transduced with Lenti-CD5CAR or CD5 Anchored scFv Viruses.

We next tested if transduction of CD5CAR- or anchored CD5CAR lentiviruses on T ALL cells results in the downregulation of CD5 expression. CCRF-CEM and MOLT-4 T-ALL cells were transduced with CD5CAR- or anchored CD5 scFv lentiviruses. CD5CAR or anchored CD5 scFv significantly down-regulated or reduced the quantity of surface CD5 expression on these leukemic cells (FIG. 14C).

### CD5CAR T-Cells Exhibit Profound Anti-Tumor Activity In Vivo.

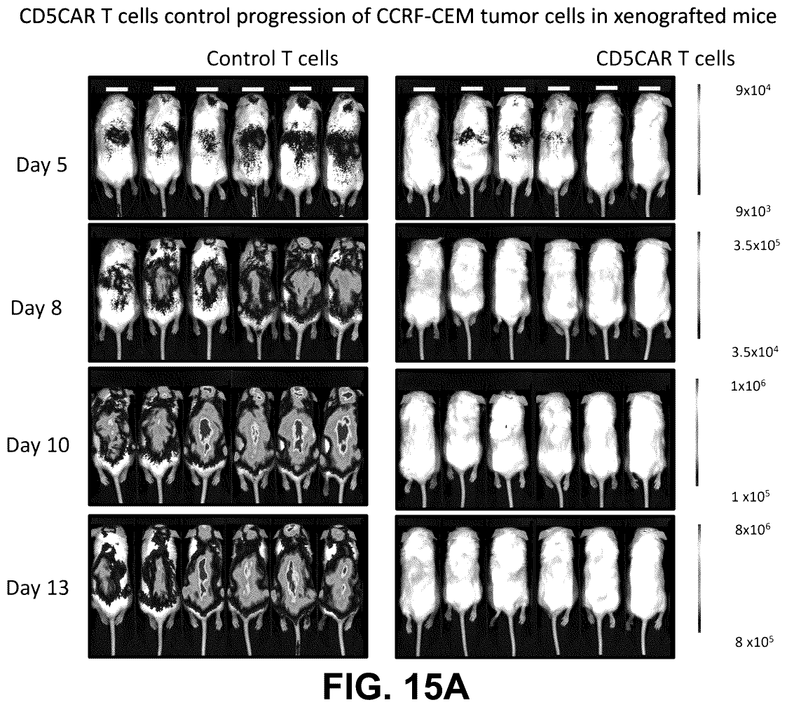

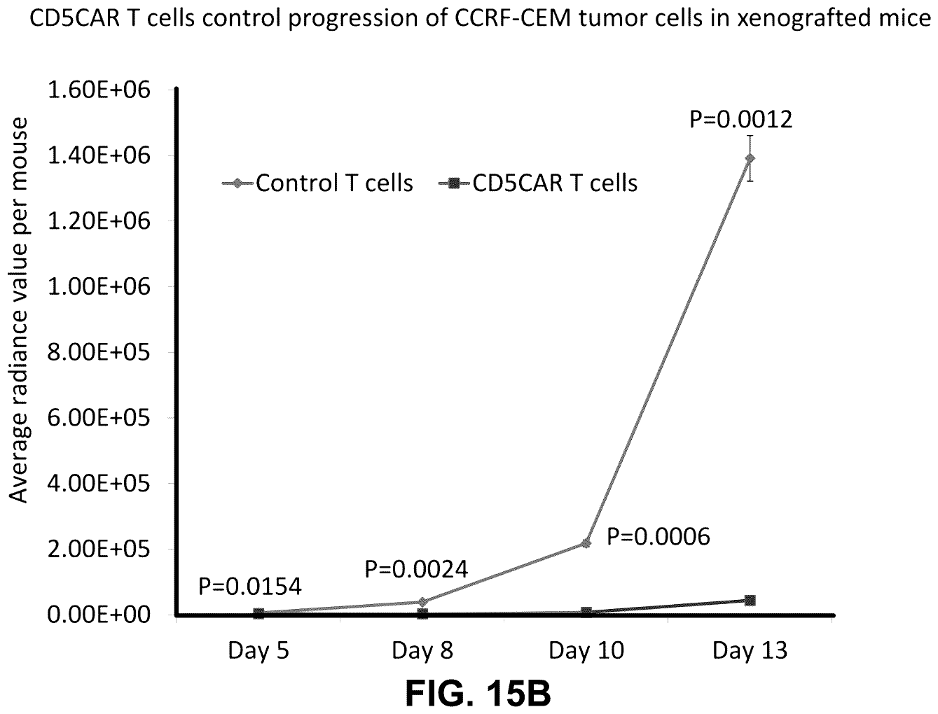

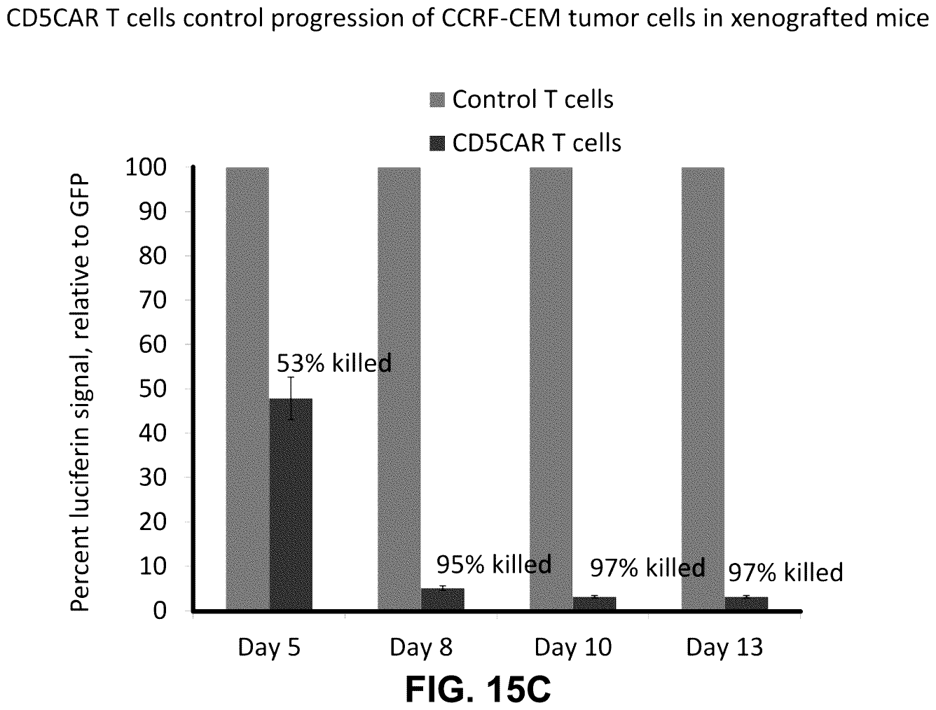

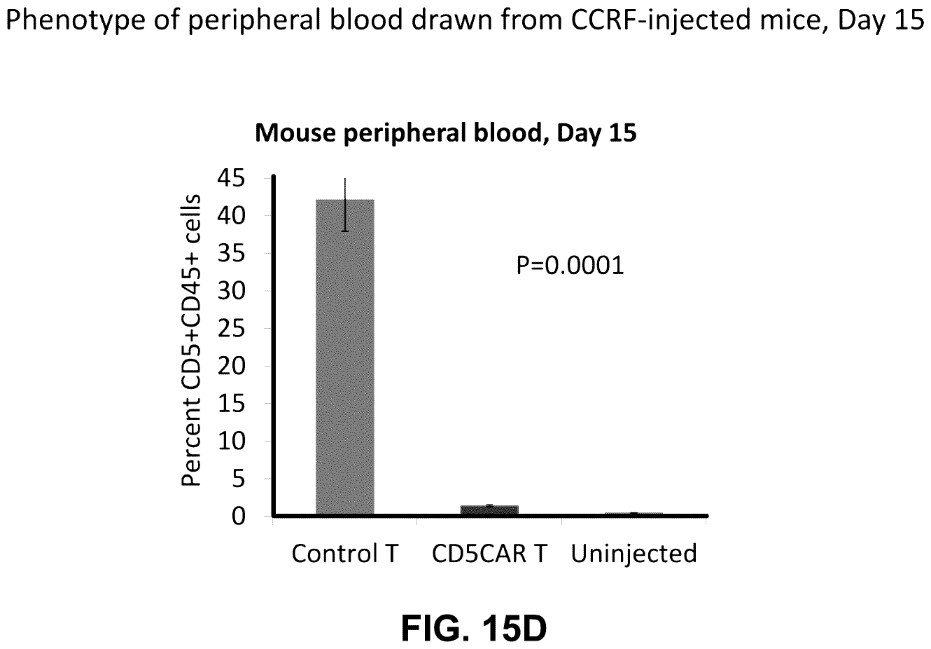

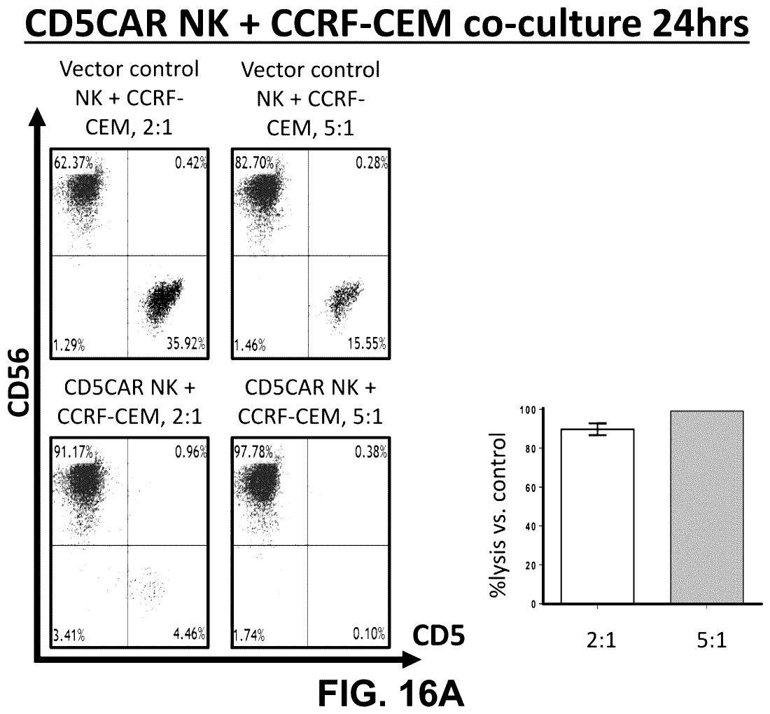

In order to evaluate the in vivo anti-tumor activity of CD5CAR T-cells as a predictor of their therapeutic efficacy in patients, we developed a xenograft mouse model using NSG mice sublethally (2.0 Gy) irradiated and intravenously injected with 1.0×10⁶ firefly luciferase-expressing CCRF-CEM cells (CD5+) to induce measurable tumor formation. On day 3 days following CCRF-CEM-Luc+ cell injection, mice were intravenously injected with 5×10⁶ CD5CAR T-cells or vector control T-cells. These injections were repeated on Day 4, Day 6, and Day 7, for a total of 20×10⁶ T-cells per mouse. On days 5, 8, 10 and 13, mice were injected subcutaneously with RediJect D-Luciferin (Perkin-Elmer) and subjected to IVIS imaging (Caliper LifeSciences) to measure tumor burden (FIG. 15). Paired T test analysis revealed a very highly significant difference between the two groups by day 13 with less light intensity and thus less tumor burden in the CD5CAR-T injected group compared to control (p<0.0012).

### Anti-CD5 Chimeric Antigen Receptor (CD5CAR) NK Cells Efficiently Eliminate CD5 Positive Hematologic Malignancies.

### Generation of the CD5NK-CAR

The anti-CD5 molecule is a modular design, comprising of a single-chain variable fragment (scFv) in conjunction with CD28 and 4-1BB domains fused to the CD3zeta signaling domain to improve signal transduction making it a third generation CAR. A strong spleen focus forming virus promoter (SFFV) was used for efficient expression of the CD5CAR molecule on the NK cell surface and the CD8 leader sequence was incorporated into the construct. The anti-CD5 scFv is attached to the intracellular signaling domains via a CD8-derived hinge (H) and transmembrane (TM) regions. This CD5CAR construct was then cloned into a lentiviral plasmid.

### Generation of CD5CAR NK Cells

The transduction efficiency of the CD5CAR was determined by flow cytometry analysis. To enrich for CD5CAR+NK cells, the highest expressing NK cells were harvested using flow cytometry. Following sorting, the expression of the CD5CAR high NK was expanded for efficacy studies in vitro and vivo.

### CD5CAR NK Cells Effectively Eliminate Human T-Cell Acute Lymphomblastic Leukemia (T-ALL) Cell Lines

CD5CAR NK cells were tested for anti-T-ALL activity in vitro using CCRF-CEM, MOLT-4 and Jurkat cell lines. All these T-ALL cell lines highly expressed CD5.

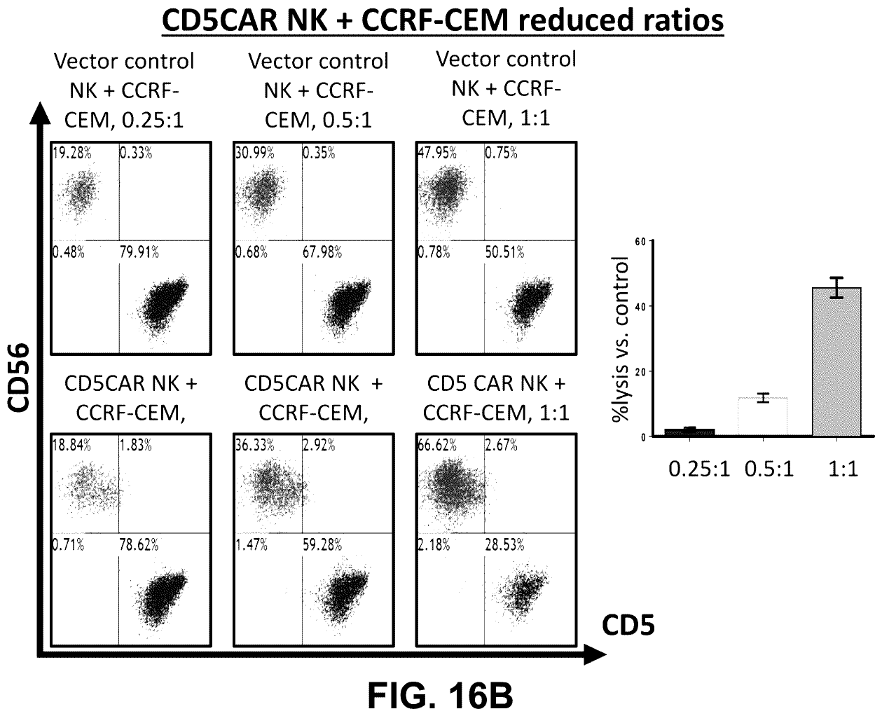

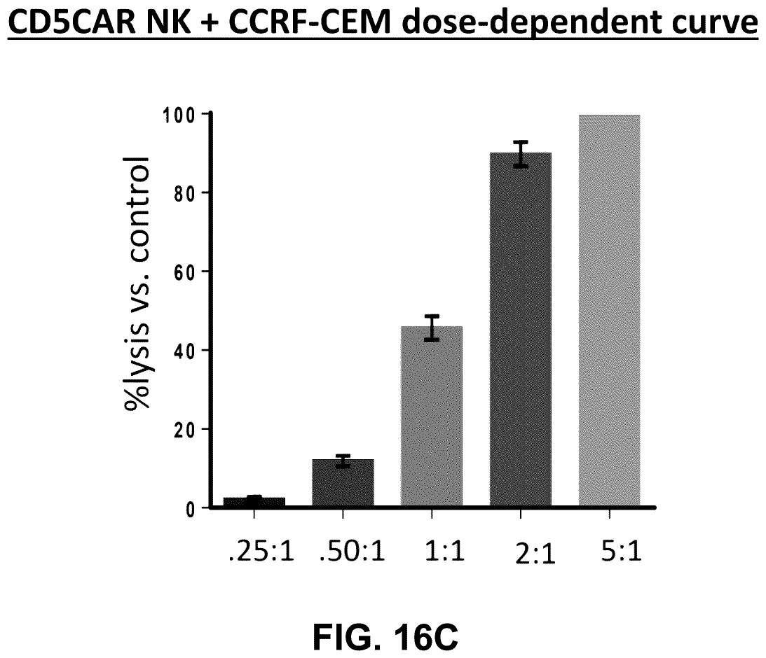

During co-culture experiments, CD5CAR NK cells demonstrated profound killing of CCRF-CEM at the low effector cell to target cell ratio (E:T) of 2:1 and 5:1. At these ratios, CD5CAR NK cells virtually eliminated CCRF-CEM cells (FIG. 16A). CD5CAR NK cells lysed CCRF-CEM leukemic cells in vitro in a dose-dependent manner at effector: target ratios of 0.25:1, 0.5:1, 1:1, 2:1 and 5:1 (FIG. 16B).

### CD5CAR NK Cells Demonstrate a Potent Anti-Leukemic Activity In Vivo.

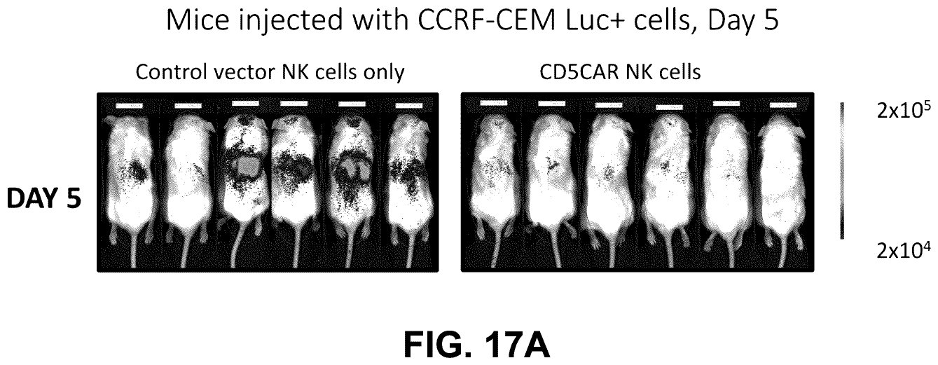

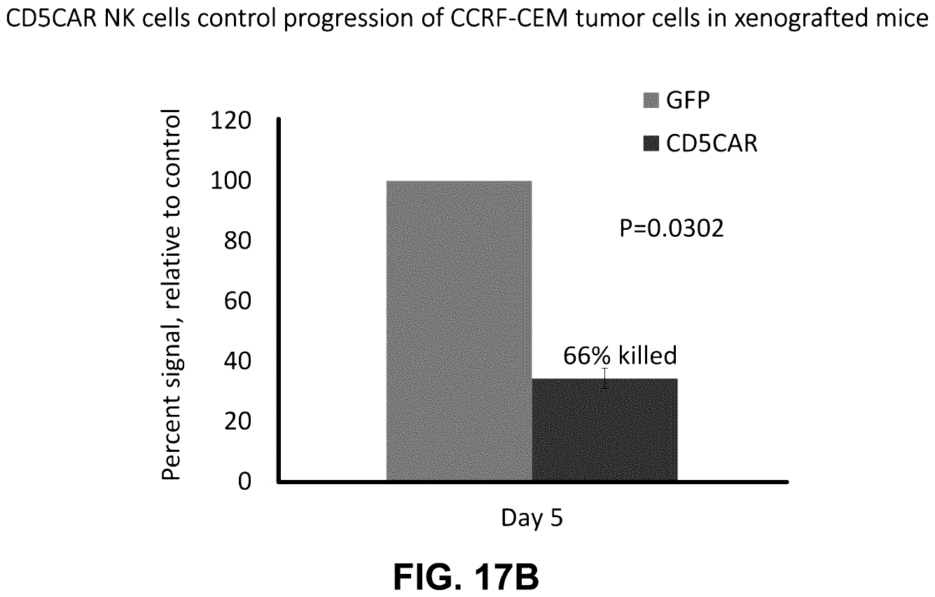

A similar strategy for CD5CAR T-cells, animal studies were employed to determine the in vivo anti-tumor activity of CD5CAR NK cells. Sublethally irradiated NSG mice were intravenously injected with 1.0×10⁶ firefly luciferase-expressing CCRF-CEM cells to induce measurable tumor formation. 3 days following CCRF-CEM-Luc+ cell injection, mice were intravenously injected with 5×10⁶ CD5CAR NK cells or vector control T-cells. These injections were repeated on Day 4 for a total of 10×10⁶ T-cells per mouse. On day 5, mice were injected subcutaneously with RediJect D-Luciferin and subjected to IVIS imaging to measure tumor burden (FIG. 17A). Average light intensity measured for the CD5CAR NK cell injected mice was compared to that of vector control NK cell injected mice (FIG. 17B). Tumor burden was two thirds lower for treated mice on day 5 after tumor injection. Paired T test analysis revealed a very highly significant difference (P=0.0302) between the two groups. These in vivo data indicate that CD5CAR NK cells significantly reduce tumor burden in CCRF-CEM-injected NSG mice in a rapid manner when compared to vector control NK cells.

### Anti-CD3 Chimeric Antigen Receptor (CD3CAR) NK Cells Efficiently Lyse CD3 Positive Hematologic Malignancies

### Generation of the CD3CAR

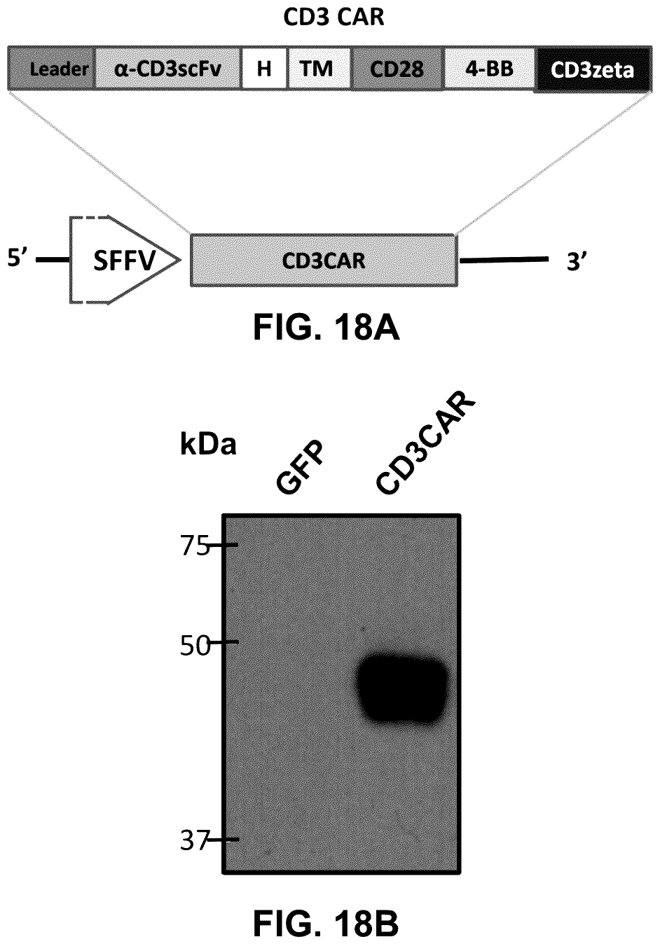

The anti-CD3 molecule is a modular design, comprising of a single-chain variable fragment (scFv) in conjunction with CD28 and 4-1BB domains fused to the CD3zeta signaling domain to improve signal transduction making it a third generation CAR. A strong spleen focus forming virus promoter (SFFV) was used for efficient expression of the CD3CAR molecule on the NK cell (NK-92) surface and the CD8 leader sequence was incorporated into the construct. The anti-CD3 scFv is attached to the intracellular signaling domains via a CD8-derived hinge (H) and transmembrane (TM) regions (FIG. 18A). This CD3CAR construct was then cloned into a lentiviral plasmid.

### Characterization of CD3CAR

Western blot analysis was performed on HEK293-F T-cells transfected with CD3CAR lentiviral plasmid and vector control plasmid. Immunoblots with anti-CD3zeta monoclonal antibody show bands of predicted size for the CD3CAR-CD3zeta fusion protein versus no bands for the vector control protein (FIG. 18B).

### Generation of CD3CAR NK Cells Using NK-92 Cells

The transduction efficiency of the CD3CAR was determined by flow cytometry analysis. To enrich for CD3CAR NK cells, the highest expressing NK cells were harvested using fluorescence-activated cell sorting (FACS). Following sorting, NK cells with relatively high expression of CD3CAR was obtained. Expression of CD3CAR following flow cytometry sorting was stable around 30% of CAR expression for subsequent NK cell expansion and cryopreservation.

CD3CAR NK Cells Effectively Eliminate CD3+ Leukemic Cells from Human Samples

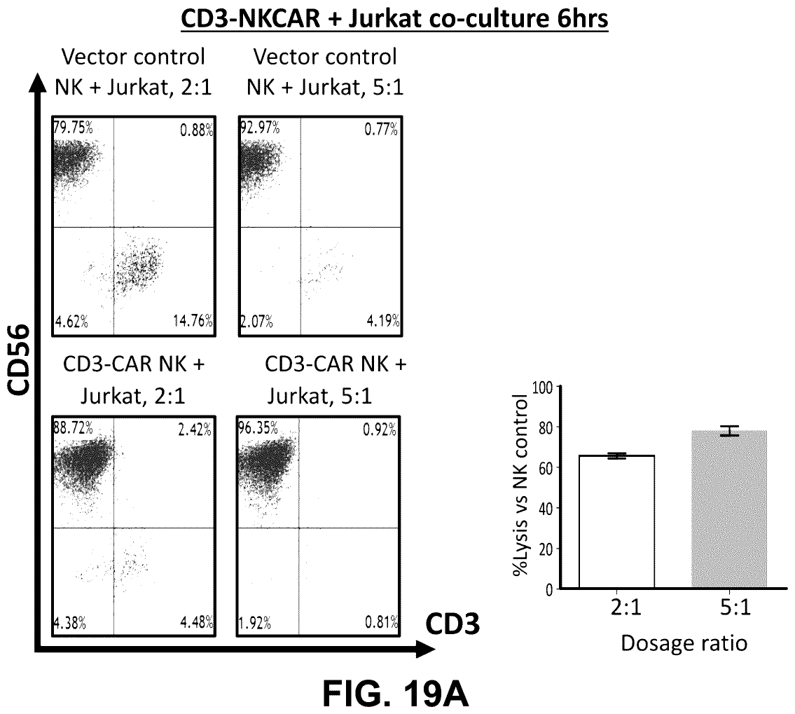

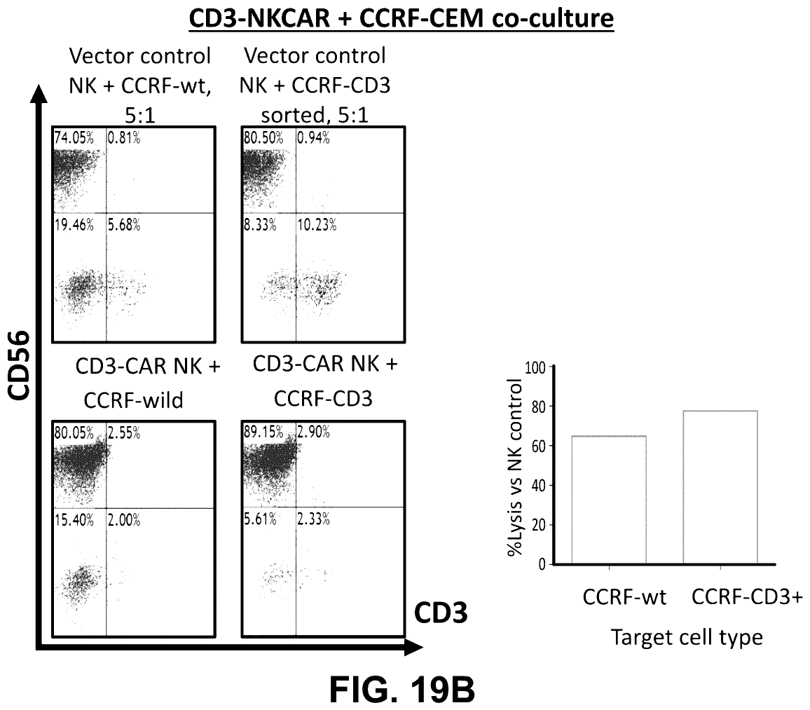

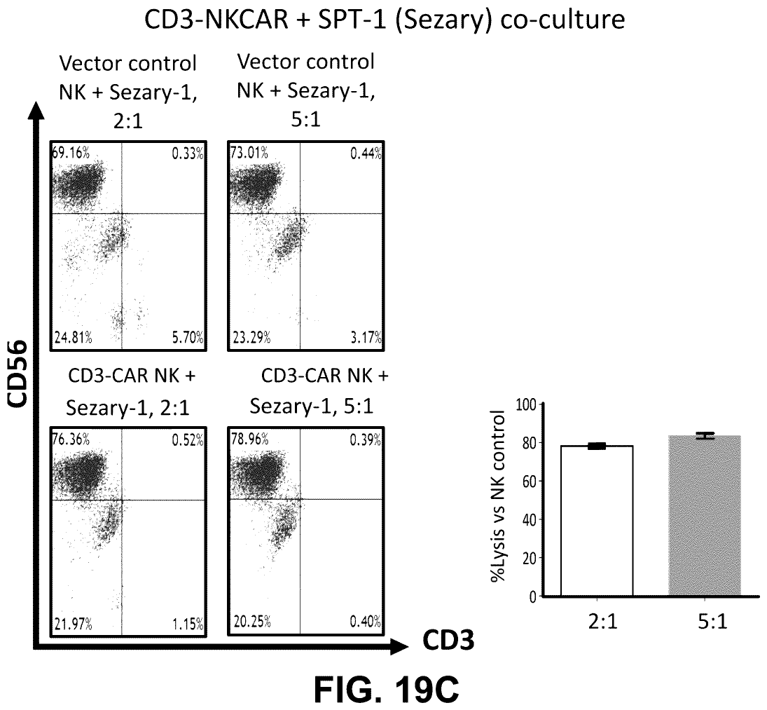

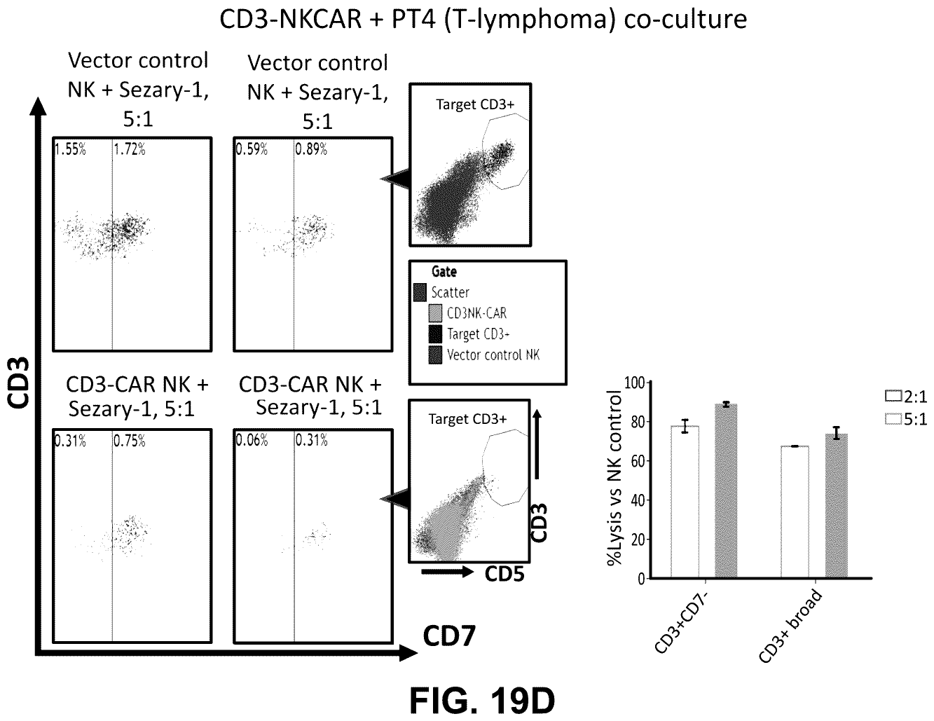

The killing ability of CD3CAR NK cells was also tested using patient samples. Flow cytometry analysis of both patient samples revealed strong and uniform CD3 expression. As analyzed by flow cytometry, co-culture of Sezary syndrome patient sample with CD3CAR T-cells effectively resulted in lysis of approximately 80% of leukemic cells at a low E:T ratio of 2:1 (FIG. 19A). Co-culture of patient sample, unclassified PTCLs with CD3CAR NK cells for 24-hours resulted in virtual ablation of CD3+ malignant cells (FIG. 19B). The CD3CAR NK cells also affected the broad CD3+ population.

### CD3CAR NK Cells Exhibit Profound Anti-Leukemic Activity In Vivo

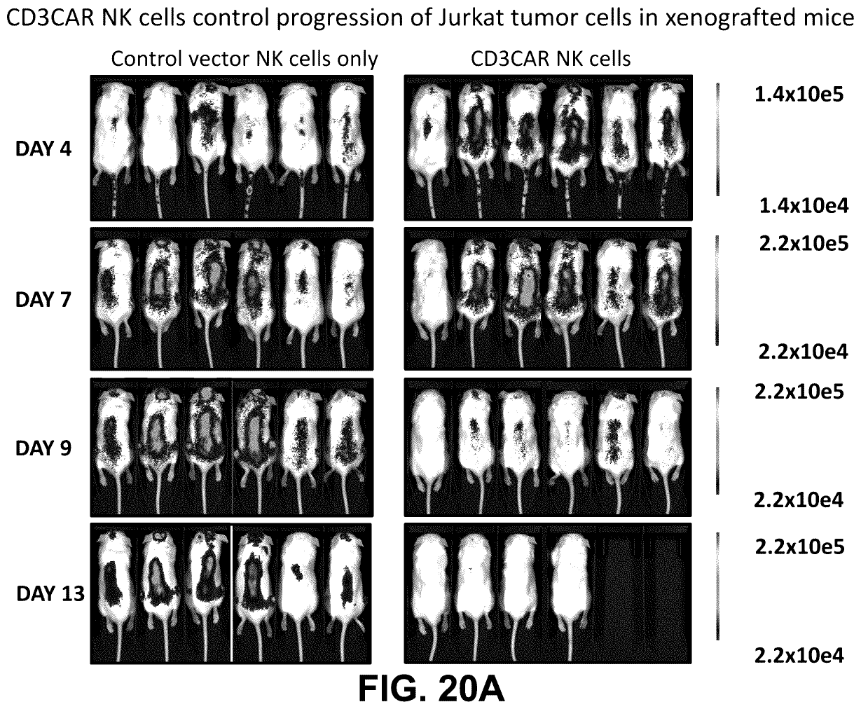

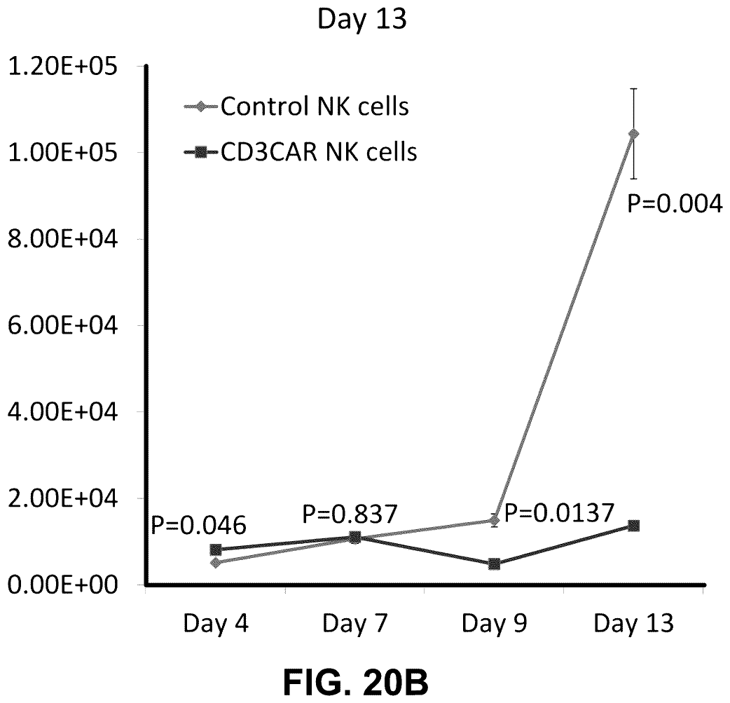

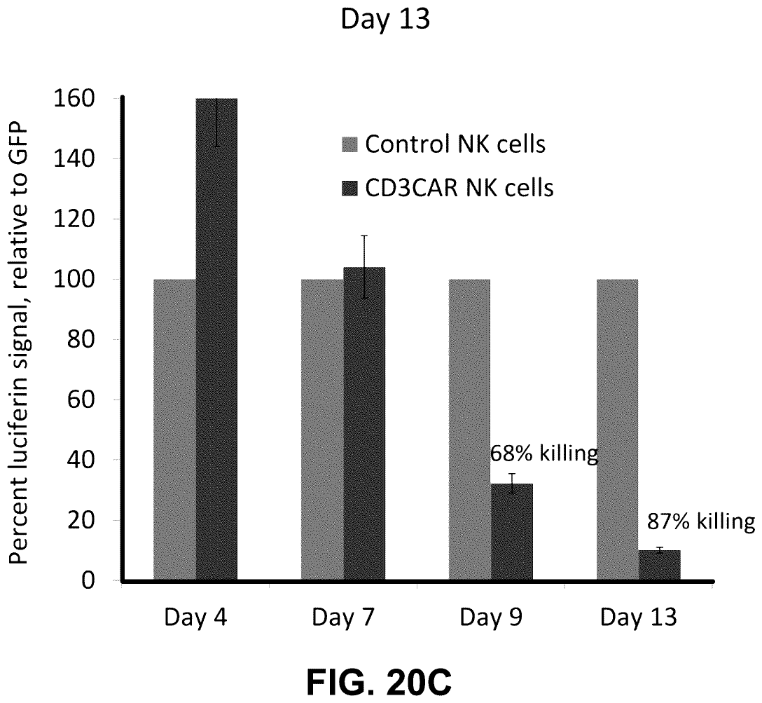

To determine the in vivo anti-tumor efficacy of CD3CAR NK cells, sublethally irradiated NSG mice were intravenously injected with 1.0×10⁶ firefly luciferase-expressing Jurkat cells, which are CD3 positive (˜80%), and measurable tumor formation was detected by Day 3 or 4. Three days following Jurkat-Luc+ cell injection, mice were intravenously injected with 5×10⁶ CD3CAR NK cells or vector control NK cells per mouse, 6 per group. These injections were repeated on Day 3, 6, 7 and 10 for a total of 25×10⁶ T cells per mouse. On days 4, 7, 9 and 13 mice were subjected to IVIS imaging to measure tumor burden (FIG. 20A). Two treated mice died due to injection procedure on day 13. Paired T test analysis revealed a highly significant difference (P=0.0137) between the two groups. We conclude that these in vivo data demonstrate that CD3CAR NK cells significantly reduce tumor burden and prolong survival in Jurkat-injected NSG mice when compared to vector control NK cells.

### CRISPR/Cas Nucleases Target to CD2, CD3, CD5 and CD7 Expressed on T or NK Cells

T or NK cells appear to share some of surface antigens, such as CD2, CD3, CD5 and CD7 with leukemia or lymphoma. CD2, CD3, CD5, and CD7 could be good targets for T and NK cells as they are expressed in most of T-cell leukemia/lymphoma.

Therefore, when one of surface antigens, CD2, CD3, CD5, and CD7 is selected as a target, this antigen is needed to delete or down-regulate in T or NK cells used to generate CAR if they share this antigen, to avoid self-killing within the CAR T or NK cell population.

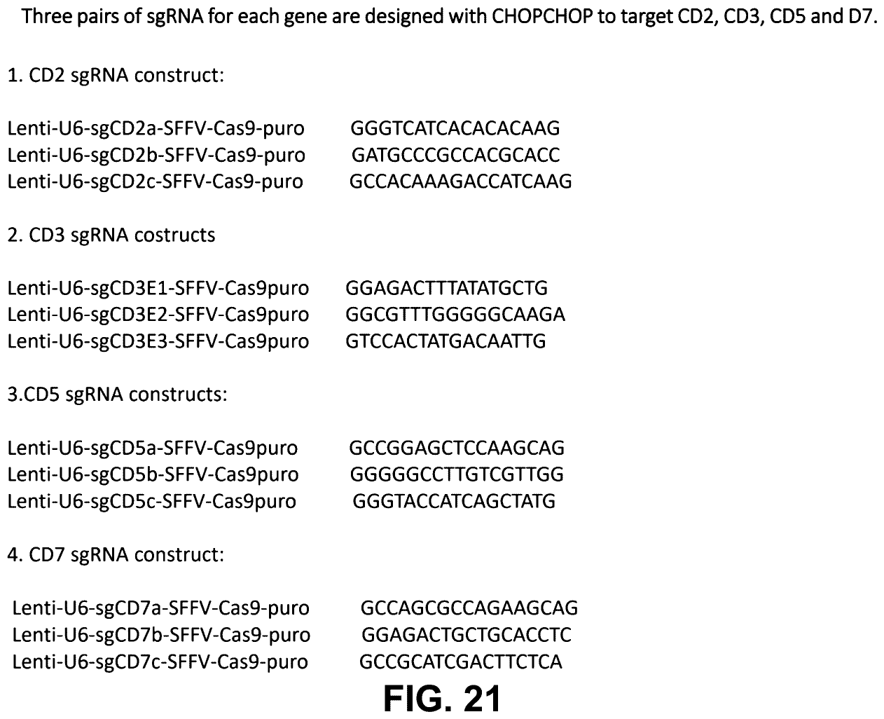

Three pairs of sgRNA were designed with CHOPCHOP to target CD2, CD3, CD5, and CD7. Gene-specific sgRNAs (FIG. 21) were then cloned into the lentiviral vector (Lenti U6-sgRNA-SFFV-Cas9-puro-wpre) expressing a human Cas9 and puromycin resistance genes linked with an E2A self-cleaving linker. The U6-sgRNA cassette is in front of the Cas9 element. The expression of sgRNA and Cas9puro is driven by the U6 promoter and SFFV promoter, respectively.

### CRISPRICas Nucleases Target to CD5 on T-Cell Lines.

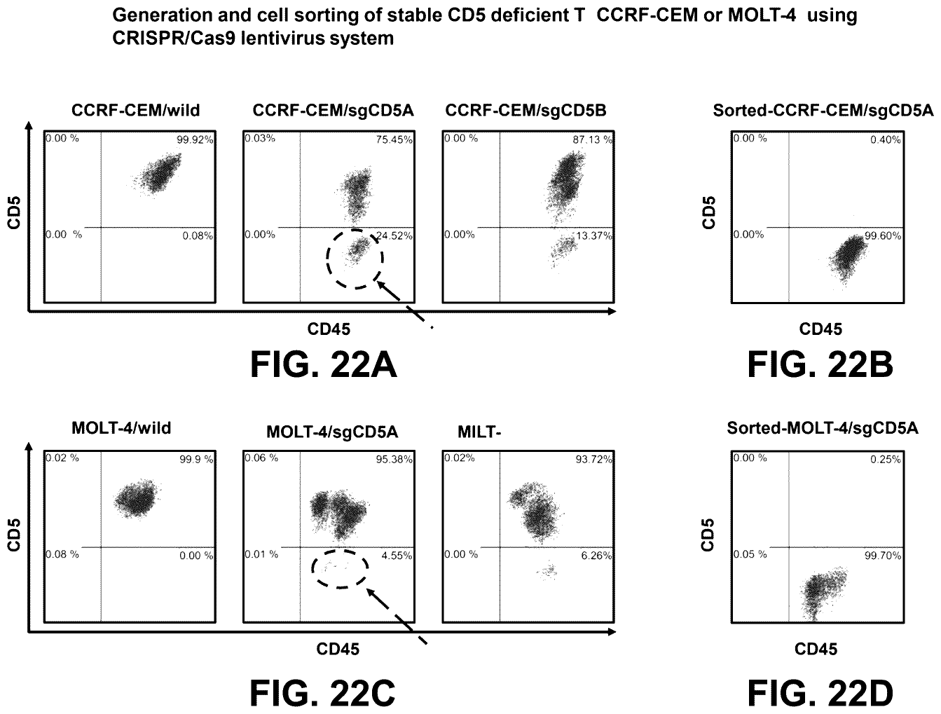

Lentiviruses carried gene-specific sgRNAs were used to transduce CCRF-CEM and MOLT cells. Initially, the loss of CD5 expression was observed in both of these T-cell lines using two different two CDISPR/Cas9 sgRNA sequences (FIGS. 22A and 22C). The most successful population in terms of the loss of CD5 expression was chosen for each cell line, and these cells were sorted, expanded normally and found to be of >99% purity CD45+ and CD5− (FIGS. 22B and 22D).

### CRISPRICas Nucleases Target to CD7 on T Cell Lines and NK Cells.

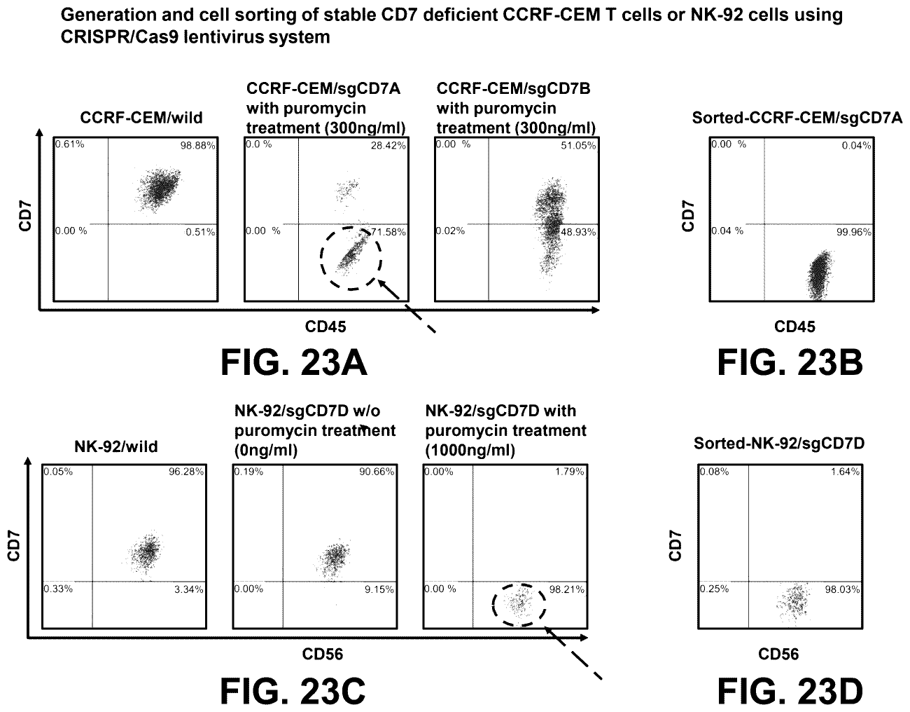

Lentiviruses carried gene-specific sgRNAs were used to transduce CCRF-CEM, MOLT cells and NK cells (FIG. 23). Flow cytometry analysis demonstrated the loss of CD7 expression in CCRF-CEM and NK-92 cells with CRISPR/Cas9 approach using two different sgRNAs (FIGS. 23A and 23B). The population (denoted by the circle and arrow) was selected for sorting, expansion and analysis in FIG. 23B. The loss of CD5 expression by flow cytometry analysis was also seen in NK-92 cells using a similar approach described above with CRISPR/Cas nucleases targeting to CD7 (FIGS. 23C and 23D) The sorted CD7 negative NK-92 cells (FIG. 23D) were expanded and used to generate CD7CAR NK cells to eliminate CD7 positive leukemic cells.

CD7CAR NK -92 Cells have a Robust Anti-Leukemic Activity

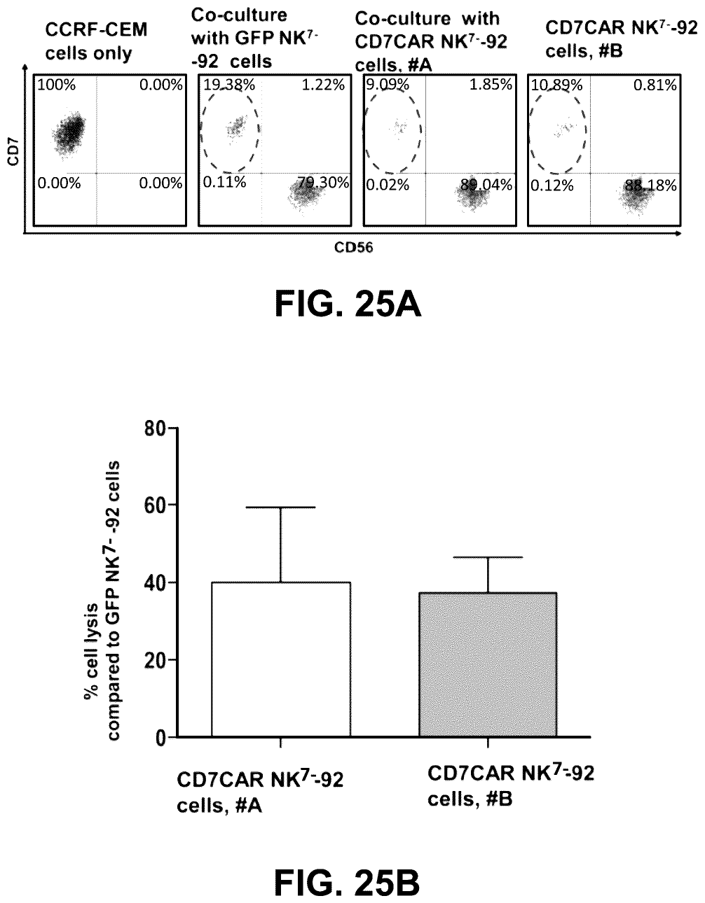

CD7 is expressed in both NK and T-ALL leukemic cells. To avoid self-killing within the CD7CAR NK-92 population, CD7 expression first needs to be inactivated. CD7 deficient NK-92 cells (NK⁷⁻ −92 cells) were generated as described in (FIG. 23D) and expanded. The expanded NK-92 cells were transduced with lentivirus expressing a CD7CAR. CD7CAR includes an anti-CD7 scFV in conjunction with CD28 and 4-BB domains fused to CD3zeta signaling domain making it a third generation CAR. CD7CAR NK-92 cells were used to test their lysis ability of leukemic cells expressing CD7. As shown in FIG. 25, CD7CAR NK γ--92 cells displayed a potent anti-leukemic activity against a T-ALL cell line, CCRF-CEM. As analyzed by flow cytometry, co-culture of CCRF-CEM cells effectively resulted in the lysis of approximately 50% of leukemic cells at E:T ratio of 5:1 (FIGS. 25A and 25B).

### CD3CAR is Used for Graft-Versus-Host Disease (GvHD).

CD3CAR is administered to a patient prior to or after a stem cell transplant. The patient is tested for elevated levels of white blood cells.

CD3CAR is administered to a patient prior to or after a bone marrow transplant. The patient is tested for elevated levels of white blood cells.

CD3CAR is administered to a patient prior to or after a tissue graft. The patient is tested for elevated levels of white blood cells.

### Organ Transplant

CD3CAR is administered to an organ transplant patient before organ transplant surgery. The patient is tested for organ rejection. The following histological signs are determined: (1) infiltrating T-cells, in some cases accompanied by infiltrating eosinophils, plasma cells, and neutrophils, particularly in telltale ratios, (2) structural compromise of tissue anatomy, varying by tissue type transplanted, and (3) injury to blood vessels.

CD3CAR is administered to an organ transplant patient after organ transplant surgery. The patient is tested for organ rejection. The following histological signs are determined: (1) infiltrating T-cells, in some cases accompanied by infiltrating eosinophils, plasma cells, and neutrophils, particularly in telltale ratios, (2) structural compromise of tissue anatomy, varying by tissue type transplanted, and (3) injury to blood vessels.

### Targeting T- Cell Malignancies Using CD2CAR

### Generation of CD2CAR Construct

We generated CD2CAR. CD2CAR consists of an anti-CD2 single-chain variable fragment (scFv) region, CD8-derived hinge (H) and transmembrane (TM) regions, and tandem CD28 and 4-1BB co-activation domains linked to the CD3ξ signaling domain. A strong spleen focus forming virus promoter (SFFV) and a CD8 leader sequence were used.

### CD2CAR NK -92 NK Cells

As a proof of concept, we next investigate that CD2CAR in NK-92 cells response to the CD2 antigen in leukemic cells as NK-92 cells only bear a low number of cells expressing CD2 antigen. The NK-92 cells were transduced with lentiviruses expressing CD2CAR and resulting CD2CAR NK-92 cells were used to test their anti-leukemic activity.

### CD2CAR NK -92 NK Cells Especially Lyse CD2+T-ALL (T-Acute Lymphoblastic Leukemia) Cells

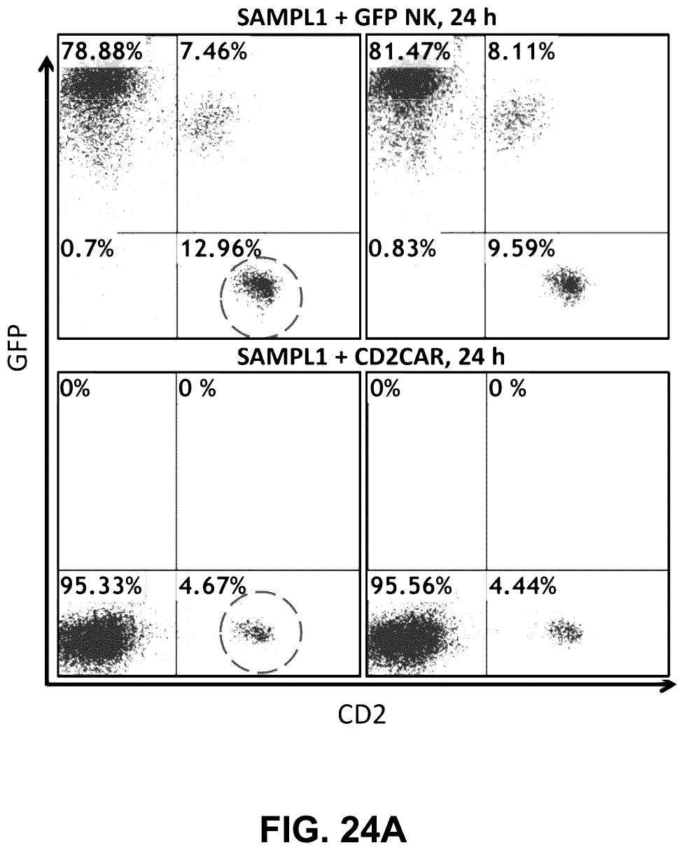

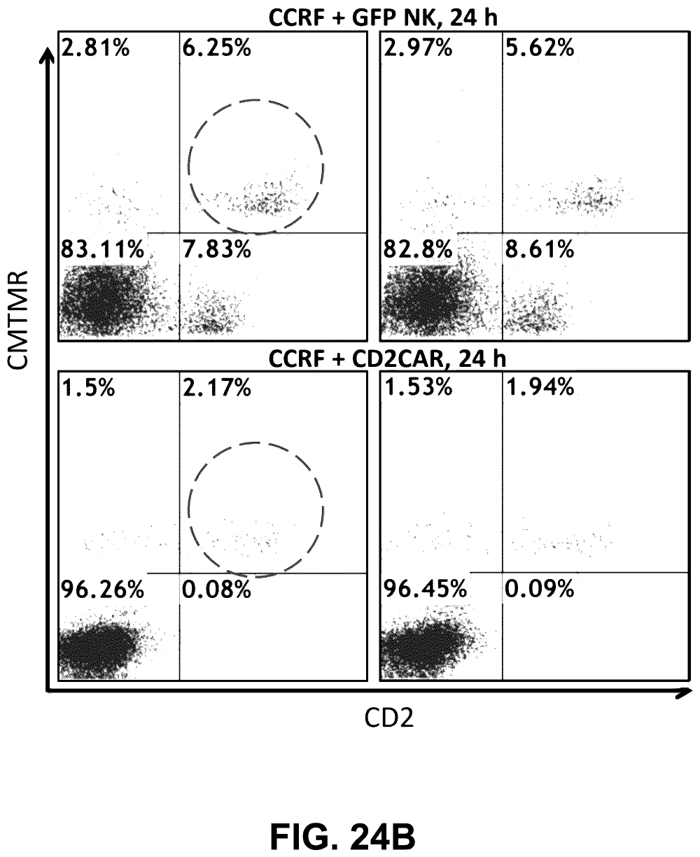

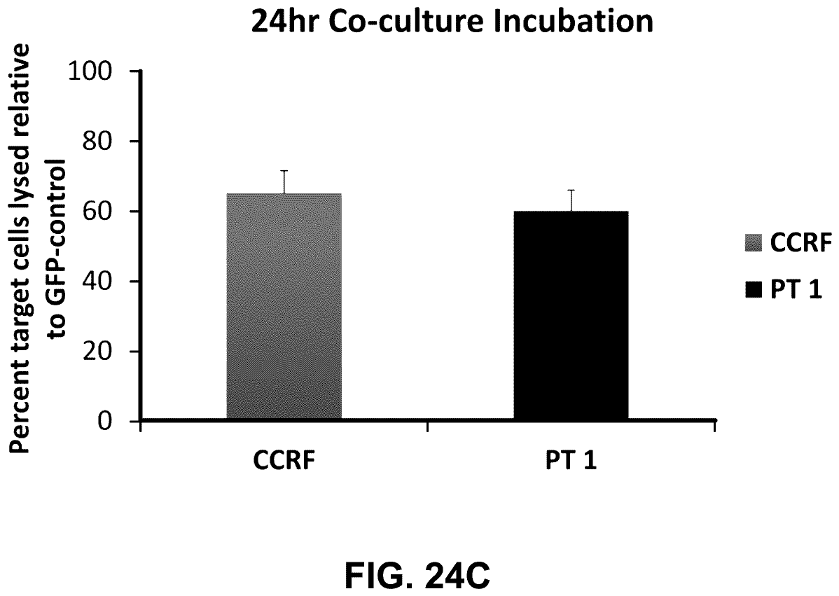

To assess CD2CAR NK92 anti-leukemic activity, we conducted co-culture assays using a T-ALL cell line, CCRF-CEM and a T-ALL primary human patient sample. We demonstrated that CD2CAR NK-92 cells consistently displayed robust lysis of leukemic cells. Following 24-hour incubation at a low effective to target cell (E:T ratio 5:1), CD2CAR NK-92 cells effectively lysed more than 60% of CCRF-CEM and human primary T-ALL sample (PT1)(FIGS. 24A-24C).

### Generating CD7 CAR T Cells Targeting CD7 Expressing Cells

The organization of CD7 CAR (also called CD7-RTX CAR). CD7 CAR contains an anti-CD7 scFv, CD8 hinge and transmembrane regions, and a CD28 co-stimulatory domain fused to the CD3zeta signaling domain. The hinge region of CD7CAR also contains two RTX-binding epitopes.

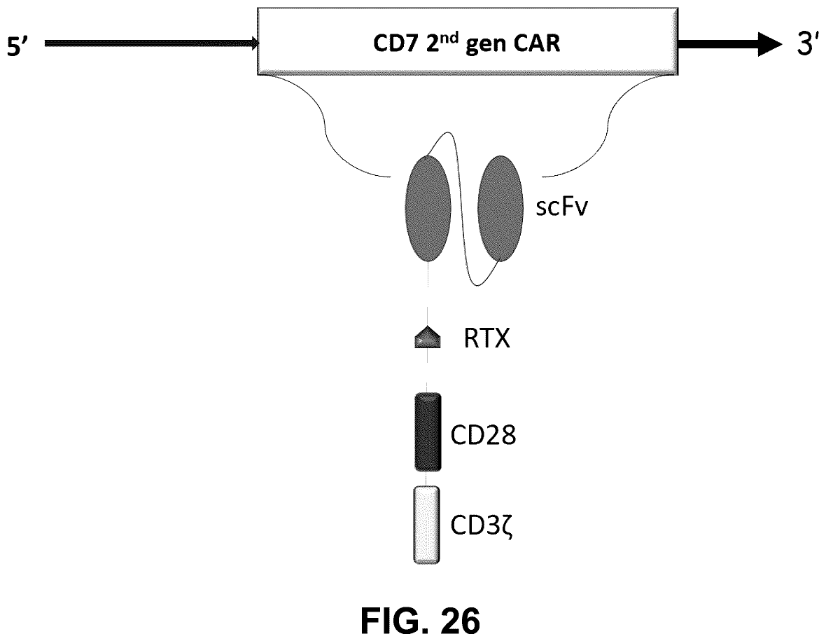

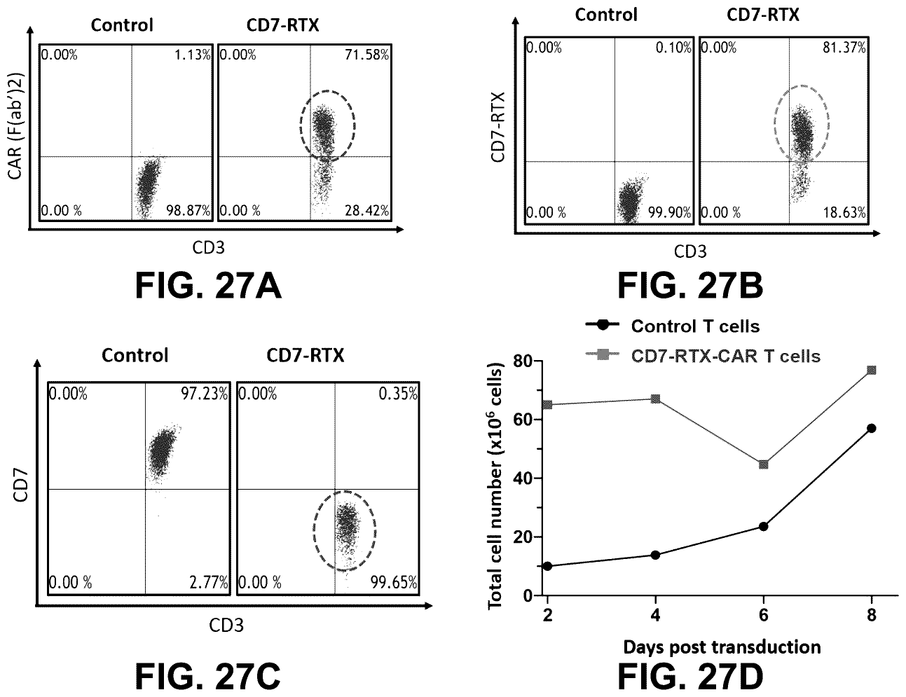

First, we confirmed the characteristics of CD7-RTX CAR. After transduction, flow cytometry analysis confirmed the expression of the CAR product, the availability of the rituximab-binding site, and the downregulation of CD7 in the transduced cells (FIG. 27). As the loss of CD7 may impair the proliferative capabilities of CD7CAR T-cells, we compared the growth of these cells to control cells and found that they expanded at a similar rate (FIG. 27), demonstrating that CD7 is not needed for T-cell proliferation. Depicted in (FIG. 27D). Characterization of CD7CAR and staining with goat-anti-mouse F(Ab′)2-Pe revealed a CAR expression of approximately ˜70% 10 day 8 post-infection. Staining with anti-human CD34 (used to detect the RTX-binding epitope) also demonstrated a transduction efficiency of ˜80% (FIG. 26B-27B). Staining with anti-human CD3 and anti-human CD7 demonstrate that the CD7CAR T-cells retain CD3 expression but lose expression of CD7 (FIG. 27C) likely due to the antibody-mediated internalization. CD7CAR T-cells can expand at rates similar to control T-cells despite losing CD7 expression (FIG. 27D).

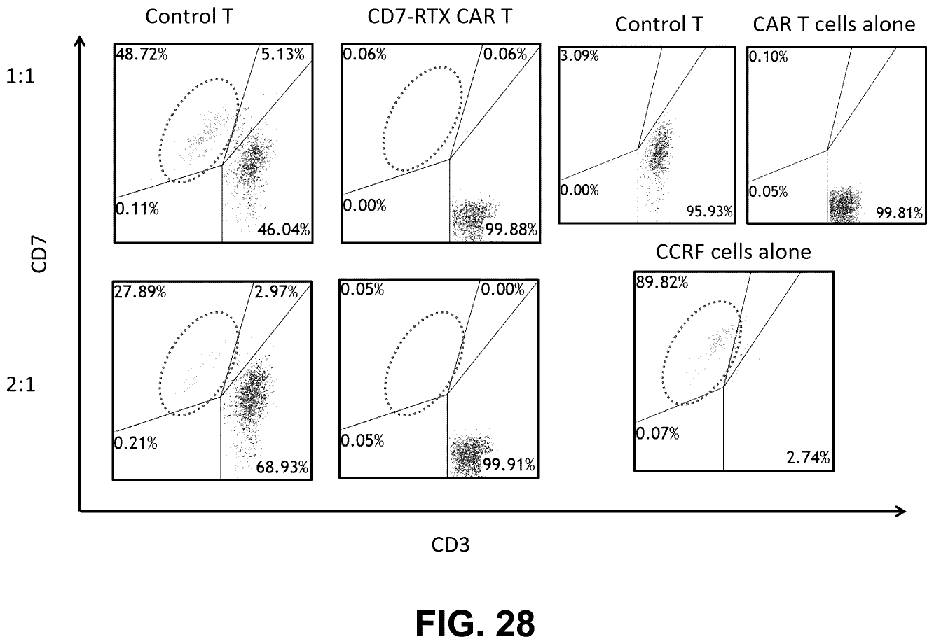

Next, second-generation CD7CAR T-cells derived from human peripheral blood that express CD28 and CD3zeta signaling moieties were co-cultured in vitro with the CCRF-CEM, Jurkat, and MOLT-4 cell lines, which consist of CD7+T-ALL cells, and showed profound elimination of leukemic cells in an 18-hour incubation time (FIG. 28). CD7CAR demonstrates potent cytotoxicity against CD7+ cell lines in vitro. CEM-CCRF cells are ˜90% CD7+(bottom right panel). Control T-cells (left panels) or CD7-RTX T-cells (right panels) were placed with CEM-CCRF cells in an 18-h co-culture at E:T ratios of 1:1 (first row), or 2:1 (second row). Target CD7+ cells are circled in each panel. The results of the co-culture experiments demonstrate that CD7CAR T-cells lyse 99.88% and 99.82% of the CEM-CCRF cells relative to control at 1:1 and 2:1 respectively (FIG. 28).

In order to evaluate in vivo anti-tumor activities, CCRF-CEM leukemic cells were introduced into xenogeneic NSG mouse mice. Prior to injection, 12 mice were irradiated with a sub-lethal dose of gamma irradiation (2.0 Gy) and assigned randomly to the treatment or control group. Twenty-four hours later, mice were given one intravenous injection of 1.0×10⁶ CCRF-CEM cells.

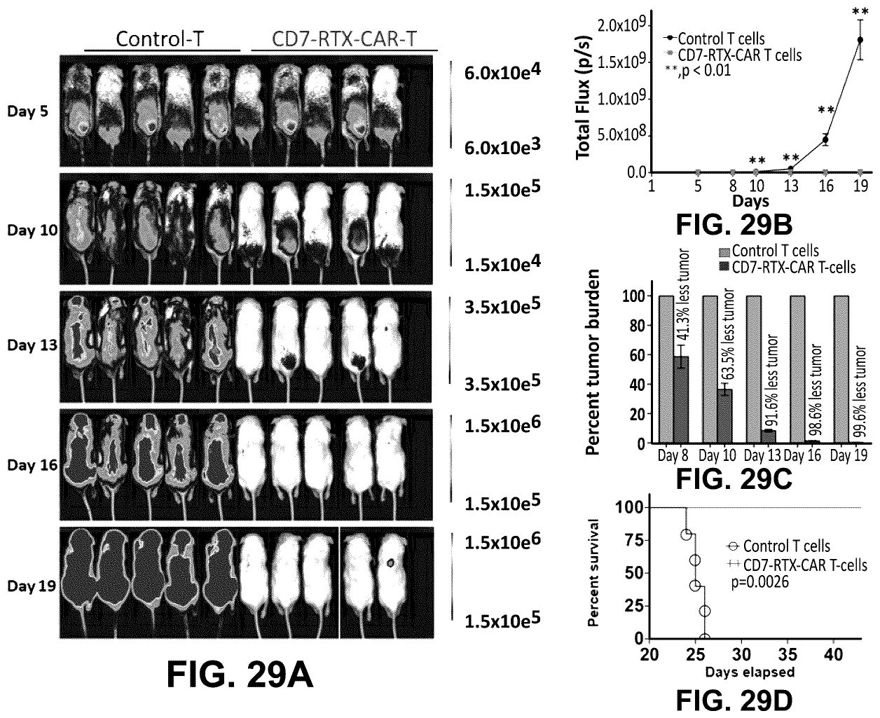

Five days following CCRF-CEM engraftment, mice were intravenously injected with 10×10⁶ CD7CAR or control T-cells. On days 5, 10, 13, 16, and 19, to evaluate tumor burden RediJect D-Luciferin (Perkin-Elmer) was injected intraperitoneally and mice were subjected to IVIS imaging to quantify luciferase activity. While flux, and thus tumor burden, continuously increased in control mice, it remained near background levels for the CD7CAR mice (FIG. 29). While control mice showed significant residual tumor population in the peripheral blood, CD7CAR-treated mice showed virtual depletion comparable to non-injected mice (FIG. 29). Additionally, CD7CAR-treated mice showed significant survival improvement (FIG. 29). CD7CAR improves outcome in in vivo model of T-ALL. NSG mice were sub-lethally irradiated and then intravenously injected on Day 1 with 1.0×10⁶ luciferase-expressing CEM-CCRF cells. Five days later, mice were injected with 10×10⁶ control or CD7CAR T-cells. Mice were injected with RediJect D-Luciferin on Days 5, 10, 13, 16, and 19 and subjected to IVIS imaging. Dorsal view. (FIGS. 28A-29A.) Total flux (photons/second) was measured and indicated a statistically significant difference in tumor burden between the two groups as early as day 8. The flux of the CD7CAR-treated mice had 41.3% (dorsal) less tumor by day 8, which increased to 99.6% (dorsal) by day 19 (FIG. 28C-29C.). While all the control mice required euthanasia due to hindlimb paralysis and hunchback by day 24-26, the CD7CAR-treated mice survived significantly longer, remaining alive on Day 43 (FIG. 28D-29D). Kaplan-Meier survival analysis curve (p=0.0026).

The CD7 CAR Engineered T Cells were Used to Treat Human Patients Having T-ALL with Unexpected Outcomes and Results.

### Background

A 31-year-old male presented with left neck swelling, a WBC (white blood count) of 352.27×10⁹/L, ETV6 mutation (40.8%), NOTCHI mutation (41.8%), and NRAS mutation (44.6%), and chromosome fusion gene negative. The diagnosis of high-risk T-ALL was made, and the patient began multiple lines of chemotherapeutic treatment regimen followed by a sibling HSCT (human stem cell transplant). Despite a disease reduction from multiple chemotherapeutic treatments, including bone marrow transplant the patient suffered a disease recurrence at day 83. The patient was then enrolled for CD7 CAR T cell therapy.

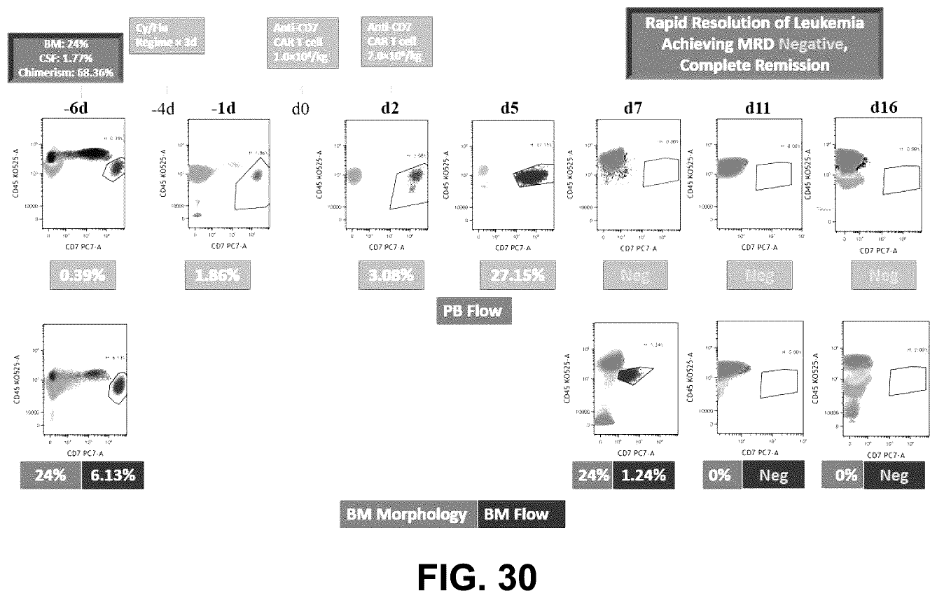

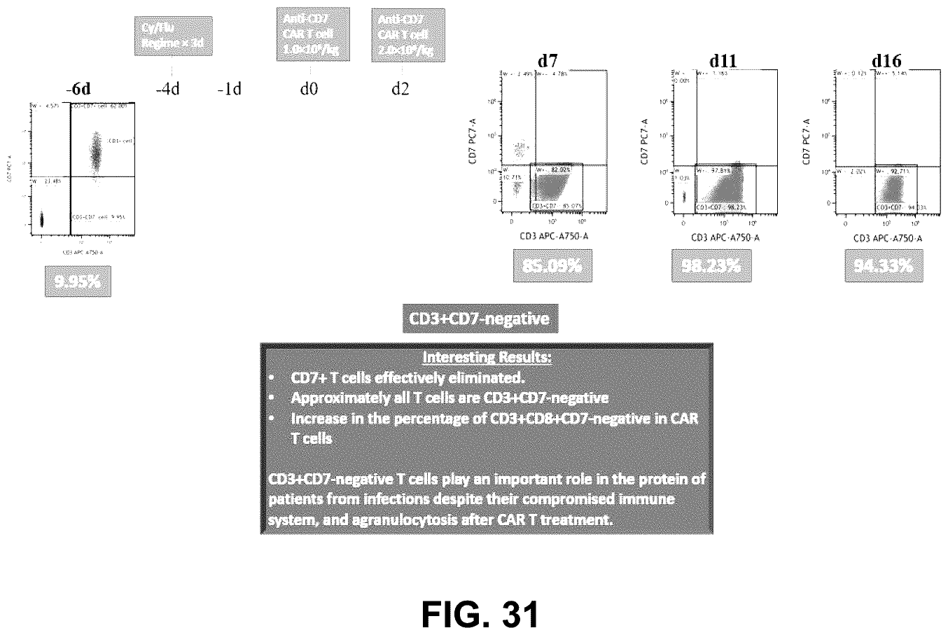

The patient's relapsed T-ALL was positive for CD7 surface antigen, and thus was a candidate for CD7 CAR T cell therapy (FIG. 30). Donor lymphocytes was collected from the original donor who provided the bone marrow stem cells, were collected 6 days prior to CAR T treatment. The basal metrics prior to CAR-T treatment were as follows: blasts in marrow 24%, cerebral spinal fluid (CSF)1.77%, and chimerism 68.36%. A precondition treatment of Fludarabine and Cytarabine chemotherapeutics was administered 4 days prior to CD7 CAR T cell treatment. Anti-CD7-CAR T cells were administered on two occurrences, first (day 0) 1×10⁶/kg CAR T cells and second (day 2) 2×10⁶/kg CAR T cells. By day 7, the patient's peripheral blood was negative for CD7+T-ALL cells. Furthermore, day 11 indicated of CD7 positive T-ALL cells from peripheral blood as well as bone marrow were completely depleted by the CAR-T cell treatment and patient peripheral blood and bone marrow chimerism reached ˜100%. As a result of CD7 CAR T cell treatment, the patient achieved rapid resolution of their leukemia, MRD negative and thus complete remission. Interestingly, virtual all CD7 positive T cells were eliminated day 11 post-CAR (FIG. 31). CD7CAR T cell treatment depleted all the CD7+ T cells, with a residual few percent of CD7 negative T cells able to replenish the absent T cell population to normal levels.

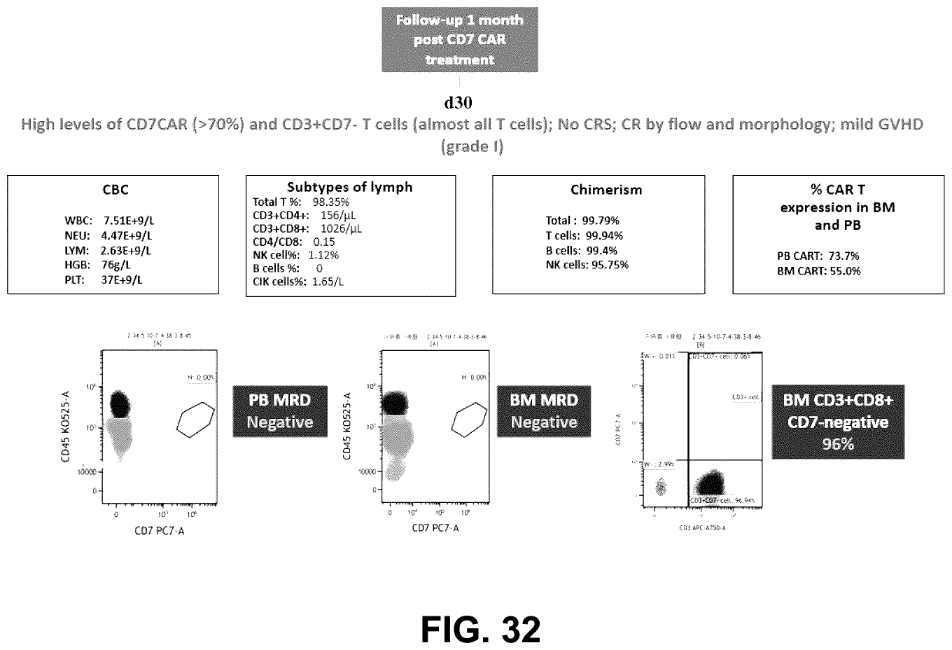

One-month post-CAR treatment (FIG. 32): The patient's CBC indicated a WBC count of 7.51×10⁹/L, NEU 4.47×10⁹/L, LYM 2.63×10⁹/L, HGB 76 g/L and PTL 37×10⁹/L. Most noteworthy, the WBC and LYM count were restored to within the normal range respectively. The lymphocyte subtypes were as follows: total T 98.35%, absolute CD3+CD4+156/μL, CD3+CD8+1026/μL, Total T-cell 1182/μL (the total T cells are within the normal range). The chimerism was as follows: Total 99.79%, T cells 99.94%, B cells 99.4%, and NK cells 95.75%. In summary, at the patients one-month post CD7 CAR T cell treatment follow-up, high levels of CD7 CAR (≥70% detected) and all T are absent of surface protein CD7. The CD7 CAR T cell treatment depleted all of the leukemia cells as well as all T cells expressing CD7 surface protein. The treatment was both efficacious and safe with the patient sustaining treatment with grade 1 cytokine release syndrome (CRS).

In one exemplary embodiment of the invention, T cells expressing CD7CAR were discovered to be effective in treating a human patient suffering from T cell acute lymphoblastic leukemia (T-ALL) expressing CD7. It is known that about 98% of a human's T cell population are CD7 positive. Therefore, it was thought that administering a CD7CAR would deplete a human's T cells and result in death. We unexpectedly discovered that the about 2% of T cells negative for CD7 surface antigen can rapidly expand and replace the eliminated CD7 positive T cell population to a relative normal range within a short time. As a result, a human receiving the CD7CAR tolerates the treatment. At the time of filing, creating CD7CAR T cells according to the claimed invention was against reason because it was thought that the CD7 antigen plays an important role in T cell based killing mechanisms, and elimination of CD7 positive T cells would have fatal results for the patient. Surprisingly, we discovered that the CAR T cell surface protein remained potent in its killing affects, despite the absence of CD7 surface protein, thus elucidating an unexpected finding. In this particular embodiment, the CD7CAR T cells were successful in treating patients with a high burden disease of T cell acute lymphoblastic leukemia. Summarily, prior to the state of the art and teachings, a person of ordinary skill would not create a CD7CAR because the art teaches targeting of CD7 antigen results in total T cell deficiency or depletion, which can be associated with on-target toxicity of severe infections and is incompatible with a patient's life. It is known that about 95% of a human's T cell population are CD7 positive. We unexpectedly discovered that the about 2-5% of T cells negative for CD7 surface antigen can rapidly expand and replace the eliminated CD7 positive T cell population to a relative normal range within a short time. As a result, a human receiving the CD7CAR tolerates the treatment.

It was surprising to observe the following: 1) CD7CAR can be used in treating relapsed/refractory T-ALL patients with remarkable outcomes; 2) CD7 CAR T cell treatment depleted all the CD7+ T cells, with a residual few percent of CD7 negative T cells replenished the absent T cell population to normal levels; and 3) while CD7 CAR T cells without the surface CD7 expression in the patient demonstrate remarkable efficacy of depletion of CD7+ leukemic cells in the peripheral leukemia cells, the T cell level in the peripheral blood is near normal. In one embodiment, a CD7 CAR targeting CD7 surface antigen can deplete the autoreactive immune cell expressing the CD7 surface antigen. The CD7+ population will be depleted (approximately ≥90 T lymphocytes) and an unexpected finding was elucidated in that the CD7-population of T lymphocytes expand to maintain the total T-cell population and prevent infection. Such an occurrence acts as an immune system reset for the T-cell immune system thus treating T-cell mediated autoimmune disorders.

In one embodiment, the engineered cell with CD7-RTX CAR includes a CD7 chimeric antigen receptor polypeptide and two CD20 binding epitopes (also called RTX-binding epitopes) in the hinge region (SEQ ID NO. 1), and corresponding nucleotides (SEQ ID NO. 2). CD7-RTX CAR can be used to deplete T cells associated with autoimmune disorders and leukemia/lymphoma expressing CD7, particularly in patients with T-acute lymphoblastic leukemia/lymphoma or T cell lymphoma or acute myeloid leukemia.

In one embodiment, the engineered cell with CD7-RTX VAC CAR (also called CD7-RTX-IL-15/IL15sushi CAR) includes a CD7 chimeric antigen receptor polypeptide, two CD20 binding epitopes (also called RTX-binding epitopes) in the hinge region, secreting IL-15/IL15sushi (SEQ ID NO. 3), and corresponding nucleotides (SEQ ID NO. 4). CD7-RTX VAC CAR can be used to deplete T cells associated with autoimmune disorders and leukemia/lymphoma expressing CD7, particularly in patients with T-acute lymphoblastic leukemia/lymphoma or T cell lymphoma or acute myeloid leukemia.

In one embodiment, the engineered cell with CD7-RTX-CD19 cCAR includes a CD7 chimeric antigen receptor peptide bearing two CD20 epitopes in the hinge region, a CD19 chimeric antigen receptor polypeptide (SEQ ID NO. 5), and corresponding nucleotides (SEQ ID NO. 6). CD7-RTX-CD19 cCAR can be used to deplete T cells or B cells associated with autoimmune disorders and leukemia/lymphoma expressing CD7 or CD19, particularly in patients with acute lymphoblastic leukemia/lymphoma or T or B cell lymphoma or acute myeloid leukemia.

In one embodiment, the engineered cell with CD7-RTX-CD19 VAC cCAR includes a CD7 chimeric antigen receptor peptide bearing two CD20 epitopes in the hinge region, a CD19 chimeric antigen receptor polypeptide, secreting IL-15/IL-15sushi (SEQ ID NO. 7), and corresponding nucleotides (SEQ ID NO. 8). CD7-RTX-CD19 cCAR can be used to In one embodiment, the engineered cell with CD7-RTX-CD20h cCAR (CD7-RTX-CD20 cCAR) includes a CD7 chimeric antigen receptor peptide bearing two CD20 epitopes in the hinge region, a CD20 chimeric antigen receptor polypeptide (SEQ ID NO. 9), and corresponding nucleotides (SEQ ID NO. 10).

In one embodiment, the engineered cell with CD7-RTX-CD20 VAC cCAR includes a CD7 chimeric antigen receptor peptide bearing two CD20 epitopes in the hinge region, a CD20 chimeric antigen receptor polypeptide, secreting IL-15/IL-15sushi (SEQ ID NO. 11), and corresponding nucleotides (SEQ ID NO. 12).

In one embodiment, the engineered cell with CD7-RTX-CD33 VAC cCAR includes a CD7 chimeric antigen receptor peptide bearing two CD20 epitopes in the hinge region, a CD33 chimeric antigen receptor polypeptide, secreting IL-15/IL-15sushi (SEQ ID NO. 13), and corresponding nucleotides (SEQ ID NO. 14).

In one embodiment, the engineered cell with CD7-RTX-CLL1 VAC cCAR includes a CD7 chimeric antigen receptor peptide bearing two CD20 epitopes in the hinge region, a CLL1 chimeric antigen receptor polypeptide, secreting IL-15/IL-15sushi (SEQ ID NO. 15), and corresponding nucleotides (SEQ ID NO. 16).

In one embodiment, the engineered cell with CD7-RTX-CS1 cCAR includes a CD7 chimeric antigen receptor peptide bearing two CD20 epitopes in the hinge region, a CS1 chimeric antigen receptor polypeptide(SEQ ID NO. 17), and corresponding nucleotides (SEQ ID NO. 18).

In one embodiment, the engineered cell with CD7-RTX-BCMA cCAR includes a CD7 chimeric antigen receptor peptide bearing two CD20 epitopes in the hinge region, a BCMA chimeric antigen receptor polypeptide (SEQ ID NO. 19), and corresponding nucleotides (SEQ ID NO. 20).

In one embodiment, the engineered cell with CD7-RTX-BCMA VAC cCAR includes a CD7 chimeric antigen receptor peptide bearing two CD20 epitopes in the hinge region, a BCMA chimeric antigen receptor polypeptide, secreting IL-15/IL-15sushi (SEQ ID NO. 21), and corresponding nucleotides (SEQ ID NO. 22).

In one embodiment, the engineered cell with CD7-RTX-CD38 cCAR (CD7-RTX-CD38a cCAR) includes a CD7 chimeric antigen receptor peptide bearing two CD20 epitopes in the hinge region, a CD38 chimeric antigen receptor polypeptide (SEQ ID NO. 23), and corresponding nucleotides (SEQ ID NO. 24).

In one embodiment, the engineered cell with pX-BCMA-CD19-IL15/IL15sushi including a chimeric antigen receptor peptide, a CD19 chimeric antigen receptor polypeptide, a secreted IL-15/IL15sushi fusion ((SEQ ID NO. 25, and 47) and corresponding nucleotides (SEQ ID NO. 26 and 48). pX-BCMA-CD19-IL15/IL15sushi can be used to deplete or reduce plasma cells and or/B cells in patients with autoimmune diseases or leukemia cells expressing BCMA and/or B cells.

In one embodiment, the engineered cell with pX-BC4-CD7 RTX -15R cCAR including a chimeric antigen receptor peptide, a BCMA chimeric antigen receptor polypeptide, a CD19 chimeric antigen receptor polypide and a secreted IL-15/IL15sushi fusion ((SEQ ID NO. 27) and corresponding nucleotides (SEQ ID NO. 28). pX-BC4-CD7 RTX -15R cCAR can be used to deplete or reduce plasma cells and/or T cells associated with autoimmune disorders.

In one embodiment, the engineered cell with pX-BC4-CD19c-Vac (also called pX-BC4-CD19c-IL-15/IL15sushi) including a chimeric antigen receptor peptide, a BCMA chimeric antigen receptor polypeptide, a CD19 chimeric antigen receptor polypide and a secreted IL-15/IL15sushi fusion ((SEQ ID NO. 29) and corresponding nucleotides (SEQ ID NO. 30). pX-BC4-CD19c-Vac CAR can be used to deplete or reduce plasma cells and or/B cells in patients with autoimmune diseases or leukemia cells expressing BCMA and/or B cells.

In one embodiment, the engineered cell with CLL1-CD33b-IL15/IL15sushi cCAR includes a chimeric antigen receptor peptide, a CLL1 chimeric antigen receptor polypeptide, a CD33 chimeric antigen receptor polypide and a secreted IL-15/IL15sushi fusion ((SEQ ID NO. 31) and corresponding nucleotides (SEQ ID NO. 32). CLL1-CD33b-IL15/IL15sushi cCAR can be used to treat patients with CLL1 or CD33 expressing acute myeloid leukemia or chronic leukemias, myelodysplastic syndrome and prevent from tumor antigen escape.

In one embodiment, the engineered cell with pX-CD7 RTX-CD19c-15R includes a chimeric antigen receptor peptide, a CD7 chimeric antigen receptor polypeptide, a CD19 chimeric antigen receptor polypide and a secreted IL-15/IL15sushi fusion ((SEQ ID NO. 33) and corresponding nucleotides (SEQ ID NO. 34). pX-CD7q-CD19c-15R can be used to delete T cells and/or B cells associated to autoimmune disorders and then reset all B and T cell immune system.

In one embodiment, the engineered cell with pX-BC4-CD7 RTX-tandem CAR includes BCMA VVH linked CD7 scFv via a linker (BCMA-CD7 tandem construct), a hinge region, a transmembrane domain, co-stimulatory domain and CD3 signaling domain. The co-stimulatory domain includes, not limited to, CD28 and 4-1BB. pX-BC4-CD7 RTX-tandem CAR sequence, SEQ ID NO. 35) and corresponding nucleotides (SEQ ID NO. 36). pX-BC4-CD7Q-tandem CAR can be used to delete plasma cells and/or T cells associated with autoimmune disorders or lymphoma/leukemia expressing either BCMA and/or CD7 antigens.

In one embodiment, the engineered cell with pX-BC4-CD19-tandem CAR includes BCMA VVH linked CD7 scFv via a linker (BCMA-CD19 tandem construct), a hinge region, a transmembrane domain, co-stimulatory domain and CD3 signaling domain. The co-stimulatory domain includes, not limited to, CD28 and 4-1BB. pX-BC4-CD19-tandem CAR has SEQ ID NO. 37, 39, 41 and corresponds nucleotides (SEQ ID NO. 38, 40, 42). pX-BC4-CD19-tandem CAR can be used to delete plasma cells and/or B cells associated with autoimmune disorders or treat patients with lymphoma/leukemia expressing either BCMA and/or CD19 antigens.

In one embodiment, the engineered cell with pX-CD19-CD7 tandem CAR includes CD19 scFv linked CD7 scFv via a linker (CD19-CD7 tandem construct), a hinge region, a transmembrane domain, co-stimulatory domain and CD3 signaling domain. The co-stimulatory domain includes, not limited to, CD28 and 4-1BB. pX-CD19-CD7-tandem CAR includes SEQ ID NO. 43, 45) and corresponding nucleotides (SEQ ID NO. 44, 46). pX-CD19-CD7-tandem CAR can be used to delete B and/or T cells associated with autoimmune disorders or lymphoma/leukemia expressing either B and/or T antigens.

In one embodiment, the engineered cell with pX-BC4-RTX Vac CAR (also pX-BC4-RTX -IL-15/IL15sushi CAR) includes a chimeric antigen receptor peptide, a BCMA chimeric antigen receptor polypeptide, and a secreted IL-15/IL15sushi fusion ((SEQ ID NO. 49, 51) and corresponding nucleotides (SEQ ID NO. 50 and 52). pX-BC4-RTX Vac CAR can be used to deplete or reduce plasma cells associated with autoimmune disorders and myeloma expressing BCMA.

In one embodiment, the engineered cell with pX-BC5-RTX Vac CAR (also pX-BC4-RTX -IL-15/IL15sushi CAR) includes a chimeric antigen receptor peptide, a BCMA chimeric antigen receptor polypeptide, and a secreted IL-15/IL15sushi fusion ((SEQ ID NO. 53, 55) and corresponding nucleotides (SEQ ID NO. 54 and 56). pX-BC5-RTX Vac CAR can be used to deplete plasma cells associated with autoimmune disorders and myeloma expressing BCMA.

In one embodiment, the engineered cell with pX-BC4-RTX CAR includes a chimeric antigen receptor peptide, a BCMA chimeric antigen receptor polypeptide (SEQ ID NO. 57, 59) and corresponding nucleotides (SEQ ID NO. 58 and 60). pX-BC4-RTX CAR can be used to deplete or reduce plasma cells associated with autoimmune disorders or myeloma expressing BCMA.

In one embodiment, the engineered cell with pX-BC5-RTX CAR includes a chimeric antigen receptor peptide, a BCMA chimeric antigen receptor polypeptide (SEQ ID NO. 61, 63) and corresponding nucleotides (SEQ ID NO. 62 and 64). pX-BC5-RTX CAR can be used to deplete or reduce plasma cells associated with autoimmune disorders and myeloma expressing BCMA.

In one embodiment, a specific chimeric antigen receptor (CAR) comprises a BCMA antigen binding domain comprising the amino acid sequence of SEQ. ID NO: 77 or SEQ. ID NO: 78, a hinge domain, a transmembrane domain, at least one co-stimulatory domain and a CD3 zeta signaling domain. In a further embodiment, the BCMA specific chimeric antigen receptor wherein said antigen binding domain comprises an amino acid sequence selected from group consistent of: SEQ ID NO: 49, 51, 53, 55, 57, 59, 61 and 63.

In one embodiment, the BCMA specific chimeric antigen receptor comprises an amino acid sequence of antigen binding domain having at least 75%, preferably 80%, 85%, 90%, 95% with the amino acid sequences, SEQ ID NO: 77 and 78.

In one embodiment, a BCMA antigen binding domain comprising the amino acid sequence of SEQ. ID NO: 77 or SEQ. ID NO: 78 can be used for the reduction or deletion of plasma cells associated with autoimmune disorders or myeloma cells expressing BCMA.

In one embodiment, a method of producing an anti-BCMA antibody comprising culturing the host cells of Chinses hamster ovary (CHO) cells comprising the amino acid sequence of SEQ. ID NO: 77 or SEQ. ID NO: 78, so that the antibody is produced, and isolated from culture. The similar methods of production and isolation of an anti-BCMA antibody are described in the Patent No.: US 10, 239, 947.

In one embodiment, the engineered cell with pX-BC4-CD7 RTX-VAC-tandem CAR (also pX-BC4-CD7-RTX -IL-15/IL15sushi tandem CAR) includes a BCMA VHH linked CD7 via a linker and a secreted IL-15/IL15sushi fusion ((SEQ ID NO. 65) and corresponding nucleotides (SEQ ID NO. 66). pX-BC4-CD7 RTX-VAC-tandem CAR can be used to treat plasma cells and/or T cells associated with autoimmune disorders and myeloma/lymphoma expressing BCMA and/or expressing CD7.

In one embodiment, the engineered cell with pX-BC4-CD19-VAC-tandem CAR includes a BCMA VHH linked CD19 via a linker and a secreted IL-15/IL15sushi fusion ((SEQ ID NO. 67 69 and 71) and corresponding nucleotides (SEQ ID NO. 68, 70 and 72). pX-BC4-CD19 RTX-VAC-tandem CAR can be used to treat plasma cells and B cells associated with autoimmune disorders and myeloma expressing BCMA and/or lymphoma/leukemia expressing CD19.

In one embodiment, the engineered cell with pX-CD19-BC4-VAC-tandem CAR includes a CD19 scFv linked VHH BCMA via a linker and a secreted IL-15/IL15sushi fusion (SEQ ID NO. 73 and 75) and corresponding nucleotides (SEQ ID NO. 74 and 76). pX-CD19-BC4-VAC-tandem CAR can be used to treat B cells and plasma cells associated with autoimmune disorders and lymphoma/leukemia expressing CD19 or/BCMA.

Resolution of CAR-CAR Interaction and Increased Insert Size by a Compound CAR-BiTE Associated with Reduction of the Viral Titer.

In one embodiment, two or more units of CARs expressing in a cell need to avoid CAR-CAR interaction.

In one embodiment, the hinge region is designed to exclude amino acids that may cause undesired intra- or intermolecular interactions. For example, the hinge region may be designed to exclude or minimize cysteine residues to prevent formation of disulfide bonds. In another embodiment, the hinge region may be designed to exclude or minimize hydrophobic residues to prevent unwanted hydrophobic interactions.”

In another embodiment, the transmembrane domain is selected or modified by amino acid substitution to avoid binding of such domains to the transmembrane domains of the same or different surface membrane proteins to minimize interactions with other members of the receptor complex.

The novel compound CAR contains a set of two-CAR polypeptides with 12 functional domains along with a self-cleavage sequence which is greater than the single CAR which contains 6 functional domains. As discussed in the previous office actions, creating a compound CAR according to the claimed invention was against reason at the time of filing because it was thought that the longer the polypeptide, the less effective it becomes for making viruses for CARs and insufficient killing. This was due to a lower level of protein expression expected with increasing size. Research has shown the level of expression drops with increasing insert size (Int J Biochem Mol Biol. 2013; 4(4): 201-208). Therefore, a person of ordinary skill in the art would not motivate to use to generate a large sized compound CAR or CAR with BiTE.

During a clinical trial with a compound CAR, it was surprisingly discovered that CAR T cells with CAR efficiencies below or around 5% can still eliminate targeted cells, leukemic cells and result in completion remission. This suggests that alternate/weaker promoters can be used in making effective cCAR T cells in accordance with the claimed invention.

Inventive Steps by Surprising Discover to Overcome the Low Titer of Viruses with a Large Sized cCAR or cCAR-BiTE

It was surprisingly finding that retroviral stable producer cell lines could resolute this problem related to the low titer of viruses with a larger sized CAR.

In particular embodiments, generation of a high titer of cCAR or cCAR-BiTE can be achieved by combination of at least one or more of the following steps:
1. Lenti or retroviral cCAR or cCAR-BiTE is transduced to a stable producer cell line wherein non-cytotoxic viral envelops, such as RD114 is substituted.
2. The CAR expression in the stable producer cell line is determined by flow cytometry assay with anti-Fab antibodies
3. Sorting the stable producer cells with high expression of cCAR or cCAR-BitTE using fluorescence activated cell sorting (FACS) with anti-FAB antibodies
4. Expansion of sorted cells with a high level of CAR expression is performed and then test their secreted retroviruses or lentiviruses.
5. Cloning a highly expressed cell by limiting dilution.
   Generation of a High Titer of Large Sized BCMA-CD19 VAC cCAR Retroviruses Using a Stable Producer Cell Line

BCMA-CD19 VAC cCAR (also called BCMA-CD19 IL-15/IL-15sushi cCAR) is a two unit CAR composed of a complete BCMA-CAR fused to a complete CD19 CAR by a self-cleaving, enabling independent expression of both CAR receptors separately on the T cell surface. Each CAR contains a CD8 leader, a CD8-derived hinge (H) and transmembrane (TM) regions, and co-activation domains linked to the CD3ξ signaling domain. An enhancer, IL-15/IL-15sushi is also separated from the first CAR and second CAR by a second cleavage site at the C-terminus. The expression of BCMA-CD19 VAC cCAR is controlled by the Moloney murine leukemia virus (MMLV), the long terminal repeat (LTR) promoter.

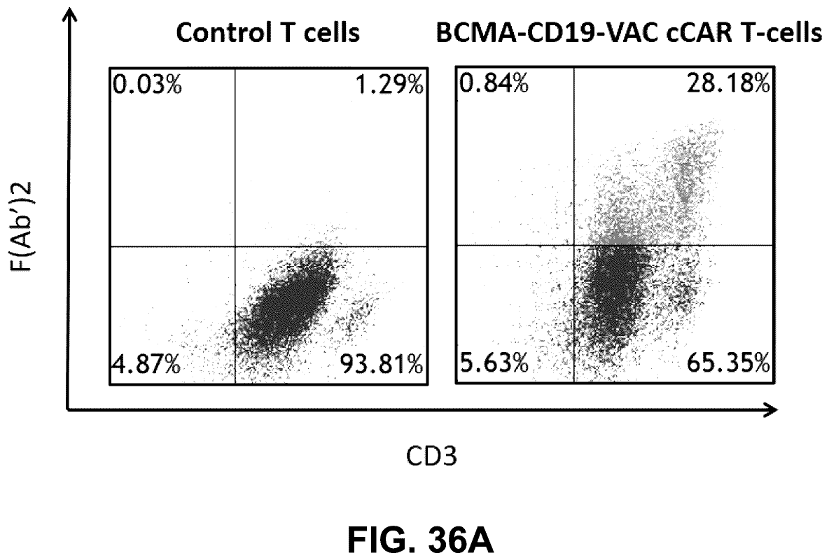

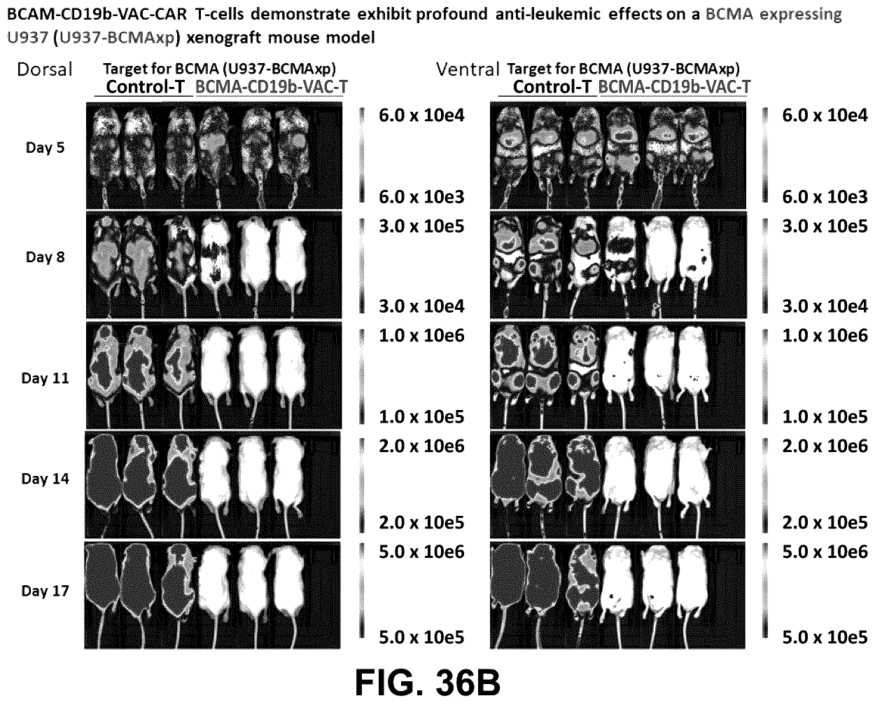

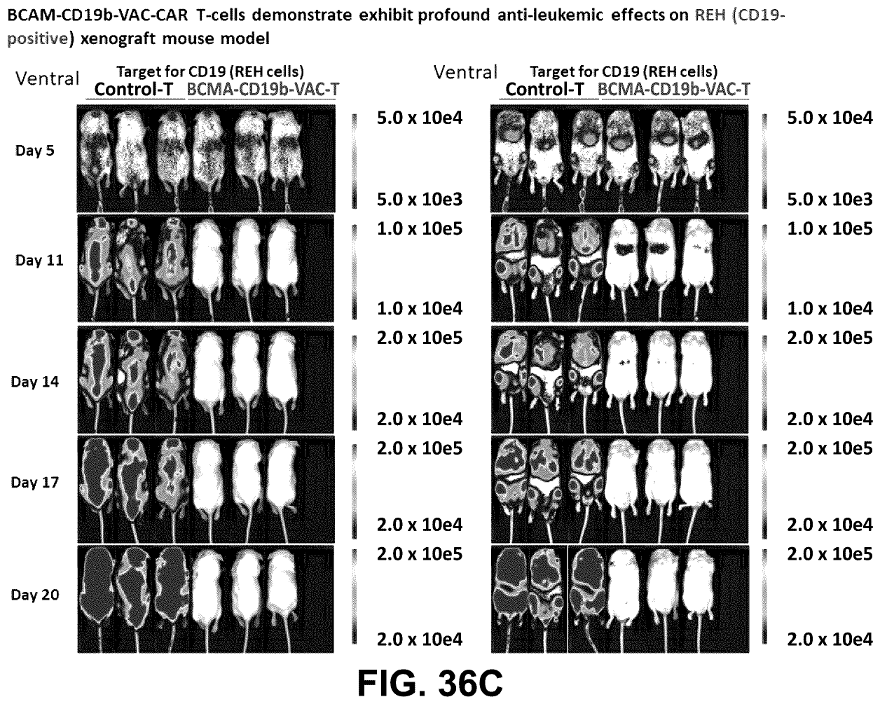

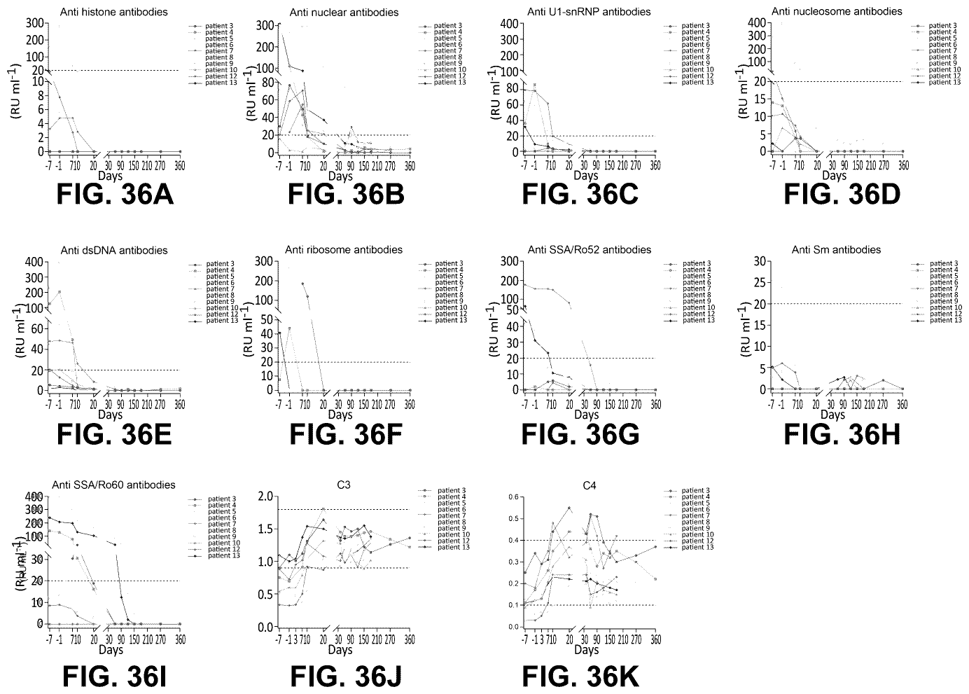

The BCMA-CD19 VAC cCAR (also called BCMA-CD19b-IL-15/IL15sushi) contract was transfected into the H29 cell line (transient), viruses from transduced H29 were then transduce on the RD114 cell line (293, envelope protein from RD114 retrovirus). This envelope protein is utilized because it is not toxic to the cell. Cell sorting was used to identify highly expressing CAR population. The sorted population of highly expressing CAR cells was then expanded. The viruses secreted from expanded cells were then tested on T-cells for CAR expression. Additionally, a single clone that expresses a high level of CAR was cloned by limiting dilution. Viruses from a highly expressed clone was tested for CAR expression by flow cytometry. Flow cytometry results showed that BCMA-CD19-VAC cCAR-T was expressed on roughly 28.18% of T-cells (FIG. 36).

In order to evaluate the in vivo anti-tumor activity of BCMA-CD19b-IL-15/IL15sushi CAR T cells on BCMA-expressing cells, we developed a xenogeneic mouse model using NSG mice sub-lethally irradiated and intravenously injected with 1×10e6 luciferase-expressing U937-BCMA cells, a human tumor cell line that is synthetically expressing BCMA (CD269) on the cell surface, to induce measurable tumor formation (FIG. 36). Five days following tumor cell injection, 3 mice each group were intravenously injected with a course of either 10×10⁶ BCMA-CD19b-IL-15/IL15sushi T cells or control T cells. On days 5, 8, 11, 14 and 17, mice were injected subcutaneously with RediJect D-Luciferin (Perkin Elmer) and subjected to IVIS imaging to measure tumor burden (FIG. 36B). Average light intensity measured for the BCMA-CD19b-IL-15/IL15sushiCAR T cell injected mice was compared to that of control T cell injected mice in order to determine the percentage of tumor cells in treated versus control mice. CAR T cell-treated mice had an 80% lower tumor burden relative to control on Day 8, whereas BCMA-CD19b-IL-15/IL15sushiCAR T cell-treated mice had a 99% lower tumor burden by Day 17, when nearly all tumor cells had been lysed in treated mice. Unpaired T test analysis revealed a very significant difference (P=0.0045) between control and the two groups by day 14 with less light intensity and thus less tumor burden in the BCMA-CD19b-IL-15/IL15sushi CAR T cells treated group compared to control. In summary, these in vivo data indicate that BCMA-CD19b-IL-15/IL15sushiCAR T cells both significantly reduce tumor burden and in U937-BCMA-injected NSG mice when compared to vector control T cells.

In order to evaluate the in vivo anti-tumor activity of BCMA-CD19b-IL-15/IL15sushi CAR T cells on CD19 expressing cells, we developed a xenogeneic mouse model using NSG mice sub-lethally irradiated and intravenously injected with 1×10e6 luciferase-expressing REH cells, a B cell derived human tumor cell line that is expressing the CD19 antigen (FIG. 36C). Five days following tumor cell injection, 3 mice each group were intravenously injected with a course of either 10×10⁶ BCMA-CD19b-IL-15/IL15sushi T cells (the same cells as the previous experiment) or control T cells. On days 5, 8, 11, 14, 17 and 20, mice were injected subcutaneously with RediJect D-Luciferin (Perkin Elmer) and subjected to IVIS imaging to measure tumor burden (FIG. 36C). Average light intensity measured for the BCMA-CD19b-IL-15/IL15sushi CAR T cell injected mice was compared to that of control T cell injected mice in order to determine the percentage of REH tumor cells in treated versus control mice. CAR T cell-treated mice had a 72% lower tumor burden relative to control on Day 8, whereas BCMA-CD19b-IL-15/IL15sushiCAR T cell-treated mice had a 99% lower tumor burden by Day 17, when nearly all tumor cells had been lysed in treated mice. Unpaired T test analysis revealed a very significant difference (P=0.0045) between control and the two groups by day 14 with less light intensity and thus less tumor burden in the BCMA-CD19b-IL-15/IL15sushi CAR T cells treated group compared to control. In summary, these in vivo data indicate that BCMA-CD19b-IL-15/IL15sushi CAR T cells both significantly reduce tumor burden and in REH-injected NSG mice when compared to vector control T cells.

Lupus nephritis (LN) is a common and severe complication of systemic lupus erythematosus (SLE). In this phase 1 open-label clinical trial, sequentially assigned biopsy-confirmed (class III-V) LN patients were received with 1.5-3×10⁶ BCMA-CD19b-IL-15/IL15sushi cCAR cells/kg post-cessation of all SLE medications and conditioning. A total of 13 SLE patients received the intervention (1.5-3×10⁶ cCAR cells/kg). All patients were negative for all autoantibodies, including those derived from long-lived plasma cells, 3-months post-cCAR. All patients achieved symptom- and medication-free complete remission (CR) with post-cCAR follow-up up to 44-months. Mean Systemic Lupus Erythematosus Disease Activity Index 2000 (SLEDAI-2K) reduced from 9.9 (baseline) to 2.4 (3 months) (or do we want to say 1.0 at 6 months), and renal function significantly improved in 10 LN patients ≤90 days post-cCAR. Complete recovery of B-cells was seen in 2-6-months post-cCAR. Complete immune reset was confirmed by B-cell receptor (BCR) deep sequencing and flow cytometry analysis. In this phase 1 trial, SLE patients given cCAR achieved medication-free CR with remarkable safety and toxicity. Depletion of disease-causing autoantibodies derived from B and long-lived plasma cells (FIG. 36D), was indicative of a complete immune reset. At 2-4 months post-treatment all 13 patients were negative for the following autoantibodies: anti-histone (AHA), anti-nuclear (ANA), anti-U1-snRNP, anti-nucleosome, anti-double-stranded DNA (dsDNA), anti-ribosome, anti-SSA/Ro52, anti-Sm, and anti-SSA/Ro60. Levels of anti-centromeric B, anti-exoribonuclease, and anti-SSB/La autoantibodies were found to be below the pathological threshold in all patients. The depletion of anti-SSA/Ro52 and anti-SSA/Ro60 indicated absence of disease-causing long-lived plasma cells and plasmablasts. All SLE patients indicated CH50 and specifically C3 and C4 recovery, post-cCAR (FIG. 36D). This therapeutic approach may be extended to treat other autoimmune diseases.

The other example CLL1-CD33-VAC cCAR-T construct bearing the large insert size does not hinder the expression, and that the single clone obtained by clone limiting dilution, expresses high levels of CAR on the T-cells was shown below.

CLL1-CD33 VAC cCAR (also called CLL1-CD33 IL-15/IL-15sushi cCAR or CLL1-CD33b VAC CAR) is a two-unit CAR composed of a complete CLL1-CAR fused to a complete CD33 CAR by a self-cleaving, enabling independent expression of both CAR receptors separately on the T cell surface. Each CAR contains a CD8 leader, a CD8-derived hinge (H) and transmembrane (TM) regions, and co-activation domains linked to the CD3ξ signaling domain. An enhancer, IL-15/IL-15sushi is also separated from the first CAR and second CAR by a second cleavage site at the C-terminus.

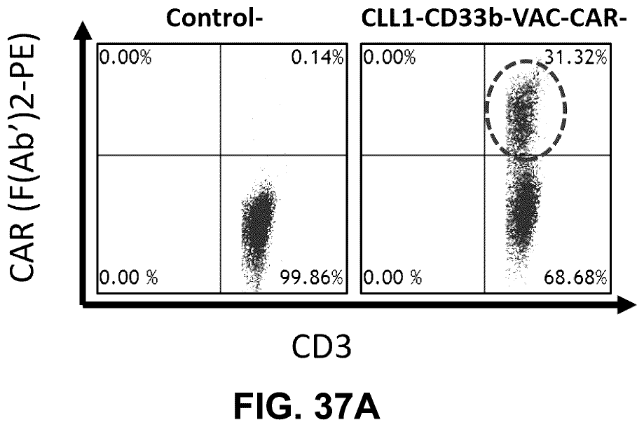

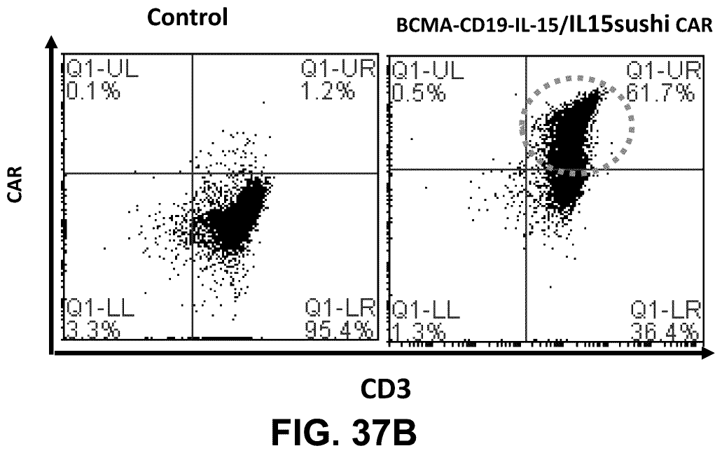

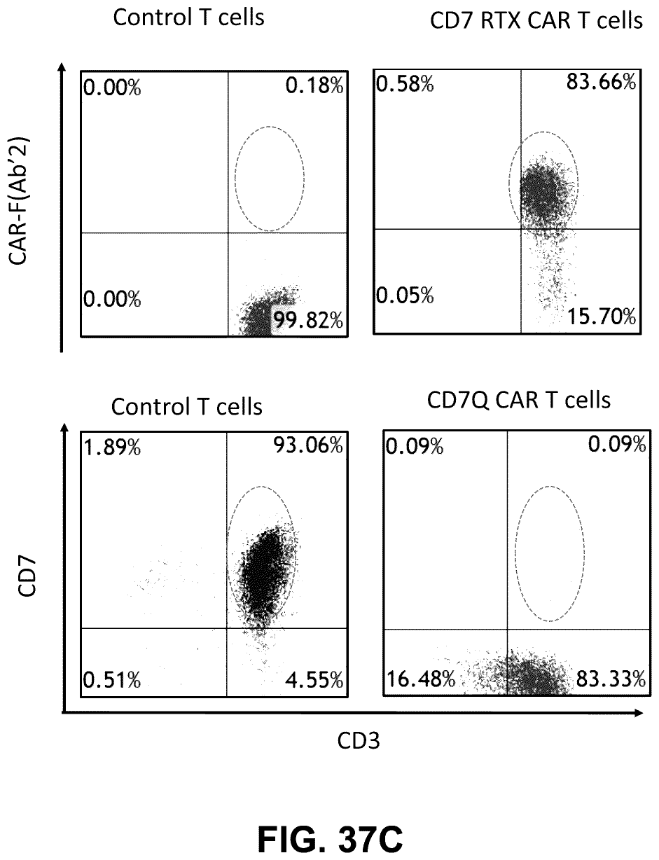

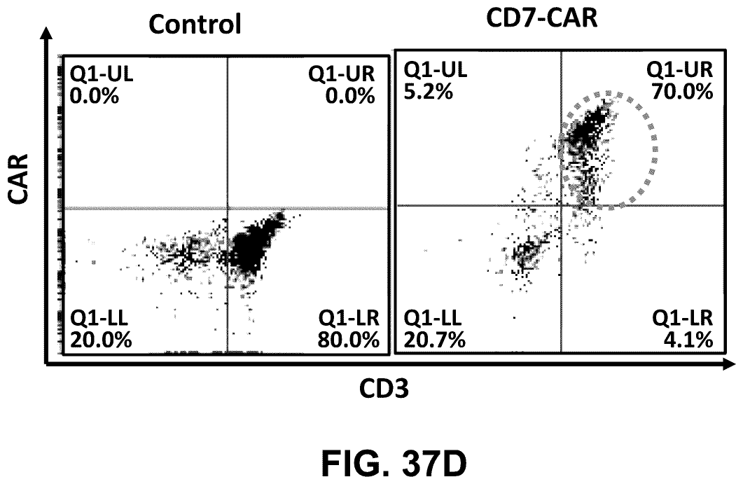

The construct of CLL1-CD33 VAC cCAR was controlled driven by the Moloney murine leukemia virus (MMLV), the long terminal repeat (LTR) promoter, and was transfected into the H29 cell line then, H29 producing-viruses were used to transduce RD114 cells. The highly expressed clones for CLL1-CD33 VAC cCAR were then screened by limiting dilution. The highly expressed clones were identified by flow cytometry analysis. It was unexpected that CLL1-CD33 VAC cCAR was expressed on roughly 31.32% of T-cells (FIG. 37A). The unexpected results were also seen when expression of CLL1-CD33-IL-15/IL-15sushi was driven by the MMLV promoter (FIG. 36A). As compared to a strong SFFV (spleen focus-forming virus) promoter that drives expression of the single polypeptide which results in the generation of both CARs (Table I). Most people had had a very low level of cCAR T expression which was below 10%. Therefore, the Moloney murine leukemia virus (MMLV), the long terminal repeat (LTR) promoter provides a better option for the generation of cCAR. It was also surprisingly found that upon treatment with cCAR engineered cells having a first chimeric antigen receptor polypeptide selective for CLL-1, and a second chimeric antigen receptor polypeptide selective for CD33, 9 of 11 patients with AML exhibited complete remission. In addition, IL-15/IL15sushi levels did not elevate substantially post-infusion, staying within the pg/mL range. No lymphoma or leukemia caused by CAR treatment was found.

In one embodiment, the engineered cell with CLL1-CD33-IL-15/IL15sushi cCAR includes a chimeric antigen receptor peptide, a CLLlchimeric antigen receptor polypeptide, a CD33 chimeric antigen receptor polypeptide and a secreted IL-15/IL15sushi fusion ((SEQ ID NO. 31) and corresponding nucleotides (SEQ ID NO. 32). CLL1-CD33-IL-15/ILl5sushi cCAR can be used to treat acute myeloid leukemia or myeloid leukemia expressing CLL1 and/or CD33.

TABLE 1
Patient Characteristics

| | | | | BM | CD33/ | | Origin | | | |
|---|---|---|---|---|---|---|---|---|---|---|
| | | Age/ | Prior | Blast | CLL1 | Cytogenetic/ | of car-t | CAR-T | CAR T | Responses |
| CAR | Patient | sex Dx | treatment | % | expression | molecular | cells | Dose | expression | MRD |
| CLL1- | P1 | 44/M | 4 | 47% | CD33⁺/ | ASXL1, TP53 | auto | 0.7 × 10⁶/ | 18.6% | MRD⁻ |
| CD33 | | AML | chemo | | CLL1⁺ | | | kg | | |
| CLL1- | P2 | 6/F | 5 | 81% | CD33⁺/ | Complex | auto | 2 × 10⁶/ | 11.8% | MRD⁻ |
| CD33 | | JMML- | chemo | | CLL1⁺ | FLT3-ITD | | kg | | |
| | | AML | | | | | | | | |
| CLL1- | P3 | 23/F | 3 TKIs | 1.63% | CD33⁺/ | t(9; 22) | auto | 1.1 × 10⁶/ | 4% | MRD⁻ |
| CD33 | | AML | for 5 | | CLL1⁺ | T315mut | | kg | | |
| | | | years | | | | | | | |
| CLL1- | P4 | 43/F | 3 | 42% | CD33⁺/ | NK | auto | 2.8 × 10⁶/ | 6% | MRD⁻ |
| CD33 | | M2 | chemo | | CLL1⁺ | FLT3-ITD | | kg | | |
| CLL1- | P5 | 32/F | 3 | 19% | CD33⁺/ | NK | auto | 2 × 10⁶/ | 4.9% | MRD⁻ |
| CD33 | | AML | chemo | | CLL1⁺ | MLL | | kg | | |
| CLL1- | P6 | 48/F | 5 | 94% | CD33⁺/ | t(8; 21) | auto | 1.3 × 10⁶/ | 4.5% | MRD⁻ |
| CD33 | | AML | chemo | | CLL1⁺ | AML1/ETO | | kg | | |
| | | | | | | CKIT | | | | |
| CLL1- | P7 | 23/F | 4 | 74% | CD33⁺/ | t(8; 21) | auto | 1 × 10⁶/ | 3.2% | NR |
| CD33 | | AML | chemo | | CLL1⁺ | AML1/ETO | | kg | | |
| | | | | | | CKIT | | | | |
| CLL1- | P8 | 27/F | 5 | 93% | CD33⁺/ | NA | auto | 2.3 × 10⁶/ | 8% | NR |
| CD33 | | AML | chemo | | CLL1⁺ | MLL AF9 | | kg | | |
| CLL1- | P9 | 42/M | 2 | 7% | CD33⁺/ | T(3; 3) | MSD | 3.7 × 10⁶/ | 14% | MRD⁻ |
| CD33 | | AML | chemo | | CLL1⁺ | RUNX1 | donor | kg | | |
| CLL1- | P10 | 30/F | 8 | 9.7% | CD33⁺/ | T(11; 19) | HSC | 1 × 10⁶/ | 11% | MRD⁻ |
| CD33 | | AML | chemo | | CLL1⁺ | MLL/ELL | donor | kg | | |
| | | | HSCT | | | | | | | |
| CLL1- | P11 | 28/F | 5 | 87% | CD33⁺/ | NA | HSC | 1 × 10⁶/ | 8% | MRD⁻ |
| CD33 | | AML | chemo | | CLL1⁺ | MLL/AF9 | donor | kg | | |
| | | | HSCT | | | | | | | |

### Example: Generation of BCMA-CD19-IL-15Sushi (Also Called BCMA-CD19 VAC cCAR) and CD7 CAR are Driven by a MMLV Promoter

The construct of BCMA-CD19 VAC cCAR and CD7 CAR were generated and their expression were also driven by the Moloney murine leukemia virus (MMLV), the long terminal repeat (LTR) promoter using a similar strategy as described above. PBMC from healthy human donors was activated 48 hours in the presence of mouse anti-human CD3 antibody. Cells (1.0×10e6) were transduced with either 1 mL control supernatant (left panels) or supernatant retroviruses of CAR (right panels). Cells were labeled for flow cytometry analysis, with either goat anti-mouse F(Ab′)2 antibody to detect CAR phenotype, and mouse anti-human CD3 and CD7 to detect the decrease in T cell phenotype populations in transduced cells. It was unexpected that BCMA-CD19-IL-15/IL15sush and CD7 RTX CAR were expressed on roughly 61% (FIG. 37B) and 83% (FIG. 37C), respectively, 2 days post-virial infection. The CD7+ T cell population was completely depleted (FIG. 37C, lower right panel).

### Example: Generation of CD7-Negative T Cells Containing a BCMA-CD7 cCAR

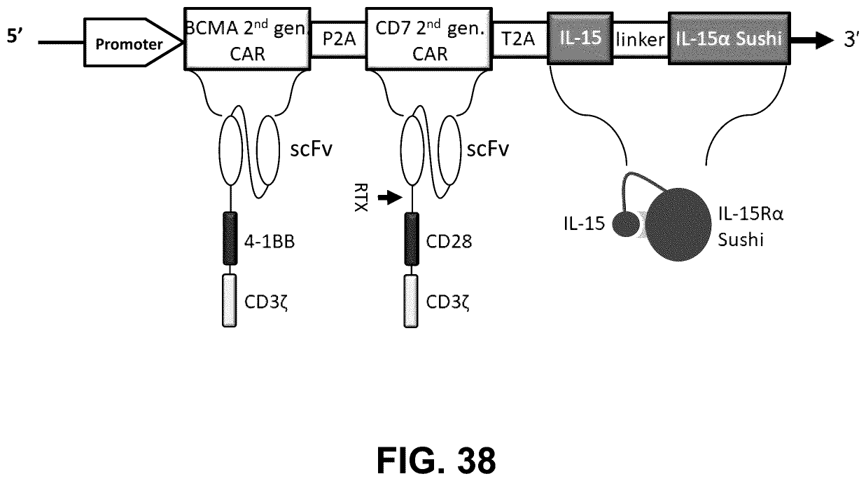

A combination of compound CAR (cCAR) is assembled on an expression vector and expression is driven by a promoter. The first BCMA CAR and the second CD7 CAR are linked with a P2A self-cleaving sequence. A safety switch (rituximab) has been incorporated on the hinge region. A cCAR and IL-15/IL15sushi domain of the IL-15 alpha receptor (BCMA-CD7-IL-15/IL-15sushi) can optionally be assembled on an expression vector and its expression is driven by a promoter (FIG. 38). The first BCMA CAR and the second CD7 CAR are linked with the P2A self-cleaving sequence, and the CD7 CAR is linked with IL-15/IL-15sushi with the T2A self-cleaving sequence. The IL-15/IL-15sushi portion is composed of IL-2 signal peptide fused to IL- and linked to sushi domain of IL-15 alpha via a 26-amino acid poly-proline linker. A safety switch (rituximab) has been incorporated on the hinge region.

BCMA-CD7-IL-15/IL-15sushi was used to generate CD7-negative T cells due to the presence of CD7 CAR in the construct.

### Retroviral Production and Production of Stably-Transduced Retrovirus-Expressing Cells

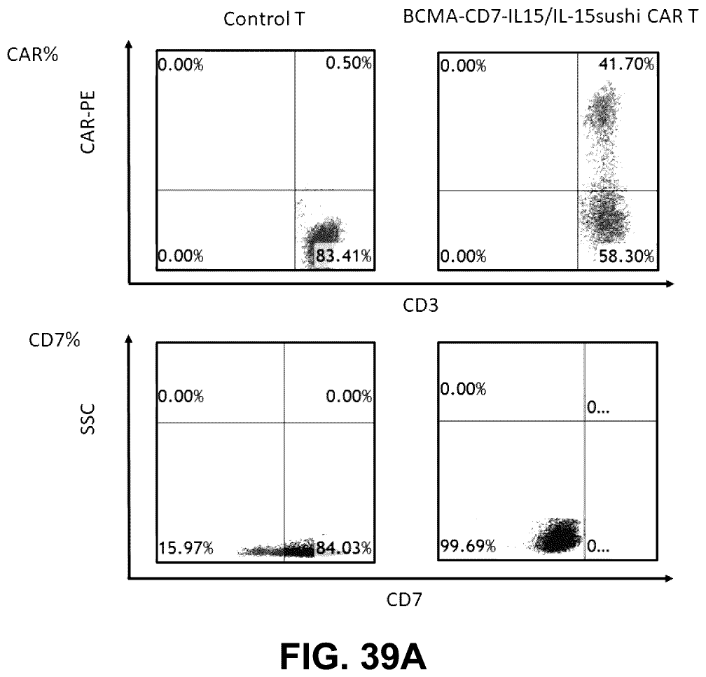

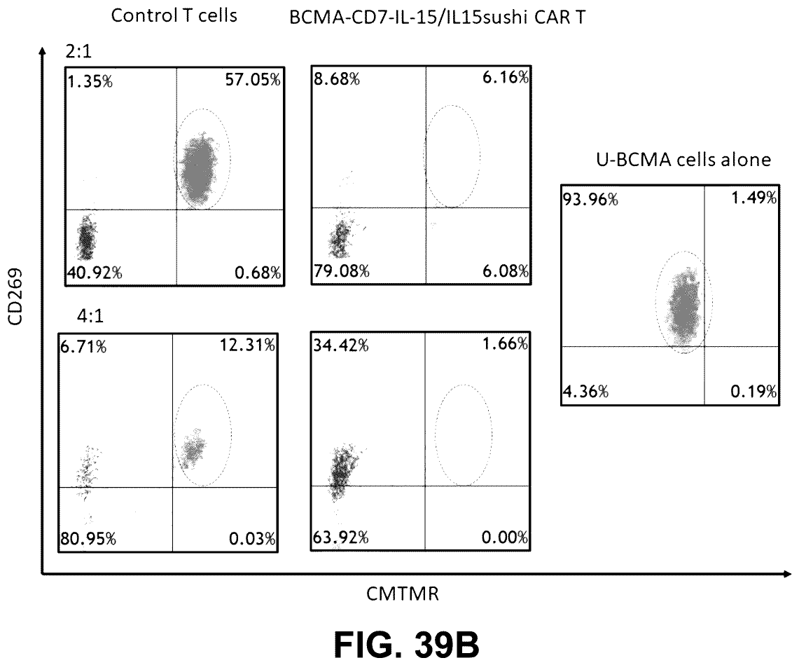

The H29 cell line is first transfected with the plasmid containing pX-BCMA-CD7-15/IL15sushi gene using Lipofectamine 2000 (Life Technologies, Carlsbad, CA) per manufacturer's protocol. After incubation of 3 days, supernatant was removed and replaced with culture media containing DMEM, 10% FBS. The supernatant was used to transduce RD114 cells, HEK293 cells that stably express the gag/pol and vsv genes necessary for viral replication, with the addition of polybrene. After 24 hours incubation, the supernatant was removed and replaced with a second harvest of viral supernatant from the H29 cells. This process was repeated a third time. Following 3 transductions, cells were expanded to larger flasks, and sorted by FACS based on goat anti-mouse F(Ab′)2 expression, and following further expansion, sorted cells were cloned by limited serial dilution. Resulting single-cell colonies were analyzed by goat anti-mouse F(Ab′)2 flow cytometry (see FIG. 39), and the highest expressing clone was expanded. Viral supernatants from expanded cells were collected, filtered through 0.45 uM syringe filter, and stored at 4° C. until use in transduction, or frozen at 80° C. for long-term. Prior to retroviral transduction, umbilical cord or peripheral blood mononuclear buffy coat cells were activated for three days in the presence of 300 IU/mL IL-2 and 1 μg/mL anti-human CD3 (Miltenyi Biotec, Germany). Cells were washed and suspended in T cell media with IL-2 at a concentration of 1×10e6/mL. For transduction, 750 μL of this cell mix was then combined with an equal volume of either control (non-transduced) media or BCMA-CD7-15/IL15sushi CAR retroviral vector, at a final 1:1 ratio. CAR transduction and validation of CAR expression in T cells are described in detail in the figure.

Activated human T cells were transduced with either control (left) or BCMA-CD7-15/IL15sushi CAR (right) viral supernatant from RD114 cells. 48 hours after the transduction, cells were harvested, washed and moved to tissue culture plates with fresh media and IL-2. After 3 days further incubation, cells were harvested and stained with goat-anti-mouse F(Ab′)2 antibody at 1:250 for 45 minutes. Cells were washed and stained with streptavidin-PE conjugate at 1:500, goat anti-mouse CD3 and CD7 antibodies, washed, suspended in 2% formalin, and analyzed by flow cytometry. Almost all T cells are CD7-negative T cells. Nearly 42% of CAR T cells expressed F(Ab′2) phenotype (top panels) (FIG. 39A).

Co-culture experiments comparing lysis of U937 expressing -BCMA(U-BCMA) cells, a human tumor cell line (U937) that is synthetically expressing CD269 (BCMA) antigen on the cell surface, by BCMA-CD7Q-IL-15/IL15sushi CAR T cells were performed at E:T ratio of 2:1 (upper panels) and 5:1 (lower panels) for 24 hours. CCRF-CEM cells were pre-labeled with CMTMR, a membrane dye, to distinguish them phenotypically from T cells. Target cells alone are shown on the left. Cells were analyzed by flow cytometry using anti-CD7 and anti-CD3 labeling. Populations encircled highlight target cell lysis. During 24-hour co-culture experiments, BCMA-CD7Q-IL-15/IL15sushi CAR T cells showed profound killing (nearly 90%) of target U-BCMA cells at a 2:1 ratio of effector:target (FIG. 39B).

### Example: Generation of CD7-Negative T Cells Containing CD7-CD19 cCAR

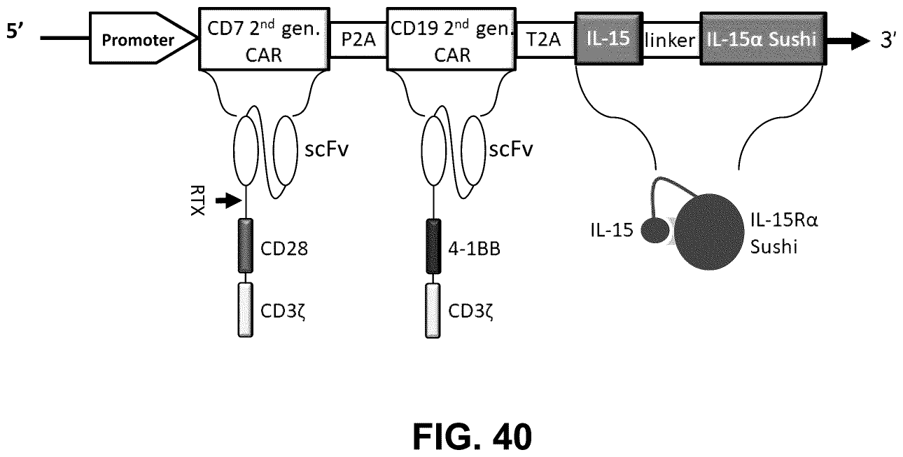

The expression of CD7-CD19-15/IL15sushi CAR is driven by the Moloney murine leukemia virus (MMLV), the long terminal repeat (LTR) promoter. The generation of compound CAR (cCAR) contains two complete CARs, each comprised of a signal domain (leader sequence) a scFv domain, a hinge domain (H), a cross-membrane domain (TM) and a co-activator factor CD28 (within the CD7 CAR only) and intracellular signal domains of 4-1BB(within the CD19 CAR only) and CD3zeta. The CD7 CAR has the safety switch immediately preceding the hinge domain (FIG. 40).

### Retroviral Production and Production of Stably Transduced Retrovirus-Expressing Cells

The H29 cell line is first transfected with the plasmid containing pX-CD7-CD19-15/IL15sushi gene using Lipofectamine 2000 (Life Technologies, Carlsbad, CA) per manufacturer's protocol. After incubation of 3 days, supernatant was removed and replaced with culture media containing DMEM, 10% FBS. The supernatant was used to transduce RD114 cells, HEK293 cells that stably express the gag/pol and vsv genes necessary for viral replication, using polybrene. After 24 hours incubation, this supernatant was removed and replaced with a second harvest of viral supernatant from the H29 cells. This process was repeated a third time. Following 3 transductions, cells were expanded to larger flasks, and sorted by FACS based on goat anti-mouse F(Ab′)2 expression, and following further expansion, sorted cells were cloned by limited serial dilution. Resulting single-cell colonies were analyzed by goat anti-mouse F(Ab′)2 flow cytometry and the highest expressing clone was expanded to produce viruses. Viruses were collected from supernatantsstored at liquid nitrogen, filtered through 0.45 uM syringe filter, and stored at 4° C. until use in transduction, or frozen at 80° C. for long-term.

Transduction of Human T Cells with Retroviral Vector from Stably-Transduced Retrovirus-Expressing Cells

Prior to retroviral transduction, umbilical cord or peripheral blood mononuclear buffy coat cells were activated for three days in the presence of 300 IU/mL IL-2 and 1 μg/mL anti-human CD3 (Miltenyi Biotec, Germany). Cells were washed and suspended in T cell media with IL-2 at a concentration of 1×10e6/mL. For transduction, 750 μL of this cell mix was then combined with an equal volume of either control (non-transduced) media or CD7-CD19-IL-15/IL-15sushi retroviral vector, at a final 1:1 ratio. CAR transduction and validation of CAR expression in T cells are described in detail in the next figure.

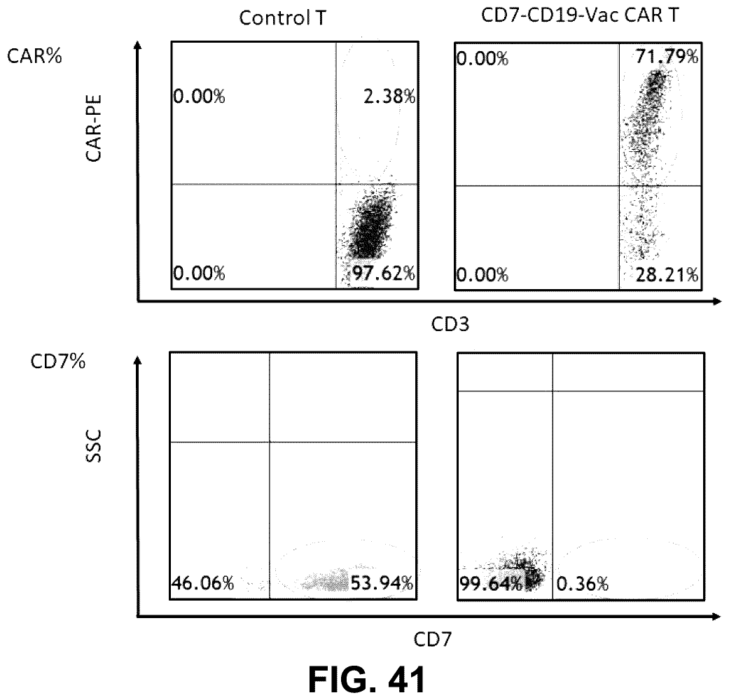

Activated human T cells were transduced with either control (left) or CD7-CD19-15/ILl5sushi CAR (right) viral supernatant from RD114 cells. 48 hours after the transduction, cells were harvested, washed and moved to tissue culture plates with fresh media and IL-2. After 3 days further incubation, cells were harvested and stained with goat-anti-mouse F(Ab′)2 antibody at 1:250 for 45 minutes. Cells were washed and stained with streptavidin-PE conjugate at 1:500, goat anti-mouse CD3 and CD7 antibodies, washed, suspended in 2% formalin, and analyzed by flow cytometry. Almost All T cells were CD7-negative and about 72% of CAR T cells expressed F(Ab′2) phenotype (top panels) (FIG. 41).

In general, the range of effective lentiviral vector constructs has been limited by the size of the insert; the longer compound CARs (inserts of about 3 to 4 kb) have resulted in much lower lentiviral titers and lower CAR T transduction efficiency than our single CARs. This issue can be surprisingly overcome when the stably producing virus cell line is used.

Using the longer retroviral vector constructs, we were able to obtain higher viral titers and therefore higher CAR T transduction efficiencies for our compound CARs after sorting the highest retrovirus-producing RD114 cells and isolating single clones. This method can also be used to make CARs even longer than cCARs, with 3 or more complete CAR units; for example, a cCAR comprised of complete anti-BCMA and anti-CD19 CAR units as well as IL-15/IL-15sushi as described above. While the initial level of retroviral vector titer might be low with constructs of 5 kb or more, the same method of sorting and isolating the highest expressing RD114 cells would still result in higher CAR T transduction efficiencies.

CD7-Negative T Cells with a CD19 CAR Showing Remarkable Killing Activities.

Tumor cells expressing either CD7 or CD19 were used to test the functional activity of each unit (CD7 or CD19) of the cCAR on the CD7-negative T cells. As these cells proliferate extensively, the effect of cCAR on their growth can be more easily determined to confirm its ability to lyse cells possessing its target antigens.

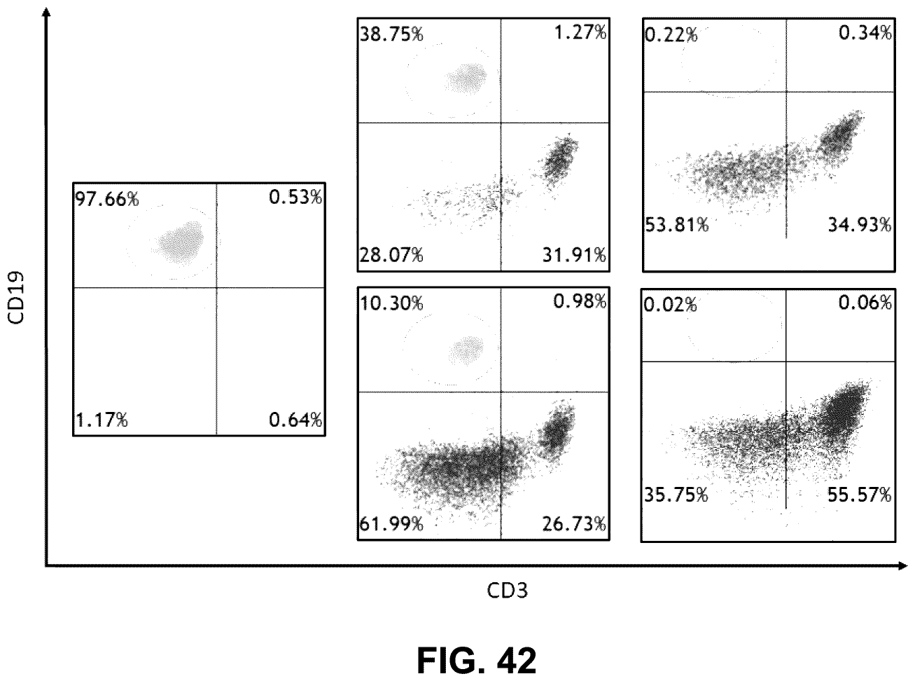

Using a cell line positive for CD19 antigen (REH), we co-cultured those cells with either control T or CD7-CD19-IL-15/IL-15sushi CAR T cells at ratios of 1:1 and 2:1 T cell:target cell ratios (FIG. 42). After 18 hours co-culture, it is unexpected that nearly all target cells had been eliminated at the 2:1 and even 1:1 ratio.

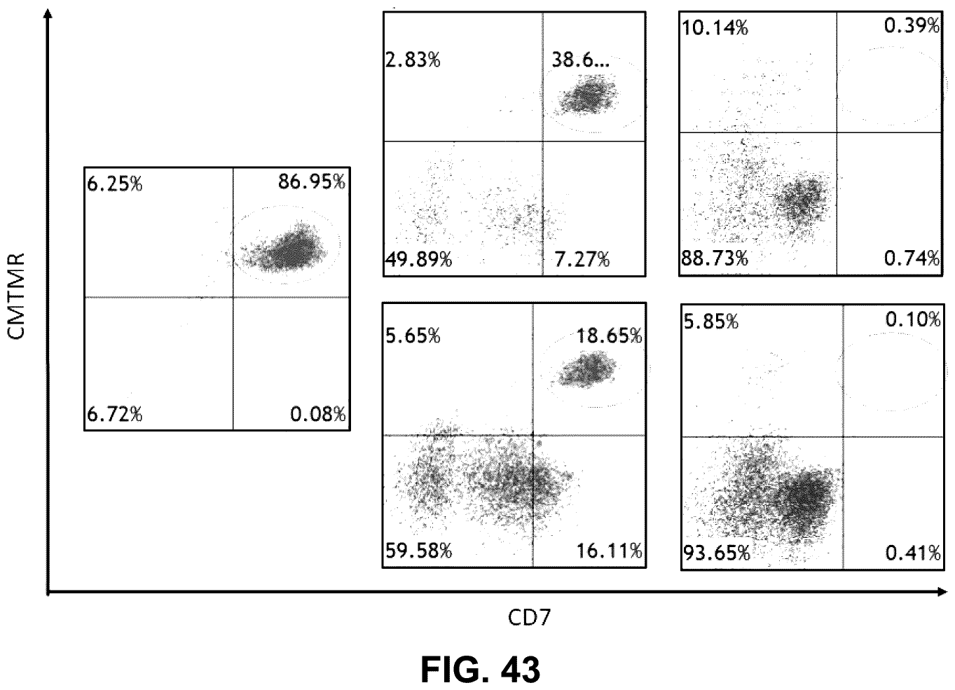

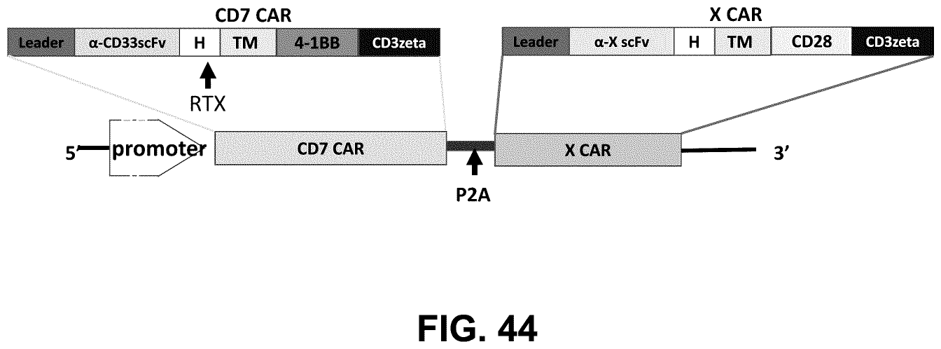

Using a cell line positive for CD7 markers (CCRF-CEM), we co-cultured those cells with either control T or CD7-CD19-IL-15/IL-15sushi CAR T cells at ratios of 1:1 and 3:1 T cell:target cell ratios (FIG. 43). After 18 hours co-culture, it is also unexpected that nearly all target cells had been eliminated at the 3:1 and even 1:1 ratio.

These results demonstrate the robust lysis ability of each component CAR unit in CD7-CD19-IL-15/IL-15sushi cCAR in the CD7-negative T cells.

### Anti-BCMA VHH Discovery Strategy

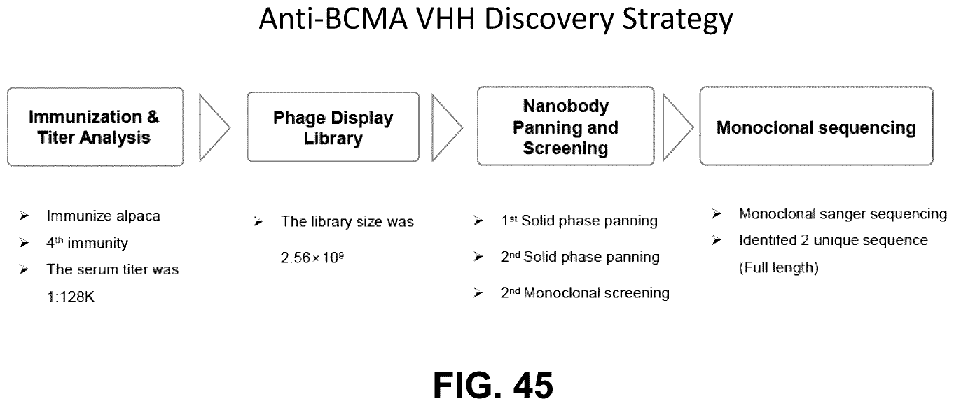

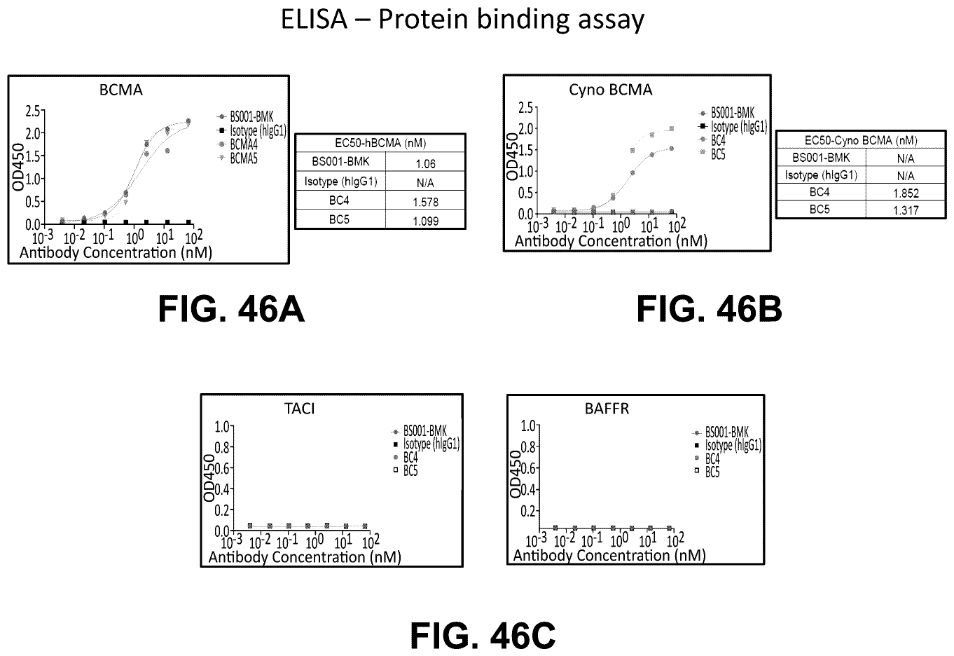

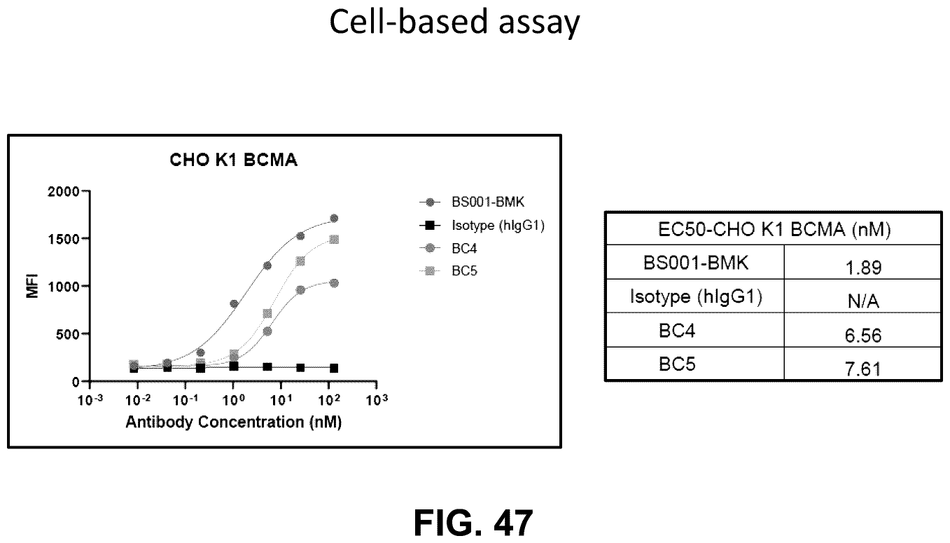

A VHH antibody (or nanobody) is the antigen binding fragment of heavy chain only antibodies. Alpacas were immunized over an 8-week period after which PBMC mRNA was isolated and processed into a VHH gene library. This library was transformed into phage-competent bacteria to generate a bacteriophage library, which was panned against BCMA to enrich for binding clones. High quality hits were identified by high-throughput enzyme-linked immunosorbent assays (ELISA) of individual VHH clones. VHH candidates with binding greater than 2-fold above average background were picked and sent for Sanger sequencing to identify unique sequences (FIG. 45A). ELISA assays were used to determine the protein binding (FIG. 46) Interaction of BCMA VHHs antibodies with NIH-CoVnb-112 with BCMA (human) or Cyno BCMA (Cynomolgus) protein (A and B) were performed. TACI and BFFR were used for negative control (C). Binding affinity of two BCMA VHHs, BC4 and BC5 as well as half maximal bindings (EC50) were 1.578 and 1.099 respectively. The ELISA was developed using a standard streptavidin-HRP and tetramethylbenzidine reaction (FIG. 46). In order to further determine the bindings of new identified VHHs, BC4 and BC5 against BCMA protein. Cell based assays were performed with CHO cells engineeringly expressing BCMA. Cell surface BCMA VHHs, BC4 and BC5 binding affinity was analyzed with BCMA-expressing CHO cells using flow cytometry analysis. Various concentrations of each antibody were used, with 2^{nd} anti-human IgG-FITC was used to detect each antibody. BC4 and BC5 VHHs specially, strongly bound the surface BCMA protein (FIG. 47). BC4 and BC5 VHHs were used to generate VHH CAR-T or NK cells.

Characterization of BC4-RTX-IL-15/IL15sushi CAR targeting plasma cells or BCMA-expressing cells.

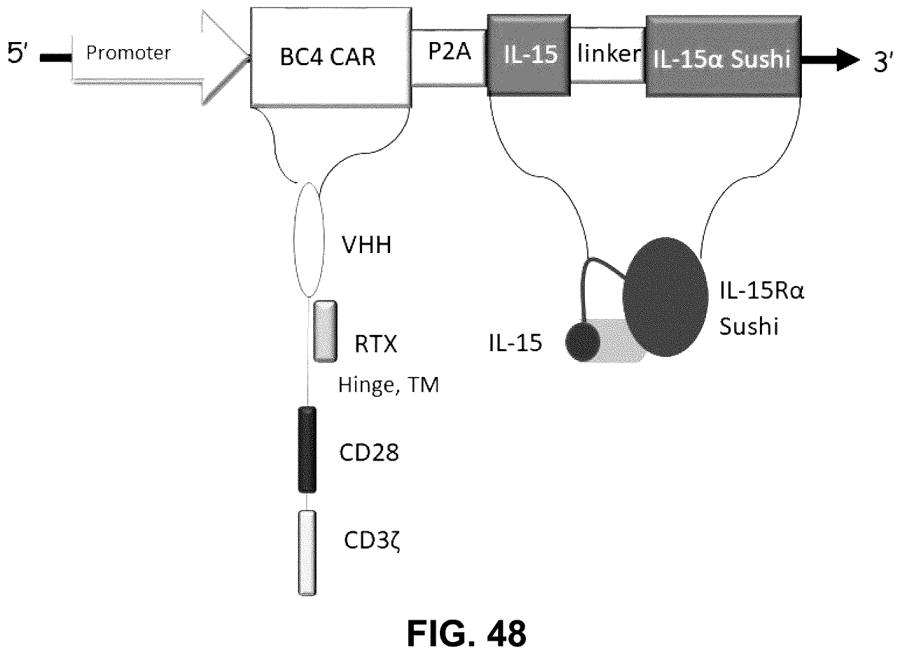

CAR T cells were generated by transduction of primary peripheral blood T-cells with the retroviral construct shown in FIG. 48. The translated CAR proteins were then expressed on the surface of the T cells, where they can recognize and bind the target proteins on the surface of tumor cells. The pharmacologic effect and mechanism of the CARs are mediated by BC4-RTX-IL-15/IL15sushi CAR T cell recognition of the antigen, which triggers cytotoxic T cell activity, further enhanced by the incorporation of CD28 co-activation domain in the construct.

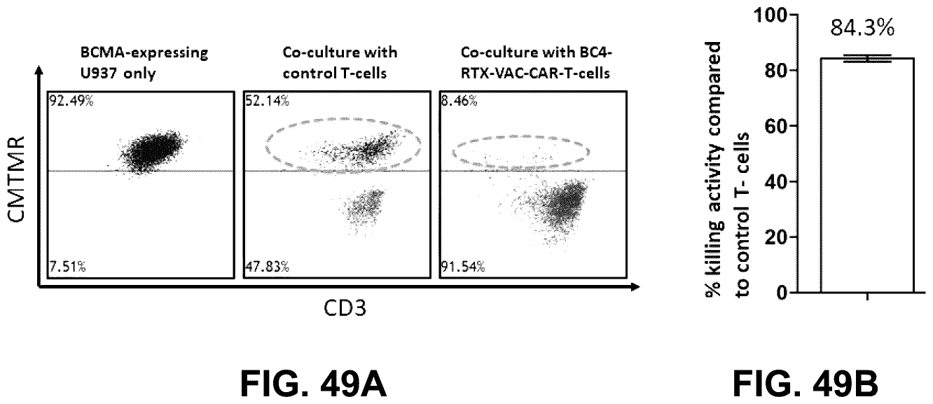

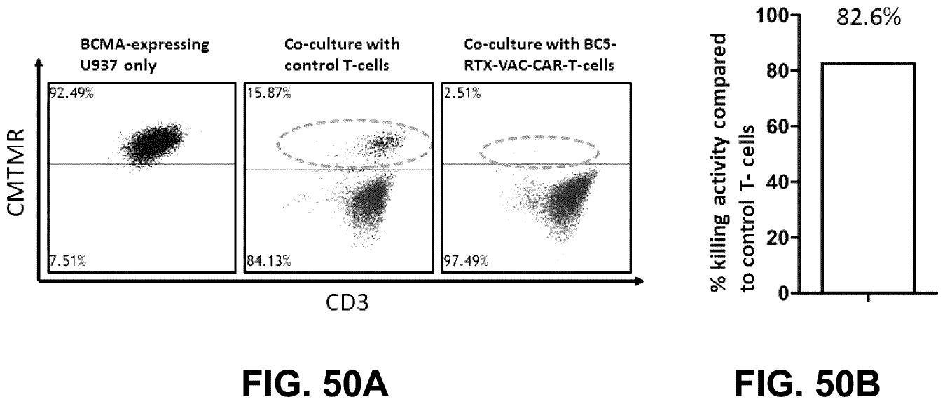

To assay the ability of the BC4-RTX-IL-15/IL15sushi CAR T cells to target CD269 (BCMA)+ cells, 18 h co-cultures with either control or BC4-RTX-IL-15/IL15sushi CAR T cells versus the synthetically antigen-expressing tumor cell line U937-BCMA was performed at E:T ratios of 2:1. U-BCMA target line, which synthetically expresses the BCMA surface antigen, were strongly ablated by BC4-RTX-IL-15/IL15sushi CAR T cells compared to control T cells (84%) in 18 hour co-cultures (FIG. 49). The results of these experiments confirm the ability of the BC4-RTX-IL-15/IL15sushi CAR T cells to effectively target and lyse BCMA-expressing target cells, and constitute an ideal candidate for the clinical treatment of multiple myeloma.

A similar method was to test the BC5-RTX-IL-15/IL15sushi CAR T cells to target plasma cells or BCMA+ cells. During 24-hour co-culture experiments, BC4-RTX-IL-15/IL-15sushi CAR T cells showed profound killing (82%) of target U937-BCMA cells at a 5:1 ratio of effector:target. The construct of BC5-RTX-IL-15/IL-15sushi used a similar way to that of BC4-RTX-IL-15/IL-15sushi. BC5-RTX-IL-15/IL-15sushi CAR T cells showed profound killing (82%) of target U937-BCMA cells at a 5:1 ratio of effector:target.

### Generation of Tandem CARs.

BCMA-CD19 (also called BC4-CD19c-Tan or BC5-CD19c-Tan) was generated as leader+VHH+linker+scFv+h+TM+Co+CD3 zeta or leader+scFv1+linker+scFv2+h+TM+Co+CD3 zeta). Note: leader, signal peptide; linker, a (GGGS)3 or (GGGS)4; H, hinge region; TM, transmembrane domain; Co, co-stimulatory domain. Tandem targeting constructs for CARs were generated in a similar manner. CAR constructs were cloned into a retroviral plasmid backbone under the promoter of (MMLV), the long terminal repeat (LTR) promoter. Retroviruses expressing CARs using a standard method were generated as described above.

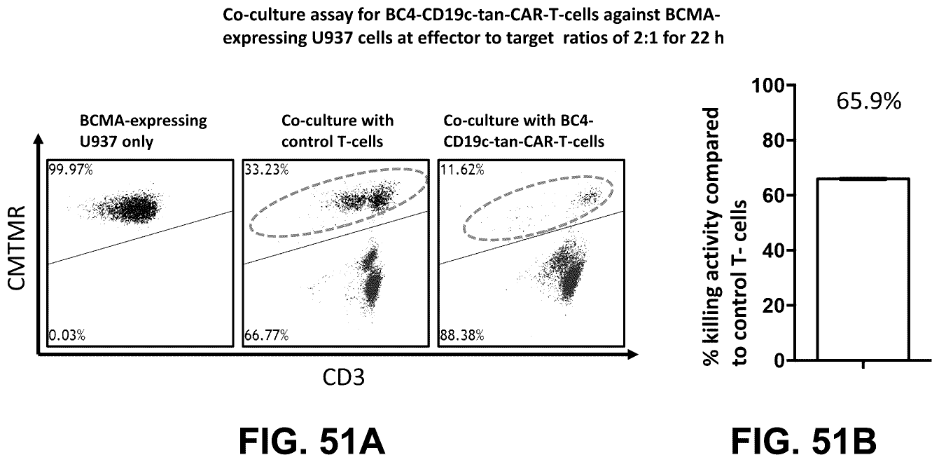

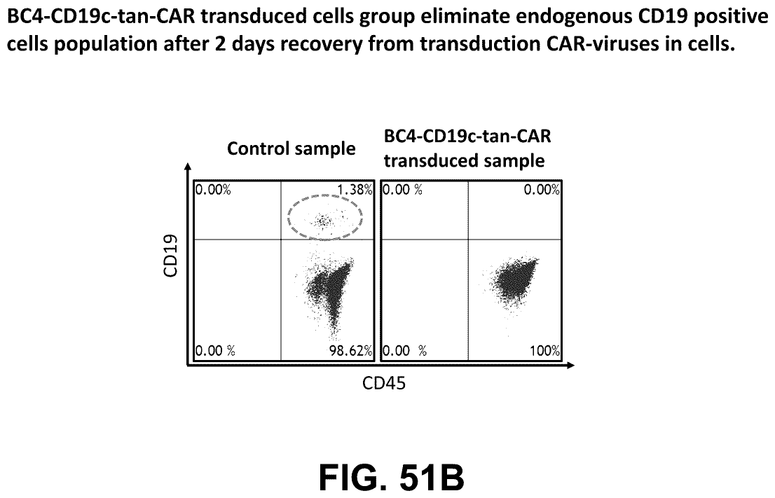

BC4-CD19c-Tan CAR viruses were then tested on T-cells for CAR expression. Flow cytometry results confirmed the expression of BC4-CD19c-Tan CAR. To assess BC4-CD19c-Tan CAR functional activity, BCMA-expressing U937 cells were used. BC4-CD19c-Tan CAR consistently displayed robust lysis of cells expressing BCMA more than 65% at ratio of E:T ratios 2:1 (FIG. 51A).

To evaluate BC4-CD19c-tan-CAR transduced T-cells can eliminate endogenous CD19 positive cells, especially B-cells, in donor cells after CAR-viruses transduction (FIG. 51B). To elucidate this question, 2 days after recovery control-T cells and CAR transduced T-cells (Day 6 after activation of T-cells) were labeled using CD45-PerCP, CD3-PE and CD19-APC antibodies for flow cytometry analysis. Flow cytometry analysis showed the control cells sample contains CD19 positive population (dots circled on left panel) while CAR virus transduced sample group could not detect CD19 positive population (right panel).

The Following Multiple Tandem Targeting Constructs for CARs were Also Generated in a Similar Manner as Described Above.

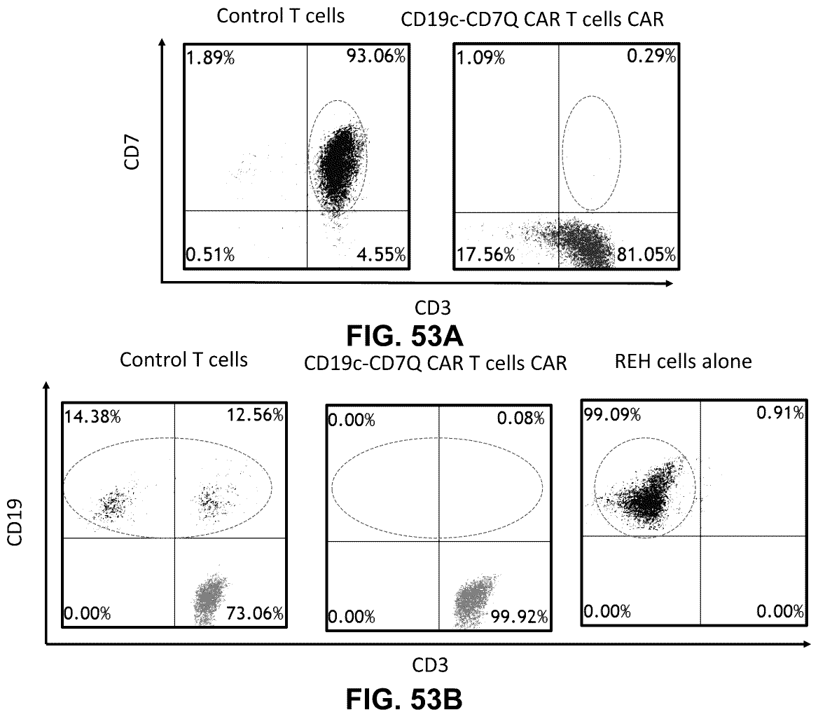

CD19c-CD7 Q CAR T cells (also called CD19c-CD7 Q-Tan CAR T cells) was generated to targeting B cells or B cell tumors and T cells or T cell tumors. The Q is also called RTX incorporated in the hinge region of the CAR containing two RTX-binding epitopes. CD19c-CD7 Q-Tan CAR viruses were then tested on T-cells for CAR expression. Flow cytometry results confirmed the expression of CAR. CD7+ cells were completely eliminated from the transduced T cell population (FIG. 53A). During 16-hour co-culture experiments, CD19c-CD7Q CAR T cells completely lysed CD19 expressing REH cells at a 2:1 ratio (FIG. 53B), demonstrating the profound efficacy of the CD19 CAR. These results indicate that both CAR units are successfully lysing their target populations (B cells and T cells).

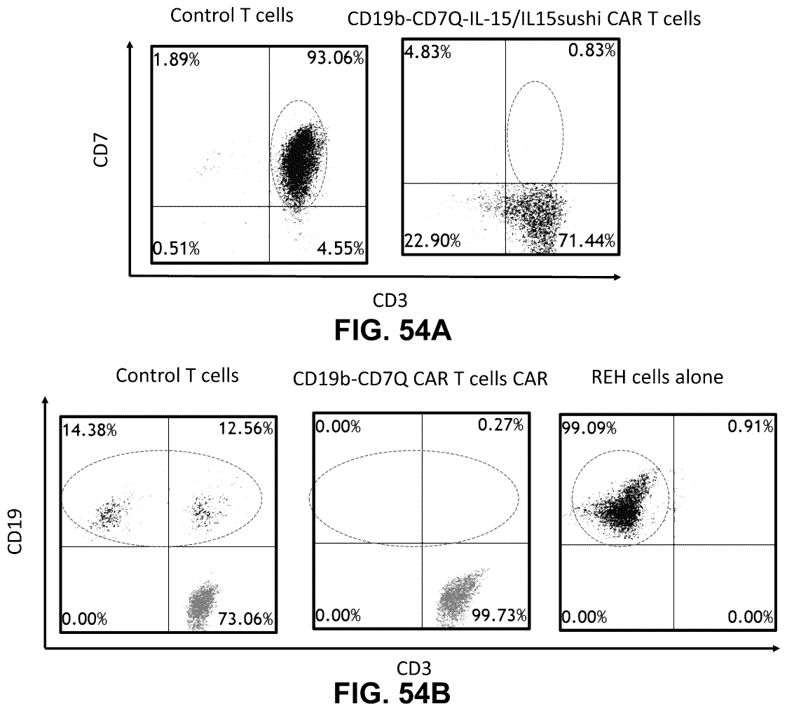

A similar method was applied to the construct of CD19b-CD7 Q VAC CAR (also CD19b-CD7 Q-IL-15/IL-15sushi CAR-Tan) targeting B cells or B cell tumors and T cells or T cell tumors. This tandem CAR is linked to the IL-15/IL-15sushi fusion protein. IL-15/IL-15sushi fusion was to enhance immune cell functions. PBMC from healthy human donors was activated 48 hours in the presence of mouse anti-human CD3 antibody. Cells (1.0×10e6) were transduced with retroviruses expressing the construct. CD7+ cells were completely eliminated from the transduced T cell population. During 16-hour co-culture experiments, CD19b-CD7Q-IL-15/IL15sushi CAR T cells completely lysed CD19 expressing REH cells at a 2:1 ratio, demonstrating the profound efficacy of the CD19 CAR. These results indicate that both CAR units are successfully lysing their target populations (FIG. 54).

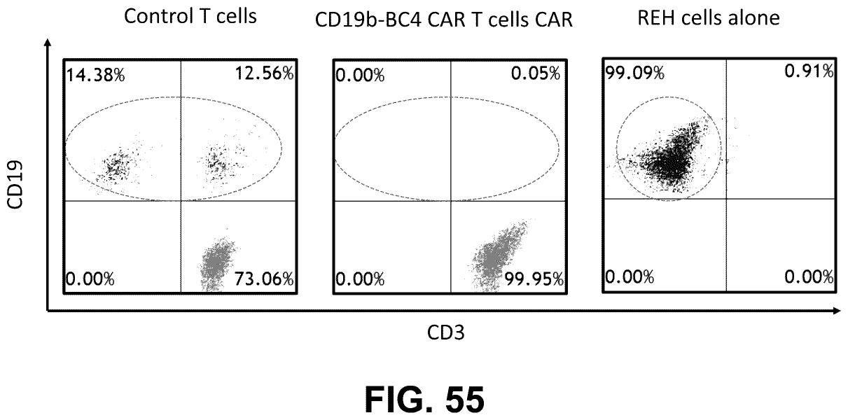

A similar finding to the CAR construct, CD19b-BC4 tandem CAR was noted. During 16-hour co-culture experiments, CD19b-BC4 CAR T cells completely lysed REH cells expressing CD19 at a 2:1 ratio, demonstrating the profound efficacy of the CD19 CAR unit (FIG. 55). In addition, CD19b-BC4 CAR T also robustly lysed U937 expressing BCMA.

### IL-15/IL-15Sushi Enhances CAR:

CD19b-IL-15/IL-15sushi CART constructs as an example provide enhanced persistency and biologic activity compared to standard CD19 CAR. CD19-IL-15/IL-15sushi construct contains CD19 CAR linked to a IL-15 fusion protein via a self-cleavage peptide. The IL-15 fusion protein consists of IL-15 linked to a soluble domain of IL-15Rα (sushi) and secreted as a soluble 1L-15/IL-15 sushi complex.

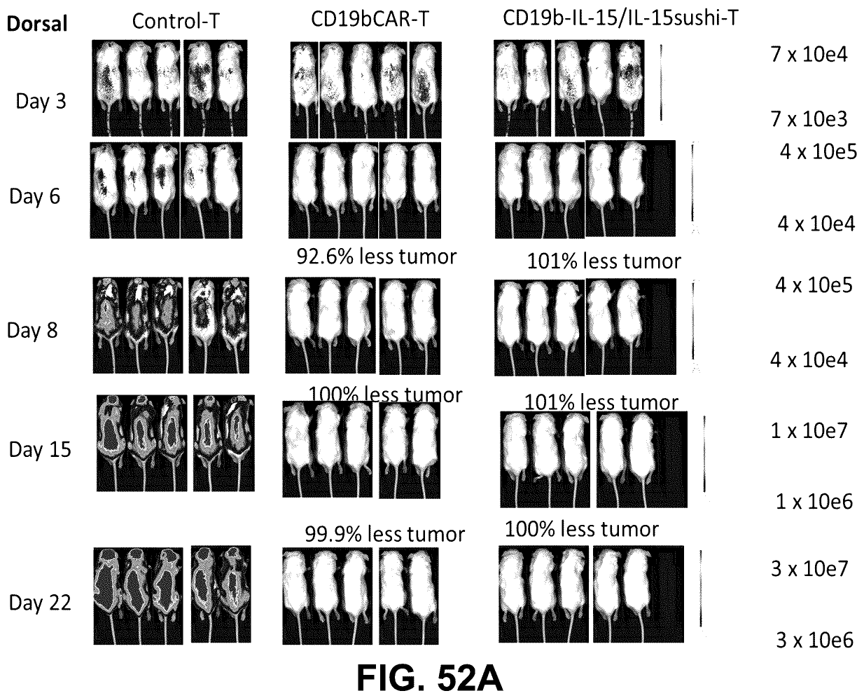

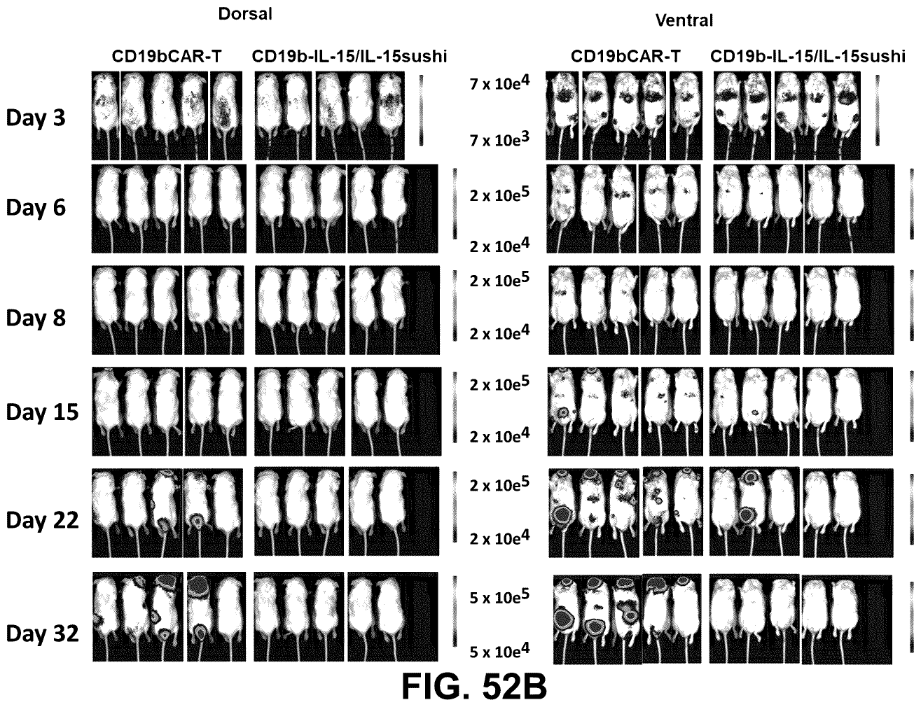

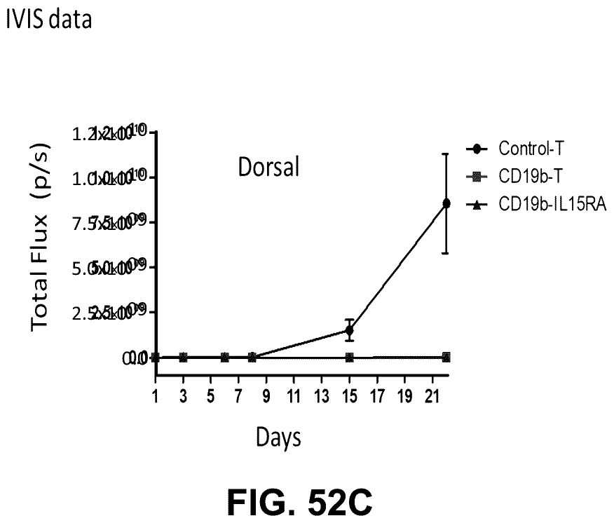

To characterize the CAR secreting IL-15/IL-15sushi as a viable option to current CAR T/NK cell paradigms we analyzed for 3 broad factors: 1) ability to kill target cells (efficacy),2) enhanced persistence for increased bioavailability and surveillance, and 3) proliferation of more potent CAR T phenotypes. The CD19b-IL-15/IL-15sushi CAR construct was able to control Reh model tumor growth in vivo with comparable and slightly better efficacy than standard CART-19 (CD19b CAR) (FIG. 52A). In a second Reh model, we showed that as time goes on, Reh tumor relapsed in standard CAR T(CD19b CAR) treatment, however, the IL-15/IL-15sushi secreting CAR persists and deletes relapsed tumor and keeps mice disease free (FIGS. 52B and 52C). Furthermore, by survival endpoints, CD19b-IL-15/IL-15sushi CAR T cells administered mice revealed distinct populations of cytotoxic T cells remaining in circulation with a higher level, compared to CD19b CAR without secreting IL-15/IL-15sushi. In addition, the population of remaining T cells in both treatment groups showed that the CAR T cells (CD19b-IL-15/IL-15sushi) secreting IL-15/IL-15sushi resulted in a more cytotoxic T cell population, comprised of a higher population of CD8+ cells.

1. A method of treating an autoimmune disorder in a patient in need thereof, the method comprising administering to said patient a dual CAR, wherein the dual CAR binds to a first antigen and a second antigen, wherein the first antigen is expressed on T-cells or NK cells and is selected from the group consisting of CD2, CD3, CD4, CD5, and CD7, and wherein said second antigen is expressed on B-cells or plasma cells and is selected from the group consisting of CD19, CD20, CD22, BCMA, CD38, CD138, CS1, and GPRC5D, and wherein said dual CAR is either a tandem CAR or compound CAR (cCAR).

2. A method of treating an autoimmune disorder in a patient, wherein the method comprises administering to said patient a CAR which binds to an antigen expressed on T-cells or NK cells, wherein said antigen is selected from the group consisting of CD2, CD3, CD4, CD5 and CD7.

3. The method according to claim 1, wherein the autoimmune disorder is T-cell mediated and/or resulting from autoreactive T-cells.

4. The method according to claim 1, wherein the autoimmune disorder is B-cell mediated and/or resulting from autoreactive B-cells.

5. The method according to claim 1, wherein the autoimmune disorder is T cell and B cell mediated and/or resulting from autoreactive immune cells including T-cells, B cells, plasma cells, or a combination thereof.

6. The method according to claim 1, wherein said dual CAR is comprised of a CD7CAR, and another CAR unit targeting an antigen selected from the group consisting of CD19, CD20, CD22, BCMA, CD38, GPRC5D, and CS1, wherein administration results in depletion of T-cells expressing CD7 surface antigen and B-cells or plasma cells, or a combination thereof in said patient.

7. The method according to claim 1, wherein the dual CAR binds to cells expressing CD7 and/or CD19.

8. The method according to claim 1, wherein the dual CAR binds to cells expressing CD7 and/or CD20.

9. The method according to claim 1, wherein the dual CAR binds to cells expressing CD7 and/or BCMA.

10. The method according to claim 1, wherein the dual CAR binds to cells expressing CD19 and/or CD20.

11. The method according to claim 1, wherein the dual CAR binds to cells expressing CD19 and/or BCMA.

12. A method of treating an autoimmune disorder in a patient in need thereof, wherein the method comprises administering to said patient a CD7CAR, wherein said CD7CAR binds to CD7 antigen expressed on T-cells and wherein administration results in depletion of T-cells expressing CD7 surface antigen in said patient.

13. The method according to claim 1, wherein said method further comprises administration of a steroid and/or an immune cell inhibitor selected from a B-cell inhibitory treatment, a plasma cell inhibitory treatment or an immunosuppression treatment to said patient in need thereof.

14. The method according to claim 1, wherein the autoimmune disorder is selected from Type I Diabetes, Multiple Sclerosis, Inflammatory Bowel Disease, Ulcerative Colitis, Crohn's disease, Celiac's disease, myasthenia gravis, Pemphigus vulgaris, Bullous pemphigoid Graves' disease, Asthma, systemic lupus erythematosus, IgA nephropathy, IgG4 related disease, membranous nephropathy, Myasthenia gravis, Neuromyelitis optica, Pemphigus vulgaris, anti-PAD4-activating rheumatoid arthritis, sensitized/preformed antibodies in solid organ transplant, Psoriasis, Guillain-Barre Syndrome (Acute inflammatory demyelinating polyneuropathy—AMOP), Chronic inflammatory demyelinating polyneuropathy (CIDP), Evans syndrome, immune thrombocytopenic purpura, rheumatoid arthritis, Sjogren's syndrome, and ANCA-associated vasculitis (AAV).

15. The method according to claim 1, wherein the autoimmune disorder is selected from Type I diabetes, Multiple Sclerosis, Inflammatory Bowel Disease, Psoriasis, Ulcerative Colitis, or Crohn's disease.

16. The method according to claim 1, wherein the autoimmune disorder is Type I Diabetes.

17. The method according to claim 1, wherein the autoimmune disorder is Multiple Sclerosis.

18. The method according to claim 1, wherein the autoimmune disorder is Inflammatory Bowel Disease, Ulcerative Colitis, or Crohn's disease.

19. The method according to claim 1, wherein the patient is in need of preventative treatment of a T-cell mediated autoimmune disorder or a combination of T-cell and B-cell mediated disorders.

20. The method according to claim 1, wherein the patient is in need of treatment of a relapse or refractory T-cell mediated autoimmune disorder or a combination of T-cell and B-cell mediated disorders.

21. A chimeric antigen receptor (CAR) comprising a BCMA antigen binding domain comprising the amino acid sequence of SEQ. ID NO: 77 or SEQ. ID NO: 78, a hinge domain, a transmembrane domain, at least one co-stimulatory domain and a CD3 zeta signaling domain.

22. The chimeric antigen receptor of claim 21, wherein said antigen binding domain comprises an amino acid sequence selected from the group consistent of SEQ ID NO: 49, 51, 53, 55, 57, 59, 61 and 63.

23. An ex vivo engineered T cell or NK cell co-expressing two distinct chimeric antigen receptor (CAR) units at the cell surface, wherein the engineered T cell or NK cell comprises a nucleotide sequence comprising from 5′ to 3′ a first polynucleotide encoding a first chimeric antigen receptor polypeptide (first CAR), a second polynucleotide encoding a second chimeric antigen receptor polypeptide (second CAR), a nucleotide encoding porcine teschovirus-1 2A (P2A), thoseaasigna virus 2A (T2A), FMDV 2A (F2A) or equine rhinitis A virus (ERAV) 2A (E2A) disposed between the first CAR and second CAR, under the transcriptional control of a single promoter, wherein:
(i.) the first CAR polypeptide comprises a first antigen recognition domain; a first signal peptide; a first hinge region; a first transmembrane domain; a first co-stimulatory domain; and a first signaling domain; and
(ii.) the second CAR comprises a second antigen recognition domain; a second signal peptide; a second hinge region; a second transmembrane domain; a second co-stimulatory domain; and a second signaling domain;
wherein the first antigen recognition domain and the second antigen recognition domain are different; wherein the engineered T cell or NK cell comprises SEQ ID NO: 5, SEQ ID NO: 9, SEQ ID NO: 17 or SEQ ID NO: 19.

24. An ex vivo engineered T cell or NK cell co-expressing two distinct chimeric antigen receptor (CAR) units at the cell surface, wherein the engineered T cell or NK cell comprises a nucleotide sequence comprising from 5′ to 3′ a first polynucleotide encoding a first chimeric antigen receptor polypeptide (first CAR), a second polynucleotide encoding a second chimeric antigen receptor polypeptide (second CAR), a nucleotide encoding porcine teschovirus-1 2A (P2A), thoseaasigna virus 2A (T2A), FMDV 2A (F2A) or equine rhinitis A virus (ERAV) 2A (E2A) disposed between the first CAR and second CAR, under the transcriptional control of a single promoter, wherein:
(i.) the first CAR polypeptide comprises a first antigen recognition domain; a first signal peptide; a first hinge region; a first transmembrane domain; a first co-stimulatory domain; and a first signaling domain; and
(ii.) the second CAR comprises a second antigen recognition domain; a second signal peptide; a second hinge region; a second transmembrane domain; a second co-stimulatory domain; and a second signaling domain;
wherein the first antigen recognition domain and the second antigen recognition domain are different;
wherein the engineered T cell or NK cell comprises an enhancer selected from the group consisting of IL-15/IL-15sushi, and wherein the engineered T cell or NK cell comprises SEQ ID NO: 7, SEQ ID NO: 11, SEQ ID NO: 13, SEQ ID NO: 15, SEQ ID NO: 21, SEQ ID NO: 23, SEQ ID NO: 25, SEQ ID NO: 27, SEQ ID NO: 29, SEQ ID NO: 33, or SEQ ID NO: 47.

25. A tandem CAR-T or NK cell comprising a single chimeric antigen polypeptide comprising two distinct antibody binding domains, wherein the antibody binding domains are linked together by a linker and share a hinge region, a transmembrane domain, a signaling domain and at least one costimulatory domain, wherein the tandem CAR comprises SEQ ID NO: 35, SEQ ID NO: 37, SEQ ID NO: 39, SEQ ID NO: 41, SEQ ID NO: 43, SEQ ID NO: 45, SEQ ID NO: 65, SEQ ID NO: 67, SEQ ID NO: 69, SEQ ID NO: 71, SEQ ID NO: 73, or SEQ ID NO: 75.

26. An ex vivo engineered T cell or NK cell co-expressing a chimeric antigen receptor (CAR) unit or two distinct chimeric antigen receptor (CAR) units at the cell surface wherein the engineered T cell or NK cell comprises a nucleotide sequence comprising from 5′ to 3′ a single promoter of MMLV promoter, wherein the engineered T cell or NK cell comprises SEQ ID NO: 1, SEQ ID NO: 3, SEQ ID NO: 25, SEQ ID NO: 47, or SEQ ID NO: 31.


SEQUENCE LISTING (78 sequences)

SEQ ID NO: 1 - protein, 553 aa, synthetic construct
  REGION 1..553: note = Description of sequence: CD7-RTX CAR amino acid sequence
MALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPP

SEQ ID NO: 2 - protein, 1673 aa, synthetic construct
  REGION 1..1673: note = Description of sequence: CD7-RTX CAR nucleotide sequence
ATGGCCTTACCAGTGACCGCCTTGCTCCTGCCGCTGGCCTTGCTGCTCCACGCCGCCAGGCCGGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAAGTTTAAAC

SEQ ID NO: 3 - protein, 848 aa, synthetic construct
  REGION 1..848: note = Description of sequence: CD7-RTX-VAC CAR amino acid sequence
MALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 4 - protein, 2555 aa, synthetic construct
  REGION 1..2555: note = Description of sequence: CD7-RTX-VAC CAR nucleotide sequence
ATGGCCTTACCAGTGACCGCCTTGCTCCTGCCGCTGGCCTTGCTGCTCCACGCCGCCAGGCCGGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGCAGCGGCGAAGGCCGCGGCAGCCTGCTGACCTGCGGCGATGTGGAAGAAAACCCGGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAAC

SEQ ID NO: 5 - protein, 1070 aa, synthetic construct
  REGION 1..1070: note = Description of sequence: CD7-RTX-CD19 cCAR amino acid sequence
MALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 6 - protein, 3221 aa, synthetic construct
  REGION 1..3221: note = Description of sequence: CD7-RTX-CD19 cCAR nucleotide sequence
ATGGCCTTACCAGTGACCGCCTTGCTCCTGCCGCTGGCCTTGCTGCTCCACGCCGCCAGGCCGGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGGCCCTGCCCGTGACCGCCCTGCTGCTGCCCCTGGCCCTGCTGCTGCACGCCGCCAGGCCCGAGGTCCAGCTGCAGCAGTCTGGACCTGAGCTGATAAAGCCTGGGGCTTCAGTGAAGATGTCCTGCAAGGCTTCTGGATACACATTCACTAGCTATGTTATGCACTGGGTGAAGCAGAAGCCTGGGCAGGGCCTTGAGTGGATTGGATATATTAATCCTTACAATGATGGTACTAAGTACAATGAGAAGTTCAAAGGCAAGGCCACACTGACTTCAGACAAATCCTCCAGCACAGCCTACATGGAGCTCAGCAGCCTGACCTCTGAGGACTCTGCGGTCTATTACTGTGCAAGAGGGACTTATTACTACGGTAGTAGGGTATTTGACTACTGGGGCCAAGGCACCACTCTCACAGTCTCCTCAGGTGGAGGGGGCTCAGGCGGAGGTGGCTCTGGGGGTGGAGGCTCGGACATTGTGATGACTCAGGCTGCACCCTCTATACCTGTCACTCCTGGAGAGTCAGTATCCATCTCCTGCAGGTCTAGTAAGAGTCTCCTGAATAGTAATGGCAACACTTACTTGTATTGGTTCCTGCAGAGGCCAGGCCAGTCTCCTCAGCTCCTGATATATCGGATGTCCAACCTTGCCTCAGGAGTCCCAGACAGGTTCAGTGGCAGTGGGTCAGGAACTGCTTTCACACTGAGAATCAGTAGAGTGGAGGCTGAGGATGTGGGTGTTTATTACTGTATGCAACATCTAGAATATCCGTTCACGTTCGGTGCTGGGACCAAGCTGGAGCTGAAACGGACCACCACCCCCGCCCCCAGGCCCCCCACCCCCGCCCCCACCATCGCCAGCCAGCCCCTGAGCCTGAGGCCCGAGGCCTGCAGGCCCGCCGCCGGCGGCGCCGTGCACACCAGGGGCCTGGACTTCGCCTGCGACATCTACATCTGGGCCCCCCTGGCCGGCACCTGCGGCGTGCTGCTGCTGAGCCTGGTGATCACCCTGTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGGGTGAAGTTCAGCAGGAGCGCCGACGCCCCCGCCTACCAGCAGGGCCAGAACCAGCTGTACAACGAGCTGAACCTGGGCAGGAGGGAGGAGTACGACGTGCTGGACAAGAGGAGGGGCAGGGACCCCGAGATGGGCGGCAAGCCCCAGAGGAGGAAGAACCCCCAGGAGGGCCTGTACAACGAGCTGCAGAAGGACAAGATGGCCGAGGCCTACAGCGAGATCGGCATGAAGGGCGAGAGGAGGAGGGGCAAGGGCCACGACGGCCTGTACCAGGGCCTGAGCACCGCCACCAAGGACACCTACGACGCCCTGCACATGCAGGCCCTGCCCCCCAGGTAAGTTTAAAC

SEQ ID NO: 7 - protein, 1364 aa, synthetic construct
  REGION 1..1364: note = Description of sequence: CD7-RTX-CD19 VAC cCAR amino acid sequence
MALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 8 - protein, 4103 aa, synthetic construct
  REGION 1..4103: note = Description of sequence: CD7-RTX-CD19 VAC cCAR nucleotide sequence
ATGGCCTTACCAGTGACCGCCTTGCTCCTGCCGCTGGCCTTGCTGCTCCACGCCGCCAGGCCGGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGGCCCTGCCCGTGACCGCCCTGCTGCTGCCCCTGGCCCTGCTGCTGCACGCCGCCAGGCCCGAGGTCCAGCTGCAGCAGTCTGGACCTGAGCTGATAAAGCCTGGGGCTTCAGTGAAGATGTCCTGCAAGGCTTCTGGATACACATTCACTAGCTATGTTATGCACTGGGTGAAGCAGAAGCCTGGGCAGGGCCTTGAGTGGATTGGATATATTAATCCTTACAATGATGGTACTAAGTACAATGAGAAGTTCAAAGGCAAGGCCACACTGACTTCAGACAAATCCTCCAGCACAGCCTACATGGAGCTCAGCAGCCTGACCTCTGAGGACTCTGCGGTCTATTACTGTGCAAGAGGGACTTATTACTACGGTAGTAGGGTATTTGACTACTGGGGCCAAGGCACCACTCTCACAGTCTCCTCAGGTGGAGGGGGCTCAGGCGGAGGTGGCTCTGGGGGTGGAGGCTCGGACATTGTGATGACTCAGGCTGCACCCTCTATACCTGTCACTCCTGGAGAGTCAGTATCCATCTCCTGCAGGTCTAGTAAGAGTCTCCTGAATAGTAATGGCAACACTTACTTGTATTGGTTCCTGCAGAGGCCAGGCCAGTCTCCTCAGCTCCTGATATATCGGATGTCCAACCTTGCCTCAGGAGTCCCAGACAGGTTCAGTGGCAGTGGGTCAGGAACTGCTTTCACACTGAGAATCAGTAGAGTGGAGGCTGAGGATGTGGGTGTTTATTACTGTATGCAACATCTAGAATATCCGTTCACGTTCGGTGCTGGGACCAAGCTGGAGCTGAAACGGACCACCACCCCCGCCCCCAGGCCCCCCACCCCCGCCCCCACCATCGCCAGCCAGCCCCTGAGCCTGAGGCCCGAGGCCTGCAGGCCCGCCGCCGGCGGCGCCGTGCACACCAGGGGCCTGGACTTCGCCTGCGACATCTACATCTGGGCCCCCCTGGCCGGCACCTGCGGCGTGCTGCTGCTGAGCCTGGTGATCACCCTGTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGGGTGAAGTTCAGCAGGAGCGCCGACGCCCCCGCCTACCAGCAGGGCCAGAACCAGCTGTACAACGAGCTGAACCTGGGCAGGAGGGAGGAGTACGACGTGCTGGACAAGAGGAGGGGCAGGGACCCCGAGATGGGCGGCAAGCCCCAGAGGAGGAAGAACCCCCAGGAGGGCCTGTACAACGAGCTGCAGAAGGACAAGATGGCCGAGGCCTACAGCGAGATCGGCATGAAGGGCGAGAGGAGGAGGGGCAAGGGCCACGACGGCCTGTACCAGGGCCTGAGCACCGCCACCAAGGACACCTACGACGCCCTGCACATGCAGGCCCTGCCCCCCAGGGGCAGCGGCGAAGGCCGCGGCAGCCTGCTGACCTGCGGCGATGTGGAAGAAAACCCGGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAAC

SEQ ID NO: 9 - protein, 1043 aa, synthetic construct
  REGION 1..1043: note = Description of sequence: CD7-RTX-CD20h (Also called CD7-RTX-CD20) cCAR amino acid sequence
MALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMDIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRGGGGSGGGGSGGGGSQVQLQQSGAEVKKPGSSVKVSCKASGYTFSSYNMHWVRQAPGQGLEWMGAIYPGNGDTSYNQKFKGRATITADESTNTAYMELSSLRSEDTAFYFCARSTYYGGDWYFDVWGQGTTVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 10 - protein, 3140 aa, synthetic construct
  REGION 1..3140: note = Description of sequence: CD7-RTX-CD20h (Also called CD7-RTX-CD20) cCAR nucleotide sequence
ATGGCCTTACCAGTGACCGCCTTGCTCCTGCCGCTGGCCTTGCTGCTCCACGCCGCCAGGCCGGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGGACATCCAGCTGACCCAGAGCCCCAGCAGCCTGAGCGCCAGCGTGGGCGACAGGGTGACCATGACCTGCAGGGCCAGCAGCAGCGTGAGCTACATCCACTGGTTCCAGCAGAAGCCCGGCAAGGCCCCCAAGCCCTGGATCTACGCCACCAGCAACCTGGCCAGCGGCGTGCCCGTGAGGTTCAGCGGCAGCGGCAGCGGCACCGACTACACCTTCACCATCAGCAGCCTGCAGCCCGAGGACATCGCCACCTACTACTGCCAGCAGTGGACCAGCAACCCCCCCACCTTCGGCGGCGGCACCAAGCTGGAGATCAAGAGGGGCGGCGGCGGCAGCGGCGGCGGCGGCAGCGGCGGCGGCGGCAGCCAGGTGCAGCTGCAGCAGAGCGGCGCCGAGGTGAAGAAGCCCGGCAGCAGCGTGAAGGTGAGCTGCAAGGCCAGCGGCTACACCTTCAGCAGCTACAACATGCACTGGGTGAGGCAGGCCCCCGGCCAGGGCCTGGAGTGGATGGGCGCCATCTACCCCGGCAACGGCGACACCAGCTACAACCAGAAGTTCAAGGGCAGGGCCACCATCACCGCCGACGAGAGCACCAACACCGCCTACATGGAGCTGAGCAGCCTGAGGAGCGAGGACACCGCCTTCTACTTCTGCGCCAGGAGCACCTACTACGGCGGCGACTGGTACTTCGACGTGTGGGGCCAGGGCACCACCGTGACCGTGAGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAAGTTTAAAC

SEQ ID NO: 11 - protein, 1337 aa, synthetic construct
  REGION 1..1337: note = Description of sequence: CD7-RTX-CD20h VAC cCAR amino acid sequence
MALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMDIQLTQSPSSLSASVGDRVTMTCRASSSVSYIHWFQQKPGKAPKPWIYATSNLASGVPVRFSGSGSGTDYTFTISSLQPEDIATYYCQQWTSNPPTFGGGTKLEIKRGGGGSGGGGSGGGGSQVQLQQSGAEVKKPGSSVKVSCKASGYTFSSYNMHWVRQAPGQGLEWMGAIYPGNGDTSYNQKFKGRATITADESTNTAYMELSSLRSEDTAFYFCARSTYYGGDWYFDVWGQGTTVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 12 - protein, 4022 aa, synthetic construct
  REGION 1..4022: note = Description of sequence: CD7-RTX-CD20h VAC cCAR nucleotide sequence
ATGGCCTTACCAGTGACCGCCTTGCTCCTGCCGCTGGCCTTGCTGCTCCACGCCGCCAGGCCGGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGGACATCCAGCTGACCCAGAGCCCCAGCAGCCTGAGCGCCAGCGTGGGCGACAGGGTGACCATGACCTGCAGGGCCAGCAGCAGCGTGAGCTACATCCACTGGTTCCAGCAGAAGCCCGGCAAGGCCCCCAAGCCCTGGATCTACGCCACCAGCAACCTGGCCAGCGGCGTGCCCGTGAGGTTCAGCGGCAGCGGCAGCGGCACCGACTACACCTTCACCATCAGCAGCCTGCAGCCCGAGGACATCGCCACCTACTACTGCCAGCAGTGGACCAGCAACCCCCCCACCTTCGGCGGCGGCACCAAGCTGGAGATCAAGAGGGGCGGCGGCGGCAGCGGCGGCGGCGGCAGCGGCGGCGGCGGCAGCCAGGTGCAGCTGCAGCAGAGCGGCGCCGAGGTGAAGAAGCCCGGCAGCAGCGTGAAGGTGAGCTGCAAGGCCAGCGGCTACACCTTCAGCAGCTACAACATGCACTGGGTGAGGCAGGCCCCCGGCCAGGGCCTGGAGTGGATGGGCGCCATCTACCCCGGCAACGGCGACACCAGCTACAACCAGAAGTTCAAGGGCAGGGCCACCATCACCGCCGACGAGAGCACCAACACCGCCTACATGGAGCTGAGCAGCCTGAGGAGCGAGGACACCGCCTTCTACTTCTGCGCCAGGAGCACCTACTACGGCGGCGACTGGTACTTCGACGTGTGGGGCCAGGGCACCACCGTGACCGTGAGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGCAGCGGCGAAGGCCGCGGCAGCCTGCTGACCTGCGGCGATGTGGAAGAAAACCCGGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAAC

SEQ ID NO: 13 - protein, 1367 aa, synthetic construct
  REGION 1..1367: note = Description of sequence: CD7-RTX-CD33 VAC cCAR amino acid sequence
MALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPMADYKDIVMTQSHKFLLVSVGDRVSITCKASQDVSTAVAWYQQKPGQSPKLLIYSASYRYTGVPDRFIGSGSGTDFTLTISSVQAEDLADYFCQQHYSTPLTFGAGTKLEIKRGGGGSGGGGSGGGGSSGGGSEVQLKESGPGLVAPSQSLSITCTVSGFPLTSYGVSWVRQPPGKGLEWLGVIWGDGSTNYHSALISRLSISKDNSKSQVFLKLNNLQTDDTATYYCARDTYYPYYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 14 - protein, 4112 aa, synthetic construct
  REGION 1..4112: note = Description of sequence: CD7-RTX-CD33 VAC cCAR nucleotide sequence
ATGGCCTTACCAGTGACCGCCTTGCTCCTGCCGCTGGCCTTGCTGCTCCACGCCGCCAGGCCGGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGGCCCTGCCCGTGACCGCCCTGCTGCTGCCCCTGGCCCTGCTGCTGCACGCCGCCAGGCCCATGGCCGACTACAAGGACATCGTGATGACCCAGAGCCACAAGTTCCTGCTGGTGAGCGTGGGCGACAGGGTGAGCATCACCTGCAAGGCCAGCCAGGACGTGAGCACCGCCGTGGCCTGGTACCAGCAGAAGCCCGGCCAGAGCCCCAAGCTGCTGATCTACAGCGCCAGCTACAGGTACACCGGCGTGCCCGACAGGTTCATCGGCAGCGGCAGCGGCACCGACTTCACCCTGACCATCAGCAGCGTGCAGGCCGAGGACCTGGCCGACTACTTCTGCCAGCAGCACTACAGCACCCCCCTGACCTTCGGCGCCGGCACCAAGCTGGAGATCAAGAGGGGCGGCGGCGGCAGCGGCGGCGGCGGCAGCGGCGGCGGCGGCAGCAGCGGCGGCGGCAGCGAGGTGCAGCTGAAGGAGAGCGGCCCCGGCCTGGTGGCCCCCAGCCAGAGCCTGAGCATCACCTGCACCGTGAGCGGCTTCCCCCTGACCAGCTACGGCGTGAGCTGGGTGAGGCAGCCCCCCGGCAAGGGCCTGGAGTGGCTGGGCGTGATCTGGGGCGACGGCAGCACCAACTACCACAGCGCCCTGATCAGCAGGCTGAGCATCAGCAAGGACAACAGCAAGAGCCAGGTGTTCCTGAAGCTGAACAACCTGCAGACCGACGACACCGCCACCTACTACTGCGCCAGGGACACCTACTACCCCTACTACGCCATGGACTACTGGGGCCAGGGCACCAGCGTGACCGTGAGCAGCACCACCACCCCCGCCCCCAGGCCCCCCACCCCCGCCCCCACCATCGCCAGCCAGCCCCTGAGCCTGAGGCCCGAGGCCTGCAGGCCCGCCGCCGGCGGCGCCGTGCACACCAGGGGCCTGGACTTCGCCTGCGACATCTACATCTGGGCCCCCCTGGCCGGCACCTGCGGCGTGCTGCTGCTGAGCCTGGTGATCACCCTGTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGGGTGAAGTTCAGCAGGAGCGCCGACGCCCCCGCCTACCAGCAGGGCCAGAACCAGCTGTACAACGAGCTGAACCTGGGCAGGAGGGAGGAGTACGACGTGCTGGACAAGAGGAGGGGCAGGGACCCCGAGATGGGCGGCAAGCCCCAGAGGAGGAAGAACCCCCAGGAGGGCCTGTACAACGAGCTGCAGAAGGACAAGATGGCCGAGGCCTACAGCGAGATCGGCATGAAGGGCGAGAGGAGGAGGGGCAAGGGCCACGACGGCCTGTACCAGGGCCTGAGCACCGCCACCAAGGACACCTACGACGCCCTGCACATGCAGGCCCTGCCCCCCAGGGGCAGCGGCGAAGGCCGCGGCAGCCTGCTGACCTGCGGCGATGTGGAAGAAAACCCGGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAAC

SEQ ID NO: 15 - protein, 1356 aa, synthetic construct
  REGION 1..1356: note = Description of sequence: CD7-RTX-CLL1 VAC cCARamino acid sequence
MALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSHKFMSTSVGDRVSITCKASQDVSTAVAWFQQKPGQSPKLLIYSPSYRYTGVPDRFTGSGSGTDFTFTISSVQAEDLAVYYCQQLYSTPYTFGGGTKLEIKGGGGSGGGGSGGGGSEVQLQQSGPELVKPGASVKMSCKASGYTFTDYYLDWVKQSHGESFEWIGRVNPYNGGTIYNQKFKGKATLTVDKSSSTAYMDLNSLTSEDSAVYYCARDHYRYDPLLDYWGQGTTLTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 16 - protein, 4079 aa, synthetic construct
  REGION 1..4079: note = Description of sequence: CD7-RTX-CLL1 VAC cCAR nucleotide sequence
ATGGCCTTACCAGTGACCGCCTTGCTCCTGCCGCTGGCCTTGCTGCTCCACGCCGCCAGGCCGGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGGCCCTGCCCGTGACCGCCCTGCTGCTGCCCCTGGCCCTGCTGCTGCACGCCGCCAGGCCCGACATCCAGATGACCCAGAGCCACAAGTTCATGAGCACCAGCGTGGGCGACAGGGTGAGCATCACCTGCAAGGCCAGCCAGGACGTGAGCACCGCCGTGGCCTGGTTCCAGCAGAAGCCCGGCCAGAGCCCCAAGCTGCTGATCTACAGCCCCAGCTACAGGTACACCGGCGTGCCCGACAGGTTCACCGGCAGCGGCAGCGGCACCGACTTCACCTTCACCATCAGCAGCGTGCAGGCCGAGGACCTGGCCGTGTACTACTGCCAGCAGCTGTACAGCACCCCCTACACCTTCGGCGGCGGCACCAAGCTGGAGATCAAGGGAGGGGGGGGATCCGGGGGAGGAGGCTCCGGCGGAGGCGGAAGCGAGGTGCAGCTGCAGCAGAGCGGCCCCGAGCTGGTGAAGCCCGGCGCCAGCGTGAAGATGAGCTGCAAGGCCAGCGGCTACACCTTCACCGACTACTACCTGGACTGGGTGAAGCAGAGCCACGGCGAGAGCTTCGAGTGGATCGGCAGGGTGAACCCCTACAACGGCGGCACCATCTACAACCAGAAGTTCAAGGGCAAGGCCACCCTGACCGTGGACAAGAGCAGCAGCACCGCCTACATGGACCTGAACAGCCTGACCAGCGAGGACAGCGCCGTGTACTACTGCGCCAGGGACCACTACAGGTACGACCCCCTGCTGGACTACTGGGGCCAGGGCACCACCCTGACCGTGAGCAGCACCACCACCCCCGCCCCCAGGCCCCCCACCCCCGCCCCCACCATCGCCAGCCAGCCCCTGAGCCTGAGGCCCGAGGCCTGCAGGCCCGCCGCCGGCGGCGCCGTGCACACCAGGGGCCTGGACTTCGCCTGCGACATCTACATCTGGGCCCCCCTGGCCGGCACCTGCGGCGTGCTGCTGCTGAGCCTGGTGATCACCCTGTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGGGTGAAGTTCAGCAGGAGCGCCGACGCCCCCGCCTACCAGCAGGGCCAGAACCAGCTGTACAACGAGCTGAACCTGGGCAGGAGGGAGGAGTACGACGTGCTGGACAAGAGGAGGGGCAGGGACCCCGAGATGGGCGGCAAGCCCCAGAGGAGGAAGAACCCCCAGGAGGGCCTGTACAACGAGCTGCAGAAGGACAAGATGGCCGAGGCCTACAGCGAGATCGGCATGAAGGGCGAGAGGAGGAGGGGCAAGGGCCACGACGGCCTGTACCAGGGCCTGAGCACCGCCACCAAGGACACCTACGACGCCCTGCACATGCAGGCCCTGCCCCCCAGGGGCAGCGGCGAAGGCCGCGGCAGCCTGCTGACCTGCGGCGATGTGGAAGAAAACCCGGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAAC

SEQ ID NO: 17 - protein, 1061 aa, synthetic construct
  REGION 1..1061: note = Description of sequence: CD7-RTX-CS1 cCAR amino acid sequence
MALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKGGGGSGGGGSGGGGSEVQLVESGGGLVQPGGSLRLSCAASGFFSRYWMSWVRQAPGKGLEWIGEINPDSSTINYAPSLKDKFIISRDNAKNSLYLQMNSLRAEDTAVYYCARPDGNYWYFDVWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 18 - protein, 3194 aa, synthetic construct
  REGION 1..3194: note = Description of sequence: CD7-RTX-CS1 cCAR nucleotide sequence
ATGGCCTTACCAGTGACCGCCTTGCTCCTGCCGCTGGCCTTGCTGCTCCACGCCGCCAGGCCGGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGGCCTTACCAGTGACCGCCTTGCTCCTGCCGCTGGCCTTGCTGCTCCACGCCGCCAGGCCGGACATCCAGATGACCCAGAGCCCCAGCAGCCTGAGCGCCAGCGTGGGCGACAGGGTGACCATCACCTGCAAGGCCAGCCAGGACGTGGGCATCGCCGTGGCCTGGTACCAGCAGAAGCCCGGCAAGGTGCCCAAGCTGCTGATCTACTGGGCCAGCACCAGGCACACCGGCGTGCCCGACAGGTTCAGCGGCAGCGGCAGCGGCACCGACTTCACCCTGACCATCAGCAGCCTGCAGCCCGAGGACGTGGCCACCTACTACTGCCAGCAGTACAGCAGCTACCCCTACACCTTCGGCCAGGGCACCAAGGTGGAGATCAAGGGAGGGGGGGGATCCGGGGGAGGAGGCTCCGGCGGAGGCGGAAGCGAGGTGCAGCTGGTGGAGAGCGGCGGCGGCCTGGTGCAGCCCGGCGGCAGCCTGAGGCTGAGCTGCGCCGCCAGCGGCTTCTTCAGCAGGTACTGGATGAGCTGGGTGAGGCAGGCCCCCGGCAAGGGCCTGGAGTGGATCGGCGAGATCAACCCCGACAGCAGCACCATCAACTACGCCCCCAGCCTGAAGGACAAGTTCATCATCAGCAGGGACAACGCCAAGAACAGCCTGTACCTGCAGATGAACAGCCTGAGGGCCGAGGACACCGCCGTGTACTACTGCGCCAGGCCCGACGGCAACTACTGGTACTTCGACGTGTGGGGCCAGGGCACCCTGGTGACCGTGAGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGGGTGAAGTTCAGCAGGAGCGCCGACGCCCCCGCCTACCAGCAGGGCCAGAACCAGCTGTACAACGAGCTGAACCTGGGCAGGAGGGAGGAGTACGACGTGCTGGACAAGAGGAGGGGCAGGGACCCCGAGATGGGCGGCAAGCCCCAGAGGAGGAAGAACCCCCAGGAGGGCCTGTACAACGAGCTGCAGAAGGACAAGATGGCCGAGGCCTACAGCGAGATCGGCATGAAGGGCGAGAGGAGGAGGGGCAAGGGCCACGACGGCCTGTACCAGGGCCTGAGCACCGCCACCAAGGACACCTACGACGCCCTGCACATGCAGGCCCTGCCCCCCAGGTAAGTTTAAAC

SEQ ID NO: 19 - protein, 1043 aa, synthetic construct
  REGION 1..1043: note = Description of sequence: CD7-RTX-BCMA cCAR amino acid sequence
MALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKGGGGSGGGGSGGGGSQIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 20 - protein, 3140 aa, synthetic construct
  REGION 1..3140: note = Description of sequence: CD7-RTX-BCMA cCAR nucleotide sequence
ATGGCCTTACCAGTGACCGCCTTGCTCCTGCCGCTGGCCTTGCTGCTCCACGCCGCCAGGCCGGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCGACGTGGTGATGACCCAGAGCCACAGGTTCATGAGCACCAGCGTGGGCGACAGGGTGAGCATCACCTGCAGGGCCAGCCAGGACGTGAACACCGCCGTGAGCTGGTACCAGCAGAAGCCCGGCCAGAGCCCCAAGCTGCTGATCTTCAGCGCCAGCTACAGGTACACCGGCGTGCCCGACAGGTTCACCGGCAGCGGCAGCGGCGCCGACTTCACCCTGACCATCAGCAGCGTGCAGGCCGAGGACCTGGCCGTGTACTACTGCCAGCAGCACTACAGCACCCCCTGGACCTTCGGCGGCGGCACCAAGCTGGACATCAAGGGAGGGGGGGGATCCGGGGGAGGAGGCTCCGGCGGAGGCGGAAGCCAGATCCAGCTGGTGCAGAGCGGCCCCGACCTGAAGAAGCCCGGCGAGACCGTGAAGCTGAGCTGCAAGGCCAGCGGCTACACCTTCACCAACTTCGGCATGAACTGGGTGAAGCAGGCCCCCGGCAAGGGCTTCAAGTGGATGGCCTGGATCAACACCTACACCGGCGAGAGCTACTTCGCCGACGACTTCAAGGGCAGGTTCGCCTTCAGCGTGGAGACCAGCGCCACCACCGCCTACCTGCAGATCAACAACCTGAAGACCGAGGACACCGCCACCTACTTCTGCGCCAGGGGCGAGATCTACTACGGCTACGACGGCGGCTTCGCCTACTGGGGCCAGGGCACCCTGGTGACCGTGAGCGCCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAAGTTTAAAC

SEQ ID NO: 21 - protein, 1337 aa, synthetic construct
  REGION 1..1337: note = Description of sequence: CD7-RTX-BCMA VAC cCAR amino acid sequence
MALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKGGGGSGGGGSGGGGSQIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 22 - protein, 4022 aa, synthetic construct
  REGION 1..4022: note = Description of sequence: CD7-RTX-BCMA VAC cCAR nucleotide sequence
ATGGCCTTACCAGTGACCGCCTTGCTCCTGCCGCTGGCCTTGCTGCTCCACGCCGCCAGGCCGGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCGACGTGGTGATGACCCAGAGCCACAGGTTCATGAGCACCAGCGTGGGCGACAGGGTGAGCATCACCTGCAGGGCCAGCCAGGACGTGAACACCGCCGTGAGCTGGTACCAGCAGAAGCCCGGCCAGAGCCCCAAGCTGCTGATCTTCAGCGCCAGCTACAGGTACACCGGCGTGCCCGACAGGTTCACCGGCAGCGGCAGCGGCGCCGACTTCACCCTGACCATCAGCAGCGTGCAGGCCGAGGACCTGGCCGTGTACTACTGCCAGCAGCACTACAGCACCCCCTGGACCTTCGGCGGCGGCACCAAGCTGGACATCAAGGGAGGGGGGGGATCCGGGGGAGGAGGCTCCGGCGGAGGCGGAAGCCAGATCCAGCTGGTGCAGAGCGGCCCCGACCTGAAGAAGCCCGGCGAGACCGTGAAGCTGAGCTGCAAGGCCAGCGGCTACACCTTCACCAACTTCGGCATGAACTGGGTGAAGCAGGCCCCCGGCAAGGGCTTCAAGTGGATGGCCTGGATCAACACCTACACCGGCGAGAGCTACTTCGCCGACGACTTCAAGGGCAGGTTCGCCTTCAGCGTGGAGACCAGCGCCACCACCGCCTACCTGCAGATCAACAACCTGAAGACCGAGGACACCGCCACCTACTTCTGCGCCAGGGGCGAGATCTACTACGGCTACGACGGCGGCTTCGCCTACTGGGGCCAGGGCACCCTGGTGACCGTGAGCGCCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGCAGCGGCGAAGGCCGCGGCAGCCTGCTGACCTGCGGCGATGTGGAAGAAAACCCGGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAAC

SEQ ID NO: 23 - protein, 1069 aa, synthetic construct
  REGION 1..1069: note = Description of sequence: CD7-RTX-CD38a VAC cCAR amino acid sequence
ALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIELTQSPSSFSVSLGDRVTITCKASEDIYNRLAWYQQKPGNAPRLLISGATSLETGVPSRFSGSGSGKDYTLSITSLQTEDVATYYCQQYWSTPTFGGGTKLEIKRAAGGGGSGGGGSGGGGSAQPAMAKVQLQESGPSLVQPSQRLSITCTVSGFSLISYGVHWVRQSPGKGLEWLGVIWRGGSTDYNAAFMSRLSITKDNSKSQVFFKMNSLQADDTAIYFCAKTLITTGYAMDYWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 24 - protein, 3221 aa, synthetic construct
  REGION 1..3221: note = Description of sequence: CD7-RTX-CD38a (also called CD7-RTX-CD38) VAC cCAR nucleotide sequence
ATGGCCTTACCAGTGACCGCCTTGCTCCTGCCGCTGGCCTTGCTGCTCCACGCCGCCAGGCCGGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGGCCCTGCCCGTGACCGCCCTGCTGCTGCCCCTGGCCCTGCTGCTGCACGCCGCCAGGCCCGACATCGAGCTGACCCAGAGCCCCAGCAGCTTCAGCGTGAGCCTGGGCGACAGGGTGACCATCACCTGCAAGGCCAGCGAGGACATCTACAACAGGCTGGCCTGGTACCAGCAGAAGCCCGGCAACGCCCCCAGGCTGCTGATCAGCGGCGCCACCAGCCTGGAGACCGGCGTGCCCAGCAGGTTCAGCGGCAGCGGCAGCGGCAAGGACTACACCCTGAGCATCACCAGCCTGCAGACCGAGGACGTGGCCACCTACTACTGCCAGCAGTACTGGAGCACCCCCACCTTCGGCGGCGGCACCAAGCTGGAGATCAAGAGGGCCGCCGGCGGCGGCGGCAGCGGCGGCGGCGGCAGCGGCGGCGGCGGCAGCGCCCAGCCCGCCATGGCCAAGGTGCAGCTGCAGGAGAGCGGCCCCAGCCTGGTGCAGCCCAGCCAGAGGCTGAGCATCACCTGCACCGTGAGCGGCTTCAGCCTGATCAGCTACGGCGTGCACTGGGTGAGGCAGAGCCCCGGCAAGGGCCTGGAGTGGCTGGGCGTGATCTGGAGGGGCGGCAGCACCGACTACAACGCCGCCTTCATGAGCAGGCTGAGCATCACCAAGGACAACAGCAAGAGCCAGGTGTTCTTCAAGATGAACAGCCTGCAGGCCGACGACACCGCCATCTACTTCTGCGCCAAGACCCTGATCACCACCGGCTACGCCATGGACTACTGGGGCCAGGGCACCACCGTGACCGTGAGCAGCACCACCACCCCCGCCCCCAGGCCCCCCACCCCCGCCCCCACCATCGCCAGCCAGCCCCTGAGCCTGAGGCCCGAGGCCTGCAGGCCCGCCGCCGGCGGCGCCGTGCACACCAGGGGCCTGGACTTCGCCTGCGACATCTACATCTGGGCCCCCCTGGCCGGCACCTGCGGCGTGCTGCTGCTGAGCCTGGTGATCACCCTGTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGGGTGAAGTTCAGCAGGAGCGCCGACGCCCCCGCCTACCAGCAGGGCCAGAACCAGCTGTACAACGAGCTGAACCTGGGCAGGAGGGAGGAGTACGACGTGCTGGACAAGAGGAGGGGCAGGGACCCCGAGATGGGCGGCAAGCCCCAGAGGAGGAAGAACCCCCAGGAGGGCCTGTACAACGAGCTGCAGAAGGACAAGATGGCCGAGGCCTACAGCGAGATCGGCATGAAGGGCGAGAGGAGGAGGGGCAAGGGCCACGACGGCCTGTACCAGGGCCTGAGCACCGCCACCAAGGACACCTACGACGCCCTGCACATGCAGGCCCTGCCCCCCAGGTAAGTTTAAAC

SEQ ID NO: 25 - protein, 1298 aa, synthetic construct
  REGION 1..1298: note = Description of sequence: pX-BCMA-CD19b-IL15/IL15sushi-28 amino acid sequence
MALPVTALLLPLALLLHAARPDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKGGGGSGGGGSGGGGSQIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 26 - protein, 3905 aa, synthetic construct
  REGION 1..3905: note = Description of sequence: pX-BCMA-CD19b-IL15/IL15sushi -28-nucleotide sequence
ATGGCCTTACCAGTGACCGCCTTGCTCCTGCCGCTGGCCTTGCTGCTCCACGCCGCCAGGCCGGACGTGGTGATGACCCAGAGCCACAGGTTCATGAGCACCAGCGTGGGCGACAGGGTGAGCATCACCTGCAGGGCCAGCCAGGACGTGAACACCGCCGTGAGCTGGTACCAGCAGAAGCCCGGCCAGAGCCCCAAGCTGCTGATCTTCAGCGCCAGCTACAGGTACACCGGCGTGCCCGACAGGTTCACCGGCAGCGGCAGCGGCGCCGACTTCACCCTGACCATCAGCAGCGTGCAGGCCGAGGACCTGGCCGTGTACTACTGCCAGCAGCACTACAGCACCCCCTGGACCTTCGGCGGCGGCACCAAGCTGGACATCAAGGGAGGGGGGGGATCCGGGGGAGGAGGCTCCGGCGGAGGCGGAAGCCAGATCCAGCTGGTGCAGAGCGGCCCCGACCTGAAGAAGCCCGGCGAGACCGTGAAGCTGAGCTGCAAGGCCAGCGGCTACACCTTCACCAACTTCGGCATGAACTGGGTGAAGCAGGCCCCCGGCAAGGGCTTCAAGTGGATGGCCTGGATCAACACCTACACCGGCGAGAGCTACTTCGCCGACGACTTCAAGGGCAGGTTCGCCTTCAGCGTGGAGACCAGCGCCACCACCGCCTACCTGCAGATCAACAACCTGAAGACCGAGGACACCGCCACCTACTTCTGCGCCAGGGGCGAGATCTACTACGGCTACGACGGCGGCTTCGCCTACTGGGGCCAGGGCACCCTGGTGACCGTGAGCGCCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGGCCCTGCCCGTGACCGCCCTGCTGCTGCCCCTGGCCCTGCTGCTGCACGCCGCCAGGCCCGAGGTCCAGCTGCAGCAGTCTGGACCTGAGCTGATAAAGCCTGGGGCTTCAGTGAAGATGTCCTGCAAGGCTTCTGGATACACATTCACTAGCTATGTTATGCACTGGGTGAAGCAGAAGCCTGGGCAGGGCCTTGAGTGGATTGGATATATTAATCCTTACAATGATGGTACTAAGTACAATGAGAAGTTCAAAGGCAAGGCCACACTGACTTCAGACAAATCCTCCAGCACAGCCTACATGGAGCTCAGCAGCCTGACCTCTGAGGACTCTGCGGTCTATTACTGTGCAAGAGGGACTTATTACTACGGTAGTAGGGTATTTGACTACTGGGGCCAAGGCACCACTCTCACAGTCTCCTCAGGTGGAGGGGGCTCAGGCGGAGGTGGCTCTGGGGGTGGAGGCTCGGACATTGTGATGACTCAGGCTGCACCCTCTATACCTGTCACTCCTGGAGAGTCAGTATCCATCTCCTGCAGGTCTAGTAAGAGTCTCCTGAATAGTAATGGCAACACTTACTTGTATTGGTTCCTGCAGAGGCCAGGCCAGTCTCCTCAGCTCCTGATATATCGGATGTCCAACCTTGCCTCAGGAGTCCCAGACAGGTTCAGTGGCAGTGGGTCAGGAACTGCTTTCACACTGAGAATCAGTAGAGTGGAGGCTGAGGATGTGGGTGTTTATTACTGTATGCAACATCTAGAATATCCGTTCACGTTCGGTGCTGGGACCAAGCTGGAGCTGAAACGGACCACCACCCCCGCCCCCAGGCCCCCCACCCCCGCCCCCACCATCGCCAGCCAGCCCCTGAGCCTGAGGCCCGAGGCCTGCAGGCCCGCCGCCGGCGGCGCCGTGCACACCAGGGGCCTGGACTTCGCCTGCGACATCTACATCTGGGCCCCCCTGGCCGGCACCTGCGGCGTGCTGCTGCTGAGCCTGGTGATCACCCTGTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGGGTGAAGTTCAGCAGGAGCGCCGACGCCCCCGCCTACCAGCAGGGCCAGAACCAGCTGTACAACGAGCTGAACCTGGGCAGGAGGGAGGAGTACGACGTGCTGGACAAGAGGAGGGGCAGGGACCCCGAGATGGGCGGCAAGCCCCAGAGGAGGAAGAACCCCCAGGAGGGCCTGTACAACGAGCTGCAGAAGGACAAGATGGCCGAGGCCTACAGCGAGATCGGCATGAAGGGCGAGAGGAGGAGGGGCAAGGGCCACGACGGCCTGTACCAGGGCCTGAGCACCGCCACCAAGGACACCTACGACGCCCTGCACATGCAGGCCCTGCCCCCCAGGGGCAGCGGCGAAGGCCGCGGCAGCCTGCTGACCTGCGGCGATGTGGAAGAAAACCCGGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAAC

SEQ ID NO: 27 - protein, 1256 aa, synthetic construct
  REGION 1..1256: note = Description of sequence: pX-BC4-CD7Q-15R amino acid sequence
MALPVTALLLPLALLLHAARPQVQLVESGGGLVQAGGSLRLSCVASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPGSGEGRGSLLTCGDVEENPGPMALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 28 - protein, 3810 aa, synthetic construct
  REGION 1..3810: note = Description of sequence: pX-BC4-CD7Q-15R nucleotide sequence
GGATCCACGCGTGCGATCGCACCATGGCCTTACCAGTGACCGCCTTGCTCCTGCCGCTGGCCTTGCTGCTCCACGCCGCCAGGCCGCAGGTGCAGCTGGTGGAGAGCGGCGGCGGCCTGGTGCAGGCCGGCGGCAGCCTGAGGCTGAGCTGCGTGGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCGGCAGCGGCGAAGGCCGCGGCAGCCTGCTGACCTGCGGCGATGTGGAAGAAAACCCGGGCCCCATGGCCTTACCAGTGACCGCCTTGCTCCTGCCGCTGGCCTTGCTGCTCCACGCCGCCAGGCCGGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAACGCGGCCGC

SEQ ID NO: 29 - protein, 1166 aa, synthetic construct
  REGION 1..1166: note = Description of sequence: pX-BC4-CD19c -Vac amino acid sequence
MALPVTALLLPLALLLHAARPQVQLVESGGGLVQAGGSLRLSCVASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 30 - protein, 3517 aa, synthetic construct
  REGION 1..3517: note = Description of sequence: pX-BC4-CD19c -Vac nucleotide sequence
ATGGCCTTACCAGTGACCGCCTTGCTCCTGCCGCTGGCCTTGCTGCTCCACGCCGCCAGGCCGCAGGTGCAGCTGGTGGAGAGCGGCGGCGGCCTGGTGCAGGCCGGCGGCAGCCTGAGGCTGAGCTGCGTGGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGGCCTTACCAGTGACCGCCTTGCTCCTGCCGCTGGCCTTGCTGCTCCACGCCGCCAGGCCGGACATCCAGATGACACAGACTACATCCTCCCTGTCTGCCTCTCTGGGAGACAGAGTCACCATCAGTTGCAGGGCAAGTCAGGACATTAGTAAATATTTAAATTGGTATCAGCAGAAACCAGATGGAACTGTTAAACTCCTGATCTACCATACATCAAGATTACACTCAGGAGTCCCATCAAGGTTCAGTGGCAGTGGGTCTGGAACAGATTATTCTCTCACCATTAGCAACCTGGAGCAAGAAGATATTGCCACTTACTTTTGCCAACAGGGTAATACGCTTCCGTACACGTTCGGAGGGGGGACCAAGCTGGAGATCACAGGTGGCGGTGGCTCGGGCGGTGGTGGGTCGGGTGGCGGCGGATCTGAGGTGAAACTGCAGGAGTCAGGACCTGGCCTGGTGGCGCCCTCACAGAGCCTGTCCGTCACATGCACTGTCTCAGGGGTCTCATTACCCGACTATGGTGTAAGCTGGATTCGCCAGCCTCCACGAAAGGGTCTGGAGTGGCTGGGAGTAATATGGGGTAGTGAAACCACATACTATAATTCAGCTCTCAAATCCAGACTGACCATCATCAAGGACAACTCCAAGAGCCAAGTTTTCTTAAAAATGAACAGTCTGCAAACTGATGACACAGCCATTTACTACTGTGCCAAACATTATTACTACGGTGGTAGCTATGCTATGGACTACTGGGGCCAAGGAACCTCAGTCACCGTCTCCTCAACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGCAGCGGCGAAGGCCGCGGCAGCCTGCTGACCTGCGGCGATGTGGAAGAAAACCCGGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAACGCGGCCGC

SEQ ID NO: 31 - protein, 1299 aa, synthetic construct
  REGION 1..1299: note = Description of sequence: CLL1-CD33b-IL15/IL15sushi-2G CAR amino acid sequence
MALPVTALLLPLALLLHAARPDIQMTQSHKFMSTSVGDRVSITCKASQDVSTAVAWFQQKPGQSPKLLIYSPSYRYTGVPDRFTGSGSGTDFTFTISSVQAEDLAVYYCQQLYSTPYTFGGGTKLEIKGGGGSGGGGSGGGGSEVQLQQSGPELVKPGASVKMSCKASGYTFTDYYLDWVKQSHGESFEWIGRVNPYNGGTIYNQKFKGKATLTVDKSSSTAYMDLNSLTSEDSAVYYCARDHYRYDPLLDYWGQGTTLTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPMADYKDIVMTQSHKFLLVSVGDRVSITCKASQDVSTAVAWYQQKPGQSPKLLIYSASYRYTGVPDRFIGSGSGTDFTLTISSVQAEDLADYFCQQHYSTPLTFGAGTKLEIKRGGGGSGGGGSGGGGSSGGGSEVQLKESGPGLVAPSQSLSITCTVSGFPLTSYGVSWVRQPPGKGLEWLGVIWGDGSTNYHSALISRLSISKDNSKSQVFLKLNNLQTDDTATYYCARDTYYPYYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 32 - protein, 3908 aa, synthetic construct
  REGION 1..3908: note = Description of sequence: CLL1-CD33b-IL15/IL15sushi-2G CAR nucleotide sequence
ATGGCCCTGCCCGTGACCGCCCTGCTGCTGCCCCTGGCCCTGCTGCTGCACGCCGCCAGGCCCGACATCCAGATGACCCAGAGCCACAAGTTCATGAGCACCAGCGTGGGCGACAGGGTGAGCATCACCTGCAAGGCCAGCCAGGACGTGAGCACCGCCGTGGCCTGGTTCCAGCAGAAGCCCGGCCAGAGCCCCAAGCTGCTGATCTACAGCCCCAGCTACAGGTACACCGGCGTGCCCGACAGGTTCACCGGCAGCGGCAGCGGCACCGACTTCACCTTCACCATCAGCAGCGTGCAGGCCGAGGACCTGGCCGTGTACTACTGCCAGCAGCTGTACAGCACCCCCTACACCTTCGGCGGCGGCACCAAGCTGGAGATCAAGGGAGGGGGGGGATCCGGGGGAGGAGGCTCCGGCGGAGGCGGAAGCGAGGTGCAGCTGCAGCAGAGCGGCCCCGAGCTGGTGAAGCCCGGCGCCAGCGTGAAGATGAGCTGCAAGGCCAGCGGCTACACCTTCACCGACTACTACCTGGACTGGGTGAAGCAGAGCCACGGCGAGAGCTTCGAGTGGATCGGCAGGGTGAACCCCTACAACGGCGGCACCATCTACAACCAGAAGTTCAAGGGCAAGGCCACCCTGACCGTGGACAAGAGCAGCAGCACCGCCTACATGGACCTGAACAGCCTGACCAGCGAGGACAGCGCCGTGTACTACTGCGCCAGGGACCACTACAGGTACGACCCCCTGCTGGACTACTGGGGCCAGGGCACCACCCTGACCGTGAGCAGCACCACCACCCCCGCCCCCAGGCCCCCCACCCCCGCCCCCACCATCGCCAGCCAGCCCCTGAGCCTGAGGCCCGAGGCCTGCAGGCCCGCCGCCGGCGGCGCCGTGCACACCAGGGGCCTGGACTTCGCCTGCGACATCTACATCTGGGCCCCCCTGGCCGGCACCTGCGGCGTGCTGCTGCTGAGCCTGGTGATCACCCTGTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGGGTGAAGTTCAGCAGGAGCGCCGACGCCCCCGCCTACCAGCAGGGCCAGAACCAGCTGTACAACGAGCTGAACCTGGGCAGGAGGGAGGAGTACGACGTGCTGGACAAGAGGAGGGGCAGGGACCCCGAGATGGGCGGCAAGCCCCAGAGGAGGAAGAACCCCCAGGAGGGCCTGTACAACGAGCTGCAGAAGGACAAGATGGCCGAGGCCTACAGCGAGATCGGCATGAAGGGCGAGAGGAGGAGGGGCAAGGGCCACGACGGCCTGTACCAGGGCCTGAGCACCGCCACCAAGGACACCTACGACGCCCTGCACATGCAGGCCCTGCCCCCCAGGGGAAGCGGAGCCACCAACTTCAGCCTGCTGAAGCAGGCCGGCGACGTGGAGGAGAACCCCGGCCCCATGGCCCTGCCCGTGACCGCCCTGCTGCTGCCCCTGGCCCTGCTGCTGCACGCCGCCAGGCCCATGGCCGACTACAAGGACATCGTGATGACCCAGAGCCACAAGTTCCTGCTGGTGAGCGTGGGCGACAGGGTGAGCATCACCTGCAAGGCCAGCCAGGACGTGAGCACCGCCGTGGCCTGGTACCAGCAGAAGCCCGGCCAGAGCCCCAAGCTGCTGATCTACAGCGCCAGCTACAGGTACACCGGCGTGCCCGACAGGTTCATCGGCAGCGGCAGCGGCACCGACTTCACCCTGACCATCAGCAGCGTGCAGGCCGAGGACCTGGCCGACTACTTCTGCCAGCAGCACTACAGCACCCCCCTGACCTTCGGCGCCGGCACCAAGCTGGAGATCAAGAGGGGCGGCGGCGGCAGCGGCGGCGGCGGCAGCGGCGGCGGCGGCAGCAGCGGCGGCGGCAGCGAGGTGCAGCTGAAGGAGAGCGGCCCCGGCCTGGTGGCCCCCAGCCAGAGCCTGAGCATCACCTGCACCGTGAGCGGCTTCCCCCTGACCAGCTACGGCGTGAGCTGGGTGAGGCAGCCCCCCGGCAAGGGCCTGGAGTGGCTGGGCGTGATCTGGGGCGACGGCAGCACCAACTACCACAGCGCCCTGATCAGCAGGCTGAGCATCAGCAAGGACAACAGCAAGAGCCAGGTGTTCCTGAAGCTGAACAACCTGCAGACCGACGACACCGCCACCTACTACTGCGCCAGGGACACCTACTACCCCTACTACGCCATGGACTACTGGGGCCAGGGCACCAGCGTGACCGTGAGCAGCACCACCACCCCCGCCCCCAGGCCCCCCACCCCCGCCCCCACCATCGCCAGCCAGCCCCTGAGCCTGAGGCCCGAGGCCTGCAGGCCCGCCGCCGGCGGCGCCGTGCACACCAGGGGCCTGGACTTCGCCTGCGACATCTACATCTGGGCCCCCCTGGCCGGCACCTGCGGCGTGCTGCTGCTGAGCCTGGTGATCACCCTGTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGGGTGAAGTTCAGCAGGAGCGCCGACGCCCCCGCCTACCAGCAGGGCCAGAACCAGCTGTACAACGAGCTGAACCTGGGCAGGAGGGAGGAGTACGACGTGCTGGACAAGAGGAGGGGCAGGGACCCCGAGATGGGCGGCAAGCCCCAGAGGAGGAAGAACCCCCAGGAGGGCCTGTACAACGAGCTGCAGAAGGACAAGATGGCCGAGGCCTACAGCGAGATCGGCATGAAGGGCGAGAGGAGGAGGGGCAAGGGCCACGACGGCCTGTACCAGGGCCTGAGCACCGCCACCAAGGACACCTACGACGCCCTGCACATGCAGGCCCTGCCCCCCAGGGGCAGCGGCGAAGGCCGCGGCAGCCTGCTGACCTGCGGCGATGTGGAAGAAAACCCGGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAAC

SEQ ID NO: 33 - protein, 1357 aa, synthetic construct
  REGION 1..1357: note = Description of sequence: pX-CD7q-CD19c-15R amino acid sequence
MALPVTALLLPLALLLHAARPGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 34 - protein, 4113 aa, synthetic construct
  REGION 1..4113: note = Description of sequence: pX-CD7q-CD19c-15R nucleotide sequence
GGATCCACGCGTGCGATCGCACCATGGCCTTACCAGTGACCGCCTTGCTCCTGCCGCTGGCCTTGCTGCTCCACGCCGCCAGGCCGGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGGCCTTACCAGTGACCGCCTTGCTCCTGCCGCTGGCCTTGCTGCTCCACGCCGCCAGGCCGGACATCCAGATGACACAGACTACATCCTCCCTGTCTGCCTCTCTGGGAGACAGAGTCACCATCAGTTGCAGGGCAAGTCAGGACATTAGTAAATATTTAAATTGGTATCAGCAGAAACCAGATGGAACTGTTAAACTCCTGATCTACCATACATCAAGATTACACTCAGGAGTCCCATCAAGGTTCAGTGGCAGTGGGTCTGGAACAGATTATTCTCTCACCATTAGCAACCTGGAGCAAGAAGATATTGCCACTTACTTTTGCCAACAGGGTAATACGCTTCCGTACACGTTCGGAGGGGGGACCAAGCTGGAGATCACAGGTGGCGGTGGCTCGGGCGGTGGTGGGTCGGGTGGCGGCGGATCTGAGGTGAAACTGCAGGAGTCAGGACCTGGCCTGGTGGCGCCCTCACAGAGCCTGTCCGTCACATGCACTGTCTCAGGGGTCTCATTACCCGACTATGGTGTAAGCTGGATTCGCCAGCCTCCACGAAAGGGTCTGGAGTGGCTGGGAGTAATATGGGGTAGTGAAACCACATACTATAATTCAGCTCTCAAATCCAGACTGACCATCATCAAGGACAACTCCAAGAGCCAAGTTTTCTTAAAAATGAACAGTCTGCAAACTGATGACACAGCCATTTACTACTGTGCCAAACATTATTACTACGGTGGTAGCTATGCTATGGACTACTGGGGCCAAGGAACCTCAGTCACCGTCTCCTCAACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGCAGCGGCGAAGGCCGCGGCAGCCTGCTGACCTGCGGCGATGTGGAAGAAAACCCGGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAACGCGGCCGC

SEQ ID NO: 35 - protein, 693 aa, synthetic construct
  REGION 1..693: note = Description of sequence: pX-BC4-CD7Q-tandem amino acid sequence
MALPVTALLLPLALLLHAARPQVQLVESGGGLVQAGGSLRLSCVASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSGGGGSGGGGSGGGGSGGGGSGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 36 - protein, 2098 aa, synthetic construct
  REGION 1..2098: note = Description of sequence: pX-BC4-CD7Q-tandem nucleotide sequence
ATGGCCTTACCAGTGACCGCCTTGCTCCTGCCGCTGGCCTTGCTGCTCCACGCCGCCAGGCCGCAGGTGCAGCTGGTGGAGAGCGGCGGCGGCCTGGTGCAGGCCGGCGGCAGCCTGAGGCTGAGCTGCGTGGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCGGTGGTGGTGGTTCTGGTGGTGGTGGTTCTGGTGGTGGTGGTTCTGGTGGTGGTGGTTCTGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAAGTTTAAACGCGGCCGC

SEQ ID NO: 37 - protein, 632 aa, synthetic construct
  REGION 1..632: note = Description of sequence: pX-BC4-CD19b-tandem amino acid sequence
MALPVTALLLPLALLLHAARPQVQLVESGGGLVQAGGSLRLSCVASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSGGGGSGGGGSGGGGSGGGGSEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 38 - protein, 1938 aa, synthetic construct
  REGION 1..1938: note = Description of sequence: pX-BC4-CD19b-tandem nucleotide sequence
GGATCCACGCGTGCGATCGCACCATGGCCTTACCAGTGACCGCCTTGCTCCTGCCGCTGGCCTTGCTGCTCCACGCCGCCAGGCCGCAGGTGCAGCTGGTGGAGAGCGGCGGCGGCCTGGTGCAGGCCGGCGGCAGCCTGAGGCTGAGCTGCGTGGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCGGTGGTGGTGGTTCTGGTGGTGGTGGTTCTGGTGGTGGTGGTTCTGGTGGTGGTGGTTCTGAGGTCCAGCTGCAGCAGTCTGGACCTGAGCTGATAAAGCCTGGGGCTTCAGTGAAGATGTCCTGCAAGGCTTCTGGATACACATTCACTAGCTATGTTATGCACTGGGTGAAGCAGAAGCCTGGGCAGGGCCTTGAGTGGATTGGATATATTAATCCTTACAATGATGGTACTAAGTACAATGAGAAGTTCAAAGGCAAGGCCACACTGACTTCAGACAAATCCTCCAGCACAGCCTACATGGAGCTCAGCAGCCTGACCTCTGAGGACTCTGCGGTCTATTACTGTGCAAGAGGGACTTATTACTACGGTAGTAGGGTATTTGACTACTGGGGCCAAGGCACCACTCTCACAGTCTCCTCAGGTGGAGGGGGCTCAGGCGGAGGTGGCTCTGGGGGTGGAGGCTCGGACATTGTGATGACTCAGGCTGCACCCTCTATACCTGTCACTCCTGGAGAGTCAGTATCCATCTCCTGCAGGTCTAGTAAGAGTCTCCTGAATAGTAATGGCAACACTTACTTGTATTGGTTCCTGCAGAGGCCAGGCCAGTCTCCTCAGCTCCTGATATATCGGATGTCCAACCTTGCCTCAGGAGTCCCAGACAGGTTCAGTGGCAGTGGGTCAGGAACTGCTTTCACACTGAGAATCAGTAGAGTGGAGGCTGAGGATGTGGGTGTTTATTACTGTATGCAACATCTAGAATATCCGTTCACGTTCGGTGCTGGGACCAAGCTGGAGCTGAAACGGACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAAGTTTAAACGCGGCCGC

SEQ ID NO: 39 - protein, 625 aa, synthetic construct
  REGION 1..625: note = Description of sequence: pX-BC4-CD19c-tandem amino acid sequence
MALPVTALLLPLALLLHAARPQVQLVESGGGLVQAGGSLRLSCVASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 40 - protein, 1917 aa, synthetic construct
  REGION 1..1917: note = Description of sequence: pX-BC4-CD19c-tandem nucleotide sequence
GGATCCACGCGTGCGATCGCACCATGGCCTTACCAGTGACCGCCTTGCTCCTGCCGCTGGCCTTGCTGCTCCACGCCGCCAGGCCGCAGGTGCAGCTGGTGGAGAGCGGCGGCGGCCTGGTGCAGGCCGGCGGCAGCCTGAGGCTGAGCTGCGTGGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCGGTGGTGGTGGTTCTGGTGGTGGTGGTTCTGGTGGTGGTGGTTCTGGTGGTGGTGGTTCTGACATCCAGATGACACAGACTACATCCTCCCTGTCTGCCTCTCTGGGAGACAGAGTCACCATCAGTTGCAGGGCAAGTCAGGACATTAGTAAATATTTAAATTGGTATCAGCAGAAACCAGATGGAACTGTTAAACTCCTGATCTACCATACATCAAGATTACACTCAGGAGTCCCATCAAGGTTCAGTGGCAGTGGGTCTGGAACAGATTATTCTCTCACCATTAGCAACCTGGAGCAAGAAGATATTGCCACTTACTTTTGCCAACAGGGTAATACGCTTCCGTACACGTTCGGAGGGGGGACCAAGCTGGAGATCACAGGTGGCGGTGGCTCGGGCGGTGGTGGGTCGGGTGGCGGCGGATCTGAGGTGAAACTGCAGGAGTCAGGACCTGGCCTGGTGGCGCCCTCACAGAGCCTGTCCGTCACATGCACTGTCTCAGGGGTCTCATTACCCGACTATGGTGTAAGCTGGATTCGCCAGCCTCCACGAAAGGGTCTGGAGTGGCTGGGAGTAATATGGGGTAGTGAAACCACATACTATAATTCAGCTCTCAAATCCAGACTGACCATCATCAAGGACAACTCCAAGAGCCAAGTTTTCTTAAAAATGAACAGTCTGCAAACTGATGACACAGCCATTTACTACTGTGCCAAACATTATTACTACGGTGGTAGCTATGCTATGGACTACTGGGGCCAAGGAACCTCAGTCACCGTCTCCTCAACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAAGTTTAAACGCGGCCGC

SEQ ID NO: 41 - protein, 639 aa, synthetic construct
  REGION 1..639: note = Description of sequence: pX-CD19b-BC4 tandem amino acid sequence
IHACDRTMALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRGGGGSGGGGSGGGGSGGGGSQVQLVESGGGLVQAGGSLRLSCVASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 42 - protein, 1915 aa, synthetic construct
  REGION 1..1915: note = Description of sequence: pX-CD19b-BC4 tandem nucleotide sequence
ATGGCCTTACCAGTGACCGCCTTGCTCCTGCCGCTGGCCTTGCTGCTCCACGCCGCCAGGCCGGAGGTCCAGCTGCAGCAGTCTGGACCTGAGCTGATAAAGCCTGGGGCTTCAGTGAAGATGTCCTGCAAGGCTTCTGGATACACATTCACTAGCTATGTTATGCACTGGGTGAAGCAGAAGCCTGGGCAGGGCCTTGAGTGGATTGGATATATTAATCCTTACAATGATGGTACTAAGTACAATGAGAAGTTCAAAGGCAAGGCCACACTGACTTCAGACAAATCCTCCAGCACAGCCTACATGGAGCTCAGCAGCCTGACCTCTGAGGACTCTGCGGTCTATTACTGTGCAAGAGGGACTTATTACTACGGTAGTAGGGTATTTGACTACTGGGGCCAAGGCACCACTCTCACAGTCTCCTCAGGTGGAGGGGGCTCAGGCGGAGGTGGCTCTGGGGGTGGAGGCTCGGACATTGTGATGACTCAGGCTGCACCCTCTATACCTGTCACTCCTGGAGAGTCAGTATCCATCTCCTGCAGGTCTAGTAAGAGTCTCCTGAATAGTAATGGCAACACTTACTTGTATTGGTTCCTGCAGAGGCCAGGCCAGTCTCCTCAGCTCCTGATATATCGGATGTCCAACCTTGCCTCAGGAGTCCCAGACAGGTTCAGTGGCAGTGGGTCAGGAACTGCTTTCACACTGAGAATCAGTAGAGTGGAGGCTGAGGATGTGGGTGTTTATTACTGTATGCAACATCTAGAATATCCGTTCACGTTCGGTGCTGGGACCAAGCTGGAGCTGAAACGGGGTGGTGGTGGTTCTGGTGGTGGTGGTTCTGGTGGTGGTGGTTCTGGTGGTGGTGGTTCTCAGGTGCAGCTGGTGGAGAGCGGCGGCGGCCTGGTGCAGGCCGGCGGCAGCCTGAGGCTGAGCTGCGTGGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAAGTTTAAACGCGGCCGC

SEQ ID NO: 43 - protein, 823 aa, synthetic construct
  REGION 1..823: note = Description of sequence: pX-CD19b-CD7Q tandem amino acid sequence
MALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRGGGGSGGGGSGGGGSGGGGSGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 44 - protein, 2511 aa, synthetic construct
  REGION 1..2511: note = Description of sequence: pX-CD19b-CD7Q tandem nucleotide sequence
GGATCCACGCGTGCGATCGCACCATGGCCTTACCAGTGACCGCCTTGCTCCTGCCGCTGGCCTTGCTGCTCCACGCCGCCAGGCCGGAGGTCCAGCTGCAGCAGTCTGGACCTGAGCTGATAAAGCCTGGGGCTTCAGTGAAGATGTCCTGCAAGGCTTCTGGATACACATTCACTAGCTATGTTATGCACTGGGTGAAGCAGAAGCCTGGGCAGGGCCTTGAGTGGATTGGATATATTAATCCTTACAATGATGGTACTAAGTACAATGAGAAGTTCAAAGGCAAGGCCACACTGACTTCAGACAAATCCTCCAGCACAGCCTACATGGAGCTCAGCAGCCTGACCTCTGAGGACTCTGCGGTCTATTACTGTGCAAGAGGGACTTATTACTACGGTAGTAGGGTATTTGACTACTGGGGCCAAGGCACCACTCTCACAGTCTCCTCAGGTGGAGGGGGCTCAGGCGGAGGTGGCTCTGGGGGTGGAGGCTCGGACATTGTGATGACTCAGGCTGCACCCTCTATACCTGTCACTCCTGGAGAGTCAGTATCCATCTCCTGCAGGTCTAGTAAGAGTCTCCTGAATAGTAATGGCAACACTTACTTGTATTGGTTCCTGCAGAGGCCAGGCCAGTCTCCTCAGCTCCTGATATATCGGATGTCCAACCTTGCCTCAGGAGTCCCAGACAGGTTCAGTGGCAGTGGGTCAGGAACTGCTTTCACACTGAGAATCAGTAGAGTGGAGGCTGAGGATGTGGGTGTTTATTACTGTATGCAACATCTAGAATATCCGTTCACGTTCGGTGCTGGGACCAAGCTGGAGCTGAAACGGGGTGGTGGTGGTTCTGGTGGTGGTGGTTCTGGTGGTGGTGGTTCTGGTGGTGGTGGTTCTGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAAGTTTAAACGCGGCCGC

SEQ ID NO: 45 - protein, 816 aa, synthetic construct
  REGION 1..816: note = Description of sequence: pX-CD19c-CD7Q-tandem amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSGGGGSGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 46 - protein, 2467 aa, synthetic construct
  REGION 1..2467: note = Description of sequence: pX-CD19c-CD7Q-tandem nucleotide sequence
ATGGCCTTACCAGTGACCGCCTTGCTCCTGCCGCTGGCCTTGCTGCTCCACGCCGCCAGGCCGGACATCCAGATGACACAGACTACATCCTCCCTGTCTGCCTCTCTGGGAGACAGAGTCACCATCAGTTGCAGGGCAAGTCAGGACATTAGTAAATATTTAAATTGGTATCAGCAGAAACCAGATGGAACTGTTAAACTCCTGATCTACCATACATCAAGATTACACTCAGGAGTCCCATCAAGGTTCAGTGGCAGTGGGTCTGGAACAGATTATTCTCTCACCATTAGCAACCTGGAGCAAGAAGATATTGCCACTTACTTTTGCCAACAGGGTAATACGCTTCCGTACACGTTCGGAGGGGGGACCAAGCTGGAGATCACAGGTGGCGGTGGCTCGGGCGGTGGTGGGTCGGGTGGCGGCGGATCTGAGGTGAAACTGCAGGAGTCAGGACCTGGCCTGGTGGCGCCCTCACAGAGCCTGTCCGTCACATGCACTGTCTCAGGGGTCTCATTACCCGACTATGGTGTAAGCTGGATTCGCCAGCCTCCACGAAAGGGTCTGGAGTGGCTGGGAGTAATATGGGGTAGTGAAACCACATACTATAATTCAGCTCTCAAATCCAGACTGACCATCATCAAGGACAACTCCAAGAGCCAAGTTTTCTTAAAAATGAACAGTCTGCAAACTGATGACACAGCCATTTACTACTGTGCCAAACATTATTACTACGGTGGTAGCTATGCTATGGACTACTGGGGCCAAGGAACCTCAGTCACCGTCTCCTCAGGTGGTGGTGGTTCTGGTGGTGGTGGTTCTGGTGGTGGTGGTTCTGGTGGTGGTGGTTCTGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAAGTTTAAACGCGGCCGC

SEQ ID NO: 47 - protein, 1298 aa, synthetic construct; identical to SEQ ID NO: 25
  REGION 1..1298: note = Description of sequence: pX-BCMA-CD19b-28-Vac amino acid sequence

SEQ ID NO: 48 - protein, 3905 aa, synthetic construct; identical to SEQ ID NO: 26
  REGION 1..3905: note = Description of sequence: pX-BCMA-CD19b-28-Vac nucleotide sequence

SEQ ID NO: 49 - protein, 714 aa, synthetic construct
  REGION 1..714: note = Description of sequence: pX-BC4Q -Vac-BB amino acid sequence
MALPVTALLLPLALLLHAARPQVQLVESGGGLVQAGGSLRLSCVASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 50 - protein, 2184 aa, synthetic construct
  REGION 1..2184: note = Description of sequence: pX-BC4Q -Vac-BB nucleotide sequence
GGATCCACGCGTGCGATCGCACCATGGCCTTACCAGTGACCGCCTTGCTCCTGCCGCTGGCCTTGCTGCTCCACGCCGCCAGGCCGCAGGTGCAGCTGGTGGAGAGCGGCGGCGGCCTGGTGCAGGCCGGCGGCAGCCTGAGGCTGAGCTGCGTGGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAACGCGGCCGC

SEQ ID NO: 51 - protein, 418 aa, synthetic construct
  REGION 1..418: note = Description of sequence: pX-BC4Q -Vac-28-amino acid sequence
MALPVTALLLPLALLLHAARPQVQLVESGGGLVQAGGSLRLSCVASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 52 - protein, 1296 aa, synthetic construct
  REGION 1..1296: note = Description of sequence: pX-BC4Q -Vac -28-nucleotide sequence
GGATCCACGCGTGCGATCGCACCATGGCCTTACCAGTGACCGCCTTGCTCCTGCCGCTGGCCTTGCTGCTCCACGCCGCCAGGCCGCAGGTGCAGCTGGTGGAGAGCGGCGGCGGCCTGGTGCAGGCCGGCGGCAGCCTGAGGCTGAGCTGCGTGGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAAGTTTAAACGCGGCCGC

SEQ ID NO: 53 - protein, 715 aa, synthetic construct
  REGION 1..715: note = Description of sequence: pX-BC5q-Vac-BB- amino acid sequence
MALPVTALLLPLALLLHAARPQLQLVESGGGLVQSGGSLRLSCSIASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 54 - protein, 2187 aa, synthetic construct
  REGION 1..2187: note = Description of sequence: pX-BC5q-Vac-BB- nucleotide sequence
GGATCCACGCGTGCGATCGCACCATGGCCTTACCAGTGACCGCCTTGCTCCTGCCGCTGGCCTTGCTGCTCCACGCCGCCAGGCCGCAGCTGCAGCTGGTGGAGAGCGGCGGCGGCCTGGTGCAGAGCGGCGGCAGCCTGAGGCTGAGCTGCAGCATCGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAACGCGGCCGC

SEQ ID NO: 55 - protein, 714 aa, synthetic construct
  REGION 1..714: note = Description of sequence: pX-BC5q-Vac-28- amino acid sequence
MALPVTALLLPLALLLHAARPQLQLVESGGGLVQSGGSLRLSCSIASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 56 - protein, 2184 aa, synthetic construct
  REGION 1..2184: note = Description of sequence: pX-BC5q-Vac-28 nucleotide sequence
GGATCCACGCGTGCGATCGCACCATGGCCTTACCAGTGACCGCCTTGCTCCTGCCGCTGGCCTTGCTGCTCCACGCCGCCAGGCCGCAGCTGCAGCTGGTGGAGAGCGGCGGCGGCCTGGTGCAGAGCGGCGGCAGCCTGAGGCTGAGCTGCAGCATCGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAACGCGGCCGC

SEQ ID NO: 57 - protein, 419 aa, synthetic construct
  REGION 1..419: note = Description of sequence: pX-BC4Q -BB amino acid sequence
MALPVTALLLPLALLLHAARPQVQLVESGGGLVQAGGSLRLSCVASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 58 - protein, 1299 aa, synthetic construct
  REGION 1..1299: note = Description of sequence: pX-BC4Q -BB nucleotide sequence
GGATCCACGCGTGCGATCGCACCATGGCCTTACCAGTGACCGCCTTGCTCCTGCCGCTGGCCTTGCTGCTCCACGCCGCCAGGCCGCAGGTGCAGCTGGTGGAGAGCGGCGGCGGCCTGGTGCAGGCCGGCGGCAGCCTGAGGCTGAGCTGCGTGGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAAGTTTAAACGCGGCCGC

SEQ ID NO: 59 - protein, 418 aa, synthetic construct; identical to SEQ ID NO: 51
  REGION 1..418: note = Description of sequence: pX-BC4Q -28-amino acid sequence

SEQ ID NO: 60 - protein, 1296 aa, synthetic construct; identical to SEQ ID NO: 52
  REGION 1..1296: note = Description of sequence: pX-BC4Q -28-nucleotide sequence

SEQ ID NO: 61 - protein, 420 aa, synthetic construct
  REGION 1..420: note = Description of sequence: pX-BC5q- BB- amino acid sequence
MALPVTALLLPLALLLHAARPQLQLVESGGGLVQSGGSLRLSCSIASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 62 - protein, 1302 aa, synthetic construct
  REGION 1..1302: note = Description of sequence: pX-BC5q--BB- nucleotide sequence
GGATCCACGCGTGCGATCGCACCATGGCCTTACCAGTGACCGCCTTGCTCCTGCCGCTGGCCTTGCTGCTCCACGCCGCCAGGCCGCAGCTGCAGCTGGTGGAGAGCGGCGGCGGCCTGGTGCAGAGCGGCGGCAGCCTGAGGCTGAGCTGCAGCATCGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAAGTTTAAACGCGGCCGC

SEQ ID NO: 63 - protein, 419 aa, synthetic construct
  REGION 1..419: note = Description of sequence: pX-BC5q--28- amino acid sequence
MALPVTALLLPLALLLHAARPQLQLVESGGGLVQSGGSLRLSCSIASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 64 - protein, 1299 aa, synthetic construct
  REGION 1..1299: note = Description of sequence: pX-BC5q--28 nucleotide sequence
GGATCCACGCGTGCGATCGCACCATGGCCTTACCAGTGACCGCCTTGCTCCTGCCGCTGGCCTTGCTGCTCCACGCCGCCAGGCCGCAGCTGCAGCTGGTGGAGAGCGGCGGCGGCCTGGTGCAGAGCGGCGGCAGCCTGAGGCTGAGCTGCAGCATCGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAAGTTTAAACGCGGCCGC

SEQ ID NO: 65 - protein, 988 aa, synthetic construct
  REGION 1..988: note = Description of sequence: pX-BC4-CD7Q-VAC-tandem amino acid sequence
MALPVTALLLPLALLLHAARPQVQLVESGGGLVQAGGSLRLSCVASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSGGGGSGGGGSGGGGSGGGGSGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 66 - protein, 3006 aa, synthetic construct
  REGION 1..3006: note = Description of sequence: pX-BC4-CD7Q-VAC-tandem nucleotide sequence
GGATCCACGCGTGCGATCGCACCATGGCCTTACCAGTGACCGCCTTGCTCCTGCCGCTGGCCTTGCTGCTCCACGCCGCCAGGCCGCAGGTGCAGCTGGTGGAGAGCGGCGGCGGCCTGGTGCAGGCCGGCGGCAGCCTGAGGCTGAGCTGCGTGGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCGGTGGTGGTGGTTCTGGTGGTGGTGGTTCTGGTGGTGGTGGTTCTGGTGGTGGTGGTTCTGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAACGCGGCCGC

SEQ ID NO: 67 - protein, 927 aa, synthetic construct
  REGION 1..927: note = Description of sequence: pX-BC4-CD19b-VAC-tandem amino acid sequence
MALPVTALLLPLALLLHAARPQVQLVESGGGLVQAGGSLRLSCVASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSGGGGSGGGGSGGGGSGGGGSEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 68 - protein, 2823 aa, synthetic construct
  REGION 1..2823: note = Description of sequence: pX-BC4-CD19b-VAC-tandem nucleotide sequence
GGATCCACGCGTGCGATCGCACCATGGCCTTACCAGTGACCGCCTTGCTCCTGCCGCTGGCCTTGCTGCTCCACGCCGCCAGGCCGCAGGTGCAGCTGGTGGAGAGCGGCGGCGGCCTGGTGCAGGCCGGCGGCAGCCTGAGGCTGAGCTGCGTGGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCGGTGGTGGTGGTTCTGGTGGTGGTGGTTCTGGTGGTGGTGGTTCTGGTGGTGGTGGTTCTGAGGTCCAGCTGCAGCAGTCTGGACCTGAGCTGATAAAGCCTGGGGCTTCAGTGAAGATGTCCTGCAAGGCTTCTGGATACACATTCACTAGCTATGTTATGCACTGGGTGAAGCAGAAGCCTGGGCAGGGCCTTGAGTGGATTGGATATATTAATCCTTACAATGATGGTACTAAGTACAATGAGAAGTTCAAAGGCAAGGCCACACTGACTTCAGACAAATCCTCCAGCACAGCCTACATGGAGCTCAGCAGCCTGACCTCTGAGGACTCTGCGGTCTATTACTGTGCAAGAGGGACTTATTACTACGGTAGTAGGGTATTTGACTACTGGGGCCAAGGCACCACTCTCACAGTCTCCTCAGGTGGAGGGGGCTCAGGCGGAGGTGGCTCTGGGGGTGGAGGCTCGGACATTGTGATGACTCAGGCTGCACCCTCTATACCTGTCACTCCTGGAGAGTCAGTATCCATCTCCTGCAGGTCTAGTAAGAGTCTCCTGAATAGTAATGGCAACACTTACTTGTATTGGTTCCTGCAGAGGCCAGGCCAGTCTCCTCAGCTCCTGATATATCGGATGTCCAACCTTGCCTCAGGAGTCCCAGACAGGTTCAGTGGCAGTGGGTCAGGAACTGCTTTCACACTGAGAATCAGTAGAGTGGAGGCTGAGGATGTGGGTGTTTATTACTGTATGCAACATCTAGAATATCCGTTCACGTTCGGTGCTGGGACCAAGCTGGAGCTGAAACGGACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAACGCGGCCGC

SEQ ID NO: 69 - protein, 920 aa, synthetic construct
  REGION 1..920: note = Description of sequence: pX-BC4-CD19c-VAC-tandem amino acid sequence
MALPVTALLLPLALLLHAARPQVQLVESGGGLVQAGGSLRLSCVASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 70 - protein, 2802 aa, synthetic construct
  REGION 1..2802: note = Description of sequence: pX-BC4-CD19c-VAC-tandem nucleotide sequence
GGATCCACGCGTGCGATCGCACCATGGCCTTACCAGTGACCGCCTTGCTCCTGCCGCTGGCCTTGCTGCTCCACGCCGCCAGGCCGCAGGTGCAGCTGGTGGAGAGCGGCGGCGGCCTGGTGCAGGCCGGCGGCAGCCTGAGGCTGAGCTGCGTGGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCGGTGGTGGTGGTTCTGGTGGTGGTGGTTCTGGTGGTGGTGGTTCTGGTGGTGGTGGTTCTGACATCCAGATGACACAGACTACATCCTCCCTGTCTGCCTCTCTGGGAGACAGAGTCACCATCAGTTGCAGGGCAAGTCAGGACATTAGTAAATATTTAAATTGGTATCAGCAGAAACCAGATGGAACTGTTAAACTCCTGATCTACCATACATCAAGATTACACTCAGGAGTCCCATCAAGGTTCAGTGGCAGTGGGTCTGGAACAGATTATTCTCTCACCATTAGCAACCTGGAGCAAGAAGATATTGCCACTTACTTTTGCCAACAGGGTAATACGCTTCCGTACACGTTCGGAGGGGGGACCAAGCTGGAGATCACAGGTGGCGGTGGCTCGGGCGGTGGTGGGTCGGGTGGCGGCGGATCTGAGGTGAAACTGCAGGAGTCAGGACCTGGCCTGGTGGCGCCCTCACAGAGCCTGTCCGTCACATGCACTGTCTCAGGGGTCTCATTACCCGACTATGGTGTAAGCTGGATTCGCCAGCCTCCACGAAAGGGTCTGGAGTGGCTGGGAGTAATATGGGGTAGTGAAACCACATACTATAATTCAGCTCTCAAATCCAGACTGACCATCATCAAGGACAACTCCAAGAGCCAAGTTTTCTTAAAAATGAACAGTCTGCAAACTGATGACACAGCCATTTACTACTGTGCCAAACATTATTACTACGGTGGTAGCTATGCTATGGACTACTGGGGCCAAGGAACCTCAGTCACCGTCTCCTCAACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAACGCGGCCGC

SEQ ID NO: 71 - protein, 927 aa, synthetic construct
  REGION 1..927: note = Description of sequence: pX-CD19b-BC4-VAC-tandem amino acid sequence
MALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRGGGGSGGGGSGGGGSGGGGSQVQLVESGGGLVQAGGSLRLSCVASKSTFTYNAMAWYRQAPGKQRELVAFIASGGNTDYADSVTGRFTISRDNAKNTMYLQMNSLKPEDTAVYYCHIERIWGGSTLRYWGQGTQVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 72 - protein, 2823 aa, synthetic construct
  REGION 1..2823: note = Description of sequence: pX-CD19b-BC4-VAC-tandem nucleotide sequence
GGATCCACGCGTGCGATCGCACCATGGCCTTACCAGTGACCGCCTTGCTCCTGCCGCTGGCCTTGCTGCTCCACGCCGCCAGGCCGGAGGTCCAGCTGCAGCAGTCTGGACCTGAGCTGATAAAGCCTGGGGCTTCAGTGAAGATGTCCTGCAAGGCTTCTGGATACACATTCACTAGCTATGTTATGCACTGGGTGAAGCAGAAGCCTGGGCAGGGCCTTGAGTGGATTGGATATATTAATCCTTACAATGATGGTACTAAGTACAATGAGAAGTTCAAAGGCAAGGCCACACTGACTTCAGACAAATCCTCCAGCACAGCCTACATGGAGCTCAGCAGCCTGACCTCTGAGGACTCTGCGGTCTATTACTGTGCAAGAGGGACTTATTACTACGGTAGTAGGGTATTTGACTACTGGGGCCAAGGCACCACTCTCACAGTCTCCTCAGGTGGAGGGGGCTCAGGCGGAGGTGGCTCTGGGGGTGGAGGCTCGGACATTGTGATGACTCAGGCTGCACCCTCTATACCTGTCACTCCTGGAGAGTCAGTATCCATCTCCTGCAGGTCTAGTAAGAGTCTCCTGAATAGTAATGGCAACACTTACTTGTATTGGTTCCTGCAGAGGCCAGGCCAGTCTCCTCAGCTCCTGATATATCGGATGTCCAACCTTGCCTCAGGAGTCCCAGACAGGTTCAGTGGCAGTGGGTCAGGAACTGCTTTCACACTGAGAATCAGTAGAGTGGAGGCTGAGGATGTGGGTGTTTATTACTGTATGCAACATCTAGAATATCCGTTCACGTTCGGTGCTGGGACCAAGCTGGAGCTGAAACGGGGTGGTGGTGGTTCTGGTGGTGGTGGTTCTGGTGGTGGTGGTTCTGGTGGTGGTGGTTCTCAGGTGCAGCTGGTGGAGAGCGGCGGCGGCCTGGTGCAGGCCGGCGGCAGCCTGAGGCTGAGCTGCGTGGCCAGCAAGAGCACCTTCACCTACAACGCCATGGCCTGGTACAGGCAGGCCCCCGGCAAGCAGAGGGAGCTGGTGGCCTTCATCGCCAGCGGCGGCAACACCGACTACGCCGACAGCGTGACCGGCAGGTTCACCATCAGCAGGGACAACGCCAAGAACACCATGTACCTGCAGATGAACAGCCTGAAGCCCGAGGACACCGCCGTGTACTACTGCCACATCGAGAGGATCTGGGGCGGCAGCACCCTGAGGTACTGGGGCCAGGGCACCCAGGTGACCGTGAGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAACGCGGCCGC

SEQ ID NO: 73 - protein, 1118 aa, synthetic construct
  REGION 1..1118: note = Description of sequence: pX-CD19b-CD7Q -VAC-tandem amino acid sequence
MALPVTALLLPLALLLHAARPEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRGGGGSGGGGSGGGGSGGGGSGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 74 - protein, 3396 aa, synthetic construct
  REGION 1..3396: note = Description of sequence: pX-CD19b-CD7Q-VAC- tandem nucleotide sequence
GGATCCACGCGTGCGATCGCACCATGGCCTTACCAGTGACCGCCTTGCTCCTGCCGCTGGCCTTGCTGCTCCACGCCGCCAGGCCGGAGGTCCAGCTGCAGCAGTCTGGACCTGAGCTGATAAAGCCTGGGGCTTCAGTGAAGATGTCCTGCAAGGCTTCTGGATACACATTCACTAGCTATGTTATGCACTGGGTGAAGCAGAAGCCTGGGCAGGGCCTTGAGTGGATTGGATATATTAATCCTTACAATGATGGTACTAAGTACAATGAGAAGTTCAAAGGCAAGGCCACACTGACTTCAGACAAATCCTCCAGCACAGCCTACATGGAGCTCAGCAGCCTGACCTCTGAGGACTCTGCGGTCTATTACTGTGCAAGAGGGACTTATTACTACGGTAGTAGGGTATTTGACTACTGGGGCCAAGGCACCACTCTCACAGTCTCCTCAGGTGGAGGGGGCTCAGGCGGAGGTGGCTCTGGGGGTGGAGGCTCGGACATTGTGATGACTCAGGCTGCACCCTCTATACCTGTCACTCCTGGAGAGTCAGTATCCATCTCCTGCAGGTCTAGTAAGAGTCTCCTGAATAGTAATGGCAACACTTACTTGTATTGGTTCCTGCAGAGGCCAGGCCAGTCTCCTCAGCTCCTGATATATCGGATGTCCAACCTTGCCTCAGGAGTCCCAGACAGGTTCAGTGGCAGTGGGTCAGGAACTGCTTTCACACTGAGAATCAGTAGAGTGGAGGCTGAGGATGTGGGTGTTTATTACTGTATGCAACATCTAGAATATCCGTTCACGTTCGGTGCTGGGACCAAGCTGGAGCTGAAACGGGGTGGTGGTGGTTCTGGTGGTGGTGGTTCTGGTGGTGGTGGTTCTGGTGGTGGTGGTTCTGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAACGCGGCCGC

SEQ ID NO: 75 - protein, 1111 aa, synthetic construct
  REGION 1..1111: note = Description of sequence: pX-CD19c-CD7Q-VAC-tandem amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSGGGGSGAQPAMAAYKDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSACPYSNPSLCSGGGGSELPTQGTFSNVSTNVSPAKPTTTACPYSNPSLCSGGGGSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMYRMQLLSCIALSLALVTNSGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQAPRRARGCRTLGLPALLLLLLLRPPATRGITCPPPMSVEHADIWVKSYSLYSRERYICNSGFKRKAGTSSLTECVLNKATNVAHWTTPSLKCIR

SEQ ID NO: 76 - protein, 3375 aa, synthetic construct
  REGION 1..3375: note = Description of sequence: pX-CD19c-CD7Q-VAC-tandem nucleotide sequence
GGATCCACGCGTGCGATCGCACCATGGCCTTACCAGTGACCGCCTTGCTCCTGCCGCTGGCCTTGCTGCTCCACGCCGCCAGGCCGGACATCCAGATGACACAGACTACATCCTCCCTGTCTGCCTCTCTGGGAGACAGAGTCACCATCAGTTGCAGGGCAAGTCAGGACATTAGTAAATATTTAAATTGGTATCAGCAGAAACCAGATGGAACTGTTAAACTCCTGATCTACCATACATCAAGATTACACTCAGGAGTCCCATCAAGGTTCAGTGGCAGTGGGTCTGGAACAGATTATTCTCTCACCATTAGCAACCTGGAGCAAGAAGATATTGCCACTTACTTTTGCCAACAGGGTAATACGCTTCCGTACACGTTCGGAGGGGGGACCAAGCTGGAGATCACAGGTGGCGGTGGCTCGGGCGGTGGTGGGTCGGGTGGCGGCGGATCTGAGGTGAAACTGCAGGAGTCAGGACCTGGCCTGGTGGCGCCCTCACAGAGCCTGTCCGTCACATGCACTGTCTCAGGGGTCTCATTACCCGACTATGGTGTAAGCTGGATTCGCCAGCCTCCACGAAAGGGTCTGGAGTGGCTGGGAGTAATATGGGGTAGTGAAACCACATACTATAATTCAGCTCTCAAATCCAGACTGACCATCATCAAGGACAACTCCAAGAGCCAAGTTTTCTTAAAAATGAACAGTCTGCAAACTGATGACACAGCCATTTACTACTGTGCCAAACATTATTACTACGGTGGTAGCTATGCTATGGACTACTGGGGCCAAGGAACCTCAGTCACCGTCTCCTCAGGTGGTGGTGGTTCTGGTGGTGGTGGTTCTGGTGGTGGTGGTTCTGGTGGTGGTGGTTCTGGCGCCCAGCCCGCCATGGCCGCCTACAAGGACATCCAGATGACCCAGACCACCAGCAGCCTGAGCGCCAGCCTGGGCGACAGAGTGACCATCAGCTGCAGCGCCAGCCAGGGCATCAGCAACTACCTGAACTGGTACCAGCAGAAGCCCGACGGCACCGTGAAGCTGCTGATCTACTACACCAGCAGCCTGCACAGCGGCGTGCCCAGCAGATTCAGCGGCAGCGGCAGCGGCACCGACTACAGCCTGACCATCAGCAACCTGGAGCCCGAGGACATCGCCACCTACTACTGCCAGCAGTACAGCAAGCTGCCCTACACCTTCGGCGGCGGCACCAAGCTGGAGATCAAGAGAGGCGGCGGCGGCAGCGGCGGCGGCGGCAGCGGCGGCGGCGGCAGCGGCGGCGGCGGCAGCGAGGTGCAGCTGGTGGAGAGCGGCGGCGGCCTGGTGAAGCCCGGCGGCAGCCTGAAGCTGAGCTGCGCCGCCAGCGGCCTGACCTTCAGCAGCTACGCCATGAGCTGGGTGAGACAGACCCCCGAGAAGAGACTGGAGTGGGTGGCCAGCATCAGCAGCGGCGGCTTCACCTACTACCCCGACAGCGTGAAGGGCAGATTCACCATCAGCAGAGACAACGCCAGAAACATCCTGTACCTGCAGATGAGCAGCCTGAGAAGCGAGGACACCGCCATGTACTACTGCGCCAGAGACGAGGTGAGAGGCTACCTGGACGTGTGGGGCGCCGGCACCACCGTGACCGTGAGCAGCGCCTGCCCCTACAGCAACCCCAGCCTGTGCAGCGGCGGCGGCGGCAGCGAGCTGCCCACCCAGGGCACCTTCAGCAACGTGAGCACCAACGTGAGCCCCGCCAAGCCCACCACCACCGCCTGTCCTTATTCCAATCCTTCCCTGTGTAGCGGCGGCGGCGGCAGCACCACGACGCCAGCGCCGCGACCACCAACACCGGCGCCCACCATCGCGTCGCAGCCCCTGTCCCTGCGCCCAGAGGCGTGCCGGCCAGCGGCGGGGGGCGCAGTGCACACGAGGGGGCTGGACTTCGCCTGTGATATCTACATCTGGGCGCCCTTGGCCGGGACTTGTGGGGTCCTTCTCCTGTCACTGGTTATCACCCTTTACTGCAGGAGTAAGAGGAGCAGGCTCCTGCACAGTGACTACATGAACATGACTCCCCGCCGCCCCGGGCCCACCCGCAAGCATTACCAGCCCTATGCCCCACCACGCGACTTCGCAGCCTATCGCTCCAGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGCAGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCGGAAGCGGAGCCACCAACTTCAGCCTGCTGAAGCAGGCCGGCGACGTGGAGGAGAACCCCGGCCCCATGTACAGAATGCAGCTGCTGAGCTGCATCGCCCTGAGCCTGGCCCTGGTGACCAACAGCGGCATCCACGTGTTCATCCTGGGCTGCTTCAGCGCCGGCCTGCCCAAGACCGAGGCCAACTGGGTGAACGTGATCAGCGACCTGAAGAAGATCGAGGACCTGATCCAGAGCATGCACATCGACGCCACCCTGTACACCGAGAGCGACGTGCACCCCAGCTGCAAGGTGACCGCCATGAAGTGCTTCCTGCTGGAGCTGCAGGTGATCAGCCTGGAGAGCGGCGACGCCAGCATCCACGACACCGTGGAGAACCTGATCATCCTGGCCAACAACAGCCTGAGCAGCAACGGCAACGTGACCGAGAGCGGCTGCAAGGAGTGCGAGGAGCTGGAGGAGAAGAACATCAAGGAGTTCCTGCAGAGCTTCGTGCACATCGTGCAGATGTTCATCAACACCAGCTCCGGCGGCGGCTCCGGCGGCGGCGGCTCCGGCGGCGGCGGCTCCGGCGGCGGCGGCTCCGGCGGCGGCTCCCTGCAGGCCCCCAGAAGAGCCAGAGGCTGCAGAACCCTGGGCCTGCCCGCCCTGCTGCTGCTGCTGCTGCTGAGACCCCCCGCCACCAGAGGCATCACCTGCCCCCCCCCCATGAGCGTGGAGCACGCCGACATCTGGGTGAAGAGCTACAGCCTGTACAGCAGAGAGAGATACATCTGCAACAGCGGCTTCAAGAGAAAGGCCGGCACCAGCAGCCTGACCGAGTGCGTGCTGAACAAGGCCACCAACGTGGCCCACTGGACCACCCCCAGCCTGAAGTGCATCAGATAAGTTTAAACGCGGCCGC

SEQ ID NO: 77 - protein, 119 aa, synthetic construct
  REGION 1..119: note = Description of sequence: BC4 VHH amino acid sequence
QVQLVESGGGLVQAGGSLRLSCVASKSTFTYNAMAWYRQAPGKQRELVAFIASGGNTDYADSVTGRFTISRDNAKNTMYLQMNSLKPEDTAVYYCHIERIWGGSTLRYWGQGTQVTVSS

SEQ ID NO: 78 - protein, 120 aa, synthetic construct
  REGION 1..120: note = Description of sequence: BC5 VHH amino acid sequence
QLQLVESGGGLVQSGGSLRLSCSIASKSTFTYNAMAWYRQAPGKQRELVAFIASGGNTDYADSVTGRFTISRDNAKNTMYLQMNSLKPEDTAVYYCHIERIWGGSTLRYWGQGTQVTVSS